(19) United States
(12) Patent Application Publication
(10) Pub. No.: US 2019/0352276 A1
(43) Pub. Date: Nov. 21, 2019

(54) INHIBITORS OF BRUTON'S TYROSINE KINASE

(71) Applicant: Joint Stock Company "Biocad", St. Petersburg (RU)

(72) Inventors: Aleksey Sergeevich Gavrilov, Sankt Petersburg (RU); Pavel Aleksandrovich Aleshunin, Sankt Petersburg (RU); Svetlana Leonidovna Gorbunova, Sankt Petersburg (RU); Mikhail Vladimirovich Rekharsky, Moscow (RU); Natalia Vladimirovna Kozhemyakina, Sankt Petersburg (RU); Anna Aleksandrovna Kukushkina, Leningradskaya obl. (RU); Anna Sergeevna Kushakova, Leningradskaya obl. (RU); Leonid Evgen`evich Mikhaylov, Sankt Petersburg (RU); Alexander Moldavsky, Sankt Petersburg (RU); Aleksandra Vladimirovna Popkova, Leningradskaya obl. (RU); Sergey Aleksandrovich Silonov, Samara (RU); Svetlana Sergeevna Smirnova, Kirovskaya obl. (RU); Pavel Andreevich Iakovlev, Saint Petersburg (RU)

(21) Appl. No.: 16/461,537

(22) PCT Filed: Nov. 16, 2017
(86) PCT No.: PCT/IB2017/057154

§ 371 Date: May 16, 2019

Related U.S. Application Data
(60) Provisional application No. 62/424,041, filed on Nov. 18, 2016.

(51) Int. Cl. C07D 401/04; C07D 401/14; C07D 403/04; C07D 403/12; C07D 471/04

(52) U.S. Cl. CPC C07D 401/04; C07D 401/14; C07D 471/04; C07D 403/12; C07D 403/04

(57) ABSTRACT

The present invention relates to a new compound of formula I: or pharmaceutically acceptable salt, solvate or stereoisomer thereof, wherein: V₁ is C or N, V₂ is C(R₂) or N, whereby if V₁ is C then V₂ is N, if V₁ is C then V₂ is C(R₂), or if V₁ is N then V₂ is C(R₂); each n, k is independently 0, 1; each R₂, R₁₁ is independently H, D, Hal, CN, NR′R″, C(O)NR′R″, C₁-C₆ alkoxy; R₃ is H, D, hydroxy, C(O)C₁-C₆ alkyl, C(O)C₂-C₆ alkenyl, C(O)C₂-C₆ alkynyl, C₁-C₆ alkyl; R₄ is H, Hal, CN, CONR′R″, hydroxy, C₁-C₆ alkyl, C₁-C₆ alkoxy; L is CH₂, NH, O or chemical bond; R₁ is selected from the group of the fragments, comprising: Fragment 1, Fragment 2, Fragment 3 each A₁, A₂, A₃, A₄ is independently CH, N, CHal; each A₅, A₆, A₇, A₈, A₉ is independently C, CH or N; R₅ is H, CN, Hal, CONR′R″, C₁-C₆ alkyl, non-substituted or substituted by one or more halogens; each R′ and R″ is independently selected from the group, comprising H, C₁-C₆ alkyl, C₁-C₆ cycloalkyl, aryl; R₆ is selected from the group: [formula II] each R₇, R₈, R₉, R₁₀ is independently vinyl, methylacetylenyl; Hal is CI, Br, I, F, which have properties of inhibitor of Bruton's tyrosine kinase (Btk), to pharmaceutical compositions containing such compounds, and their use as pharmaceuticals for treatment of diseases and disorder.

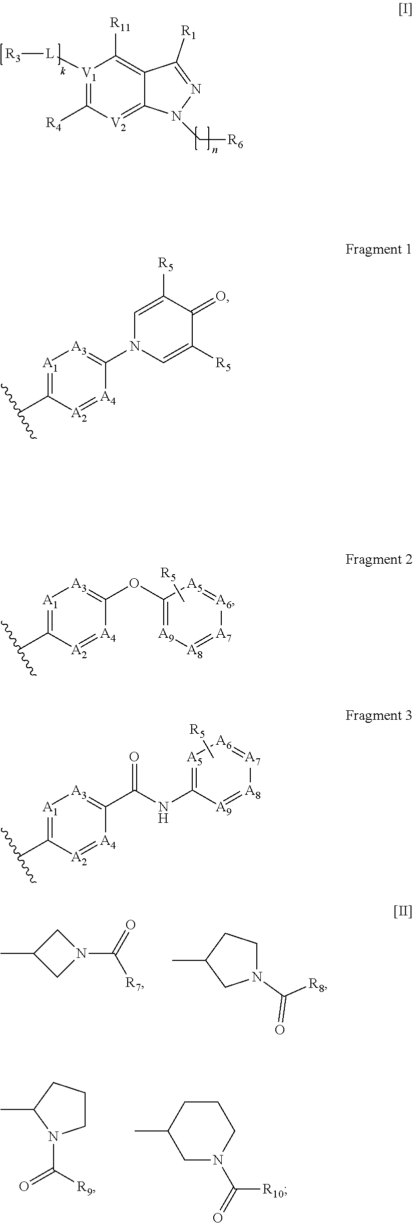

### FIELD OF THE INVENTION

The present invention relates to new inhibitors of Bruton's tyrosine kinase, to their preparations, to pharmaceutical compositions containing such compounds, and to the use of such compounds or such compositions as pharmaceuticals for treatment of diseases and disorders.

### BACKGROUND OF THE INVENTION

Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

Btk is a key regulator of B-cell development, activation, signaling, and survival (Kurosaki, Curr. Op. Imm., 2000, 276-281; Schaeffer and Schwartzberg, Curr. Op. Imm. 2000, 282-288). In addition, Btk plays a role in a number of other hematopoetic cell signaling pathways, e.g., Toll like receptor (TLR) and cytokine receptor-mediated TNF-α production in macrophages, IgE receptor (FcERI) signaling in Mast cells, inhibition of Fas/APO-1 apoptotic signaling in B-lineage lymphoid cells, and collagen-stimulated platelet aggregation. See, e.g., Jeffries, et al., (2003), Journal of Biological Chemistry 278:26258-26264; Horwood, et al., (2003), The Journal of Experimental Medicine 197:1603-1611; Iwaki et al. (2005), Journal of Biological Chemistry 280(48):40261-40270; Vassilev et al. (1999), Journal of Biological Chemistry 274(3): 1646-1656, and Quek et al. (1998), Current Biology 8(20):1137-1140.

With the regulatory role reported for Btk in FcεR-mediated mast cell activation, Btk inhibitors may also show potential in the treatment of allergic responses (Gilfillan et al (2009), Immunological Reviews 288:149-169).

The reported role for Btk in the regulation of proliferation and apoptosis of B cells indicates the potential for Btk inhibitors in the treatment of tumors of blood and lymphatic system, such as B-cell lymphomas. Inhibition of Btk seems to be relevant in particular for B-cell lymphomas due to chronic active BCR signaling (Davis et al (2010), Nature, 463:88-94).

Studies of ibrutinib, a covalent selective inhibitor of Bruton's tyrosine kinase, have demonstrated significant antitumor activity of the drug against mantle cell lymphoma and chronic lymphocytic leukemia, as well as acceptable tolerability (Robert Roskoski Jr. (2016), Pharmacol. Res., 113: 395-408; O. Foluso et al (2016), Clin. Lymphoma Myeloma Leuk., 16(2): 63-69). Also ibrutinib treats graft-versus-host disease (GVHD) (Miklos et al. (2017), Blood 2017:blood-2017-07-793786). However, off-target interactions of Ibrutinib with EGFR and other TEC family kinases can cause the adverse drug reactions (ADR), such as bleeding, rash, diarrhea and atrial fibrillation (Wu et al. (2016), Journal of Hematology & Oncology, 9:80). According to pharmacokinetic studies, Ibrutinib is prone to first-pass clearance to form a major metabolite, it is 15 times less active than the parent substance (Bose et al, (2016), Expert Opinion on Drug Metabolism & Toxicology).

Therefore, clinical need exists in new compounds affecting Bruton's tyrosine kinase (Btk) with favorable characteristics of a potential medication.

### DESCRIPTION OF THE INVENTION

The terms used in the description of this invention appear below.

Optionally substituted in one, two, three, or several positions means the specified group can be substituted by a radical or any combination of radicals in one, two, three, or from one to six positions.

“Alkyl” means an aliphatic straight chain or branched chain hydrocarbon group having from 1 to 12 carbon atoms, more preferably from 1 to 6 carbon atoms. Branched chain means alkyl chain having one or more “lower alkyl” substituents. Examples of alkyl groups include, but are not limited to, methyl, ethyl, n-propyl, iso-propyl, n-butyl, iso-butyl, sec-butyl, tert-butyl, n-pentyl, 2-pentyl, 3-pentyl, neo-pentyl, n-hexyl. Alkyl may have substituents which may be same or different structure.

“Cycloalkyl” means a saturated carbocyclic ring that contains from 3 to 10 carbon ring atoms. Examples of cycloalkyl groups include, but are not limited to, monocyclic groups, such as cyclopropyl, cyclobutyl, cyclopentyl, cyclohexyl, cycloheptyl, cyclooctyl, cyclononyl or cyclodecyl, bicyclic groups, such as bicycloheptyl or bicyclooctyl. Cycloalkyl may have substituents which may be same or different structure.

“Alkenyl” means a straight chain or branched chain hydrocarbon group having from 2 to 12 carbon atoms, more preferably from 2 to 6 carbon atoms that contains one or more carbon-carbon double bound. Alkenyl may have substituents which may be same or different structure.

“Alkynyl” means a straight chain or branched chain hydrocarbon group having from 2 to 12 carbon atoms, more preferably from 2 to 6 carbon atoms that contains one or more carbon-carbon triple bound. Alkynyl may have substituents which may be same or different structure.

“Aryl” means an aromatic monocyclic or polycyclic system having from 6 to 14 carbon atoms, more preferably from 6 to 10 carbon atoms. Examples of aryl groups include, but are not limited to, phenyl, phenylene, benzenetriyl, indanyl, naphthyl, naphthylene, naphthalenetriyl and anthrylene. Aryl may have cyclic system substituents which may be same or different structure. Aryl can be annelated with a nonaromatic cyclic system or heterocycle.

“Alkyloxy” or “Alkoxy” means an alkyl-O— group, wherein alkyl is defined in this section. Examples of alkoxy groups include, but are not limited to, methyloxy, ethyloxy, n-propyloxy, iso-propyloxy, n-butyloxy, tert-butyloxy and iso-butyloxy.

“Amino group” means RₖRₚN— group.

“Aminocarbonyl” means —C(═O)NRₖRₚ group.

Examples of Rₖ and Rₚ include, but not limited to, substituents selected from the group containing hydrogen, alkyl, alkenyl, alkynyl, cycloalkyl, aryl, heterocyclyl, heteroaryl, or Rₖ and Rₚ together with nitrogen atom, to which they are attached, form a 4-7-membered heterocyclyl or heteroaryl.

“Lower alkyl” means a straight chain or branched chain alkyl having from 1 to 4 carbon atoms.

“Halo” or “Halogen” (Hal) means fluoro, chloro, bromo and iodo.

“Heterocycle”, “heterocyclyl”, “heterocyclic ring” means a monocyclic or polycyclic system having from 3 to 11 carbon atoms, of which one or more carbon atoms are substituted by one or more heteroatoms, such as nitrogen, oxygen, sulfur. Heterocycle may be fused with aryl or heteroaryl. Heterocycle may have one or more substituents which may be same or different structure. Nitrogen and sulfur atoms of heterocycle could be oxidized to N-oxide, S-oxide or S-dioxide. Heterocycle may be fully saturated, partially saturated and unsaturated. Examples of heterocycle include, but are not limited to, azetidine, pyrrolidine, piperidine, 2,8-diazaspiro[4.5]decane, piperazine, morpholine, and others.

“Heteroaryl” means an aromatic monocyclic or polycyclic system having from 5 to 11 carbon atoms, preferably from 5 to 10, of which one or more carbon atoms are substituted by one or more heteroatoms, such as nitrogen, sulfur or oxygen. Nitrogen atom of heterocycle could be oxidized to N-oxide. Heteroaryl may have one or more substituents which may be same or different structure. Examples of heteroaryl are pyrrolyl, furanyl, thienyl, pyridyl, pyrazinyl, pyrimidinyl, pyridazinyl, isoxazolyl, isothiazolyl, tetrazolyl, oxazolyl, thiazolyl, pyrazolyl, furazanyl, triazolyl, 1,2,4-thiadiazolyl, quinoxalinyl, phthalazinyl, imidazo[1,2-a]pyridinyl, imidazo[2,1-b]thiazolyl, benzofurazanyl, indolyl, azaindolyl, benzimidazolyl, benzothiazenyl, quinolinyl, imidazolyl, pyrazolyl, thienopyridyl, quinazolinyl, naphthyridinyl, thienopyrimidinyl, pyrrolopyridinyl, imidazopyridyl, isoquinolinyl, benzoazaindolyl, 1,2,4-triazinyl, thienopyrrolyl, furopyrrolyl, and the like.

“Partially saturated” means a ring system including at least one double or triple bond. The term “partly saturated” relates to rings having many sites for saturation and does not include aryl and heteroaryl systems as they defined above.

The term “oxo” used in this document relates to the radical ═O.

“Substituent” means a chemical radical attached to a scaffold (fragment).

“Solvate” is a molecular aggregate that consists of the compound of the present invention, or its pharmaceutically acceptable salt, with one or more solvent molecules. The solvent molecules are molecules of common pharmaceutical solvents, known to be safe for recipients, e.g. water, ethanol, ethylene glycol, etc. Other solvents, such as methanol, methyl-tert-butyl ether, ethyl acetate, methyl acetate, (R)-propylene glycol or (S)-propylene glycol, 1,4-butanediol, and the like, can be used to form intermediate solvates for obtaining preferable solvates.

“Hydrate” means a solvate with water as the solvent.

Solvates and/or hydrates preferably exist in crystalline form.

Terms “bond”, “chemical bond”, or “single bond” refer to a chemical bonding of two atoms or two moieties (i.e., groups, fragments) when the atoms joined by the bond are considered to be part of larger substructure.

The term “chiral” refers to molecules that have the property of being incompatible with their mirror image, whereas the term “achiral” refers to molecules that have the property of being compatible with their mirror image.

The term “stereoisomers” refers to compounds that have identical chemical composition and the same structure, but differ in the spatial arrangement of atoms or their groups. Stereoisomers may include geometric isomers, enantiomers, diastereomers.

The term “diastereomer” refers to a stereoisomer with two or more centers of chirality, and such molecules are not mirror images of each other. Diastereomers have different physical properties, for example, melting points, boiling points, spectral properties and reactivity. Mixtures of diastereomers could be separated using high-resolution analytical techniques, such as electrophoresis and chromatography.

The term “enantiomers” refers to two stereoisomers of a compound being mirror images of one another and not compatible in space.

The terms “racemic mixture” and “racemate” refer to an equimolar mixture of two enantiomers that are not optical active. Enantiomers can be isolated from the racemic mixture separately by chiral resolution, such as, for example, supercritical fluid chromatography (SFC).

The compounds of the invention may contain asymmetric or chiral centers and, therefore, exist in different stereoisomeric forms. It is contemplated that all stereoisomeric forms of the compounds of the invention, including but not limited to diastereomers, enantiomers and atropisomers, as well as mixtures thereof, such as racemic mixtures, are part of the present invention. Many organic compounds exist in optically active forms, i. e. they have the ability to rotate the plane of linearly polarized light. When describing an optically active compound, the prefixes R and S are used to designate the absolute configuration of the molecule with respect to its chiral center(s). A particular stereoisomer can also be defined as an enantiomer, and a mixture of such isomers is often referred to as an enantiomeric mixture.

The term “atropisomers” refers to compounds having spatial isomerism caused by the absence of rotation around a simple bond, for example, in diphenyls, dinaphthyls and others.

The term “excipient” is used herein to describe any ingredient other than the compound(s) of the invention.

“Pharmaceutical composition” means a composition, comprising a compound of the invention and one or more pharmaceutically acceptable excipients. Examples of excipients include, but are not limited to, pharmaceutically acceptable and pharmacologically compatible fillers, solvents, diluents, carriers, auxiliary, distributing and sensing agents, delivery agents, such as preservatives, stabilizers, filler, disintegrators, moisteners, emulsifiers, suspending agents, thickeners, sweeteners, flavouring agents, aromatizing agents, antibacterial agents, fungicides, lubricants, and prolonged delivery controllers, the choice and suitable proportions of which depend on the type and way of administration and dosage. Examples of suitable suspending agents are ethoxylated isostearyl alcohol, polyoxyethene, sorbitol and sorbitol ether, microcrystalline cellulose, aluminum metahydroxide, bentonite, agar-agar and tragacant and their mixtures as well. Protection against action of microorganisms can be provided by various antibacterial and antifungal agents, such as, for example, parabens, chlorobutanole, sorbic acid, and similar compounds. Composition may also contain isotonic agents, such as, for example, sugars, sodium chloride, and similar compounds. Prolonged action of composition may be achieved by agents slowing down absorption of active ingredient, for example, aluminum monostearate and gelatine. Examples of suitable carriers, solvents, diluents and delivery agents include water, ethanol, polyalcohols and their mixtures, natural oils (such as olive oil) and organic esters (such as ethyl oleate) for injections. Examples of fillers are lactose, milk-sugar, sodium citrate, calcium carbonate, calcium phosphate and the like. Examples of disintegrators and distributors are starch, alginic acid and its salts, silicates and the like. Examples of suitable lubricants are magnesium stearate, sodium lauryl sulfate, talc and polyethylene glycol of high molecular weight. Pharmaceutical composition for peroral, sublingual, transdermal, intramuscular, intravenous, subcutaneous, local or rectal administration of active ingredient, alone or in combination with another active compound may be administered to human and animals in a standard administration form, in a mixture with traditional pharmaceutical carriers. Suitable standard administration forms include peroral forms such as tablets, gelatin capsules, pills, powders, granules, chewing-gums and peroral solutions or suspensions; sublingual and transbuccal administration forms; aerosols; implants; local, transdermal, subcutaneous, intramuscular, intravenous, intranasal or intraocular forms and rectal administration forms.

“Pharmaceutically acceptable salt” means relatively nontoxic both organic and inorganic salts of acids and bases disclosed in this invention. These salts could be prepared in situ in the processes of synthesis, isolation or purification of compounds or they could be prepared specially. In particular, salts of bases specially could be prepared from purified base of the disclosed compound and suitable organic or mineral acid. Examples of salts prepared in this manner include hydrochlorides, hydrobromides, sulfates, bisulfates, phosphates, nitrates, acetates, oxalates, valeriates, oleates, palmitates, stearates, laurates, borates, benzoates, lactates, p-toluenesulfonates, citrates, maleates, fumarates, succinates, tartrates, methane sulphonates, malonates, salicylates, propionates, ethane sulphonates, benzene sulfonates, sulfamates and the like (Detailed description of such salts properties is given in: Berge S. M., et al., “Pharmaceutical Salts” J. Pharm. Sci. 1977, 66: 1-19). Aminoacids may be selected from aminoacids-lysine, ornithine and arginine.

“Medicament”—is a compound (or a mixture of compounds as a pharmaceutical composition) in the form of tablets, capsules, injections, ointments and other ready forms intended for restoration, improvement or modification of physiological functions in humans and animals, and for treatment and prophylaxis of diseases, for diagnostics, anesthesia, contraception, cosmetology and others.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

“Prophylaxis”, “prophylactic therapy” (“preventive therapy”) refers to a set of measures aimed at preventing the onset, eliminating risk factors, or early detecting a disease or disorder, its exacerbation, relapse, complications or other consequences.

In one aspect, the subject of treatment, or patient, is a mammal, preferably a human subject. Said subject may be either male or female, of any age.

“Disorder” means any condition that would benefit from treatment with the compound of the present invention. This means chronic and acute disorders or diseases including those pathological conditions that predispose the mammal to the disorder in question. Non-limiting examples of disorders to be treated herein include benign and malignant tumors; leukemias and lymphoid malignancies; breast, ovarian, stomach, endometrial, salivary gland, lung, kidney, colon, thyroid, pancreas, prostate or bladder cancer; neuronal, glial, astrocytal, hypothalamic and other glandular, macrophagal, epithelial, stromal and blastocoelic disorders; inflammatory, angiogenic and immunologic disorders. Preferable disorders to be treated with the compound of the invention are tumors of blood and chronic lymphoproliferative diseases, cancer, autoimmune diseases.

“Therapeutically effective amount” refers to that amount of the therapeutic agent being administered which will relieve to some extent one or more of the symptoms of the disease/disorder being treated.

The terms “inhibits”, “inhibiting”, or “inhibitor” of a kinase, as used herein, refer to suppression/inhibition of enzymatic phosphotransferase activity.

The term “irreversible inhibitor”, as used herein, refers to a compound that, upon contact with a target protein (e.g., a kinase) causes the formation of a new covalent bond with or within the protein, whereby one or more of the target protein's biological activities (e.g., phosphotransferase activity) is diminished or abolished notwithstanding the subsequent presence or absence of the irreversible inhibitor.

The term “irreversible Btk inhibitor”, as used herein, refers to an Btk inhibitor that can form a covalent bond with an amino acid residue of Btk.

The term “biopharmaceutical,” which may also be referred to as a biologic medical product or biologic, is intended to refer to any medicinal product manufactured in, extracted from, or semi-synthesized from biological sources. Exemplary biopharmaceuticals include vaccines, blood, or blood components, allergenics, somatic cells, gene therapies, tissues, recombinant therapeutic protein, and living cells used in cell therapy. Biopharmaceuticals can comprise sugars, proteins, or nucleic acids, or be combinations of these substances, or may be living cells or tissues. They may be isolated from natural sources, such as human, animal, or microorganism, or produced by means of biological processes involving recombinant DNA technology. Non-limiting examples of the biopharmaceuticals include peptides, carbohydrates, lipids, monoclonal antibodies, biosimilars, biologies, non-IgG antibody-like structures such as but not limited to heterologous antibodies, diabodies, triabodies, and tetrabodies, other multivalent antibodies including scFv2/BITEs, streptabodies, and tandem diabodies, or combinations thereof. Optionally the biopharmaceuticals may be covalently linked to toxins, radioactive materials or another biological molecule, including proteins, peptides, nucleic acids, and carbohydrates. The aforementioned biological molecules include, but are not limited to, molecules of bacterial origin, viral origin, mammalian origin, or recombinant origin.

As used herein, the words “comprise,” “have,” “include,” or variations such as “comprises,” “comprising,” “has,” “having,” “includes” or “including”, and all grammatical variations thereof will be understood to imply the inclusion of a stated integer or group of integers but not the exclusion of any other integer or group of integers.

### DETAILED DESCRIPTION OF THE INVENTION

The present invention relates to a compound of Formula I:

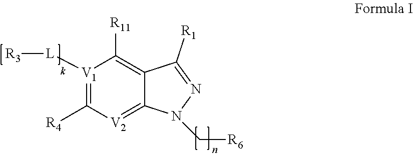

or pharmaceutically acceptable salt, solvate or stereoisomer thereof,
wherein:

### V₁ is C or N,

V₂ is C(R₂) or N,
whereby if V₁ is C then V₂ is N,
if V₁ is C then V₂ is C(R₂), or
if V₁ is N then V₂ is C(R₂);
each n, k is independently 0, 1;
each R₂, R₁₁ is independently H, D, Hal, CN, NR′R″, C(O)NR′R″, C₁-C₆ alkoxy;
R₃ is H, D, hydroxy, C(O)C₁-C₆ alkyl, C(O)C₂-C₆ alkenyl, C(O)C₂-C₆ alkynyl, C₁-C₆ alkyl;
R₄ is H, Hal, CN, CONR′R″, hydroxy, C₁-C₆ alkyl, C₁-C₆ alkoxy;
L is CH₂, NH, O or chemical bond;
R₁ is selected from the group of the fragments, comprising:

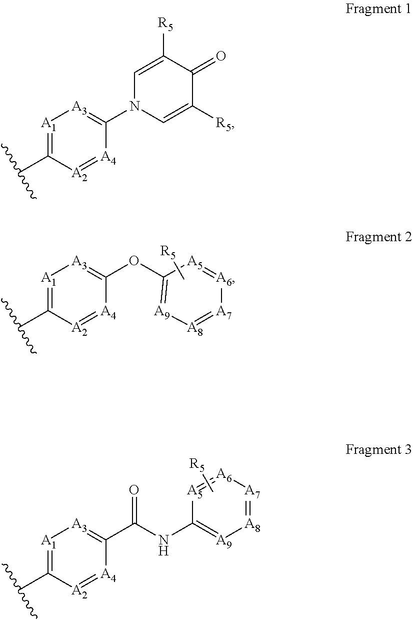

each A₁, A₂, A₃, A₄ is independently CH, N, CHal;
each A₅, A₆, A₇, A₈, A₉ is independently C, CH or N;
R₅ is H, CN, Hal, CONR′R″, C₁-C₆ alkyl, non-substituted or substituted by one or more halogens;
each R′ and R″ is independently selected from the group, comprising H, C₁-C₆ alkyl, C₁-C₆ cycloalkyl, aryl;
R₆ is selected from the group:

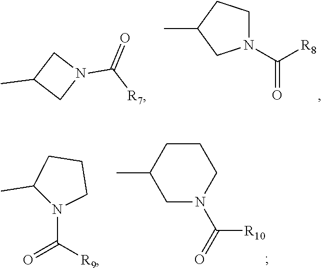

each R₇, R₈, R₉, R₁₀ is independently vinyl, methylacetylenyl;

### Hal is Cl, Br, I, F.

In another embodiment, the present invention relates to a compound of Formula I wherein R₁ is selected from the group including:

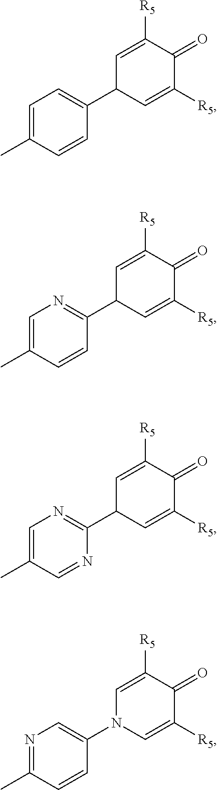

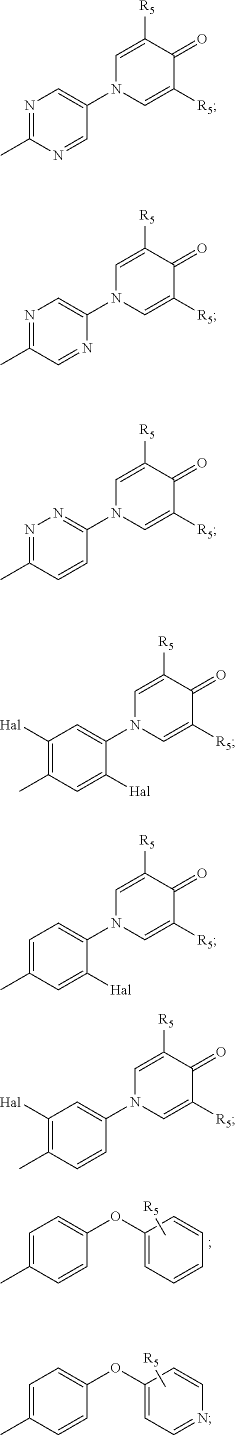

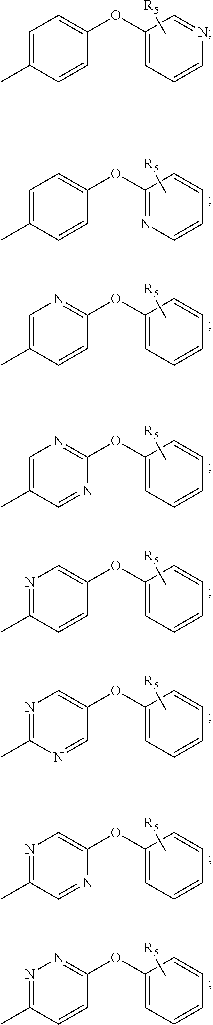

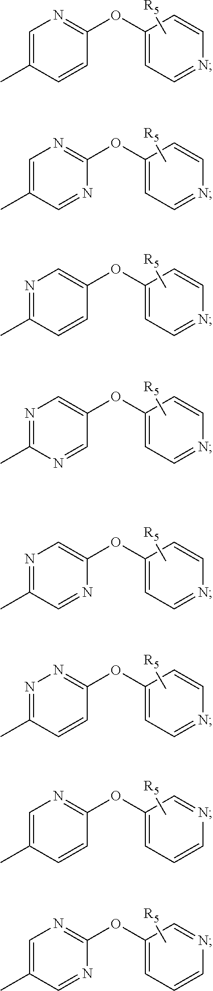

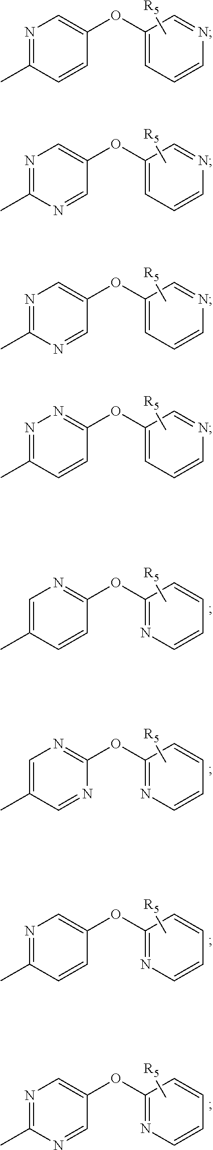

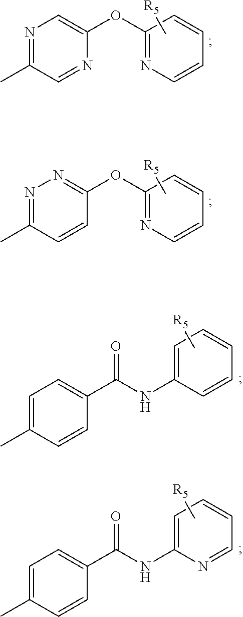

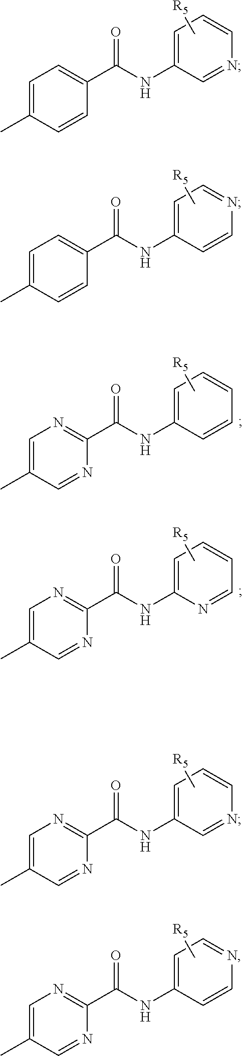

wherein R₅, Hal have the above meanings.

In another embodiment, the present invention relates to a compound of Formula II:

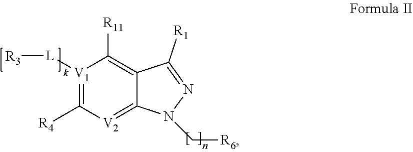

or pharmaceutically acceptable salt, solvate or stereoisomer thereof,
wherein:

### V₁ is C or N,

V₂ is C(R₂) or N,
whereby if V₁ is C then V₂ is N,
if V₁ is C then V₂ is C(R₂), or
if V₁ is N then V₂ is C(R₂);
each n, k is independently 0, 1;
R₂ is Hal, CN, NR′R″, C(O)NR′R″, C₁-C₆ alkoxy;
R₁₁ is H, Hal, CN, NR′R″, C(O)NR′R″, C₁-C₆ alkoxy;
L is CH₂, NH, O or chemical bond;
R₃ is H, hydroxy, C(O)C₁-C₆ alkyl, C(O)C₂-C₆ alkenyl, C(O)C₂-C₆ alkynyl, C₁-C₆ alkyl;
R₄ is H, Hal, CN, CONR′R″, hydroxy, C₁-C₆ alkyl, C₁-C₆ alkoxy;
R₁ is selected from the group of the fragments, comprising:

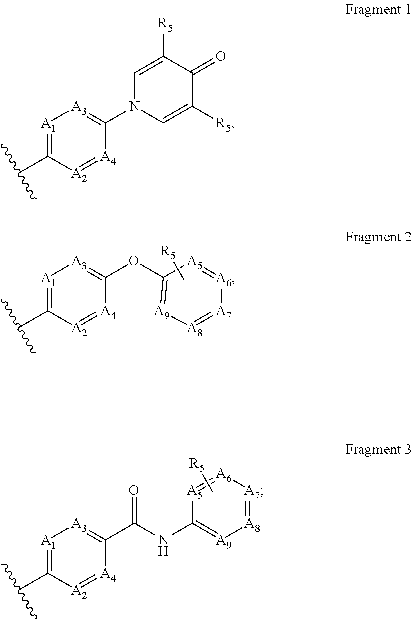

each A₁, A₂, A₃, A₄ is independently CH, N, CHal;
each A₅, A₆, A₇, A₈, A₉ is independently C, CH or N;
R₅ is H, CN, Hal, CONR′R″, C₁-C₆ alkyl, non-substituted or substituted by one or more halogens;
each R′ and R″ is independently selected from the group, comprising H, C₁-C₆ alkyl, C₁-C₆ cycloalkyl, aryl;
R₆ is selected from the group:

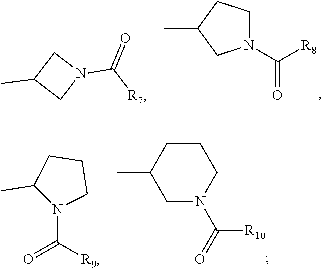

each R₇, R₈, R₉, R₁₀ is independently vinyl, methylacetylenyl;

### Hal is Cl, Br, I, F.

In another embodiment, the present invention relates to a compound of Formula III:

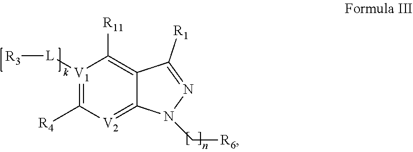

or pharmaceutically acceptable salt, solvate or stereoisomer thereof,
wherein:

### V₁ is C or N,

V₂ is C(R₂) or N,
whereby if V₁ is C then V₂ is N,
if V₁ is C then V₂ is C(R₂), or
if V₁ is N then V₂ is C(R₂);
each n, k is independently 0, 1;
R₂ is Hal, CN, NR′R″, C(O)NR′R″, C₁-C₆ alkoxy;
R₁ is H, Hal, CN, NR′R″, C(O)NR′R″, C₁-C₆ alkoxy;
L is CH₂, NH, O or chemical bond;
R₃ is H, hydroxy, C(O)C₁-C₆ alkyl, C(O)C₂-C₆ alkenyl, C(O)C₂-C₆ alkynyl, C₁-C₆ alkyl;
R₄ is H, Hal, CN, CONR′R″, hydroxy, C₁-C₆ alkyl, C₁-C₆ alkoxy;
wherein if V₁ is C, V₂ is N,
then at least one of R₃, R₄, R₁₁ is not H;
R₁ is selected from the group of the fragments, comprising:

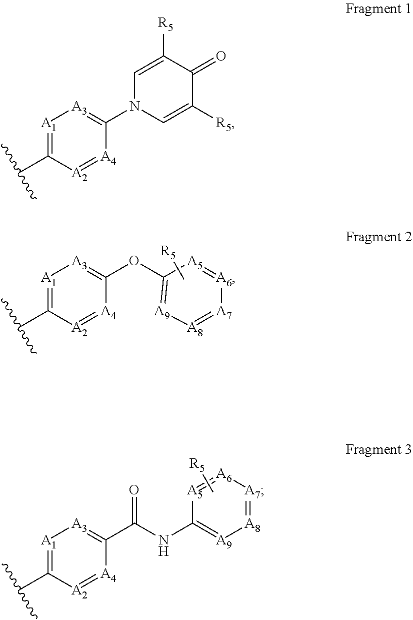

each A₁, A₂, A₃, A₄ is independently CH, N, CHal;
each A₅, A₆, A₇, A₈, A₉ is independently C, CH or N;
R₅ is H, CN, Hal, CONR′R″, C₁-C₆ alkyl, non-substituted or substituted by one or more halogens;
each R′ and R″ is independently selected from the group, comprising H, C₁-C₆ alkyl, C₁-C₆ cycloalkyl, aryl;
R₆ is selected from the group:

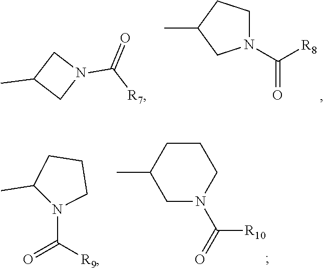

each R₇, R₈, R₉, R₁₀ is independently vinyl, methylacetylenyl;

### Hal is Cl, Br, I, F;

In another embodiment, the present invention relates to a compound of Formula IV:

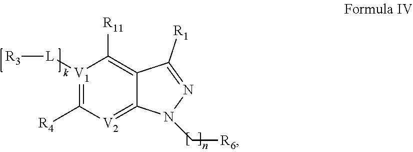

or pharmaceutically acceptable salt, solvate or stereoisomer thereof,
wherein:

### V₁ is C or N,

V₂ is C(R₂) or N,
whereby if V₁ is C then V₂ is N,
if V₁ is C then V₂ is C(R₂), or
if V₁ is N then V₂ is C(R₂);
each n, k is independently 0, 1;
R₂ is Hal, CN, NR′R″, C(O)NR′R″, C₁-C₆ alkoxy;
R₁₁ is H, Hal, CN, NR′R″, C(O)NR′R″, C₁-C₆ alkoxy; L is CH₂, NH, O or chemical bond;
R₃ is H, hydroxy, C(O)C₁-C₆ alkyl, C(O)C₂-C₆ alkenyl, C(O)C₂-C₆ alkynyl, C₁-C₆ alkyl;
R₄ is H, Hal, CN, CONR′R″, hydroxy, C₁-C₆ alkyl, C₁-C₆ alkoxy;
wherein if V₁ is C, V₂ is N,
then at least one of R₃, R₄, R₁₁ is not H;
R₁ is selected from the group of the fragments, comprising:

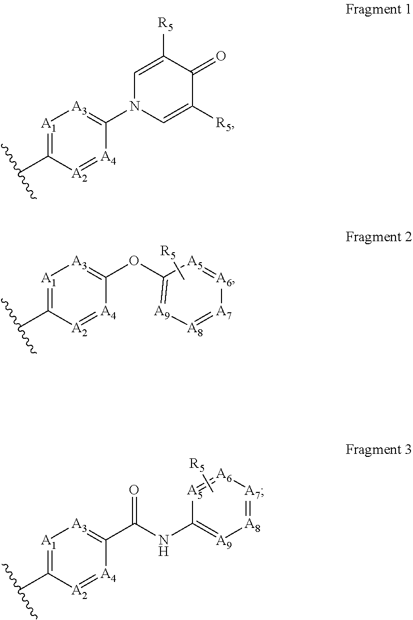

each A₁, A₂, A₃, A₄ is independently CH, N, CHal;
each A₅, A₆, A₇, A₈, A₉ is independently C, CH or N,
and at least one of A₅, A₆, A₇, A₈, A₉ is N;
R₅ is H, CN, Hal, CONR′R″, C₁-C₆ alkyl, non-substituted or substituted by one or more halogens;
each R′ and R″ is independently selected from the group, comprising H, C₁-C₆ alkyl, C₁-C₆ cycloalkyl, aryl;
R₆ is selected from the group:

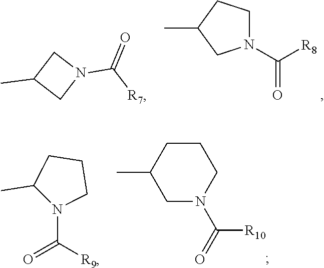

each R₇, R₈, R₉, R₁₀ is independently vinyl, methylacetylenyl;

### Hal is Cl, Br, I, F;

In another embodiment, the present invention relates to a compound of Formula V:

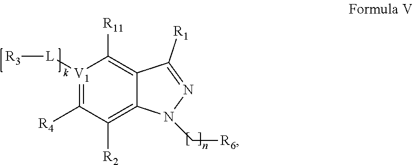

or pharmaceutically acceptable salt, solvate or stereoisomer thereof,
wherein:

### V₁ is C or N,

each n, k is independently 0, 1;
R₂ is H, Hal, CN, NR′R″, C(O)NR′R″, C₁-C₆ alkoxy;
R₃ is H, hydroxy, C(O)C₁-C₆ alkyl, C(O)C₂-C₆ alkenyl, C(O)C₂-C₆ alkynyl, C₁-C₆ alkyl;
R₄ is H, Hal, CN, CONR′R″, hydroxy, C₁-C₆ alkyl, C₁-C₆ alkoxy; L is CH₂, NH, O or chemical bond;
R₁ is selected from the group of the fragments, comprising:

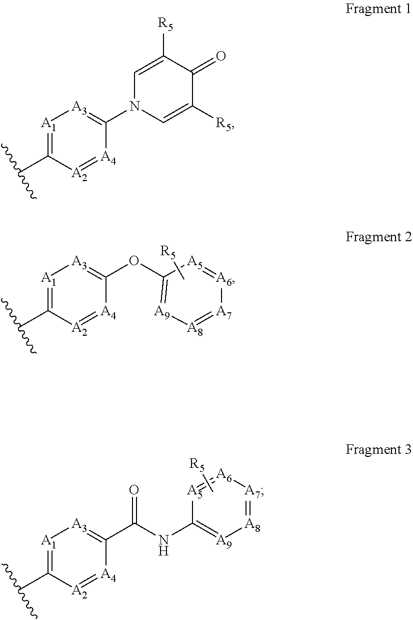

each A₁, A₂, A₃, A₄ is independently CH, N, CHal;
each A₅, A₆, A₇, A, A₉ is independently C, CH or N;
R₅ is H, CN, Hal, CONR′R″, C₁-C₆ alkyl, non-substituted or substituted by one or more halogens;
each R′ and R″ is independently selected from the group, comprising H, C₁-C₆ alkyl, C₁-C₆ cycloalkyl, aryl;
R₆ is selected from the group:

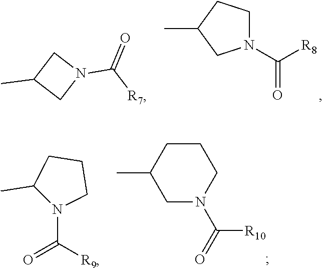

each R₇, R₈, R₉, R₁₀ is independently vinyl, methylacetylenyl;
R₁₁ is H, Hal, CN, NR′R″, C(O)NR′R″, C₁-C₆ alkoxy;

### Hal is Cl, Br, I, F;

In another embodiment, the present invention relates to a compound of Formula VI:

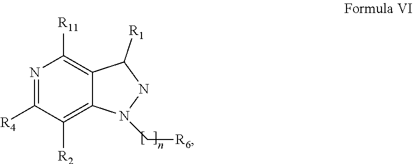

or pharmaceutically acceptable salt, solvate or stereoisomer thereof,
wherein:
R₁ is selected from the group of the fragments, comprising:

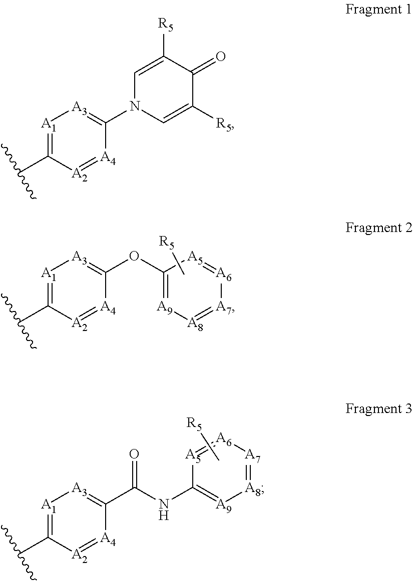

each A₁, A₂, A₃, A₄ is independently CH, N, CHal;
each A₅, A₆, A₇, A₈, A₉ is independently C, CH or N;
R₅ is H, CN, Hal, CONR′R″, C₁-C₆ alkyl, non-substituted or substituted by one or more halogens;
each R′ and R″ is independently selected from the group, comprising H, C₁-C₆ alkyl, C₁-C₆ cycloalkyl, aryl;
R₂ is Hal, CN, NR′R″, C(O)NR′R″, C₁-C₆ alkoxy;
R₄ is H, Hal, CN, CONR′R″, hydroxy, C₁-C₆ alkyl, C₁-C₆ alkoxy;
n is 0, 1, 2;
R₆ is selected from the group:

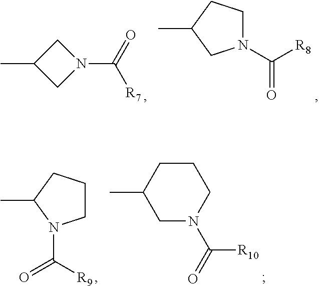

R₇ is vinyl, methylacetylenyl;
each R₈, R₉, R₁₀ is independently methylacetylenyl
R₁₁ is H, Hal, CN, NR′R″, C(O)NR′R″, C₁-C₆ alkoxy;

### Hal is Cl, Br, I, F;

In another embodiment, the present invention relates to a compound of Formula VII:

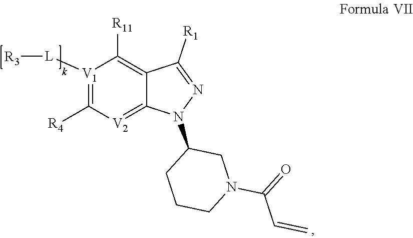

or pharmaceutically acceptable salts or solvates;
wherein:

### V₁ is C or N,

V₂ is C(R₂) or N,
whereby if V₁ is C then V₂ is N,
if V₁ is C then V₂ is C(R₂), or
if V₁ is N then V₂ is C(R₂);
k independently is 0, 1;

### R₂ is H, D, Hal, CN, NR′R″, C(O)NR′R″;

R₃ is H, hydroxy, C(O)C₁-C₆ alkyl, C(O)C₂-C₆ alkenyl, C(O)C₂-C₆ alkynyl, C₁-C₆ alkyl;
R₄ is H, Hal, CN, CONR′R″, hydroxy, C₁-C₆ alkyl, C₁-C₆ alkoxy;
L is CH₂, NH, O or chemical bond;
R₁ is selected from the group of the fragments, comprising:

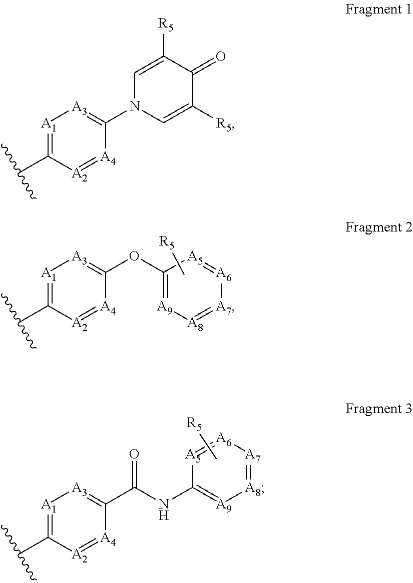

each A₁, A₂, A₃, A₄ is independently CH, N, CHal;
each A₅, A₆, A₇, A₈, A₉ is independently C, CH or N;
R₅ is H, CN, Hal, CONR′R″, C₁-C₆ alkyl, non-substituted or substituted by one or more halogens;
each R′ and R″ is independently selected from the group, comprising H, C₁-C₆ alkyl, C₁-C₆ cycloalkyl, aryl;
R₁₁ is H, Hal, CN, NR′R″, C(O)NR′R″, C₁-C₆ alkoxy;

### Hal is Cl, Br, I, F;

In another embodiment the present invention relates to the compound of Formula II, compound of Formula III, compound of Formula IV, compound of Formula V, compound of Formula VI, compound of Formula VII, wherein R₁ is selected from the group including:

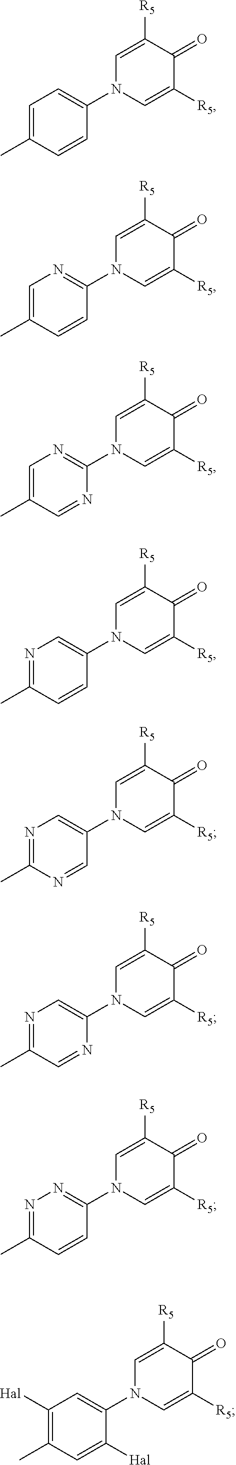

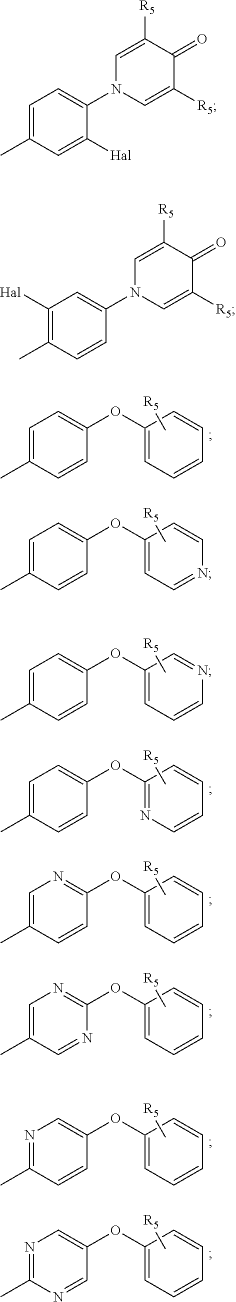

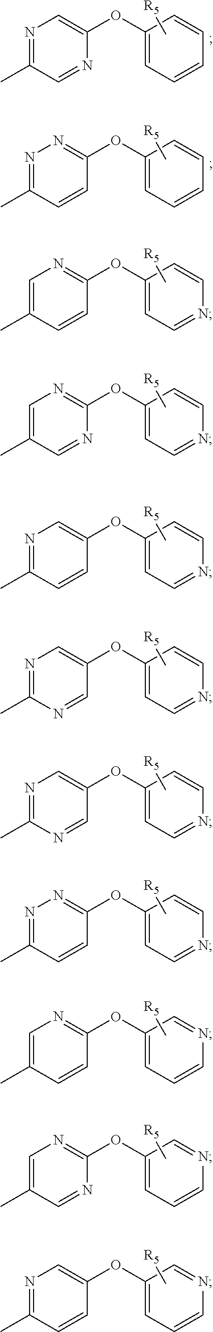

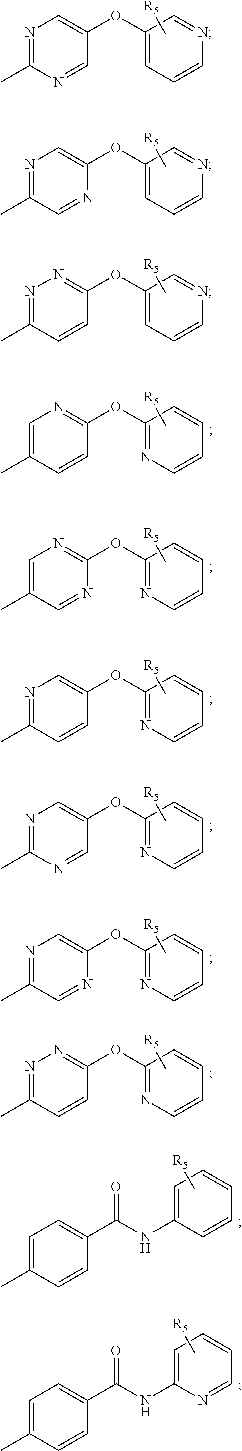

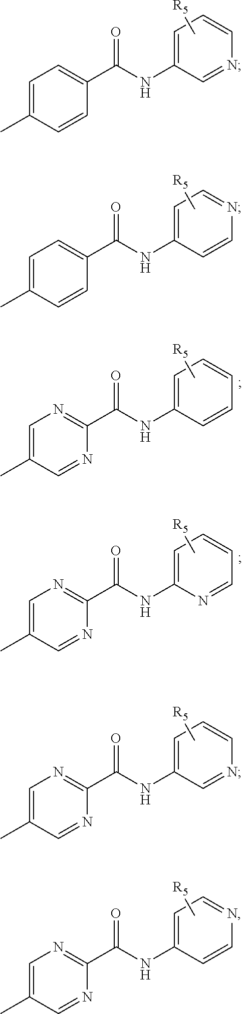

wherein R₅, Hal have the above meanings.

Compounds, described in the present invention, may be formed as, and/or used as, pharmaceutically acceptable salts. The type of pharmaceutical acceptable salts, include, but are not limited to: acid addition salts, formed by reacting the free base form of the compound with a pharmaceutically acceptable inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfónic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-3-hydroxy-2-ene-1-carboxylic acid, 3-phenylpropionic acid, trimethylacetic acid, tert-butylacetic acid, lauryl sulfuric acid, gluconic acid, glutamic acid, hydroxynaphthoic acid, salicylic acid, stearic acid, muconic acid, and the like.

The corresponding counterions of the pharmaceutically acceptable salts may be analyzed and identified using various methods including, but not limited to, ion exchange chromatography, ion chromatography, capillary electrophoresis, inductively coupled plasma, atomic absorption spectroscopy, mass spectrometry, or any combination thereof.

The salts are recovered by using at least one of the following techniques: filtration, precipitation with a non-solvent followed by filtration, evaporation of the solvent, or, in the case of aqueous solutions, lyophilization. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

Compounds described herein may be in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause one crystal form to dominate.

The screening and characterization of the pharmaceutically acceptable salts, polymorphs and/or solvates may be accomplished using a variety of techniques including, but not limited to, thermal analysis, x-ray diffraction, spectroscopy, vapor sorption, and microscopy. Thermal analysis methods address to analysis of thermo chemical degradation or thermo physical processes including, but not limited to, polymorphic transitions, and such methods are used to analyze the relationships between polymorphic forms, to determine weight loss, to find the glass transition temperature, or for excipient compatibility studies. Such methods include, but are not limited to, Differential scanning calorimetry (DSC), Modulated Differential Scanning Calorimetry (MDCS), Thermogravimetric analysis (TGA), Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UVIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

In another embodiment of the present invention relates to the compounds selected from the group including:

| Structure | Name | Compound No. |
|---|---|---|
| 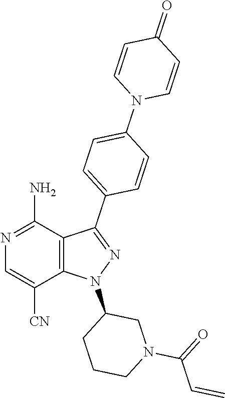 [SMILES: C=CC(=O)N1CCC[C@@H](n2nc(-c3ccc(-n4ccc(=O)cc4)cc3)c3c(N)ncc(C#N)c32)C1] | (R)-1-(1-acryloylpiperidin-3-yl)-4- amino-3-(4-(4-oxopyridin-1(4H)- yl)phenyl)-1H-pyrazolo[4,3- c]pyridin-7-carbonitrile | BCD-BTK-4 |
| 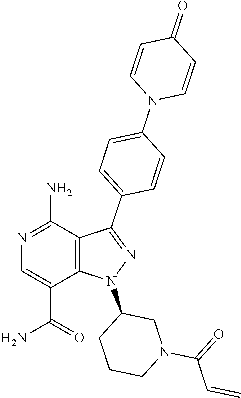 [SMILES: C=CC(=O)N1CCC[C@@H](n2nc(-c3ccc(-n4ccc(=O)cc4)cc3)c3c(N)ncc(C(N)=O)c32)C1] | (R)-1-(1-acryloylpiperidin-3-yl)-4- amino-3-(4-(4-oxopyridin-1(4H)- yl)phenyl)-1H-pyrazolo[4,3- c]pyridin-7-carboxamide | BCD-BTK-6 |
| 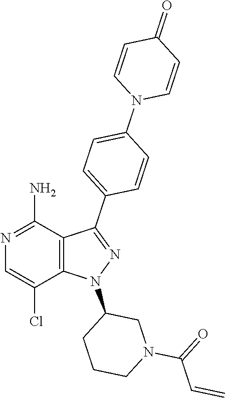 [SMILES: C=CC(=O)N1CCC[C@@H](n2nc(-c3ccc(-n4ccc(=O)cc4)cc3)c3c(N)ncc(Cl)c32)C1] | (R)-1-(4-(1-(1-acryloylpiperidin-3- yl)-4-amino-7-chloro-1H- pyrazolo[4,3-c]pyridin-3- yl)phenyl)pyridin-4(1H)-one | BCD-BTK-9 |
| 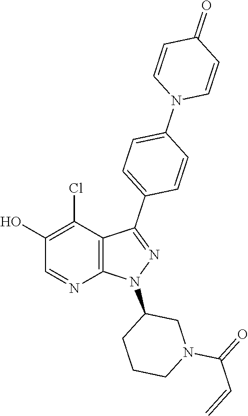 [SMILES: C=CC(=O)N1CCC[C@@H](n2nc(-c3ccc(-n4ccc(=O)cc4)cc3)c3c(Cl)c(O)cnc32)C1] | (R)-1-(4-(1-(1-acryloylpiperidin-3- yl)-4-chloro-5-hydroxy-1H- pyrazolo[3,4-b]pyridin-3-yl) phenyl)pyridin-4(1H)-one | BCD-BTK-13 |
| 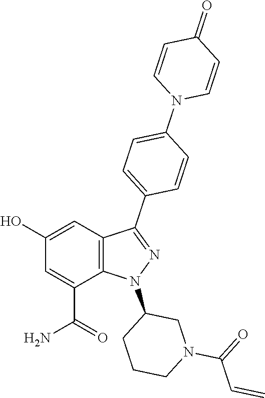 [SMILES: C=CC(=O)N1CCC[C@@H](n2nc(-c3ccc(-n4ccc(=O)cc4)cc3)c3cc(O)cc(C(N)=O)c32)C1] | (R)-1-(1-acryloylpiperidin-3-yl)-5- hydroxy-3-(4-(4-oxopyridin-1(4H)- yl)phenyl)-1H-indazol-7- carboxamide | BCD-BTK-18 |
| 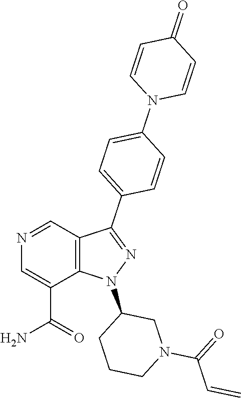 [SMILES: C=CC(=O)N1CCC[C@@H](n2nc(-c3ccc(-n4ccc(=O)cc4)cc3)c3cncc(C(N)=O)c32)C1] | (R)-1-(1-acryloylpiperidin-3-yl)-3- (4-(4-oxopyridin-1(4H)-yl)phenyl)- 1H-pyrazolo[4,3-c]pyridin-7- carboxamide | BCD-BTK-24 |
| 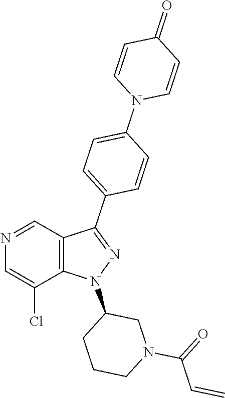 [SMILES: C=CC(=O)N1CCC[C@@H](n2nc(-c3ccc(-n4ccc(=O)cc4)cc3)c3cncc(Cl)c32)C1] | (R)-1-(4-(1-(1-acryloylpiperidin-3- yl)-7-chloro-1H-pyrazolo[4,3- c]pyridin-3-yl)phenyl)pyridin- 4(1H)-one | BCD-BTK-30 |
| 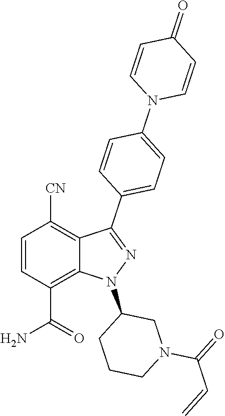 [SMILES: C=CC(=O)N1CCC[C@@H](n2nc(-c3ccc(-n4ccc(=O)cc4)cc3)c3c(C#N)ccc(C(N)=O)c32)C1] | (R)-1-(1-acryloylpiperidin-3-yl)-4- cyano-3-(4-(4-oxopyridin-1(4H)- yl)phenyl)-1H-indazol-7- carboxamide | BCD-BTK-35 |
| 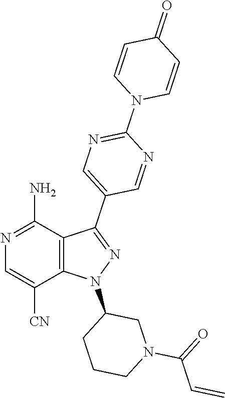 [SMILES: C=CC(=O)N1CCC[C@@H](n2nc(-c3cnc(-n4ccc(=O)cc4)nc3)c3c(N)ncc(C#N)c32)C1] | (R)-1-(1-acryloylpiperidin-3-yl)-4- amino-3-(2-(4-oxopyridin-1(4H)- yl)pyrimidin-5-yl)-1H-pyrazolo[4,3- c]pyridin-7-carbonitrile | BCD-BTK-36 |
| 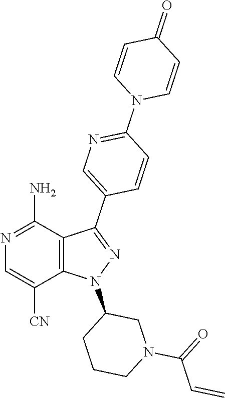 [SMILES: C=CC(=O)N1CCC[C@@H](n2nc(-c3ccc(-n4ccc(=O)cc4)nc3)c3c(N)ncc(C#N)c32)C1] | (R)-1-(1-acryloylpiperidin-3-yl)-4- amino-3-(4-oxo-4H-[1,2′-bipyridin]- 5′-yl)-1H-pyrazolo[4,3-c]pyridin-7- carbonitrile | BCD-BTK-38 |
| 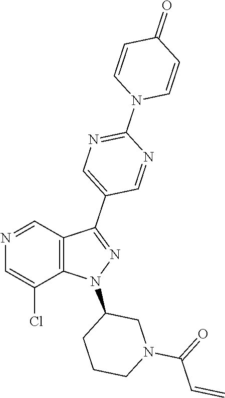 [SMILES: C=CC(=O)N1CCC[C@@H](n2nc(-c3cnc(-n4ccc(=O)cc4)nc3)c3cncc(Cl)c32)C1] | (R)-1-(5-(1-(1-acryloylpiperidin-3- yl)-7-chloro-1H-pyrazolo[4,3- c]pyridin-3-yl)pyrimidin-2-yl)pyridin- 4(1H)-one | BCD-BTK-54 |
| 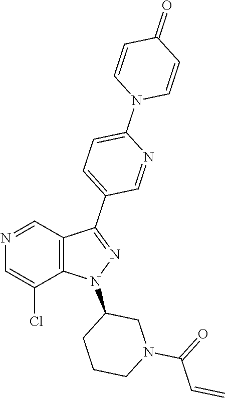 [SMILES: C=CC(=O)N1CCC[C@@H](n2nc(-c3ccc(-n4ccc(=O)cc4)nc3)c3cncc(Cl)c32)C1] | (R)-5′-(1-(1-acryloylpiperidin-3-yl)- 7-chloro-1H-pyrazolo[4,3-c]pyridin- 3-yl)-4H-[1,2′-bipyridin]-4-one | BCD-BTK-56 |
| 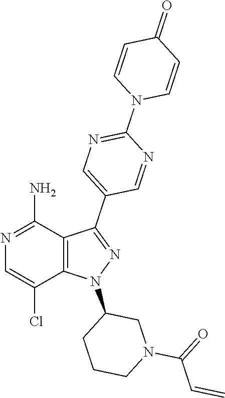 [SMILES: C=CC(=O)N1CCC[C@@H](n2nc(-c3cnc(-n4ccc(=O)cc4)nc3)c3c(N)ncc(Cl)c32)C1] | (R)-1-(5-(1-(1-acryloylpiperidin-3- yl)-4-amino-7-chloro-1H-pyrazolo[4,3- c]pyridin-3-yl)pyrimidin-2-yl)pyridin- 4(1H)-one | BCD-BTK-74 |
| 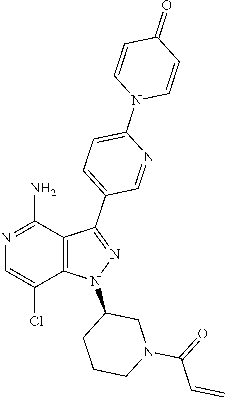 [SMILES: C=CC(=O)N1CCC[C@@H](n2nc(-c3ccc(-n4ccc(=O)cc4)nc3)c3c(N)ncc(Cl)c32)C1] | (R)-5′-(1-(1-acryloylpiperidin-3-yl)- 4-amino-7-chloro-1H-pyrazolo[4,3- c]pyridin-3-yl)-4H-[1,2′-bipyridin]- 4-one | BCD-BTK-76 |
| 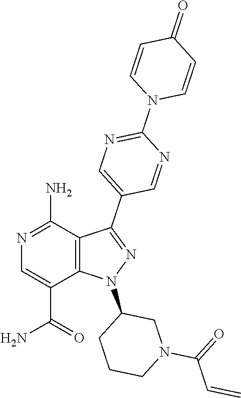 [SMILES: C=CC(=O)N1CCC[C@@H](n2nc(-c3cnc(-n4ccc(=O)cc4)nc3)c3c(N)ncc(C(N)=O)c32)C1] | (R)-1-(1-acryloylpiperidin-3-yl)-4- amino-3-(4-oxo-4H-[1,2′-bipyridin]- 5′-yl)-1H-pyrazolo[4,3-c]pyridin-7- carboxamide | BCD-BTK-86 |
| 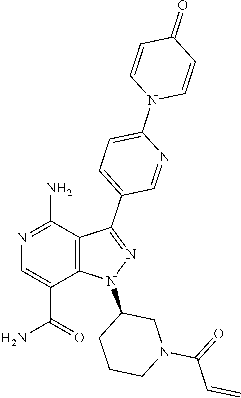 [SMILES: C=CC(=O)N1CCC[C@@H](n2nc(-c3ccc(-n4ccc(=O)cc4)nc3)c3c(N)ncc(C(N)=O)c32)C1] | (R)-1-(1-acryloylpiperidin-3-yl)-4- amino-3-(4-oxo-4H-[1,2′-bipyridin]- 5′-yl)-1H-pyrazolo[4,3-c]pyridin-7- carboxamide | BCD-BTK-88 |
| 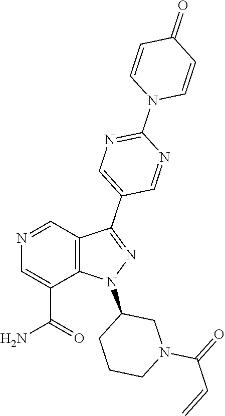 [SMILES: C=CC(=O)N1CCC[C@@H](n2nc(-c3cnc(-n4ccc(=O)cc4)nc3)c3cncc(C(N)=O)c32)C1] | (R)-1-(1-acryloylpiperidin-3-yl)-3- (2-(4-oxopyridin-1(4H)-yl)pyrimidin- 5-yl)-1H-pyrazolo[4,3-c]pyridin-7- carboxamide | BCD-BTK-98 |
| 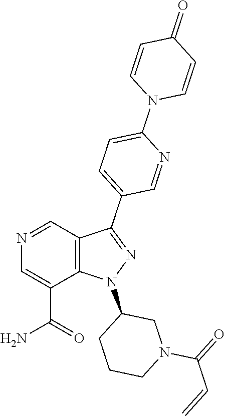 [SMILES: C=CC(=O)N1CCC[C@@H](n2nc(-c3ccc(-n4ccc(=O)cc4)nc3)c3cncc(C(N)=O)c32)C1] | (R)-1-(1-acryloylpiperidin-3-yl)-3- (4oxo-4H[1,2′-bipyridin]-5′-yl)-1H- pyrazolo[4,3-c]pyridin-7-carboxamide | BCD-BTK-100 |
| 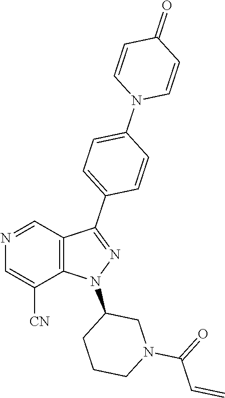 [SMILES: C=CC(=O)N1CCC[C@@H](n2nc(-c3ccc(-n4ccc(=O)cc4)cc3)c3cncc(C#N)c32)C1] | (R)-1-(1-acryloylpiperidin-3-yl)-3- (4-(pyridin-4-yloxy)phenyl)-1H- pyrazolo[4,3-c]pyridin-7-carbonitrile | BCD-BTK-104 |
| 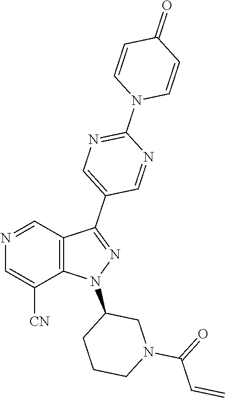 [SMILES: C=CC(=O)N1CCC[C@@H](n2nc(-c3cnc(-n4ccc(=O)cc4)nc3)c3cncc(C#N)c32)C1] | (R)-1-(1-acryloylpiperidin-3-yl)-3- (2-(4-oxopyridin-1(4H)-yl)pyrimidin- 5-yl)-1H-pyrazolo[4,3-c]pyridin-7- carbonitrile | BCD-BTK-105 |
| 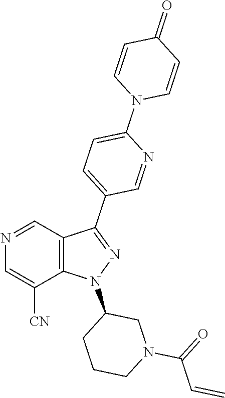 [SMILES: C=CC(=O)N1CCC[C@@H](n2nc(-c3ccc(-n4ccc(=O)cc4)nc3)c3cncc(C#N)c32)C1] | (R)-1-(1-acryloylpiperidin-3-yl)-3- (4-oxo-4H[1,2′-bipyridin]-5′-yl)- 1H-pyrazolo[4,3-c]pyridin-7- carbonitrile | BCD-BTK-107 |
| 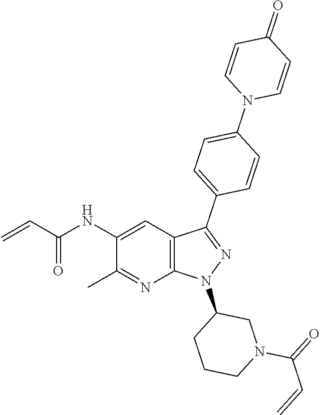 [SMILES: C=CC(=O)Nc1cc2c(-c3ccc(-n4ccc(=O)cc4)cc3)nn([C@@H]3CCCN(C(=O)C=C)C3)c2nc1C] | (R)-N-(1-(1-acryloylpiperidin-3-yl)- 6-methyl-3-(4-(4-oxopyridin-1(4H)- yl)phenyl)-1H-pyrazolo[3,4- b]pyridin5-yl)acrylamide | BCD-BTK-117 |
| 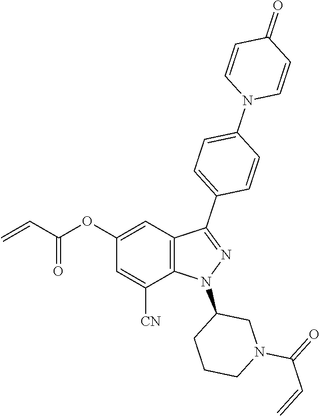 [SMILES: C=CC(=O)Oc1cc(C#N)c2c(c1)c(-c1ccc(-n3ccc(=O)cc3)cc1)nn2[C@@H]1CCCN(C(=O)C=C)C1] | (R)-1-(1-acryloylpiperidin-3-yl)-7- cyano-3-(4-(4-oxopyridin-1(4H)-yl) phenyl)-1H-indazol-5-yl acrylate | BCD-BTK-118 |
| 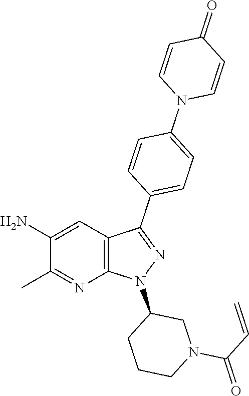 [SMILES: C=CC(=O)N1CCC[C@@H](n2nc(-c3ccc(-n4ccc(=O)cc4)cc3)c3cc(N)c(C)nc32)C1] | (R)-1-(4-(1-(1-acryloylpiperidin-3- yl)-5-amino-6-methyl-1H- pyrazolo[3,4-b]pyridin-3-yl)phenyl) pyridin-4(1H)-one | BCD-BTK-119 |
| 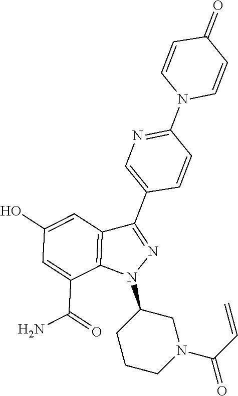 [SMILES: C=CC(=O)N1CCC[C@@H](n2nc(-c3ccc(-n4ccc(=O)cc4)nc3)c3cc(O)cc(C(N)=O)c32)C1] | (R)-1-(1-acryloylpiperidin-3-yl)-5- hydroxy-3-(4-oxo-4H-[1,2′- bipyridin]-5′-yl)-1H-indazol-7- carboxamide | BCD-BTK-120 |
| 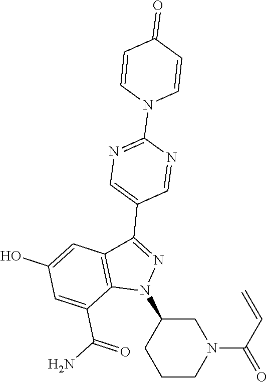 [SMILES: C=CC(=O)N1CCC[C@@H](n2nc(-c3cnc(-n4ccc(=O)cc4)nc3)c3cc(O)cc(C(N)=O)c32)C1] | (R)-1-(1-acryloylpiperidin-3-yl)-5- hydroxy-3-(2-(4-oxopyridin-1(4H)- yl)pyrimidin-5-yl)-1H-indazol-7- carboxamide | BCD-BTK-121 |
| 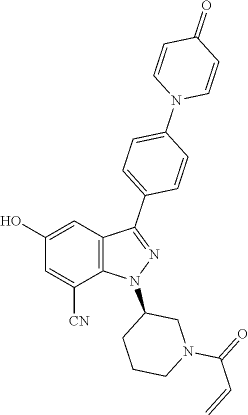 [SMILES: C=CC(=O)N1CCC[C@@H](n2nc(-c3ccc(-n4ccc(=O)cc4)cc3)c3cc(O)cc(C#N)c32)C1] | (R)-1-(1-acryloylpiperidin-3-yl)-5- hydroxy-3-(4-(4-oxopyridin-1(4H)- yl)phenyl)-1H-indazol-7-carbonitrile | BCD-BTK-122 |
| 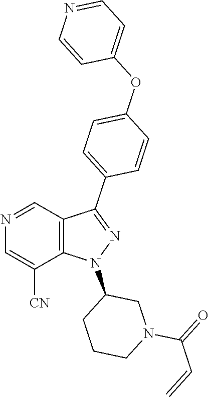 [SMILES: C=CC(=O)N1CCC[C@@H](n2nc(-c3ccc(Oc4ccncc4)cc3)c3cncc(C#N)c32)C1] | (R)-1-(1-acryloylpiperidin-3-yl)-3- (4(pyridin-4-yloxy)phenyl)-1H- pyrazolo[4,3-c]pyridin-7-carbonitrile | BCD-BTK-123 |
| 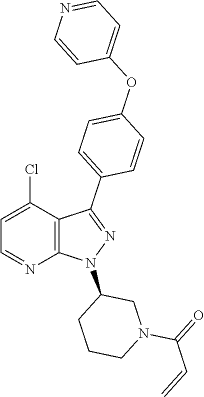 [SMILES: C=CC(=O)N1CCC[C@@H](n2nc(-c3ccc(Oc4ccncc4)cc3)c3c(Cl)ccnc32)C1] | (R)-1-(1-acryloylpiperidin-3-yl)-4- chloro-3-(4-(pyridin-4-yloxy)phenyl)- 1H-pyrazolo[3,4-b]pyridine | BCD-BTK-124 |
| 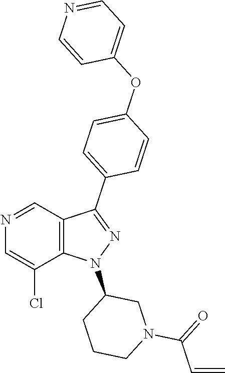 [SMILES: C=CC(=O)N1CCC[C@@H](n2nc(-c3ccc(Oc4ccncc4)cc3)c3cncc(Cl)c32)C1] | (R)-1-(1-acryloylpiperidin-3-yl)-7- chloro-3-(4-(pyridin-4-yloxy)phenyl)- 1H-pyrazolo[4,3-c]pyridine | BCD-BTK-125 |
| 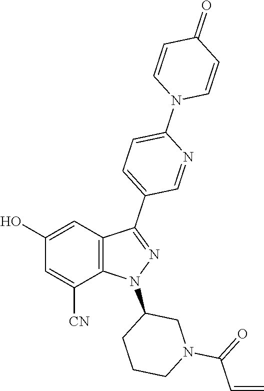 [SMILES: C=CC(=O)N1CCC[C@@H](n2nc(-c3ccc(-n4ccc(=O)cc4)nc3)c3cc(O)cc(C#N)c32)C1] | (R)-1-(1-acryloylpiperidin-3-yl)-5- hydroxy-3-(6-(4-oxopyridin-1(4H)- yl)pyridin-3-yl)-1H-indazol-7- carbonitrile | BCD-BTK-127 |
| 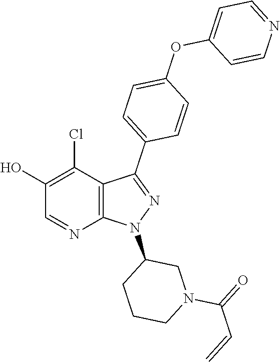 [SMILES: C=CC(=O)N1CCC[C@@H](n2nc(-c3ccc(Oc4ccncc4)cc3)c3c(Cl)c(O)cnc32)C1] | (R)-1-(1-acryloylpiperidin-3-yl)-5- hydroxy-4-chloro-3-(4-(pyridin-4- yloxy)phenyl)-1H-pyrazolo[3,4- b]pyridine | BCD-BTK-129 |
| 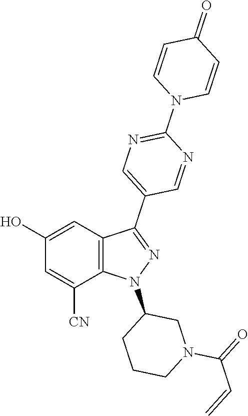 [SMILES: C=CC(=O)N1CCC[C@@H](n2nc(-c3cnc(-n4ccc(=O)cc4)nc3)c3cc(O)cc(C#N)c32)C1] | (R)-1-(1-acryloylpiperidin-3-yl)-5- hydroxy-3-(2-(4-oxopyridin-1(4H)- yl)pyrimidin-5-yl)-1H-indazol-7- carbonitrile | BCD-BTK-130 |
| 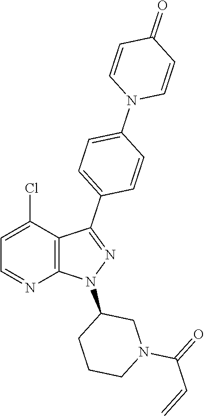 [SMILES: C=CC(=O)N1CCC[C@@H](n2nc(-c3ccc(-n4ccc(=O)cc4)cc3)c3c(Cl)ccnc32)C1] | (R)-1-(4-(1-(1-acryloylpiperidin-3- yl)-4-chloro-1H-pyrazolo[3,4- b]pyridin-3-yl)phenyl)pyridin-4(1H)- one | BCD-BTK-131 |
| 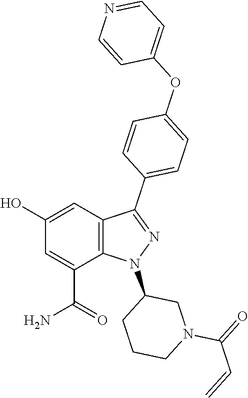 [SMILES: C=CC(=O)N1CCC[C@@H](n2nc(-c3ccc(Oc4ccncc4)cc3)c3cc(O)cc(C(N)=O)c32)C1] | (R)-1-(1-acryloylpiperidin-3-yl)-5- hydroxy-3-(4-pyridin-4-yloxy)phenyl)- 1H-indazol-7-carboxamide | BCD-BTK-133 |
| 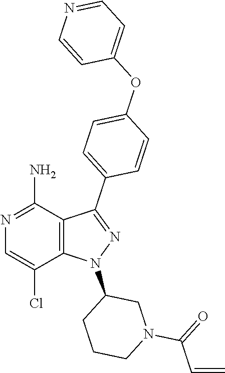 [SMILES: C=CC(=O)N1CCC[C@@H](n2nc(-c3ccc(Oc4ccncc4)cc3)c3c(N)ncc(Cl)c32)C1] | (R)-1-(1-acryloylpiperidin-3-yl)-4- amino-3-(4-(pyridin-4-yloxy)phenyl)- 1H-pyrazolo[4,3-c]pyridin-7- carbonitrile | BCD-BTK-134 |
| 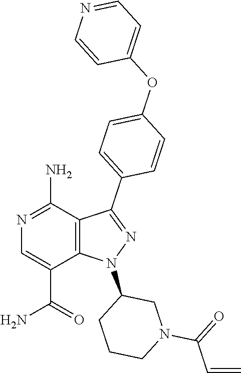 [SMILES: C=CC(=O)N1CCC[C@@H](n2nc(-c3ccc(Oc4ccncc4)cc3)c3c(N)ncc(C(N)=O)c32)C1] | (R)-1-(1-acryloylpiperidin-3-yl)-4- amino-3-(4-(pyridin-4-yloxy)phenyl)- 1H-pyrazolo[4,3-c]pyridin-7- carboxamide | BCD-BTK-135 |
| 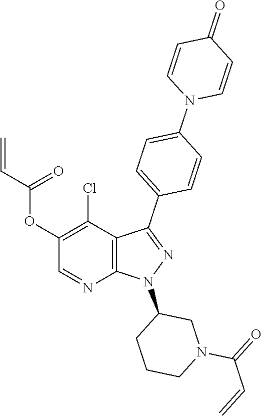 [SMILES: C=CC(=O)Oc1cnc2c(c(-c3ccc(-n4ccc(=O)cc4)cc3)nn2[C@@H]2CCCN(C(=O)C=C)C2)c1Cl] | (R)-1-(1-acryloylpiperidin-3-yl)-4- chloro-3-(4-(4-oxopyridin-1(4H) phenyl)-1H-pyrazolo[3,4-b]pyridin- 5-yl acrylate | BCD-BTK-136 |
| 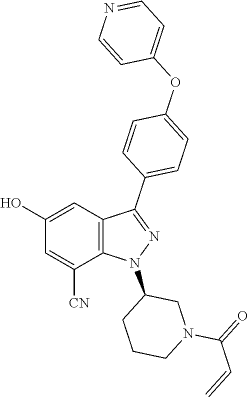 [SMILES: C=CC(=O)N1CCC[C@@H](n2nc(-c3ccc(Oc4ccncc4)cc3)c3cc(O)cc(C#N)c32)C1] | (R)-1-(1-acryloylpiperidin-3-yl)-5- hydroxy-3-(4-pyridin-4-yloxy)phenyl)- 1H-indazol-7-carbonitrile | BCD-BTK-137 |
| 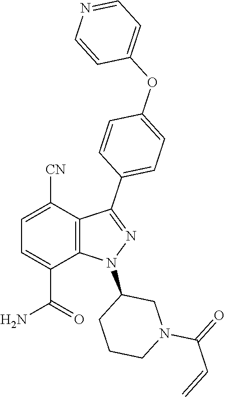 [SMILES: C=CC(=O)N1CCC[C@@H](n2nc(-c3ccc(Oc4ccncc4)cc3)c3c(C#N)ccc(C(N)=O)c32)C1] | (R)-1-(1-acryloylpiperidin-3-yl)-4- cyano-3-(4-(pyridin-4-yloxy)phenyl)- 1H-indazol-7-carboxamide | BCD-BTK-138 |
| 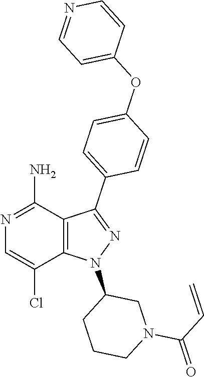 [SMILES: C=CC(=O)N1CCC[C@@H](n2nc(-c3ccc(Oc4ccncc4)cc3)c3c(N)ncc(Cl)c32)C1] | (R)-1-(1-acryloylpiperidin-3-yl)-7- chloro-3-(4-(pyridin-4-yloxy)phenyl)- 1H-pyrazolo[4,3-c]pyridin-4-amine | BCD-BTK-139 |
| 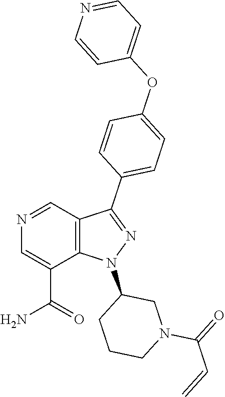 [SMILES: C=CC(=O)N1CCC[C@@H](n2nc(-c3ccc(Oc4ccncc4)cc3)c3cncc(C(N)=O)c32)C1] | (R)-1-(1-acryloylpiperidin-3-yl)-3- (4-(pyridin-4-yloxy)phenyl)-1H- pyrazolo[4,3-c]pyridin-7-carboxamide | BCD-BTK-140 |
| 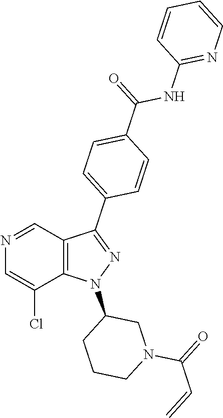 [SMILES: C=CC(=O)N1CCC[C@@H](n2nc(-c3ccc(C(=O)Nc4ccccn4)cc3)c3cncc(Cl)c32)C1] | (R)-4-(1-(1-(1-acryloylpiperidin-3- yl)-7-chloro-1H-pyrazolo[4,3- c]pyridin-3-yl)-N-(pyridin-2-yl) benzamide | BCD-BTK-201 |
| 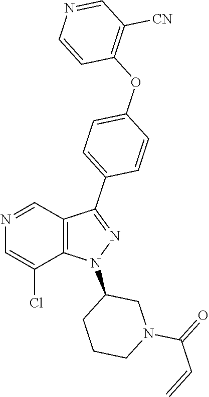 [SMILES: C=CC(=O)N1CCC[C@@H](n2nc(-c3ccc(Oc4ccncc4C#N)cc3)c3cncc(Cl)c32)C1] | (R)-4-(4-(1-(1-acryloylpiperidin-3- yl)-7-chloro-1H-pyrazolo[4,3- c]pyridin-3-yl)phenoxy)nicotinonitrile | BCD-BTK-202 |
| 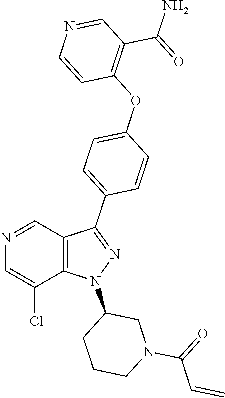 [SMILES: C=CC(=O)N1CCC[C@@H](n2nc(-c3ccc(Oc4ccncc4C(N)=O)cc3)c3cncc(Cl)c32)C1] | (R)-4-(4-(1-(1-acryloylpiperidin-3- yl)-7-chloro-1H-pyrazolo[4,3-c]pyridin- 3-yl)phenoxy)nicotinamide | BCD-BTK-203 |
| 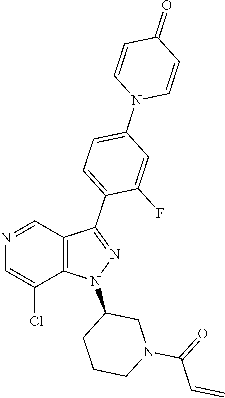 [SMILES: C=CC(=O)N1CCC[C@@H](n2nc(-c3ccc(-n4ccc(=O)cc4)cc3F)c3cncc(Cl)c32)C1] | (R)-1-(4-(1-(1-acryloylpiperidin-3- yl)-7-chloro-1H-pyrazolo[4,3-c]pyridin- 3-yl)-3-fluorophenyl)pyridin-4(1H)- one | BCD-BTK-204 |
| 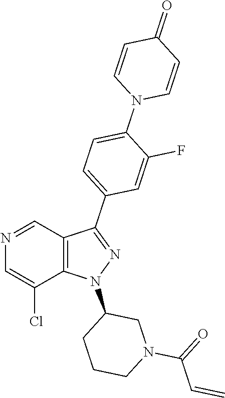 [SMILES: C=CC(=O)N1CCC[C@@H](n2nc(-c3ccc(-n4ccc(=O)cc4)c(F)c3)c3cncc(Cl)c32)C1] | (R)-1-(4-(1-(1-acryloylpiperidin-3- yl)-7-chloro-1H-pyrazolo[4,3-c]pyridin- 3-yl)-2-fluorophenyl)pyridin-4(1H)- one | BCD-BTK-205 |
| 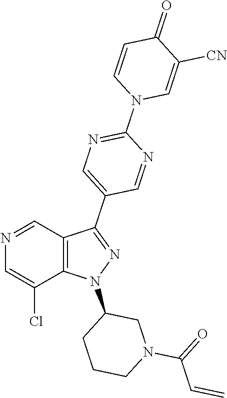 [SMILES: C=CC(=O)N1CCC[C@@H](n2nc(-c3cnc(-n4ccc(=O)c(C#N)c4)nc3)c3cncc(Cl)c32)C1] | (R)-1-(5-(1-(1-acryloylpiperidin-3- yl)-7-chloro-1H-pyrazolo[4,3-c]pyridin- 3-yl)pyrimidin-2-yl)-4-oxo-1,4- dihydropyridine-3-carbonitrile | BCD-BTK-206 |
| 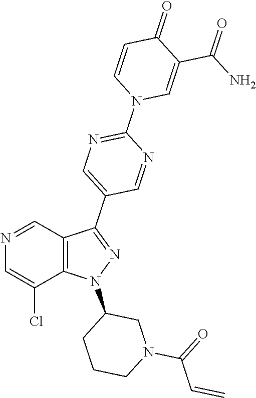 [SMILES: C=CC(=O)N1CCC[C@@H](n2nc(-c3cnc(-n4ccc(=O)c(C(N)=O)c4)nc3)c3cncc(Cl)c32)C1] | (R)-1-(5-(1-(1-acryloylpiperidin-3- yl)-7-chloro-1H-pyrazolo[4,3-c]pyridin- 3-yl)pyrimidin-2-yl)-4-oxo-1,4- dihydropyridine-3-carboxamide | BCD-BTK-207 |
| 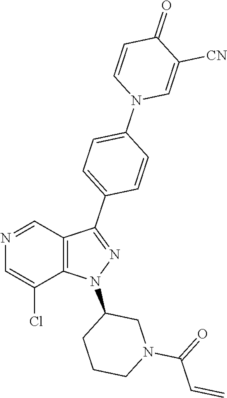 [SMILES: C=CC(=O)N1CCC[C@@H](n2nc(-c3ccc(-n4ccc(=O)c(C#N)c4)cc3)c3cncc(Cl)c32)C1] | (R)-1-(4-(1-(1-acryloylpiperidin-3- yl)-7-chloro-1H-pyrazolo[4,3-c]pyridin- 3-yl)phenyl)-4-oxo-1,4-dihydropyridine- 3-carbonitrile | BCD-BTK-208 |
| 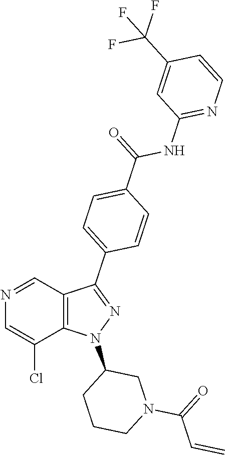 [SMILES: C=CC(=O)N1CCC[C@@H](n2nc(-c3ccc(C(=O)Nc4cc(C(F)(F)F)ccn4)cc3)c3cncc(Cl)c32)C1] | (R)-4-(1-(1-acryloylpiperidin-3-yl)- 7-chloro-1H-pyrazolo[4,3-c]pyridin- 3-yl)-N-(4-(trifluoromethyl)pyridin-2- yl)benzamide | BCD-BTK-210 |
| 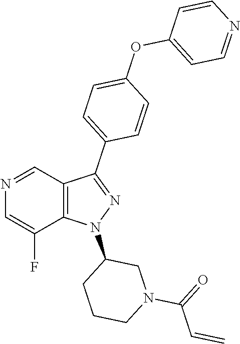 [SMILES: C=CC(=O)N1CCC[C@@H](n2nc(-c3ccc(Oc4ccncc4)cc3)c3cncc(F)c32)C1] | (R)-1-(3-(7-fluoro-3-(4-(pyridin-4- yloxy)phenyl)-1H-pyrazolo[4,3- c]pyridin-1-yl)piperidin-1-yl)prop-2- en-1-one | BCD-BTK-211 |
| 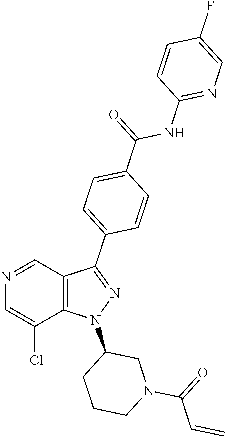 [SMILES: C=CC(=O)N1CCC[C@@H](n2nc(-c3ccc(C(=O)Nc4ccc(F)cn4)cc3)c3cncc(Cl)c32)C1] | (R)-4-(1-(1-acryloylpiperidin-3-yl)-7- chloro-1H-pyrazolo[4,3-c]pyridin-3- yl)-N-(5-fluoropyridin-2-yl)benzamide | BCD-BTK-212 |
| 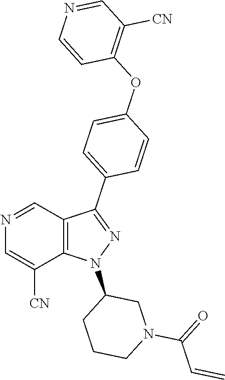 [SMILES: C=CC(=O)N1CCC[C@@H](n2nc(-c3ccc(Oc4ccncc4C#N)cc3)c3cncc(C#N)c32)C1] | (R)-1-(1-acryloylpiperidin-3-yl)-3- (4-(3-cyanopyridin-4-yloxy)phenyl)- 1H-pyrazolo[4,3-c]pyridine-7- carbonitrile | BCD-BTK-213 |
| 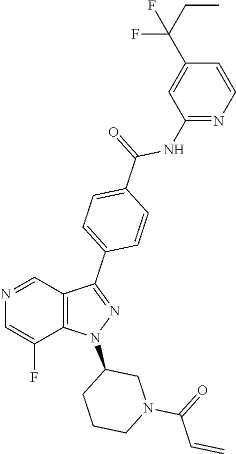 [SMILES: C=CC(=O)N1CCC[C@@H](n2nc(-c3ccc(C(=O)Nc4cc(C(F)(F)CC)ccn4)cc3)c3cncc(F)c32)C1] | (R)-4-(1-(1-acryloylpiperidin-3-yl)-7- fluoro-1H-pyrazolo[4,3-c]pyridin-3- yl)-N-(4-(1,1-difluoropropyl)pyridin- 2-yl)benzamide | BCD-BTK-214 |
| 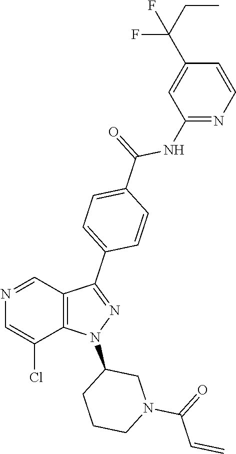 [SMILES: C=CC(=O)N1CCC[C@@H](n2nc(-c3ccc(C(=O)Nc4cc(C(F)(F)CC)ccn4)cc3)c3cncc(Cl)c32)C1] | (R)-4-(1-(1-acryloylpiperidin-3-yl)-7- chloro-1H-pyrazolo[4,3-c]pyridin-3- yl)-N-(4-(1,1-difluoropropyl)pyridin- 2-yl)benzamide | BCD-BTK-215 |
| 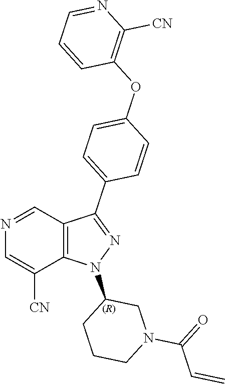 | (R)-1-(1-acryloylpiperidin-3-yl)-3- (4-(2-cyanopyridin-3-yloxy)phenyl)- 1H-pyrazolo[4,3-c]pyridine-7- carbonitrile | BCD-BTK-216 |
| 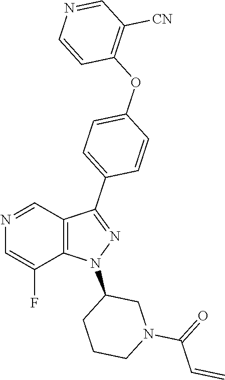 [SMILES: C=CC(=O)N1CCC[C@@H](n2nc(-c3ccc(Oc4ccncc4C#N)cc3)c3cncc(F)c32)C1] | (R)-4-(4-(1-(1-acryloylpiperidin-3- yl)-7-fluoro-1H-pyrazolo[4,3-c]pyridin- 3-yl)phenoxy)nicotinonitrile | BCD-BTK-217 |
| 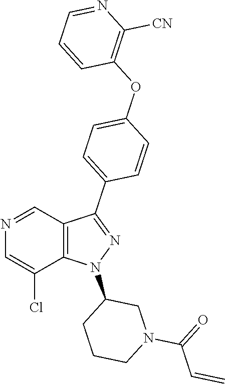 [SMILES: C=CC(=O)N1CCC[C@@H](n2nc(-c3ccc(Oc4cccnc4C#N)cc3)c3cncc(Cl)c32)C1] | 3-(4-{7-chloro-1-[1-(prop-2- enoyl)piperidin-3-yl]-1H- pyrazolo[4,3-c]pyridin-3-yl} phenoxy)pyridine-2-carbonitrile | BCD-BTK-218 |
| 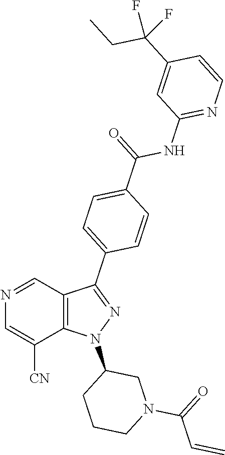 [SMILES: C=CC(=O)N1CCC[C@@H](n2nc(-c3ccc(C(=O)Nc4cc(C(F)(F)CC)ccn4)cc3)c3cncc(C#N)c32)C1] | 4-{7-cyano-1-[1-(prop-2-enoyl) piperidin-3-yl]-1H-pyrazolo[4,3- c]pyridin-3-yl}-N-[4-(1,1- difluoropropyl)pyridin-2-yl] benzamide | BCD-BTK-219 |
| 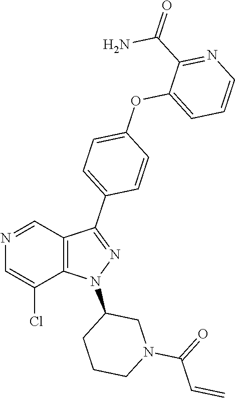 [SMILES: C=CC(=O)N1CCC[C@@H](n2nc(-c3ccc(Oc4cccnc4C(N)=O)cc3)c3cncc(Cl)c32)C1] | 3-(4-{7-chloro-1-[(3R)-1-(prop-2- enoyl)piperidin-3-yl]-1H- pyrazolo[4,3-c]pyridin-3- yl}phenoxy)pyridine-2-carboxamide | BCD-BTK-220 |
| 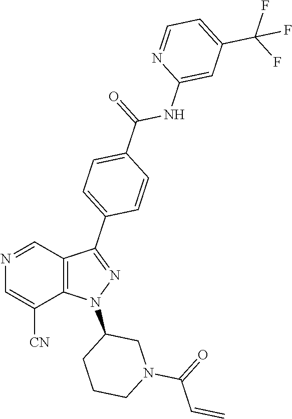 [SMILES: C=CC(=O)N1CCC[C@@H](n2nc(-c3ccc(C(=O)Nc4cc(C(F)(F)F)ccn4)cc3)c3cncc(C#N)c32)C1] | 4-{7-cyano-1-[1-(prop-2- enoyl)piperidin-3-yl]-1H- pyrazolo[4,3-c]pyridin-3-yl}-N-[4- (trifluoromethyl)pyridin-2- yl]benzamide | BCD-BTK-221 |
| 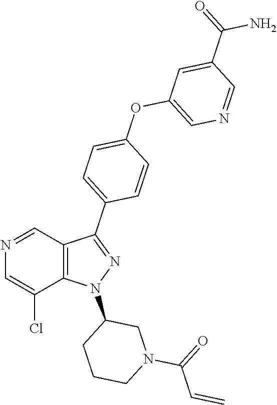 [SMILES: C=CC(=O)N1CCC[C@@H](n2nc(-c3ccc(Oc4cncc(C(N)=O)c4)cc3)c3cncc(Cl)c32)C1] | 5-(4-{7-chloro-1-[(3R)-1-(prop-2- enoyl)piperidin-3-yl]-1H- pyrazolo[4,3-c]pyridin-3- yl}phenoxy)pyridine-3-carboxamide | BCD-BTK-222 |
| 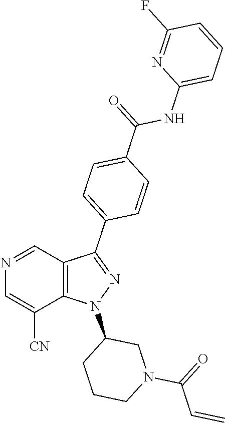 [SMILES: C=CC(=O)N1CCC[C@@H](n2nc(-c3ccc(C(=O)Nc4cccc(F)n4)cc3)c3cncc(C#N)c32)C1] | 4-{7-cyano-1-[(3R)-1-(prop-2- enoyl)piperidin-3-yl]-1H- pyrazolo[4,3-c]pyridin-3-yl}-N- (6-fluoropyridin-2-yl)benzamide | BCD-BTK-223 |
| 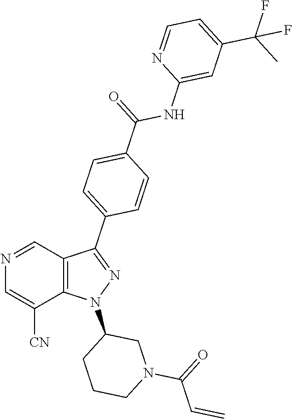 [SMILES: C=CC(=O)N1CCC[C@@H](n2nc(-c3ccc(C(=O)Nc4cc(C(C)(F)F)ccn4)cc3)c3cncc(C#N)c32)C1] | 4-{7-cyano-1-[(3R)-1-(prop-2- enoyl)piperidin-3-yl]-1H- pyrazolo[4,3-c]pyridin-3-yl}-N- [4-(1,1-difluoroethyl)pyridin-2- yl]benzamide | BCD-BTK-224 |
| 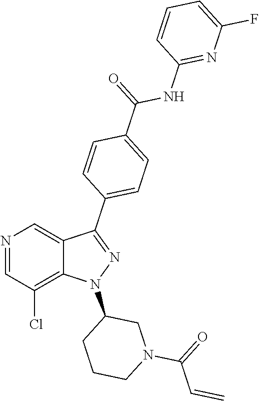 [SMILES: C=CC(=O)N1CCC[C@@H](n2nc(-c3ccc(C(=O)Nc4cccc(F)n4)cc3)c3cncc(Cl)c32)C1] | (R)-4-(1-(1-acryloylpiperidin-3-yl)- 7-chloro-1H-pyrazolo[4,3-c]pyridin- 3-yl)-N-(6-fluoropyridin-2- yl)benzamide | BCD-BTK-225 |
| 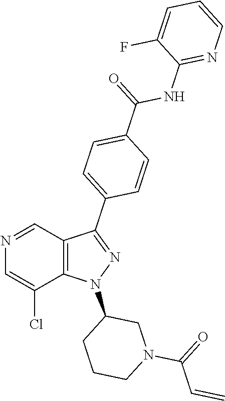 [SMILES: C=CC(=O)N1CCC[C@@H](n2nc(-c3ccc(C(=O)Nc4ncccc4F)cc3)c3cncc(Cl)c32)C1] | (R)-4-(1-(1-acryloylpiperidin-3-yl)- 7-chloro-1H-pyrazolo[4,3-c]pyridin- 3-yl)-N-(3-fluoropyridin-2- yl)benzamide | BCD-BTK-226 |
| 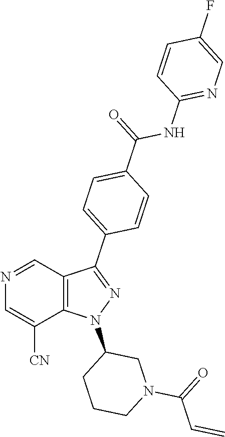 [SMILES: C=CC(=O)N1CCC[C@@H](n2nc(-c3ccc(C(=O)Nc4ccc(F)cn4)cc3)c3cncc(C#N)c32)C1] | (R)-4-(1-(1-acryloylpiperidin-3-yl)- 7-cyano-1H-pyrazolo[4,3-c]pyridin- 3-yl)-N-(5-fluoropyridin-2- yl)benzamide | BCD-BTK-227 |
| 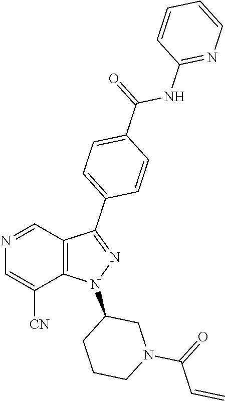 [SMILES: C=CC(=O)N1CCC[C@@H](n2nc(-c3ccc(C(=O)Nc4ccccn4)cc3)c3cncc(C#N)c32)C1] | (R)-4-(1-(1-acryloylpiperidin-3-yl)- 7-cyano-1H-pyrazolo[4,3-c]pyridin- 3-yl)-N-(pyridin-2-yl)benzamide | BCD-BTK-228 |
| 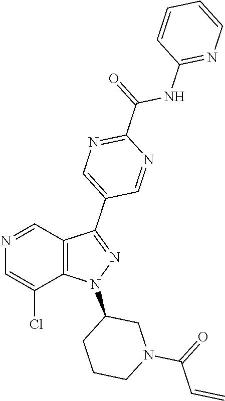 [SMILES: C=CC(=O)N1CCC[C@@H](n2nc(-c3cnc(C(=O)Nc4ccccn4)nc3)c3cncc(Cl)c32)C1] | (R)-5-(1-(1-acryloylpiperidin-3-yl)- 7-chloro-1H-pyrazolo[4,3-c]pyridin- 3-yl)-N-(pyridin-2-yl)pyrimidine-2- carboxamide | BCD-BTK-229 |
| 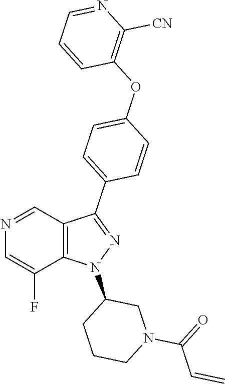 [SMILES: C=CC(=O)N1CCC[C@@H](n2nc(-c3ccc(Oc4cccnc4C#N)cc3)c3cncc(F)c32)C1] | (R)-3-(4-(1-(1-acryloylpiperidin-3- yl)-7-fluoro-1H-pyrazolo[4,3- c]pyridin-3-yl)phenoxy)picolinonitrile | BCD-BTK-230 |
| 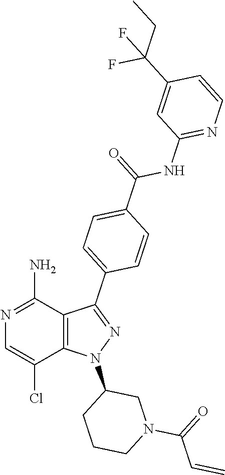 [SMILES: C=CC(=O)N1CCC[C@@H](n2nc(-c3ccc(C(=O)Nc4cc(C(F)(F)CC)ccn4)cc3)c3c(N)ncc(Cl)c32)C1] | (R)-4-(1-(1-acryloylpiperidin-3-yl)-4- amino-7-chloro-1H-pyrazolo[4,3- c]pyridin-3-yl)-N-(4-(1,1- difluoropropyl)pyridin-2-yl)benzamide | BCD-BTK-231 |
| 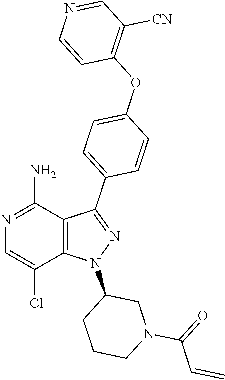 [SMILES: C=CC(=O)N1CCC[C@@H](n2nc(-c3ccc(Oc4ccncc4C#N)cc3)c3c(N)ncc(Cl)c32)C1] | (R)-4-(4-(1-(1-acryloylpiperidin-3- yl)-4-amino-7-chloro-1H- pyrazolo[4,3-c]pyridin-3- yl)phenoxy)nicotinonitrile | BCD-BTK-232 |
| 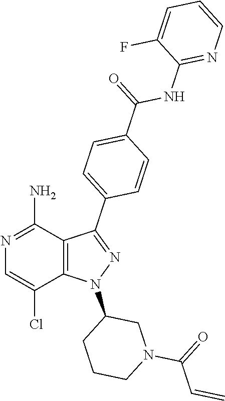 [SMILES: C=CC(=O)N1CCC[C@@H](n2nc(-c3ccc(C(=O)Nc4ncccc4F)cc3)c3c(N)ncc(Cl)c32)C1] | (R)-4-(1-(1-acryloylpiperidin-3-yl)-4- amino-7-chloro-1H-pyrazolo[4,3- c]pyridin-3-yl)-N-(3-fluoropyridin- 2-yl)benzamide | BCD-BTK-233 |
| 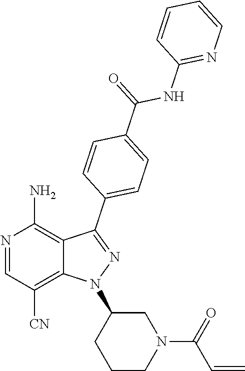 [SMILES: C=CC(=O)N1CCC[C@@H](n2nc(-c3ccc(C(=O)Nc4ccccn4)cc3)c3c(N)ncc(C#N)c32)C1] | (R)-4-(1-(1-acryloylpiperidin-3-yl)- 4-amino-7-cyano-1H-pyrazolo[4,3- c]pyridin-3-yl)-N-(pyridine-2- yl)benzamide | BCD-BTK-234 |
| 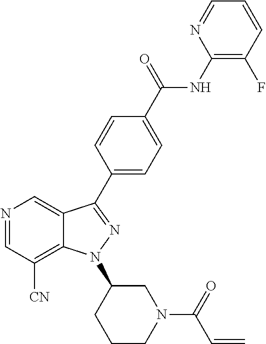 [SMILES: C=CC(=O)N1CCC[C@@H](n2nc(-c3ccc(C(=O)Nc4ncccc4F)cc3)c3cncc(C#N)c32)C1] | 4-{7-cyano-1-[(3R)-1-(prop-2- enoyl)piperidin-3-yl]-1H- pyrazolo[4,3-c]pyridin-3-yl}-N-(3- fluoropyridin-2-yl)benzamide | BCD-BTK-235 |
| 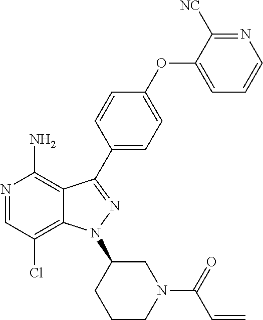 [SMILES: C=CC(=O)N1CCC[C@@H](n2nc(-c3ccc(Oc4cccnc4C#N)cc3)c3c(N)ncc(Cl)c32)C1] | 3-(4-{4-amino-7-chloro-1-[(3R)-1- (prop-2-enoyl)piperidin-3-yl]-1H- pyrazolo[4,3-c]pyridin-3-yl}phenoxy) pyridine-2-carbonitrile | BCD-BTK-236 |
| 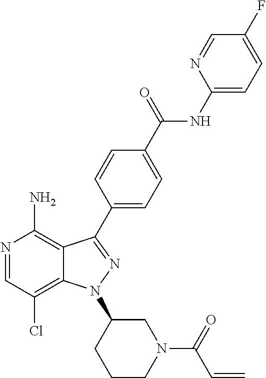 [SMILES: C=CC(=O)N1CCC[C@@H](n2nc(-c3ccc(C(=O)Nc4ccc(F)cn4)cc3)c3c(N)ncc(Cl)c32)C1] | 4-{4-amino-7-chloro-1-[(3R)-1- (prop-2-enoyl)piperidin-3-yl]-1H- pyrazolo[4,3-c]pyridin-3-yl}-N-(5- fluoropyridin-2-yl)benzamide | BCD-BTK-237 |
| 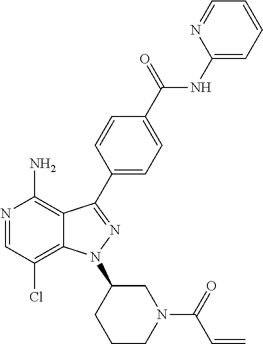 [SMILES: C=CC(=O)N1CCC[C@@H](n2nc(-c3ccc(C(=O)Nc4ccccn4)cc3)c3c(N)ncc(Cl)c32)C1] | 4-{4-amino-7-chloro-1-[(3R)-1- (prop-2-enoyl)piperidin-3-yl]-1H- pyrazolo[4,3-c]pyridin-3-yl}-N- (pyridin-2-yl)benzamide | BCD-BTK-238 |
| 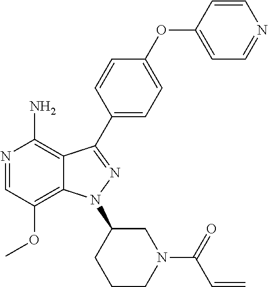 [SMILES: C=CC(=O)N1CCC[C@@H](n2nc(-c3ccc(Oc4ccncc4)cc3)c3c(N)ncc(OC)c32)C1] | (R)-1-(1-acryloylpiperidin-3-yl)-4- amine-7-methoxy-3-(4-(pyridin-4- yloxy)phenyl)-1H-pyrazolo[4,3- c]pyridine | BCD-BTK-239 |
| 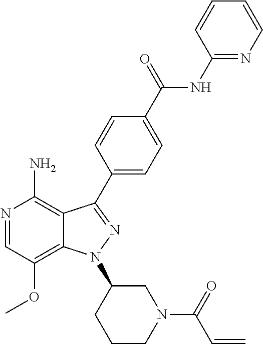 [SMILES: C=CC(=O)N1CCC[C@@H](n2nc(-c3ccc(C(=O)Nc4ccccn4)cc3)c3c(N)ncc(OC)c32)C1] | (R)-4-(1-(1-acryloylpiperidin-3-yl)-4- amino-7-methoxy-1H-pyrazolo[4,3- c]pyridin-3-yl)-N-(pyridin-2- yl)benzamide | BCD-BTK-240 |
| 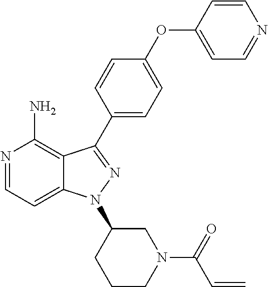 [SMILES: C=CC(=O)N1CCC[C@@H](n2nc(-c3ccc(Oc4ccncc4)cc3)c3c(N)nccc32)C1] | (R)-1-(1-acryloylpiperidin-3-yl)-4- amine-3-(4-(pyridin-4- yloxy)phenyl)-1H-pyrazolo[4,3- c]pyridine | BCD-BTK-241 |
| 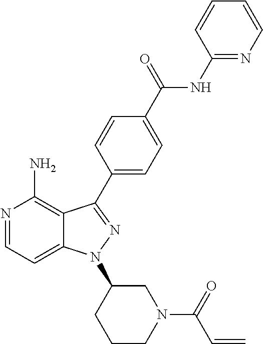 [SMILES: C=CC(=O)N1CCC[C@@H](n2nc(-c3ccc(C(=O)Nc4ccccn4)cc3)c3c(N)nccc32)C1] | (R)-4-(1-(1-acryloylpiperidin-3-yl)-4- amino-1H-pyrazolo[4,3-c]pyridin- 3-yl)-N-(pyridin-2-yl)benzamide | BCD-BTK-242 |
| 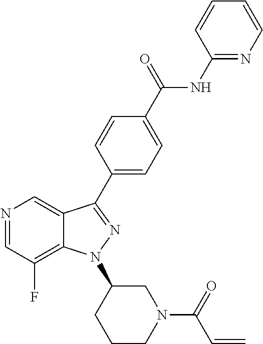 [SMILES: C=CC(=O)N1CCC[C@@H](n2nc(-c3ccc(C(=O)Nc4ccccn4)cc3)c3cncc(F)c32)C1] | (R)-4-(1-(1-acryloylpiperidin-3-yl)-7- fluoro-1H-pyrazolo[4,3-c]pyridin-3- yl)-N-(pyridin-2-yl)benzamide | BCD-BTK-243 |
| 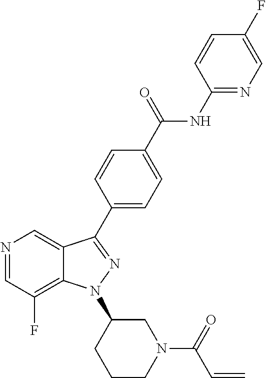 [SMILES: C=CC(=O)N1CCC[C@@H](n2nc(-c3ccc(C(=O)Nc4ccc(F)cn4)cc3)c3cncc(F)c32)C1] | (R)-4-(1-(1-acryloylpiperidin-3-yl)-7- fluoro-1H-pyrazolo[4,3-c]pyridin-3- yl)-N-(5-fluoropyridin-2-yl)benzamide | BCD-BTK-244 |
| 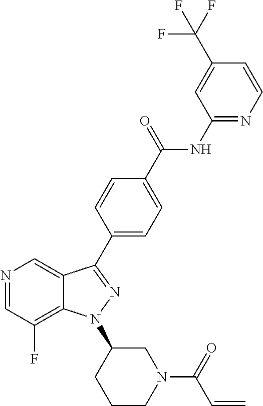 [SMILES: C=CC(=O)N1CCC[C@@H](n2nc(-c3ccc(C(=O)Nc4cc(C(F)(F)F)ccn4)cc3)c3cncc(F)c32)C1] | (R)-4-(1-(1-acryloylpiperidin-3-yl)-7- fluoro-1H-pyrazolo[4,3-c]pyridin-3- yl)-N-(4-(trifluoromethyl)pyridin-2- yl)benzamide | BCD-BTK-245 |
| 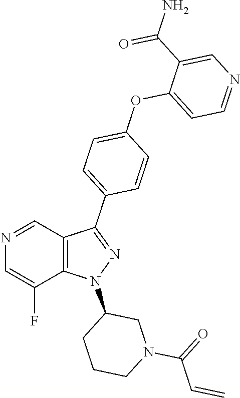 [SMILES: C=CC(=O)N1CCC[C@@H](n2nc(-c3ccc(Oc4ccncc4C(N)=O)cc3)c3cncc(F)c32)C1] | (R)-4-(4-(1-(1-acryloylpiperidin-3- yl)-7-fluoro-1H-pyrazolo[4,3- c]pyridin-3-yl)phenoxy)nicotinamide | BCD-BTK-246 |
| 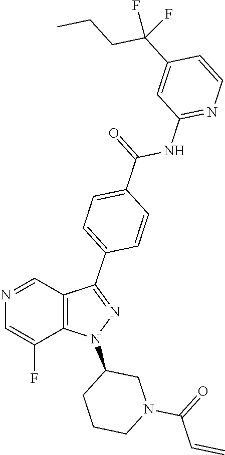 [SMILES: C=CC(=O)N1CCC[C@@H](n2nc(-c3ccc(C(=O)Nc4cc(C(F)(F)CCC)ccn4)cc3)c3cncc(F)c32)C1] | (R)-4-(1-(1-acryloylpiperidin-3-yl)-7- fluoro-1H-pyrazolo[4,3-c]pyridin-3- yl)-N-(4-(1,1-difluorobutyl)pyridin-2- yl)benzamide | BCD-BTK-247 |
| 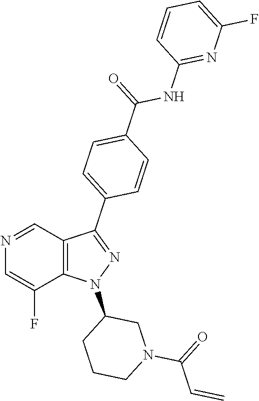 [SMILES: C=CC(=O)N1CCC[C@@H](n2nc(-c3ccc(C(=O)Nc4cccc(F)n4)cc3)c3cncc(F)c32)C1] | (R)-4-(1-(1-acryloylpiperidin-3-yl)-7- fluoro-1H-pyrazolo[4,3-c]pyridin-3- yl)-N-(6-fluoropyridin-2- yl)benzamide | BCD-BTK-248 |
| 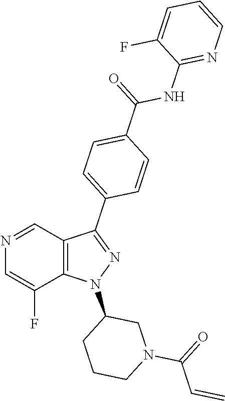 [SMILES: C=CC(=O)N1CCC[C@@H](n2nc(-c3ccc(C(=O)Nc4ncccc4F)cc3)c3cncc(F)c32)C1] | (R)-4-(1-(1-acryloylpiperidin-3-yl)-7- fluoro-1H-pyrazolo[4,3-c]pyridin-3- yl)-N-(3-fluoropyridin-2- yl)benzamide | BCD-BTK-249 |
| 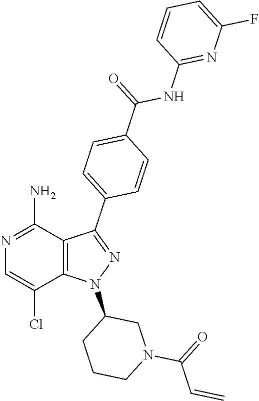 [SMILES: C=CC(=O)N1CCC[C@@H](n2nc(-c3ccc(C(=O)Nc4cccc(F)n4)cc3)c3c(N)ncc(Cl)c32)C1] | (R)-4-(1-(1-acryloylpiperidin-3-yl)-4- amino-7-chloro-1H-pyrazolo[4,3- c]pyridin-3-yl)-N-(6-fluoropyridin- 2-yl)benzamide | BCD-BTK-250 |
| 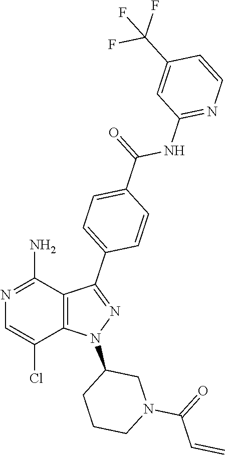 [SMILES: C=CC(=O)N1CCC[C@@H](n2nc(-c3ccc(C(=O)Nc4cc(C(F)(F)F)ccn4)cc3)c3c(N)ncc(Cl)c32)C1] | (R)-4-(1-(1-acryloylpiperidin-3-yl)-4- amino-7-chloro-1H-pyrazolo[4,3- c]pyridin-3-yl)-N-(4- (trifluoromethyl)pyridin-2- yl)benzamide | BCD-BTK-251 |
| 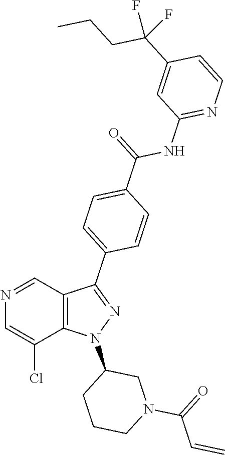 [SMILES: C=CC(=O)N1CCC[C@@H](n2nc(-c3ccc(C(=O)Nc4cc(C(F)(F)CCC)ccn4)cc3)c3cncc(Cl)c32)C1] | (R)-4-(1-(1-acryloylpiperidin-3-yl)- 7-chloro-1H-pyrazolo[4,3-c]pyridin- 3-yl)-N-(4-(1,1- difluorobutyl)pyridin-2- yl)benzamide | BCD-BTK-252 |
| 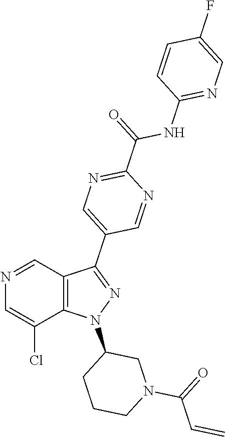 [SMILES: C=CC(=O)N1CCC[C@@H](n2nc(-c3cnc(C(=O)Nc4ccc(F)cn4)nc3)c3cncc(Cl)c32)C1] | (R)-5-(1-(1-acryloylpiperidin-3-yl)- 7-chloro-1H-pyrazolo[4,3-c]pyridin- 3-yl)-N-(5-fluoropyridin-2- yl)pyrimidine-2-carboxamide | BCD-BTK-253 |
| 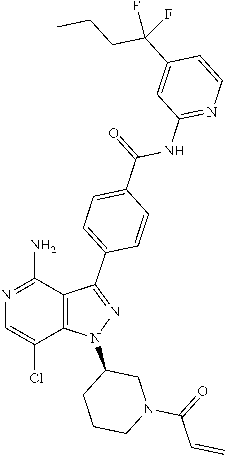 [SMILES: C=CC(=O)N1CCC[C@@H](n2nc(-c3ccc(C(=O)Nc4cc(C(F)(F)CCC)ccn4)cc3)c3c(N)ncc(Cl)c32)C1] | (R)-4-(1-(1-acryloylpiperidin-3-yl)- 4-amino-7-chloro-1H-pyrazolo[4,3- c]pyridin-3-yl)-N-(4-(1,1- difluorobutyl)pyridin-2- yl)benzamide | BCD-BTK-254 |
| 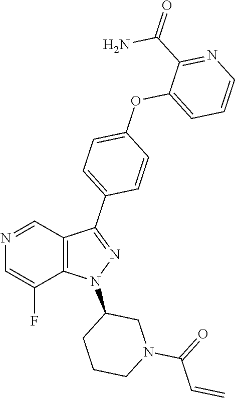 [SMILES: C=CC(=O)N1CCC[C@@H](n2nc(-c3ccc(Oc4cccnc4C(N)=O)cc3)c3cncc(F)c32)C1] | (R)-3-(4-(1-(1-acryloylpiperidin-3- yl)-7-fluoro-1H-pyrazolo[4,3- c]pyridin-3-yl)phenoxy)picolinamide | BCD-BTK-255 |
| 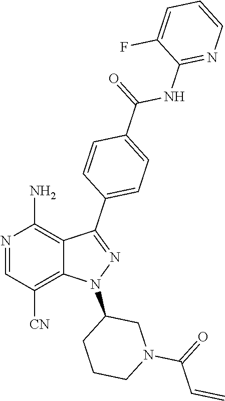 [SMILES: C=CC(=O)N1CCC[C@@H](n2nc(-c3ccc(C(=O)Nc4ncccc4F)cc3)c3c(N)ncc(C#N)c32)C1] | 4-{4-amino-7-cyano-1-[(3R)-1- (prop-2-enoyl)piperidin-3-yl]-1H- pyrazolo[4,3-c]pyridin-3-yl}-N-(3- fluoropyridin-2-yl)benzamide | BCD-BTK-258 |
| 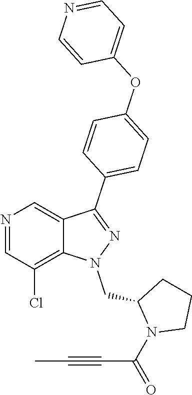 [SMILES: CC#CC(=O)N1CCC[C@H]1Cn1nc(-c2ccc(Oc3ccncc3)cc2)c2cncc(Cl)c21] | (S)-1-(2-((7-chloro-3-(4-(pyridin-4- yloxy)phenyl)-1H-pyrazolo[4,3- c]pyridin-1-yl)methyl)pyrrolidin-1- yl)but-2-yn-1-one | BCD-BTK-259 |
| 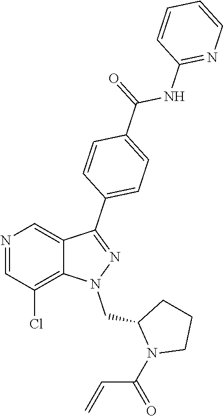 [SMILES: C=CC(=O)N1CCC[C@H]1Cn1nc(-c2ccc(C(=O)Nc3ccccn3)cc2)c2cncc(Cl)c21] | (S)-4-(1-((1-acryloylpyrrolidin-2- yl)methyl)-7-chloro-1H- pyrazolo[4,3-c]pyridin-3-yl)-N- phenylbenzamide | BCD-BTK-260 |
| 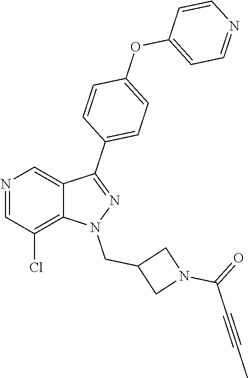 [SMILES: CC#CC(=O)N1CC(Cn2nc(-c3ccc(Oc4ccncc4)cc3)c3cncc(Cl)c32)C1] | 1-(3-((7-chloro-3-(4-(pyridin-4- yloxy)phenyl)-1H-pyrazolo[4,3- c]pyridin-1-yl)methyl)azetidin-1- yl)but-2-yn-1-one | BCD-BTK-261 |
| 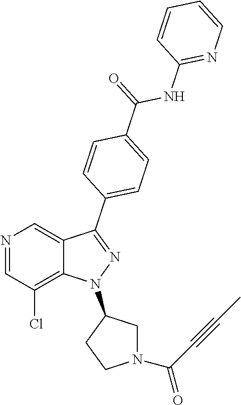 [SMILES: CC#CC(=O)N1CC[C@@H](n2nc(-c3ccc(C(=O)Nc4ccccn4)cc3)c3cncc(Cl)c32)C1] | (R)-4-(1-(1-(but-2-ynoyl)pyrrolidin- 3-yl)-7-chloro-1H-pyrazolo[4,3- c]pyridin-3-yl)-N-(pyridin-2- yl)benzamide | BCD-BTK-262 |
| 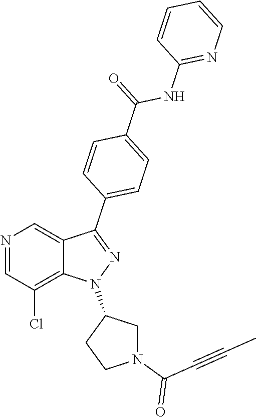 [SMILES: CC#CC(=O)N1CC[C@H](n2nc(-c3ccc(C(=O)Nc4ccccn4)cc3)c3cncc(Cl)c32)C1] | (S)-4-(1-(1-(but-2-ynoyl)pyrrolidin- 3-yl)-7-chloro-1H-pyrazolo[4,3- c]pyridin-3-yl)-N-(pyridin-2- yl)benzamide | BCD-BTK-271 |
| 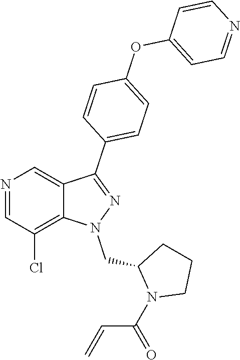 [SMILES: C=CC(=O)N1CCC[C@H]1Cn1nc(-c2ccc(Oc3ccncc3)cc2)c2cncc(Cl)c21] | (S)-1-(2-((7-chloro-3-(4-(pyridin-4- yloxy)phenyl)-1H-pyrazolo[4,3- c]pyridin-1-yl)methyl)pyrrolidin-1- yl)prop-2-en-1-one | BCD-BTK-263 |
| 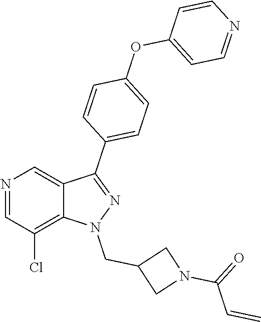 [SMILES: C=CC(=O)N1CC(Cn2nc(-c3ccc(Oc4ccncc4)cc3)c3cncc(Cl)c32)C1] | 1-(3-((7-chloro-3-(4-(pyridin-4- yloxy)phenyl)-1H-pyrazolo[4,3- c]pyridin-1-yl)methyl)azetidin-1- yl)prop-2-en-1-one | BCD-BTK-264 |
| 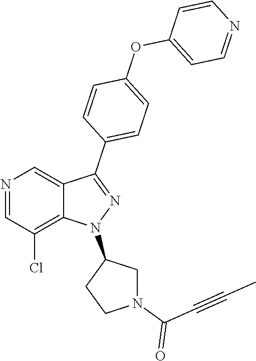 [SMILES: CC#CC(=O)N1CC[C@@H](n2nc(-c3ccc(Oc4ccncc4)cc3)c3cncc(Cl)c32)C1] | (R)-1-(3-(7-chloro-3-(4-(pyridin-4- yloxy)phenyl)-1H-pyrazolo[4,3- c]pyridin-1-yl)pyrrolidin-1-yl)but-2- yn-1-one | BCD-BTK-265 |
| 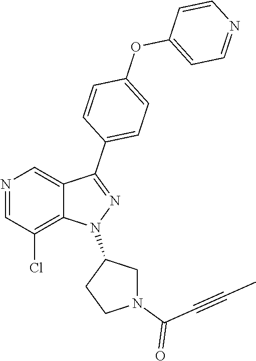 [SMILES: CC#CC(=O)N1CC[C@H](n2nc(-c3ccc(Oc4ccncc4)cc3)c3cncc(Cl)c32)C1] | (S)-1-(3-(7-chloro-3-(4-(pyridin-4- yloxy)phenyl)-1H-pyrazolo[4,3- c]pyridin-1-yl)pyrrolidin-1-yl)but-2- yn-1-one | BCD-BTK-270 |
| 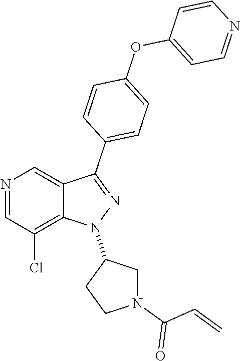 | (S)-1-(3-(7-chloro-3-(4-(pyridin-4- yloxy)phenyl)-1H-pyrazolo[4,3- c]pyridin-1-yl)pyrrolidin-1-yl)prop- 2-en-1-one | BCD-BTK-266 |
| 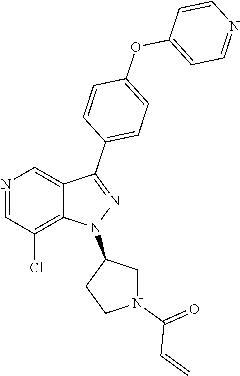 [SMILES: C=CC(=O)N1CC[C@@H](n2nc(-c3ccc(Oc4ccncc4)cc3)c3cncc(Cl)c32)C1] | (R)-1-(3-(7-chloro-3-(4-(pyridin-4- yloxy)phenyl)-1H-pyrazolo[4,3- c]pyridin-1-yl)pyrrolidin-1-yl)prop- 2-en-1-one | BCD-BTK-272 |
| 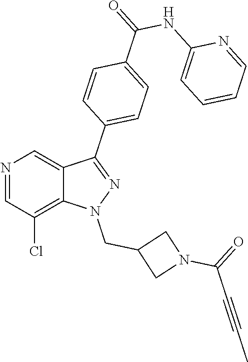 [SMILES: CC#CC(=O)N1CC(Cn2nc(-c3ccc(C(=O)Nc4ccccn4)cc3)c3cncc(Cl)c32)C1] | 4-(1-((1-(but-2-ynoyl)azetidin-3- yl)methyl)-7-chloro-1H-pyrazolo[4,3- c]pyridin-3-yl)-N-(pyridin-2- yl)benzamide | BCD-BTK-267 |
| 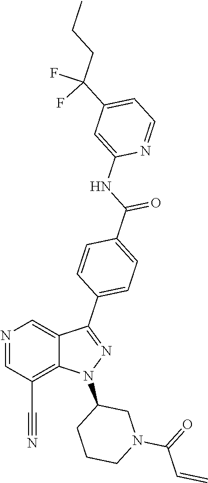 [SMILES: C=CC(=O)N1CCC[C@@H](n2nc(-c3ccc(C(=O)Nc4cc(C(F)(F)CCC)ccn4)cc3)c3cncc(C#N)c32)C1] | (R)-4-(1-(1-acryloylpiperidin-3-yl)- 7-cyano-1H-pyrazolo[4,3-c]pyridin- 3-yl)-N-(4-(1,1- difluorobutyl)pyridin-2-yl)benzamide | BCD-BTK-268 |
| 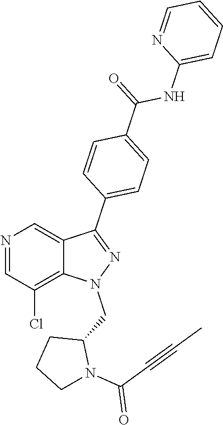 [SMILES: CC#CC(=O)N1CCC[C@@H]1Cn1nc(-c2ccc(C(=O)Nc3ccccn3)cc2)c2cncc(Cl)c21] | (R)-4-(1-((1-(but-2-ynoyl)pyrrolidin- 2-yl)methyl)-7-chloro-1H- pyrazolo[4,3-c]pyridin-3-yl)-N- (pyridin-2-yl)benzamide | BCD-BTK-269 |
| 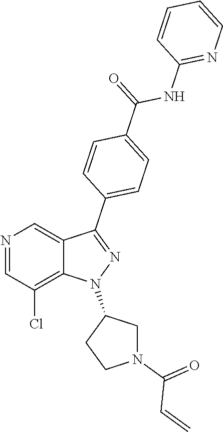 [SMILES: C=CC(=O)N1CC[C@H](n2nc(-c3ccc(C(=O)Nc4ccccn4)cc3)c3cncc(Cl)c32)C1] | (S)-4-(1-(1-acryloylpyrrolidin-3-yl)- 7-chloro-1H-pyrazolo[4,3-c]pyridin- 3-yl)-N-(pyridin-2-yl)benzamide | BCD-BTK-273 |
| 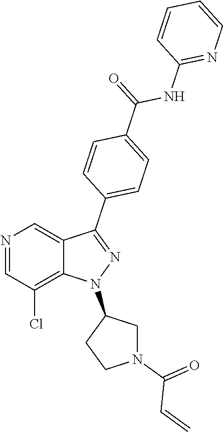 [SMILES: C=CC(=O)N1CC[C@@H](n2nc(-c3ccc(C(=O)Nc4ccccn4)cc3)c3cncc(Cl)c32)C1] | (R)-4-(1-(1-acryloylpyrrolidin-3-yl)- 7-chloro-1H-pyrazolo[4,3-c]pyridin- 3-yl)-N-(pyridin-2-yl)benzamide | BCD-BTK-275 |
| 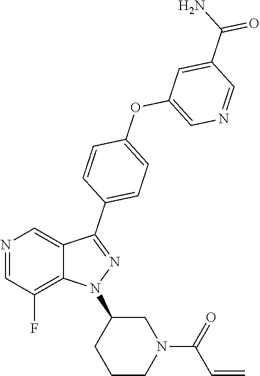 [SMILES: C=CC(=O)N1CCC[C@@H](n2nc(-c3ccc(Oc4cncc(C(N)=O)c4)cc3)c3cncc(F)c32)C1] | (R)-5-(4-(1-(1-acryloylpiperidin-3- yl)-7-fluoro-1H-pyrazolo[4,3- c]pyridin-3- yl)phenoxy)nicotinamide | BCD-BTK-274 |
| 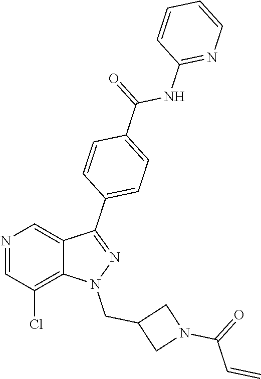 [SMILES: C=CC(=O)N1CC(Cn2nc(-c3ccc(C(=O)Nc4ccccn4)cc3)c3cncc(Cl)c32)C1] | 4-(7-chloro-1-{[1-(prop-2- enoyl)azetidin-3-yl]methyl}-1H- pyrazolo[4,3-c]pyridin-3-yl)-N- (pyridin-2-yl)benzamide | BCD-BTK-276 |
| 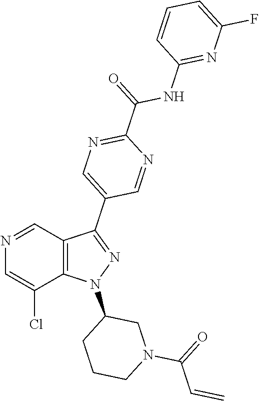 [SMILES: C=CC(=O)N1CCC[C@@H](n2nc(-c3cnc(C(=O)Nc4cccc(F)n4)nc3)c3cncc(Cl)c32)C1] | (R)-5-(1-(1-acryloylpiperidin-3-yl)- 7-chloro-1H-pyrazolo[4,3-c]pyridin- 3-yl)-N-(6-fluoropyridin-2- yl)pyrimidine-2-carboxamide | BCD-BTK-277 |
| 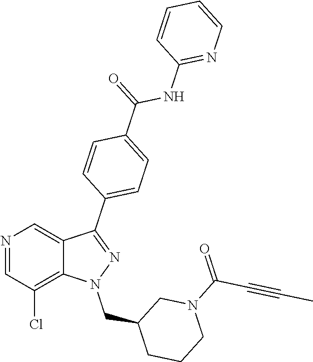 [SMILES: CC#CC(=O)N1CCC[C@@H](Cn2nc(-c3ccc(C(=O)Nc4ccccn4)cc3)c3cncc(Cl)c32)C1] | (R)-4-(1-((1-(but-2-ynoyl)piperidin- 3-yl)methyl)-7-chloro-1H- pyrazolo[4,3-c]pyridin-3-yl)-N- (pyridin-2-yl)benzamide | BCD-BTK-278 |
| 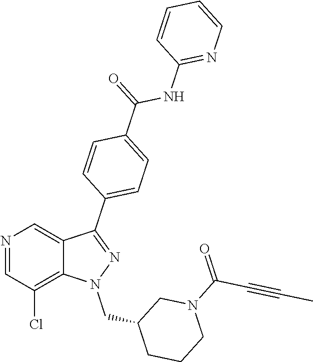 [SMILES: CC#CC(=O)N1CCC[C@H](Cn2nc(-c3ccc(C(=O)Nc4ccccn4)cc3)c3cncc(Cl)c32)C1] | (S)-4-(1-((1-(but-2-ynoyl)piperidin- 3-yl)methyl)-7-chloro-1H- pyrazolo[4,3-c]pyridin-3-yl)-N- (pyridin-2-yl)benzamide | BCD-BTK-283 |
| 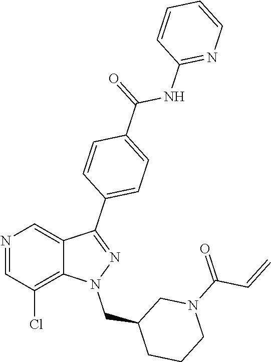 [SMILES: C=CC(=O)N1CCC[C@@H](Cn2nc(-c3ccc(C(=O)Nc4ccccn4)cc3)c3cncc(Cl)c32)C1] | (R)-4-(1-((1-acryloylpiperidin-3- yl)methyl)-7-chloro-1H- pyrazolo[4,3-c]pyridin-3-yl)-N- (pyridin-2-yl)benzamide | BCD-BTK-279 |
| 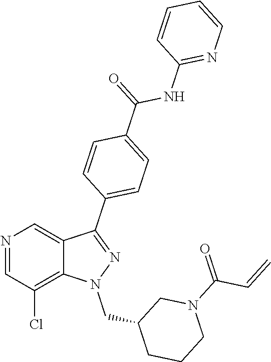 [SMILES: C=CC(=O)N1CCC[C@H](Cn2nc(-c3ccc(C(=O)Nc4ccccn4)cc3)c3cncc(Cl)c32)C1] | (S)-4-(1-((1-acryloylpiperidin-3- yl)methyl)-7-chloro-1H- pyrazolo[4,3-c]pyridin-3-yl)-N- (pyridin-2-yl)benzamide | BCD-BTK-290 |
| 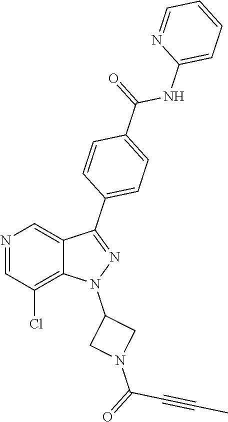 [SMILES: CC#CC(=O)N1CC(n2nc(-c3ccc(C(=O)Nc4ccccn4)cc3)c3cncc(Cl)c32)C1] | 4-(1-(1-(but-2-ynoyl)azetidin-3-yl)- 7-chloro-1H-pyrazolo[4,3-c]pyridin- 3-yl)-N-(pyridin-2-yl)benzamide | BCD-BTK-280 |
| 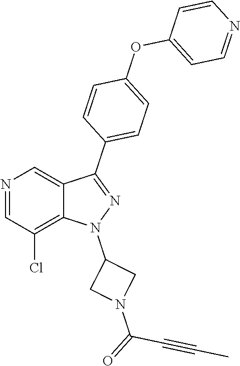 [SMILES: CC#CC(=O)N1CC(n2nc(-c3ccc(Oc4ccncc4)cc3)c3cncc(Cl)c32)C1] | 1-(3-(7-chloro-3-(4-(pyridin-4- yloxy)phenyl)-1H-pyrazolo[4,3- c]pyridin-1-yl)azetidin-1-yl)but-2- yn-1-one | BCD-BTK-281 |
| 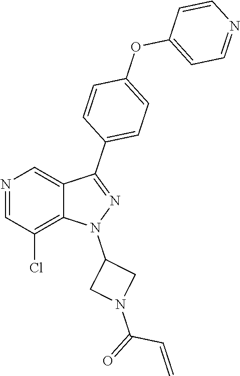 [SMILES: C=CC(=O)N1CC(n2nc(-c3ccc(Oc4ccncc4)cc3)c3cncc(Cl)c32)C1] | 1-(3-(7-chloro-3-(4-(pyridin-4- yloxy)phenyl)-1H-pyrazolo[4,3- c]pyridin-1-yl)azetidin-1-yl)prop-2- en-1-one | BCD-BTK-282 |
| 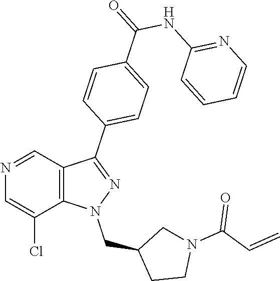 [SMILES: C=CC(=O)N1CC[C@@H](Cn2nc(-c3ccc(C(=O)Nc4ccccn4)cc3)c3cncc(Cl)c32)C1] | (R)-4-(1-((1-acryloylpyrrolidin-3- yl)methyl)-7-chloro-1H- pyrazolo[4,3-c]pyridin-3-yl)-N- (pyridin-2-yl)benzamide | BCD-BTK-284 |
| 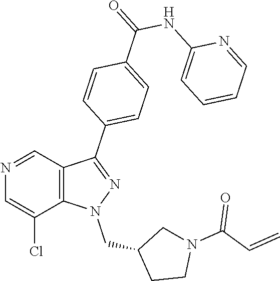 [SMILES: C=CC(=O)N1CC[C@H](Cn2nc(-c3ccc(C(=O)Nc4ccccn4)cc3)c3cncc(Cl)c32)C1] | (S)-4-(1-((1-acryloylpyrrolidin-3- yl)methyl)-7-chloro-1H- pyrazolo[4,3-c]pyridin-3-yl)-N- (pyridin-2-yl)benzamide | BCD-BTK-285 |
| 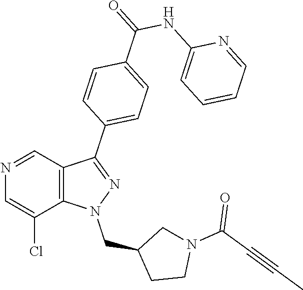 [SMILES: CC#CC(=O)N1CC[C@@H](Cn2nc(-c3ccc(C(=O)Nc4ccccn4)cc3)c3cncc(Cl)c32)C1] | (R)-4-(1-((1-(but-2-ynoyl)pyrrolidin- 3-yl)methyl)-7-chloro-1H- pyrazolo[4,3-c]pyridin-3-yl)-N- (pyridin-2-yl)benzamide | BCD-BTK-286 |
| 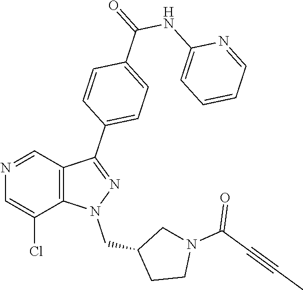 [SMILES: CC#CC(=O)N1CC[C@H](Cn2nc(-c3ccc(C(=O)Nc4ccccn4)cc3)c3cncc(Cl)c32)C1] | (S)-4-(1-((1-(but-2-ynoyl)pyrrolidin- 3-yl)methyl)-7-chloro-1H- pyrazolo[4,3-c]pyridin-3-yl)-N- (pyridin-2-yl)benzamide | BCD-BTK-287 |
| 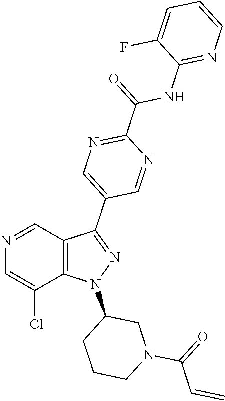 [SMILES: C=CC(=O)N1CCC[C@@H](n2nc(-c3cnc(C(=O)Nc4ncccc4F)nc3)c3cncc(Cl)c32)C1] | (R)-5-(1-(1-acryloylpiperidin-3-yl)- 7-chloro-1H-pyrazolo[4,3-c]pyridin- 3-yl)-N-(3-fluoropyridin-2- yl)pyrimidine-2-carboxamide | BCD-BTK-288 |
| 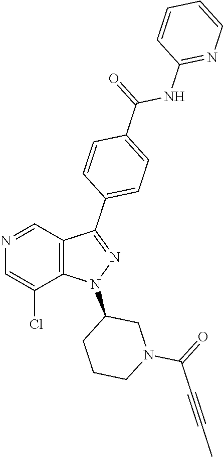 [SMILES: CC#CC(=O)N1CCC[C@@H](n2nc(-c3ccc(C(=O)Nc4ccccn4)cc3)c3cncc(Cl)c32)C1] | (R)-4-(1-(1-(but-2-ynoyl)piperidin- 3-yl)-7-chloro-1H-pyrazolo[4,3- c]pyridin-3-yl)-N-(pyridin-2- yl)benzamide | BCD-BTK-289 |
| 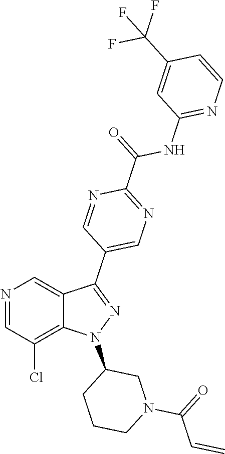 [SMILES: C=CC(=O)N1CCC[C@@H](n2nc(-c3cnc(C(=O)Nc4cc(C(F)(F)F)ccn4)nc3)c3cncc(Cl)c32)C1] | (R)-5-(1-(1-acryloylpiperidin-3-yl)- 7-chloro-1H-pyrazolo[4,3-c]pyridin- 3-yl)-N-(4-(trifluoromethyl)pyridin-2- yl)pyrimidine-2-carboxamide | BCD-BTK-291 |
| 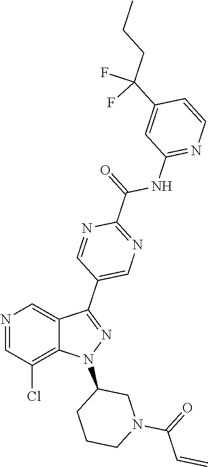 [SMILES: C=CC(=O)N1CCC[C@@H](n2nc(-c3cnc(C(=O)Nc4cc(C(F)(F)CCC)ccn4)nc3)c3cncc(Cl)c32)C1] | (R)-5-(1-(1-acryloylpiperidin-3-yl)- 7-chloro-1H-pyrazolo[4,3-c]pyridin- 3-yl)-N-(4-(1,1-difluorobutyl)pyridin- 2-yl)pyrimidine-2-carboxamide | BCD-BTK-292 |
| 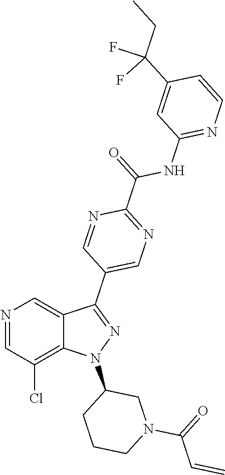 [SMILES: C=CC(=O)N1CCC[C@@H](n2nc(-c3cnc(C(=O)Nc4cc(C(F)(F)CC)ccn4)nc3)c3cncc(Cl)c32)C1] | (R)-5-(1-(1-acryloylpiperidin-3-yl)- 7-chloro-1H-pyrazolo[4,3-c]pyridin- 3-yl)-N-(4-(1,1-difluoropropyl)pyridin- 2-yl)pyrimidine-2-carboxamide | BCD-BTK-293 |
| 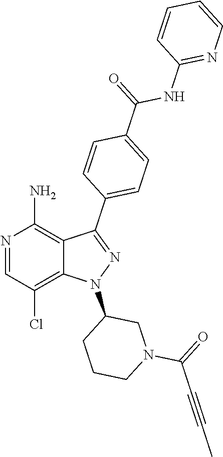 [SMILES: CC#CC(=O)N1CCC[C@@H](n2nc(-c3ccc(C(=O)Nc4ccccn4)cc3)c3c(N)ncc(Cl)c32)C1] | (R)-4-(4-amino-1-(1-(but-2- ynoyl)piperidin-3-yl)-7-chloro-1H- pyrazolo[4,3-c]pyridin-3-yl)-N- (pyridin-2-yl)benzamide | BCD-BTK-295 |

In one aspect the present invention relates to methods for preparation of compound of formula I:

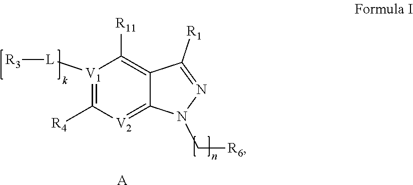

or pharmaceutically accepted salt, solvate or stereoisomer thereof;
wherein:

### V₁ is C or N,

V₂ is C(R₂) or N,
whereby if V₁ is C then V₂ is N,
if V₁ is C then V₂ is C(R₂), or
if V₁ is N then V₂ is C(R₂);
n, k independently is 0, 1;
R₂, R₁ independently is H, D, Hal, CN, NR′R″, C(O)NR′R″, C₁-C₆ alkoxy;
R₃ is H, D, hydroxy, C(O)C₁-C₆ alkyl, C(O)C₂-C₆ alkenyl, C(O)C₂-C₆ alkynyl, C₁-C₆ alkyl;
R₄ is H, Hal, CN, CONR′R″, hydroxy, C₁-C₆ alkyl, C₁-C₆ alkoxy;
L is CH₂, NH, O or chemical bond;
R₁ is selected from the group of the fragments, comprising:

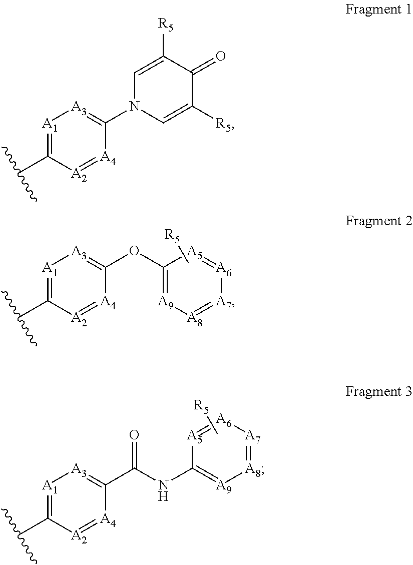

A₁, A₂, A₃, A₄ is independently CH, N, CHal;
A₅, A₆, A₇, A₈, A₉ is independently C, CH or N;
R₅ is H, CN, Hal, CONR′R″, C₁-C₆ alkyl, non-substituted or substituted by one or more halogens;
R′ and R″ is independently selected from the group, comprising H, C₁-C₆ alkyl, C₁-C₆ cycloalkyl, aryl;
R₆ is selected from the group:

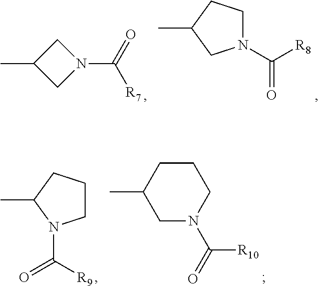

R₇, R₈, R₉, R₁₀ is independently vinyl, methylacetylenyl;

### Hal is Cl, Br, I, F;

that includes:
1) interaction of compound of formula A

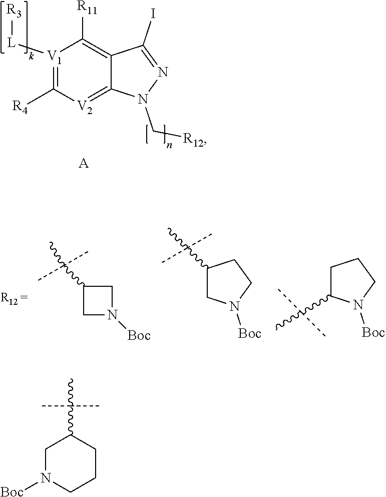

wherein V₁, V₂, L, R₃, R₄, R₁₁, n, k have the same meanings as defined above,
via the Suzuki-Miyaura reaction in an appropriate solvent, with compound of formula X1, X2, X3

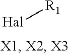

wherein R₁ has the meanings as defined above,
in the presence of palladium salts, phosphorus-containing ligands, and inorganic or organic bases, which forms compound of formula B

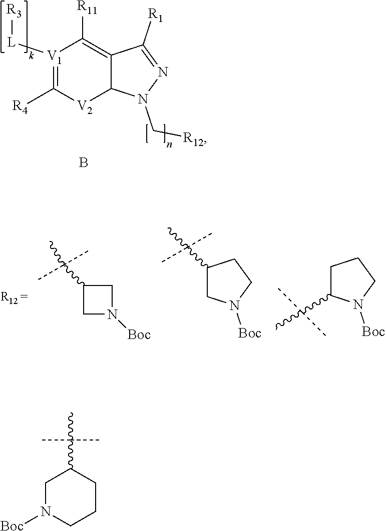

wherein V₁, V₂, L, R₁, R₃, R₄, R₁₁, n, k have the meanings as defined above, and
2) interaction of the resulting compound of formula B with inorganic or organic acid in an appropriate solvent, which forms a salt of compound of formula C

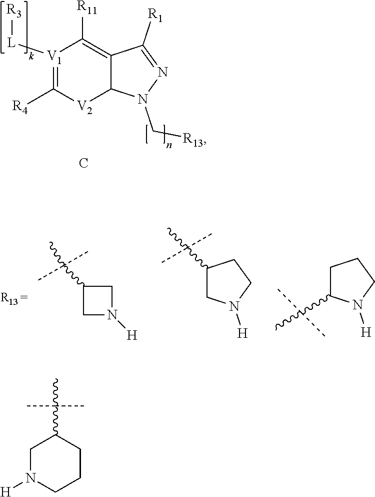

wherein V₁, V₂, L, R₁, R₃, R₄, R₁₁, n, k have the meanings as defined above, and
3) interaction of the resulting salt of compound of formula C with an acylating agent in an appropriate solvent in the presence of organic base, which forms compound of formula I.

In yet another aspect the present invention relates to methods for preparation of compound of formula III:

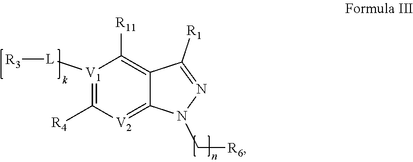

or pharmaceutically accepted salt, solvate or stereoisomer thereof;
wherein:

### V₁ is C or N,

V₂ is C(R₂) or N,
whereby if V₁ is C then V₂ is N,
if V₁ is C then V₂ is C(R₂), or
if V₁ is N then V₂ is C(R₂);
n, k independently is 0, 1;
R₂ is Hal, CN, NR′R″, C(O)NR′R″, C₁-C₆ alkoxy;
R₁₁ is H, Hal, CN, NR′R″, C(O)NR′R″, C₁-C₆ alkoxy;
L is CH₂, NH, O or chemical bond;
R₃ is H, hydroxy, C(O)C₁-C₆ alkyl, C(O)C₂-C₆ alkenyl, C(O)C₂-C₆ alkynyl, C₁-C₆ alkyl;
R₄ is H, Hal, CN, CONR′R″, hydroxy, C₁-C₆ alkyl, C₁-C₆ alkoxy; and if V₁ is C, V₂ is N,
then at least one of R₃, R₄, R₁₁ is not H;
R₁ is selected from the group of the fragments, comprising:

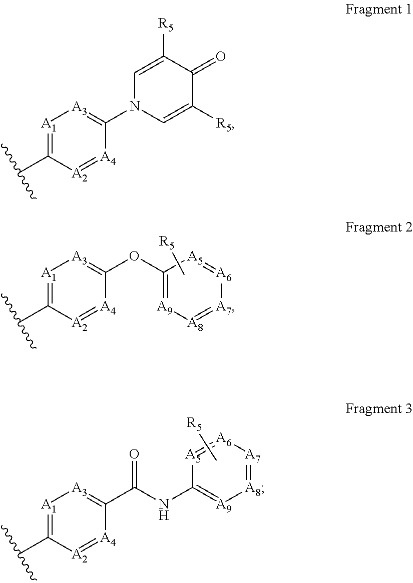

A₁, A₂, A₃, A₄ is independently CH, N, CHal;
A₅, A₆, A₇, A₈, A₉ is independently C, CH or N;
R₅ is H, CN, Hal, CONR′R″, C₁-C₆ alkyl, non-substituted or substituted by one or more halogens;
R′ and R″ is independently selected from the group, comprising H, C₁-C₆ alkyl, C₁-C₆ cycloalkyl, aryl;
R₆ is selected from the group:

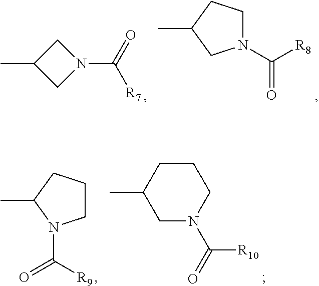

R₇, R₈, R₉, R is independently vinyl, methylacetylenyl;

### Hal is Cl, Br, I, F.

that includes:
1) interaction of compound of formula D

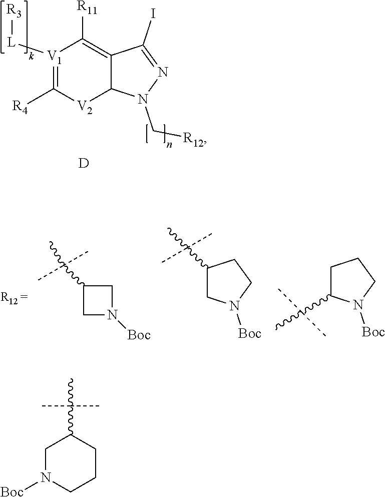

wherein V₁, V₂, L, R₃, R₄, R₁₁, n, k have the same meanings as defined above,
via the Suzuki-Miyaura reaction in an appropriate solvent, with compound of formula X1, X2, X3

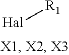

wherein R₁ has the meanings as defined above,
in the presence of palladium salts, phosphorus-containing ligands, and inorganic or organic bases, which forms compound of formula E

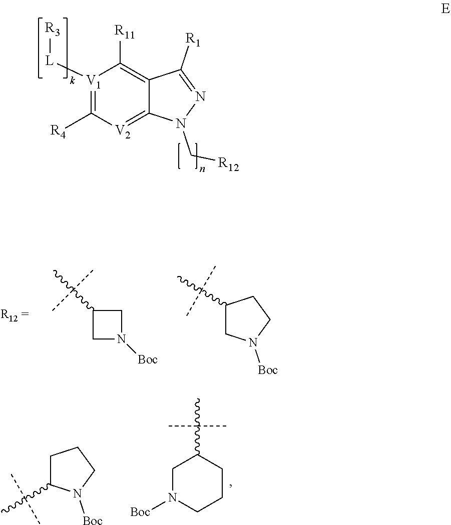

wherein V₁, V₂, L, R₁, R₃, R₄, R₁₁, n, k have the meanings as defined above, and
2) interaction of the resulting compound of formula E with inorganic or organic acid in an appropriate solvent, which forms a salt of compound of formula F

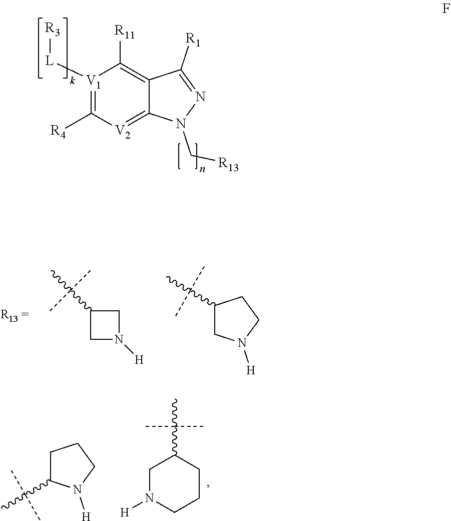

wherein V₁, V₂, L, R₁, R₃, R₄, R₁₁, n, k have the meanings as defined above, and
3) interaction of the resulting salt of compound of formula F with an acylating agent in an appropriate solvent in the presence of organic base, which forms compound of formula III.

The present invention also relates to a method for inhibiting of biological activity of Bruton's tyrosine kinase (Btk) in a subject, comprising contacting the Bruton's tyrosine kinase with the compound described herein.

Irreversible Btk inhibitor compounds can be used for the manufacture of a medicament for treating any of the foregoing conditions (e.g., autoimmune and inflammatory diseases, allergic disorders, immune disorders, tumors of blood and lymphatic system, cancer).

Generally, an irreversible Btk inhibitor compound used in the methods described herein is identified or characterized in an in vitro assay, e.g., an acellular biochemical assay or a cellular functional assay. Such assays are useful to determine an in vitro IC₅₀ for an irreversible Btk inhibitor compound.

In some embodiments, the irreversible Btk inhibitor compound used for the methods described herein inhibits Btk or a Btk homolog kinase activity with an in vitro IC₅₀ of less than 10 LVM (e.g., less than 1, less than 0.5, less than 0.4, less than 0.3, less than 0.1, less than 0.08, less than 0.06, less than 0.05, less than 0.04, less than 0.03, less than 0.02, less than 0.01, less than 0.008, less than 0.006, less than 0.005, less than 0.004, less than 0.003, less than 0.002, less than 0.001, less than 0.00099, less than 0.00098, less than 0.00097, less than 0.00096, less than 0.00095, less than 0.00094, less than 0.00093, less than 0.00092, or less than 0.00090 μM).

In one embodiment, the present invention relates to a pharmaceutical composition that comprises a therapeutically effective amount of at least one of the compounds described herein, or pharmaceutically acceptable salt, solvate thereof, and one or more pharmaceutically acceptable excipients. In another one embodiment, the pharmaceutical composition of the present invention is intended to treat or prevent a disease or disorder mediated by Bruton's tyrosine kinase (Btk).

In another one embodiment, the present invention relates to a pharmaceutical composition for the prevention or treatment of a disease or disorder mediated by Bruton's tyrosine kinase (Btk), that comprises a therapeutically effective amount of the compound described herein, or pharmaceutically acceptable salt thereof, and one or more pharmaceutically acceptable excipients.

In another one embodiment, the pharmaceutical composition of the present invention is intended to treat or prevent tumors of blood and lymphatic system, immune disorders, cancer, autoimmune and inflammatory diseases, or allergic disorders. In some embodiments, pharmaceutical composition of the present invention is intended to treat or prevent chronic lymphocytic leukemia, mantle cell lymphoma, follicular lymphoma, diffuse large B-cell lymphoma, Waldenstrom macroglobulinemia, B-cell prolymphocytic leukemia, central nervous system lymphoma, multiple myeloma, pancreatic cancer, graft-versus-host disease, chronic graft-versus-host disease, rheumatoid arthritis, systemic lupus erythematosus, asthma, atopic dermatitis.

The pharmaceutical composition of the present invention comprises, by way of example, from about 10% to about 100% of active ingredients, preferably from about 20% to about 60% of active ingredients. It is to be understood that each dosage unit may not comprise an effective amount of an active ingredient or ingredients, because the sufficient effective amount can be achieved by multiple dosing.

A typical composition is prepared by mixing the compound described herein with a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which compound of the present invention is being applied. Solvents are generally selected based on solvents recognized by persons skilled in the art as safe to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The compositions may also include one or more buffers, stabilizing agents, surfactants, wefting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., compound of the invention or pharmaceutical composition thereof) or aid in the manufacturing of the pharmaceutical product (i.e., medicament).

The pharmaceutical compositions also include solvates and hydrates of compounds of the present invention, or stabilized form of the compound (e.g., complex with a cyclodextrin derivative or other known complexation agent).

The pharmaceutical compositions of the invention may be formulated for an oral route administration. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches. Formulations for oral administration preferably comprise tablets and capsules.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The pharmaceutical compositions of the invention could be use for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. Intratumoral delivery, e.g. intratumoral injection, may also be advantageous. Regional perfusion is also contemplated.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like.

The compounds of the invention can also be administered intranasally or by inhalation, typically in the form of a dry powder (either alone, as a mixture, or as a mixed component particle, for example, mixed with a suitable pharmaceutically acceptable excipient) from a dry powder inhaler, as an aerosol spray from a pressurised container, pump, spray, atomiser (preferably an atomiser using electrohydrodynamics to produce a fine mist), or nebuliser, with or without the use of a suitable propellant, or as nasal drops.

The pressurised container, pump, spray, atomizer, or nebuliser generally contains a solution or suspension of a compound of the invention comprising, for example, a suitable agent for dispersing, solubilising, or extending release of the active, a propellant(s) as solvent.

Prior to use in a dry powder or suspension formulation, the drug product is generally micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules, blisters and cartridges for use in an inhaler or insufflator may be formulated to contain a powder mix of the compound of the invention, a suitable powder base and a performance modifier.

A suitable solution formulation for use in an atomiser using electrohydrodynamics to produce a fine mist may contain a suitable dose of the compound of the invention per actuation and the actuation volume may for example vary from 1 μL to 100 μL.

Suitable flavours, such as menthol and levomenthol, or sweeteners, such as saccharin or saccharin sodium, may be added to those formulations of the invention intended for inhaled/intranasal administration.

In the case of dry powder inhalers and aerosols, the dosage unit is determined by means of a valve which delivers a metered amount. Units in accordance with the invention are typically arranged to administer a metered dose or “puff” of a compound of the invention. The overall daily dose will typically be administered in a single dose or, more usually, as divided doses throughout the day.

Formulations may be formulated to be immediate and/or modified release. Modified release formulations include delayed-, sustained-, pulsed-, controlled-, targeted and programmed release.

In one embodiment, the present invention relates to the method for treating diseases or disorders mediated by Bruton's tyrosine kinase (Btk) that comprises the step of administering a therapeutically effective amount of any compound described above, or a pharmaceutical composition of the present invention to a subject in need of such treatment.

In another one embodiment, the present invention relates to the method for treating a disease or disorder mediated by Bruton's tyrosine kinase (Btk), which is either a tumor of blood and lymphatic system, immune disorders, cancer, autoimmune and inflammatory disease, or allergic disorder, that comprises the step of administering a therapeutically effective amount of any compound described herein, or a pharmaceutical composition of the present invention to a subject in need of such treatment.

In another one embodiment, the present invention relates to the described above method for treating a subject with chronic lymphocytic leukemia, mantle cell lymphoma, follicular lymphoma, diffuse large B-cell lymphoma, Waldenstrom macroglobulinemia, B cell prolymphocytic leukemia, central nervous system lymphoma, multiple myeloma, pancreatic cancer, graft-versus-host disease, chronic graft-versus-host disease, rheumatoid arthritis, systemic lupus erythematosus, asthma, atopic dermatitis.

The compounds of the invention may be administered alone or in combination with one or more other drugs or biopharmaceuticals (or as any combination thereof). The pharmaceutical compositions, methods and uses of the invention thus also encompass embodiments of combinations (co-administration) with other active agents.

As used herein, the terms “co-administration”, “co-administered” and “in combination with” referring to the compounds with one or more other therapeutic agents, is intended to mean, and does refer to and include the following:
simultaneous administration of such combination of compound of the invention and therapeutic agent(s) to a patient in need of treatment, when such components are formulated together into a single dosage form which releases said components at substantially the same time to said patient,
substantially simultaneous administration of such combination of compound of the invention and therapeutic agent(s) to a patient in need of treatment, when such components are formulated apart from each other into separate dosage forms which are taken at substantially the same time by said patient, whereupon said components are released at substantially the same time to said patient,
sequential administration of such combination of compound of the invention and therapeutic agent(s) to a patient in need of treatment, when such components are formulated apart from each other into separate dosage forms which are taken at consecutive times by said patient with a significant time interval between each administration, whereupon said components are released at substantially different times to said patient; and
sequential administration of such combination of compound of the invention and therapeutic agent(s) to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

As well known to those skilled in the art, therapeutically effective dosages may vary when the drugs are used in combination treatment. Methods for experimentally determining therapeutically effective dosages of drugs and other agents for use in combination treatment regimens are described in the literature. For example, the use of metronomic dosing, i.e., providing more frequent, lower doses in order to minimize toxic side effects, has been described in the literature. Combination treatment further includes periodic treatments that start and stop at various times to assist with the clinical management of the patient. For combination therapies described herein, dosages of the co-administered compounds will of course vary depending on the type of co-drug employed, on the specific drug employed, on the condition or disorder being treated and so forth.

In addition, compounds described herein may also be used in combination with procedures that may provide additional or synergistic benefit to the subject. By way of example only, subjects are expected to find therapeutic and/or prophylactic benefit in the methods described herein, wherein pharmaceutical composition of the present invention and/or combinations with other therapeutics are combined with genetic testing to determine whether that individual is a carrier of a mutant gene that is known to be correlated with certain diseases or conditions.

Where the subject is suffering from or at risk of suffering from an autoimmune disease, an inflammatory disease, or an allergy disorder, an irreversible Btk inhibitor compound of the present invention can be used in with one or more of the following therapeutic agents in any combination: immunosuppressants (e.g., tacrolimus, rapamycin (sirolimus), everolimus, cyclosporin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-y, interleukin-2, allergy vaccines, antihistamines, antileukotrienes, beta-agonists, theophylline, or anticholinergics.

Where the subject is suffering from or at risk of suffering from a tumor of blood and lymphatic system (e.g., chronic lymphocytic leukemia), the subject can be treated with an irreversible Btk inhibitor compound in any combination with one or more other anti-cancer agents. In some embodiments, one or more of the anti-cancer agents are proapoptotic agents. Examples of anti-cancer agents include, but are not limited to, any of the following: gossypol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, doxorubicin, vincristine, etoposide, gemcitabine, imatinib, geldanamycin, 17-N-Allylamino-17-Demethoxygeldanamycin (17-AAG), flavopiridol, LY294002, bortezomib, trastuzumab, BAY 11-7082, PKC412, or PDl 84352, paclitaxel, docetaxel, compounds that have the basic taxane skeleton as a common structure feature.

Further examples of anti-cancer agents for use in combination with an irreversible Btk inhibitor compound include inhibitors of mitogen-activated protein kinase signaling, e.g., UO 126, PD98059, PD 184352, PD0325901, ARRY-142886, SB239063, SP600125, BAY 43-9006, wortmannin, or LY294002; Syk inhibitors; mTOR inhibitors; and antibodies (e.g., rituximab).

Other anti-cancer agents that can be employed in combination with an irreversible Btk inhibitor compound include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rIL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamme hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porffmer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfui; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with an irreversible Btk inhibitor compound include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amnibicin; amsacrine; anagrelide; anastrozole; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; butbionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorhis; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethymorsperrnine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; oracin; oral cytokine inducer; ormaplatin; osaterone; oxaliplatin; oxaunomycin; palauamine; palinitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiiomycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer; inhibitors of Bcl-2 protein family; phosphatidylinositol-3-kinase inhibitors; clarithromycin; erythromycin; azithromycin.

Yet other anticancer agents that can be employed in combination with an irreversible Btk inhibitor compound include alkylating agents, antimetabolites, natural products, or hormones, (e.g., nitrogen mustards, mechloroethamine, cyclophosphamide, chlorambucil, etc.), alkyl sulfonates (e.g., busulfan), nitrosoureas (e.g., carmustine, lomusitne, etc.), or triazenes (dacarbazine, etc.).

Examples of antimetabolites include, but are not limited to, folic acid analog (e.g., methotrexate), or pyrimidine analogs (e.g., Cytarabine), purine analogs (e.g., mercaptopurine, thioguanine, pentostatin, fludarabine).

Examples of natural products useful in combination with an irreversible Btk inhibitor compound include, but are not limited to, vinca alkaloids (e.g., vinblastin, vincristine), epipodophyllotoxins (e.g., etoposide), antibiotics (e.g., daunorubicin, doxorubicin, bleomycin, clarithromycin), enzymes (e.g., L-asparaginase), or biological response modifiers (e.g., interferon alpha).

Examples of hormones and antagonists useful in combination with an irreversible Btk inhibitor compound include, but are not limited to, adrenocorticosteroids (e.g., prednisone, prednisolone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide), aromatase inhibitor (e.g., anastrozole). Other agents that can be used in the methods and compositions described herein for the treatment or prevention of cancer include platinum coordination complexes (e.g., cisplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), growth hormone antagonist (e.g., octreotide).

Examples of anti-cancer agents which act by arresting cells in the G2-M phases due to stabilized microtubules and which can be used in combination with an irreversible Btk inhibitor compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-I (Parker Hughes Institute, also known as DDE-261 and WHI-261), HlO (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-I (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, hianocine (also known as NSC-698666), 3-1AABE (Cytoskeleton/Mt. Sinai School of Medicine), A-204197 (Abbott), T-607 (Tularik, also known as T-900607), RPR-(Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Where the subject is suffering from or at risk of suffering from a thromboembolic disorder (e.g., stroke), the subject can be treated with an irreversible Btk inhibitor compound in any combination with one or more other anti-thromboembolic agents. Examples of anti-thromboembolic agents include, but are not limited to, any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YMI 50), ticlopidine, clopidogrel, CS-747 (prasugrel, LY640315), ximelagatran, or BIBR 1048.

It is understood that the compounds of the invention may be used in methods for treating, as described above, in treatment, as described above, and/or in the manufacture of a medicament for the therapeutic applications described above.

In one embodiment, the present invention relates to use of the compound described herein or a pharmaceutical composition of the present invention in the treatment of diseases or disorders mediated by Bruton's tyrosine kinase (Btk) in a subject in need thereof.

In another one embodiment, the present invention relates to the use of the compound described herein or a pharmaceutical composition of the invention in the treatment of a disease or disorder mediated by Bruton's tyrosine kinase (Btk), which is either a tumor of blood and lymphatic system, immune disorders, cancer, autoimmune and inflammatory disease, or allergic disorder, that comprises the step of administering a therapeutically effective amount of any compound described herein, or a pharmaceutical composition of the present invention to a subject in need thereof.

In another one embodiment, the present invention relates to the use of the compound described herein or a pharmaceutical composition of the present invention in the treatment of a subject with chronic lymphocytic leukemia, mantle cell lymphoma, follicular lymphoma, diffuse large B-cell lymphoma, Waldenstrom macroglobulinemia, B cell prolymphocytic leukemia, central nervous system lymphoma, multiple myeloma, pancreatic cancer, graft-versus-host disease, chronic graft-versus-host disease, rheumatoid arthritis, systemic lupus erythematosus, asthma, atopic dermatitis. In all of these embodiments, the subject may be human.

The compounds of the invention will be administered in an effective amount for treatment of the condition in question, i.e., at dosages and for periods of time necessary to achieve a desired result. A therapeutically effective amount may vary according to factors such as the particular condition being treated, the age, sex and weight of the patient, and whether the compounds are being administered as a stand-alone treatment or in combination with one or more additional treatments.

Dosage regimens may be adjusted to provide the optimum desired response. For example, a single dose may be administered, several divided doses may be administered over time or the dose may be proportionally reduced or increased as indicated by the exigencies of the therapeutic situation. It is especially advantageous to formulate oral compositions in dosage unit form for ease of administration and uniformity of dosage. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier. The specification for the dosage unit forms of the invention are generally dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the person skilled in the art would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of this invention may be based on a variety of factors, including the type of disease, the age, weight, sex, medical condition of the patient, the severity of the condition, the route of administration, and the particular compound employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present invention encompasses intra-patient dose-escalation as determined by the person skilled in the art. Determining appropriate dosages and regimens are well-known in the relevant art and would be understood to be encompassed by the person skilled in the art once provided the teachings disclosed herein.

Generally, standard daily dosage for an adult human is in the range from 0.02 mg to 5000 mg or from about 1 mg to about 1500 mg.

Once improvement of the patient's conditions has occurred, a maintenance dose is administered, if necessary. Subsequently, the dosage or the frequency of administration, or both, can be reduced, as a function of the symptoms, to a level at which the improved disease or disorder is retained. Patients may be required periodic treatment on a long-term basis upon any relapse of symptoms.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disorder or condition to be treated, the method of administration, the requirements of the individual subject, the severity of the disorder or condition being treated, and the judgment of the physician.

An effective amount for tumor therapy may be measured by its ability to slow down disease progression and/or ameliorate symptoms in a patient, and preferably to reverse disease progression. The ability of a compound of the present invention to inhibit the foregoing diseases may be evaluated by in vitro assays, e.g. as described in the examples, as well as in suitable animal models that are predictive of the efficacy in such disorders. Suitable dosage regimens will be selected in order to provide an optimum therapeutic response in each particular situation, for example, administered as a single tablet or capsule with possible adjustment of the dosage as indicated by the exigencies of each case.

In order that this invention may be better understood, the following examples are set forth. These examples are for purposes of illustration only and are not to be construed as limiting the scope of the invention in any manner.

All publications, patents, and patent applications cited in this specification are incorporated herein by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended embodiments.

### Examples

Abbreviations in this description, including those shown in illustrative schemes and the examples described below are well-know for an average person skilled in the art. Some of the abbreviations are as follows:

XPhos—2-dicyclohexylphosphino-2′,4′,6′-triisopropylbiphenyl
DMF dimethylformamide
HATU—1-[bis(dimethylamino)methylene]-1H-1,2,3-triazolo[4,5-b]pyridinium-3-oxide hexafluorophosphate
DIPEA—diisopropylethylamine
BOC-anhydride—di-tert-butyldicarbonate
THF—tetrahydrofuran
DMSO—dimethylsulfoxide
Pd₂(dba)₃—tris (dibenzylideneacetone) dipalladium (0)
Pd(dppf)Cl₂—[1,1′-bis(diphenylphosphino)ferrocene]dichloropalladium (II)
TBDMSCl—tert-butyldimethylsilyl chloride
Pd(PPh₃)₄—tetrakis (triphenylphosphine) palladium (0)

### Example 1. General Method for Synthesis of Compound of Formula I

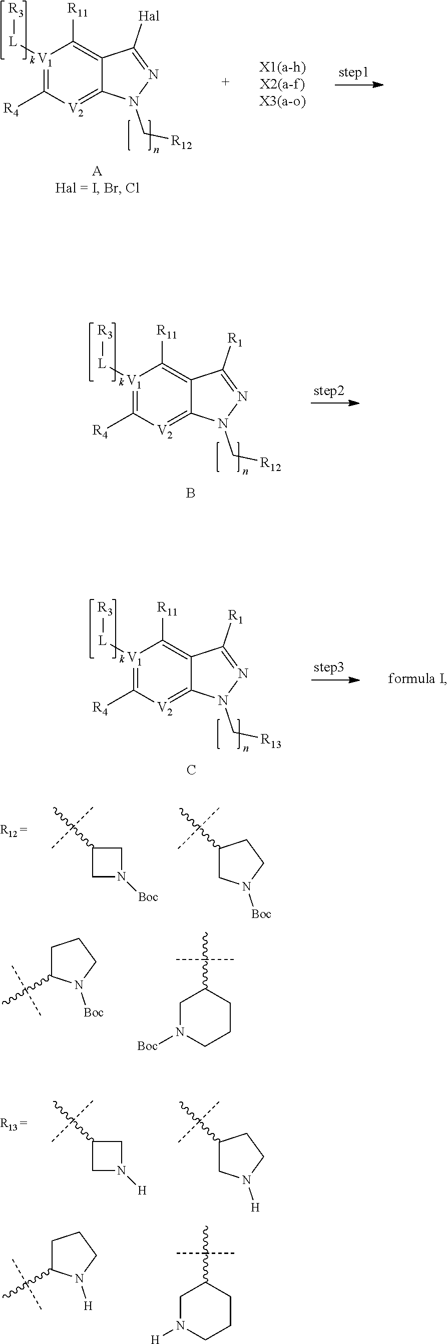

wherein V₁, V₂, L, R₁, R₃, R₄, R₁₁, n, k have the above meanings.

General method for synthesis of compound of formula III.

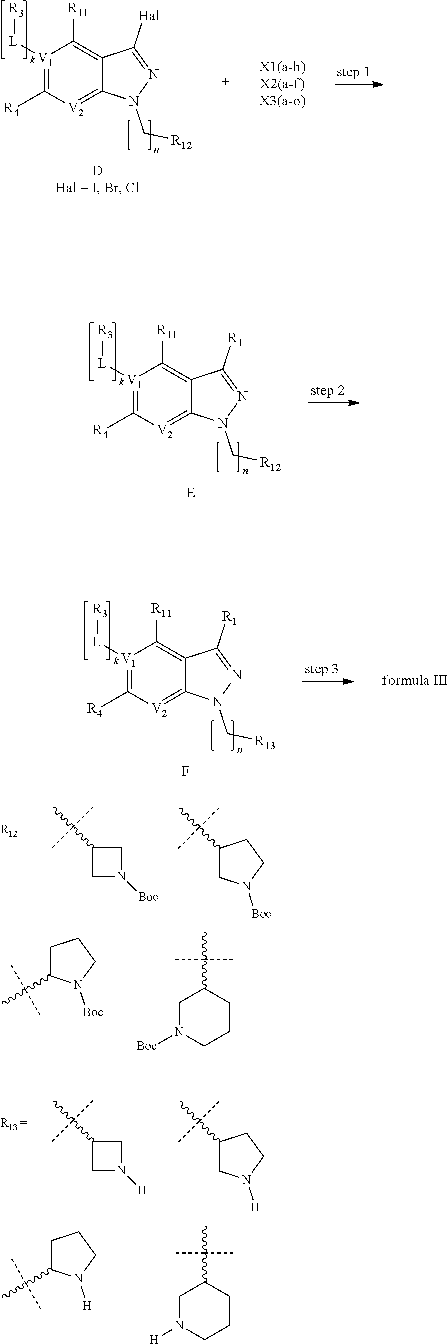

wherein V₁, V₂, L, R₁, R₃, R₄, R₁₁, n, k have the above meanings.

Step 1: Synthesis of Compounds B (E).

In a three-neck flask, equipped with a stirrer and thermometer, mix under nitrogen in the specified order: 20 mL of 1,4-dioxane; (0.002 mol) of necessary compound X1, X2 or X3; 0.759 g (0.003 mol) of bis(pinacolato)diboron; 0.190 g (0.0004 mol) of XPhos; 0.588 g (0.006 mol) of dry potassium acetate; 0.067 g (0.0002 mol) of palladium(II) acetate. While stirring, pass an inert gas (argon or nitrogen) through the mixture for 15 minutes. Stir the resulting reaction mass under the inert gas at 80-90° C. for 3-5 hours; use the TLC method to ensure the completeness of the reaction. When the reaction is complete, cool the reaction mixture to 40° C. Add a solution of 1.7 g (0.016 mol) of sodium carbonate in 10 mL of water, 0.231 g (0.0002 mol) of tetrakis(triphenylphosphine)palladium, and 0.002 mol of a corresponding compound A(D). Stir the resulting mixture at 80-90° C. for 3-5 hours; use the TLC method to ensure the completeness of the reaction. When the reaction is complete, allow the mass to cool and filter it through celite; wash the celite with 10 mL of ethyl acetate and 10 mL of water; concentrate the filtrate under vacuum using a rotary evaporator. To the resulting residue add 30 mL of water and extract with 30 mL of ethyl acetate five times. Wash the organic layer with water and NaCl solution, dry with sodium sulfate, distill off the solvent. Purify the resulting product by column chromatography, eluent: from ethyl acetate to ethyl acetate:methanol (from 99:1 to 9:1) The resulting product is compound B(E) with 30% to 70% yield.

Step 2: Synthesis of Compounds C(F).

In a three-neck flask, equipped with a stirrer and thermometer, mix under nitrogen in the specified order: 10 mL of 1,4-dioxane, 0.002 mol of necessary compound B(E), and 4 mL of 4M hydrogen chloride in 1,4-dioxane. Allow the mixture to stand at room temperature. After 16 hours, distill off the solvent. The resulting residue is a hydrochloride of a corresponding compound C(F), which is taken to the next step without additional purification.

Step 3: Synthesis of Compounds of Formula I and Formula III.

Depending on the structure and physicochemical characteristics of the resulting compounds, the synthesis can be performed as follows:

Variation 1.

In a three-neck flask, equipped with a stirrer and thermometer, mix under an inert gas in the specified order: 20 mL of dry dichloromethane (or dimethylformamide (DMF)), 0.0005 mol of compound C(F) hydrochloride, and (0.0015 mol) of diisopropylethylamine. Cool the mixture to −30° C. and add at this temperature 0.00051 mol of acryloyl chloride. Allow the reaction mass to stand at room temperature. After 1 hour, concentrate the solvent under vacuum using a rotary evaporator; add 50 mL of ethyl acetate and 50 mL of water. Separate the ethyl acetate from the aqueous layer; wash the aqueous layer once again with ethyl acetate, and combine the ethyl acetate extracts. After that, wash it with 10% citric acid and NaCl solution. Dry the ethyl acetate with sodium sulfate and distill off the solvent. Purify the resulting product by column chromatography, eluent hexane:ethyl acetate (from 3:7 to 0:100). The resulting product is compound of formula I with 5% to 50% yield. Final purification is performed using Akta Explorer 100 with the Inertsil ODS-3 column, R—10 m, L*d—250*30 mm. The compound III is synthetized in a similar manner.

Variation 2.

In a three-neck flask, equipped with a stirrer and thermometer, mix under an inert gas in the specified order: 20 mL of dry dichloromethane (or DMF), 0.0005 mol of compound C(F) hydrochloride respectively, and (0.004 mol) of diisopropylethylamine. Cool the mixture to −20° C. and add at this temperature 0.00205 mol of acryloyl chloride. Allow the reaction mass to stand at room temperature. After 1 hour, remove the solvent under vacuum; add 50 mL of ethyl acetate and 50 mL of water. Separate ethyl acetate from the aqueous layer; wash the aqueous layer once again with ethyl acetate, and combine the ethyl acetate extracts. After that, wash it with 10% citric acid and NaCl solution. Dry the ethyl acetate with sodium sulfate and distill off the solvent. Purify the resulting product by column chromatography, eluent hexane:ethyl acetate (from 3:7 to 0:90). The resulting product is compound of formula I with 5% to 60% yield. The compound III is synthetized in a similar manner with 5% to 60% yield.

Variation 3.

Add HATU 0.55 mmol, and DIPEA 0.73 mmol to a suspension of tetrolic acid 0.38 mmol in dry methylene chloride (20 ml). Cool the reaction mass to 0° C. and add a suspension of 0.38 mmol of the hydrochloride of compound C (F) in dry methylene chloride, if the solubility allows, so that the temperature of the mixture does not exceed 5° C. After the addition, leave the reaction mixture at room temperature for 1 hour, then remove the solvent under vacuum and add 50 ml of ethyl acetate and 50 ml of water. Separate ethyl acetate from the aqueous layer, wash the aqueous layer again with ethyl acetate, and combine ethyl acetate, then wash with 10% citric acid solution and NaCl solution. Dry the ethyl acetate over sodium sulfate and distill off the solvent. Isolate the final product by column chromatography, eluent: hexane:ethyl acetate (3:7 to 1:9). A compound of formula I is obtained with 5% to 60% yield. In case of precipitation, filter it off, otherwise wash the reaction mixture with water and NaCl, distill off the solvent. The product is purified by column chromatography. Compound III is synthesized in a similar manner with 5 to 60% yield.

### Example 2. General Method for Synthesis of Compounds X1, X2, X3

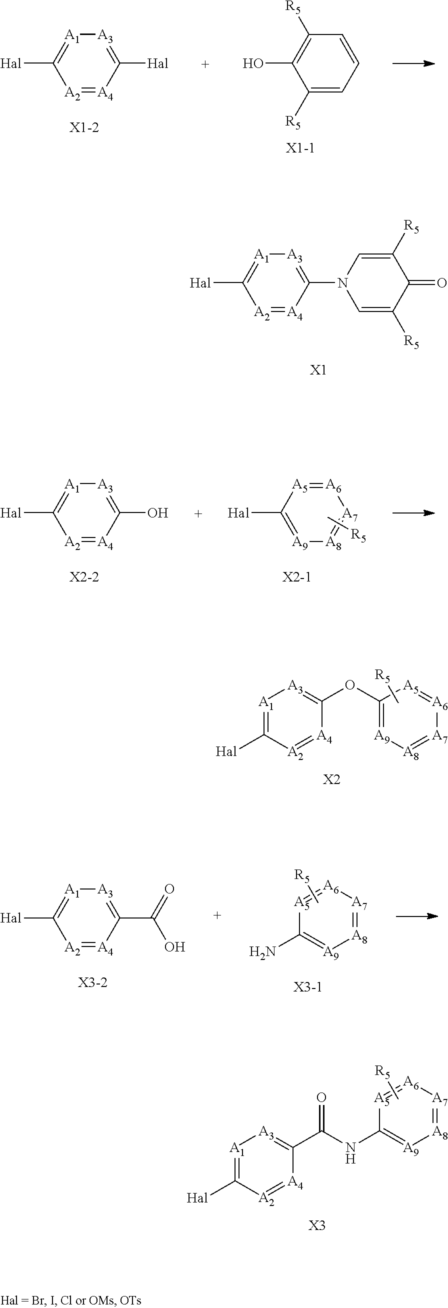

wherein A₁, A₂, A₃, A₄, A₅, A₆, A₇, A₈, A₉, R₅ have the foregoing meanings.

Compounds X1.

In a round-bottom flask, equipped with a stirrer, thermometer and reflux condenser, mix in the specified order: 200 mL of DMF, 0.1 mol of a corresponding phenyl dihalide X1-2, 0.1 mol of a corresponding hydroxypyridine X1-1, and 0.2 mol of cesium or potassium carbonate. Stir the mixture at 100° C. under an inert gas for 2-6 hours; use the TLC method to ensure the completeness of the reaction. After that, distill off most of the solvent using a rotary evaporator; add 200 mL of ethyl acetate, and filter the resulting suspension through celite. Evaporate the filtrate. Purify the resulting product by column chromatography, eluent ethyl acetate:methanol (9:1). The resulting product is compound X1 with 60% to 80% yield.

Compounds X2.

In a round-bottom flask, equipped with a stirrer, thermometer and reflux condenser, mix in the specified order: 10 mL of dry DMF, 0.01 mol of a corresponding pyridine halide X2-1, 0.011 mol of a corresponding phenol halide X2-2, and 0.012 mol of cesium or potassium carbonate. Heat the mixture to 40-80° C., allow to stand at this temperature for 18 hours; use the TLC method to ensure the completeness of the reaction. After that, filter the reaction mass through celite. Add the filtrate to 100 mL of water, and filter the precipitate. Wash the filter cake twice with 20 mL of water, and allow to dry in air. The resulting product is compound X2 with 80% to 95% yield.

Compounds X3.

In a round-bottom flask, equipped with a stirrer, thermometer and reflux condenser, mix in the specified order: 20 mL of dichloromethane, 0.0106 mol of a corresponding halogen benzoic acid X3-2, and 0.02 mol of thionyl chloride. Add 2-3 drops of DMIF to the mixture and boil it for 1.5 hours until the precipitate is dissolved. After that, distill off the solvent; re-evaporate with 20 mL of toluene, and dissolve the resulting solid precipitate in 10 mL of dichloromethane. With constant stirring, add the resulting solution at 0° C. to a pre-prepared solution of a corresponding aminopyridine X3-1 in 10 mL of pyridine. Stir the mixture for another 1 hour, distill off the solvent, and treat the residue with water. Filter the precipitate, wash with 20 mL of water, and allow to dry in air. The resulting product is compound X3 with 40% to 80% yield.

### Example 3. Methods for Synthesis of Intermediates

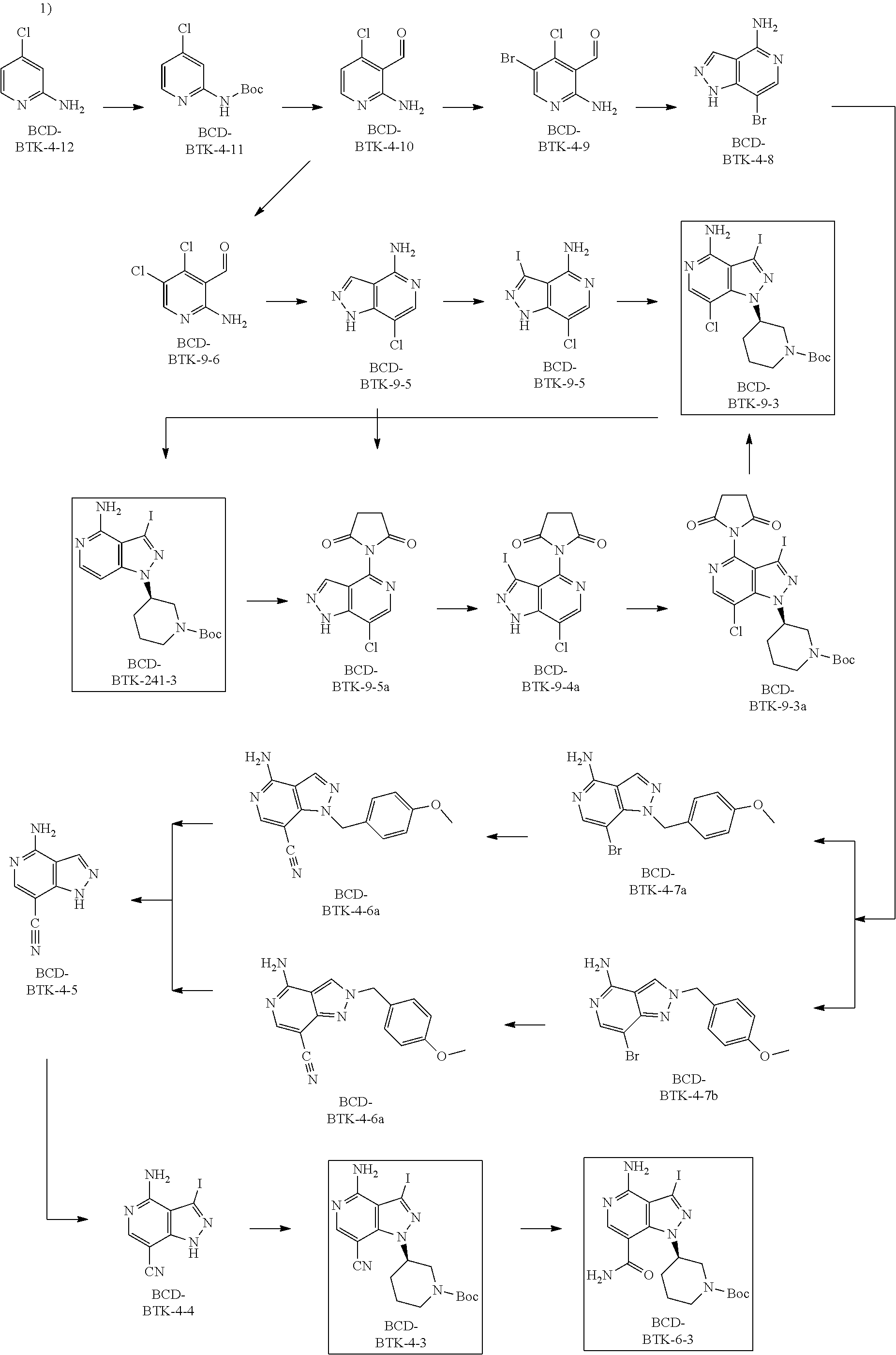

BCD-BTK-4-11.

In a round-bottom flask, equipped with a stirrer, thermometer and reflux condenser, dissolve 20.6 g (0.158 mol) of 2-amino-4-chloropyridine in tert-butanol, add 38.5 g (0.175 mol) of BOC anhydride. Stir the mixture for 5 hours at 40° C. Remove excess solvent by distillation in a rotary evaporator at 40° C.; treat the residue with hexane. Cool the resulting suspension to 0° C., and filter the precipitate. Yield: 28 g (77%).

BCD-BTK-4-10.

In a round-bottom flask, equipped with a stirrer, thermometer and reflux condenser, mix in the specified order: 135 mL of dry tetrahydrofuran (THF), 20 g (0.169 mol) of N,N,N′,N′-tetramethylethylenediamine, and 15.7 g (0.068 mol) of BCD-BTK-4-11. Cool the resulting mixture to −78° C.; add, dropwise, 68 mL of 2.5M n-butyllithium in hexane, maintaining the temperature. After that, allow the reaction mass to stand for another 30 minutes. Add 15 g (0.2 mol) of DMF, maintaining the temperature at −78° C. After 1 hour, stop the cooling and allow the reaction mixture to warm to room temperature. Allow to stand for another hour. Add, while cooling, 30 mL of methanol and 150 mL of NH₄Cl aqueous solution. Allow to stand for 30 minutes. To the reaction mass add 1000 mL of water, 500 mL of dichloromethane, and transfer the resulting mixture to a separatory funnel. Separate the organic layer; re-extract the aqueous layer using 200 mL of dichloromethane. Combine the organic layers, wash with water, and dry with sodium sulfate. Distill off dichloromethane using a rotary evaporator; dissolve the residue in 200 mL of dichloromethane. To the resulting mixture add 50 mL of 4M HCl in 1,4-dioxane, while stirring and cooling. Allow the mixture to stand at room temperature for 5 hours; use the TLC method to ensure the completeness of the reaction. Add another 200 mL of dichloromethane, and neutralize excess acid with 2M NaOH. Separate dichloromethane, wash with water, and dry with sodium sulfate. Purify the resulting product by column chromatography, eluent dichloromethane. Yield: 9.6 g (55%).

BCD-BTK-4-9.

In a round-bottom flask, equipped with a stirrer, thermometer and reflux condenser, mix in the specified order: 40 mL of dry dichloroethane, 6 g (0.037 mol) of BCD-BTK-4-10, and 7.5 g (0.0417 mol) of N-bromosuccinimide. Stir the mixture under nitrogen at 50-60° C. for 2 hours; use the TLC method to ensure the completeness of the reaction. When the reaction is complete, cool the mixture to −10° C., and filter the precipitate. Wash the filter cake once with cooled dichloroethane and three times with 50 mL of water. Allow the washed precipitate to dry in air until the mass is constant. Yield: 7.1 g (79%).

BCD-BTK-4-8.

In a thick walled flask with a threaded neck, mix 150 mL of DMSO, 14.5 g (0.062 mol) of BCD-BTK-4-9, and 12.5 g (0.248 mol) of hydrazine hydrate. Screw the cap on tightly, and heat the flask to 130-140° C. for 4 hours. After that, concentrate the reaction mass using a rotary evaporator. To the residue add 100 mL of water and cool to −5° C. Filter the precipitate with chilled water, and allow to dry in air. Yield: 10.8 g (81%).

BCD-BTK-9-6.

In a round-bottom flask, equipped with a stirrer, thermometer and reflux condenser, mix in the specified order: 40 mL of dry dichloroethane, 6 g (0.037 mol) of BCD-BTK-4-10, and 5.56 g (0.0417 mol) of N-chlorosuccinimide. Stir the mixture under nitrogen at 50-60° C. for 3 hours; use the TLC method to ensure the completeness of the reaction. When the reaction is complete, cool the mixture to −10° C., and filter the precipitate. Wash the filter cake once with cooled dichloroethane and three times with 50 mL of water. Allow the washed precipitate to dry in air until the mass is constant. Yield: 5 g (71%).

BCD-BTK-9-5.

In a thick walled flask with a threaded neck, mix 150 mL of DMSO, 5 g (0.026 mol) of BCD-BTK-4-9, and 5.23 g (0.105 mol) of hydrazine hydrate. Screw the cap on tightly, and heat the flask to 130-140° C. for 4 hours. After that, distill off the solvent using a rotary evaporator. To the residue add 100 mL of water and cool to −5° C. Filter the precipitate with chilled water, and allow to dry in air. Yield: 3.54 g (81%).

BCD-BTK-9-5a.

In a round-bottom flask mix in the specified order: 4.4 g (44 mmol) of succinic anhydride and 3.36 g (20 mmol) of the compound BCD-BTK-9-5. Stir the mixture at 160° C. for 20 minutes and add to the mixture 6 g of ice and 30 ml of water. Filter the precipitate, wash with water and dry under vacuum at 40° C. Yield: 3.5 g (70%).

BCD-BTK-9-4a.

In a round-bottom flask, equipped with a stirrer, thermometer and reflux condenser, mix under nitrogen in the specified order: 25 ml of DMF, 3.5 g (0.014 mol) of the compound BCD-BTK-9-5a and 3.6 g (0.016 mol) N-iodosuccinimide. Mix at 40° C. for 5 hours, use the TLC method to ensure the completeness of the reaction. When the reaction is complete, pour 100 ml of water and filter the precipitate; wash it two times and dry at 40° C. under vacuum. Yield: 4.00 g (77%).

BCD-BTK-9-3a.

In a round-bottom flask, equipped with a stirrer, thermometer and reflux condenser, mix under nitrogen in the specified order: 25 ml of dry THF, 2.52 g (0.0067 mol) of the compound BCD-BTK-9-4a, 3.93 g (0.015 mol) of triphenylphosphine and 3.0 g (0.015 mol) of (S)-3-hydroxy-1-(t-butoxycarbonyl)-piperidine and mix for 15 minutes. Then cool the mixture to 0° C. and add dropwise 3.0 g (0.015 mol) of diisopropyl azodicarboxylate keeping the temperature at 0° C. After that warm the mixture up to 20° C. and mix for 6 hours, use the TLC method to ensure the completeness of the reaction. When the reaction is complete, distill off the solvent. The product is purified by column chromatography, eluent: ethyl acetate-hexane 8:2. Yield: 2.5 g (67%).

BCD-BTK-9-4.

In a round-bottom flask, equipped with a stirrer, thermometer and reflux condenser, mix in the specified order: 150 mL of 1,4-dioxane, 7 g (0.04118 mol) of BCD-BTK-9-5, and 10.039 g (0.152 mol) of KOH. Add, while cooling with water, 21 g (0.0822 mol) of iodine. Stop cooling, and stir the mixture at 70-75° C. for 3 hours; use the TLC method to ensure the completeness of the reaction. When the reaction is complete, add the reaction mass to 600 mL of water, and extract with 100 mL of ethyl acetate five times. Wash the organic layer with 200 mL of water and dry with sodium sulfate. Remove the solvent; purify the resulting product by column chromatography, eluent dichloromethane:ethyl acetate (from 98:2 to 9:1). Concentrate the resulting product with pooled fractions; wash the residue three times with 50 mL of hexane. Yield: 1.2 g (10%).

BCD-BTK-9-3. Variation 1.

In a round-bottom flask, equipped with a stirrer, thermometer and reflux condenser, mix under nitrogen in the specified order: 25 mL of dry methanol, 2.06 g (0.0037 mol) of BCD-BTK-9-3a and 1 ml of hydrazine-hydrate and mix for 6 hours, use the TLC method to ensure the completeness of the reaction. When the reaction is complete, distill off the solvent at room temperature and add 20 ml of water. Filter the precipitate, wash with water and dry. Yield: 1.76 g (79%).

Variation 2.

In a round-bottom flask, equipped with a stirrer, thermometer and reflux condenser, mix in the specified order: 20 mL of dry THF, 0.55 g (0.00177 mol) of BCD-BTK-9-4, 0.624 g (0.00355 mol) of triphenylphosphine, and 0.721 (0.00355 mol) of (S)-3-hydroxy-1-(tert-butoxycarbonyl)piperidine. Stir under nitrogen for 15 minutes. Cool the reaction mass to 0° C.; add, dropwise, 0.624 g (0.00355 mol) of diethyl azodicarboxylate, maintaining the temperature. After that, allow the reaction mass to warm to room temperature, and stir for 1.5 hours; use the TLC method to ensure the completeness of the reaction. When the reaction is complete, distill off the solvent, and treat the residue with a mixture of hexane/ethyl acetate (7:3). Filter off and discard the resulting precipitate; concentrate the mother liquor, and purify the resulting residue by column chromatography, eluent hexane:ethyl acetate (from 95:5 to 7:3). Yield: 0.58 g (68%).

BCD-BTK-241-3.

In a steel autoclave mix in the specified order: 15 ml of methanol, 0.2 ml of aqueous ammonia, 1.06 g (0.0022 mol) of BCD-BTK-9-3 and 0.05 g of 10% palladium-on-carbon and hydrogenize at 2-3 atm. for 6 hours. After the reaction is complete, distill off the solvent at room temperature and add 20 ml of water. Filtrate the precipitate, wash with water and dry. Yield: 0.88 g (89%).

BCD-BTK-4-7a and BCD-BTK-4-7b.

In a round-bottom flask, equipped with a stirrer, thermometer and reflux condenser, mix under nitrogen in the specified order: 120 mL of dry THF, 7 g (0.0325 mol) of BCD-BTK-4-8, 12.066 g (0.0455 mol) of triphenylphosphine, and 4.767 g (0.0342 mol) of p-methoxybenzyl alcohol. Stir for 15 minutes. Cool the reaction mass to 0° C.; add, dropwise, 6.867 g (0.39 mol) of diethyl azodicarboxylate, maintaining the temperature. After that, allow the reaction mass to warm to room temperature, and stir for 2 hours; use the TLC method to ensure the completeness of the reaction. When the reaction is complete, distill off the solvent; purify the resulting product by column chromatography, eluent dichloromethane:ethyl acetate (from 9:1 to 1:1). BCD-BTK-4-7a is purified first (yield: 4.53 g (41.8%)), BCD-BTK-4-7b is purified after that (yield: 2.3 g (21.2%)).

BCD-BTK-4-6a.

In a round-bottom flask, equipped with a stirrer, thermometer and reflux condenser, mix under nitrogen in the specified order: 20 mL of dry DMF, 4.375 g (0.013 mol) of BCD-BTK-4-7a, 2 g (0.0168 mol) of zinc cyanide, and 0.759 g (0.00065 mol) of tetrakis(triphenylphosphine)palladium. Heat the mixture to 130° C. for 5 hours; use the TLC method to ensure the completeness of the reaction. When the reaction is complete, cool the mixture, filter through celite, and distill off the solvent. To the residue add a 1:1 mixture of acetone/hexane, boil for 5-10 minutes, and cool to −10° C. Filter the suspension, wash the precipitate with hexane, and allow to dry in air. Yield: 2.7 g (75%).

BCD-BTK-4-6b.

In a round-bottom flask, equipped with a stirrer, thermometer and reflux condenser, mix in the specified order: 10 mL of dry DMF, 2.3 g (0.0068 mol) of BCD-BTK-4-7b, 1.054 g (0.00888 mol) of zinc cyanide, and 0.399 g (0.00034 mol) of tetrakis(triphenylphosphine)palladium. Heat the reaction mixture to 130° C. under nitrogen for 3 hours; use the TLC method to ensure the completeness of the reaction. When the reaction is complete, cool the mixture, filter through celite, and distill off the solvent. To the residue add a 1:1 mixture of acetone/hexane, boil for 5-10 minutes, and cool to −10° C. Filter the suspension, wash with hexane, and allow to dry in air. Yield: 1.5 g (79%).

BCD-BTK-4-5.

In a round-bottom flask, equipped with a stirrer, thermometer and reflux condenser, mix in the specified order: 20 mL of trifluoroacetic acid, 2.7 g (0.00918 mol) of BCD-BTK-4-6a, and 1.5 g (0.0051 mol) of BCD-BTK-4-6b. Allow the mixture to stand at 60° C. for 3-5 hours. Distill off trifluoroacetic acid as completely as possible, and dissolve the residue in 100 mL of water. Extract the aqueous solution containing small amount of precipitate with hexane; discard the organic layer; neutralize the aqueous layer to pH 6-7. Filter the resulting precipitate, and wash it once with 50 mL of a 1:1 mixture of acetone:hexane, twice with 20 mL of water; allow to dry in air. Yield: 2.1 g (92%).

BCD-BTK-4-4.

In a round-bottom flask, equipped with a stirrer, thermometer and reflux condenser, mix in the specified order: 20 mL of DMF, 2.1 g (0.011 mol) of BCD-BTK-4-5, and 3 g (0.013 mol) of N-iodosuccinimide. Heat the mixture to 70° C. and allow to stand at this temperature for 2 hours; use the TLC method to ensure the completeness of the reaction. When the reaction is complete, add the mixture to 100 mL of water; filter the suspension, wash the filter cake with water and a 1:1 mixture of acetone/water. Allow to dry in air. Yield: 3 g (93%).

BCD-BTK-4-3.

In a round-bottom flask, equipped with a stirrer, thermometer and reflux condenser, mix in the specified order: 15 mL of dry THF, 1 g (0.00333 mol) of BCD-BTK-4-4, 1.766 g (0.0066 mol) of triphenylphosphine, and 1.355 (0.0066 mol) of (S)-3-hydroxy-1-(tert-butoxycarbonyl)piperidine. Stir under nitrogen for 15 minutes. Cool the reaction mass to 0° C.; add, dropwise, 1.173 g (0.0066 mol) of diethyl azodicarboxylate, maintaining the temperature. After that, allow the reaction mass to warm to room temperature, and stir for 3 hours; use the TLC method to ensure the completeness of the reaction. When the reaction is complete, distill off the solvent, and treat the residue with a mixture of hexane/ethyl acetate (7:3). Filter off and discard the resulting precipitate; concentrate the mother liquor, and purify the resulting product by column chromatography, eluent dichloromethane:ethyl acetate (from 9:1 to 7:3). Yield: 0.61 g (39%).

BCD-BTK-6-3.

In a round-bottom flask, equipped with a stirrer, thermometer and reflux condenser, mix in the specified order: 5 mL of DMSO, 0.6 g (0.00128 mol) of BCD-BTK-4-3, 0.355 g (0.00257 mol) of potassium carbonate, and 0.845 g (0.0076 mol) of 30% hydrogen peroxide in water. Stir the mixture at 30° C. for 4 hours; use the TLC method to ensure the completeness of the reaction. When the reaction is complete, add 25 mL of water to the reaction mass, and extract with 20 mL of ethyl acetate five times. Combine the organic extracts, wash them with NaCl solution, dry with sodium sulfate, and distill off the solvent. Purify the resulting product by column chromatography, eluent ethyl acetate. Yield: 0.55 g (90%).

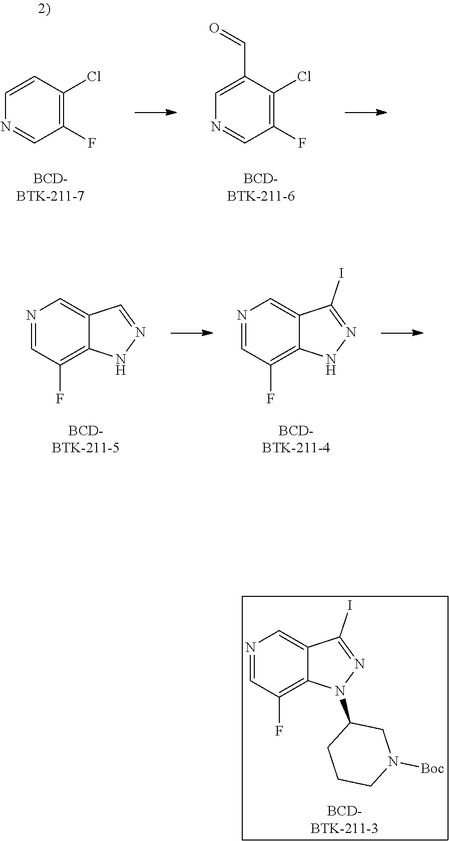 [SMILES: Fc1cnccc1Cl ; O=Cc1cncc(F)c1Cl ; Fc1cncc2cn[nH]c12 ; Fc1cncc2c(I)n[nH]c12 ; CC(C)(C)OC(=O)N1CCC[C@@H](n2nc(I)c3cncc(F)c32)C1]

BCD-BTK-211-6.

In a round-bottom flask, equipped with a stirrer, thermometer and reflux condenser, add under nitrogen 50 ml of dry THF, cool to −78° C. and add dropwise in the specified order: 9.57 ml of 2.5M butyl lithium in hexane, 2.42 g (0.02 mol) diisopropylamine and a solution of 3 g (0.022 mol) of the compound BCD-BTK-211-7 in 20 ml of dry THF. Keep the mixture at −78° C. for 5.5 hours. Then add 1.75 g (0.023 mol) of DMF maintaining the temperature. After that keep the mixture at room temperature for 1 hour. Then add while cooling the reaction mixture 10 ml of methanol and 30 ml of aqueous NH₄Cl and keep for 30 minutes. Add 200 ml of water, 120 ml of dichloromethane and transfer the emulsion into a separation funnel. Separate the organic layer, re-extract the water layer with 60 ml of dichloromethane. Combine the organic layers, wash with water and dry with sodium sulfate. Distill off dichloromethane. Purify the resulting product by column chromatography, eluent hexane:ethyl acetate (95:5). Yield: 1.8 g (50%).

BCD-BTK-211-5.

In a thick-walled flask with a threaded neck, mix 25 mL of DMSO, 1.6 g (0.0099 mol) of BCD-BTK-211-6, and 2 g (0.039 mol) of hydrazine hydrate and heat up to 130-140° C. for 6 hours. After that distill off the solvent using a rotary evaporator. Add 50 ml of water and 25 ml of ethyl acetate to the residue and transfer the emulsion into a separation funnel. Separate the organic layer, re-extract the water layer with 25 ml of ethylacetate. Combine the organic layers, wash with water and dry with sodium sulfate. Distill off ethyl acetate. Purify the resulting product by column chromatography, eluent hexane:ethyl acetate (7:3). Yield: 0.85 g (62%).

BCD-BTK-211-4.

In a round-bottom flask, equipped with a stirrer, thermometer and reflux condenser, mix under nitrogen in the specified order: 10 mL of DMF, 0.81 g (0.0059 mol) of BCD-BTK-211-5 and 1.6 g (0.07 mol) of N-iodosuccinimide. Stir at 80° C. for 3 hours, use the TLC method to ensure the completeness of the reaction. When the reaction is complete, add 80 ml of water and filtrate the precipitate, wash it with water 2 times and dry at 40° C. under vacuum. Yield: 1.3 g (85%).

BCD-BTK-211-3.

In a round-bottom flask, equipped with a stirrer, thermometer and reflux condenser, mix under nitrogen in the specified order: 25 mL of dry THF, 1.25 mL (0.0047 mol) of BCD-BTK-211-4, 2.5 g (0.0094 mol) of triphenylphosphine and 1.19 g (0.0094 mol) of (S)-3-hydroxy-1-(tert-butoxycarbonyl)piperidine and stir for 15 minutes. Then cool the reaction mass to 0° C. and add, dropwise, 1.93 g (0.0094 mol) of diisopropylazodicarboxylate keeping that temperature. After that heat the reaction mass to room temperature and stir for 10 hours, use the TLC method to ensure the completeness of the reaction. When the reaction is complete, distill of the solvent, treat the residue with a mixture of hexane-ethyl acetate 9:1. Filter and discard the resulting precipitate; concentrate the mother liquor. Purify the resulting product by column chromatography, eluent hexane:ethyl acetate (from 9:1 to 8:2). Yield: 1.18 g (56%).

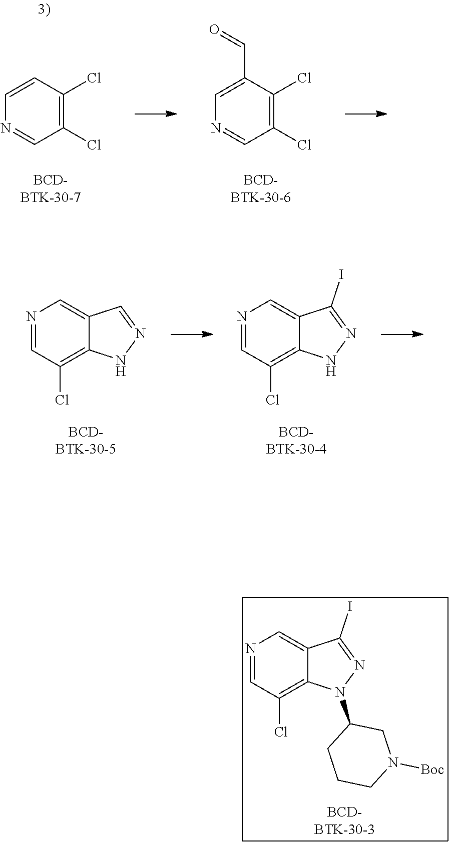 [SMILES: Clc1ccncc1Cl ; O=Cc1cncc(Cl)c1Cl ; Clc1cncc2cn[nH]c12 ; Clc1cncc2c(I)n[nH]c12 ; CC(C)(C)OC(=O)N1CCC[C@@H](n2nc(I)c3cncc(Cl)c32)C1]

BCD-BTK-30-6.

In a round-bottom flask, equipped with a stirrer, thermometer and reflux condenser, mix under nitrogen in the specified order: 200 mL of dry THF and 12.308 g (0.12 mol) of diisopropylamine. Cool the resulting mixture to −40° C.; add, dropwise, 48.7 mL of 2.5M butyllithium in hexane. Allow the mixture to stand at this temperature. After 30 minutes, cool the mixture to −78° C.; add, dropwise, solution of 15.7 g (0.068 mol) of BCD-BTK-30-7 in 100 mL of dry THF. After that, allow the reaction mass to stand for 2.5 hours. Add 15 g (0.2 mol) of DMF, maintaining the temperature at −78° C. Stop the cooling and allow the reaction mixture to warm to room temperature. Allow to stand for another hour. Add, while cooling the reaction mixture, 30 mL of methanol and 150 mL of NH₄Cl aqueous solution. Allow to stand for 30 minutes. To the reaction mass add 1000 mL of water, 500 mL of dichloromethane, and transfer the emulsion to a separatory funnel. Separate the organic layer; re-extract the aqueous layer using 200 mL of dichloromethane. Combine the organic layers, wash with water, and dry with sodium sulfate. Distill off dichloromethane. Yield: 17 g (96%).

BCD-BTK-30-5.

In a round-bottom flask, equipped with a stirrer, thermometer and reflux condenser, mix under nitrogen in the specified order: 20 mL of ethanol, 2.7 g (0.0152 mol) of BCD-BTK-30-6, and 3.07 g (0.06 mol) of hydrazine hydrate. Stir the mixture while boiling for 4 hours; use the TLC method to ensure the completeness of the reaction. When the reaction is complete, pour the mixture into 100 mL of water and cool it to 0° C. Filter the resulting precipitate, wash twice with 20 mL of water, and dry under vacuum at 40° C. Yield: 1 g (43%).

BCD-BTK-30-4.

In a round-bottom flask, equipped with a stirrer, thermometer and reflux condenser, mix under nitrogen in the specified order: 10 mL of DMF, 1 g (0.0064 mol) of BCD-BTK-30-5, and 1.9 g (0.0083 mol) of N-iodosuccinimide. Stir the mixture at 80° C. for 3 hours; use the TLC method to ensure the completeness of the reaction. When the reaction is complete, pour the mixture into 100 mL of water; filter the resulting precipitate, wash twice with water, and dry under vacuum at 40° C. Yield: 1.75 g (94%).

BCD-BTK-30-3.

In a round-bottom flask, equipped with a stirrer, thermometer and reflux condenser, mix under nitrogen in the specified order: 25 mL of dry THF, 1 g (0.00956 mol) of BCD-BTK-30-4, 5.064 g (0.01912 mol) of triphenylphosphine, and 3.889 g (0.01912 mol) of (S)-3-hydroxy-1-(tert-butoxycarbonyl)piperidine. Stir for 15 minutes. Cool the reaction mass to 0° C.; add, dropwise, 3.907 g (0.01912 mol) of diisopropyl azodicarboxylate, maintaining the temperature. After that, allow the reaction mass to warm to room temperature, and stir for 10 hours; use the TLC method to ensure the completeness of the reaction. When the reaction is complete, distill off the solvent, and treat the residue with a mixture of hexane/ethyl acetate (9:1). Filter and discard the resulting precipitate; concentrate the mother liquor. Purify the resulting product by column chromatography, eluent hexane:ethyl acetate (from 9:1 to 7:3). Yield: 1.8 g (41%).

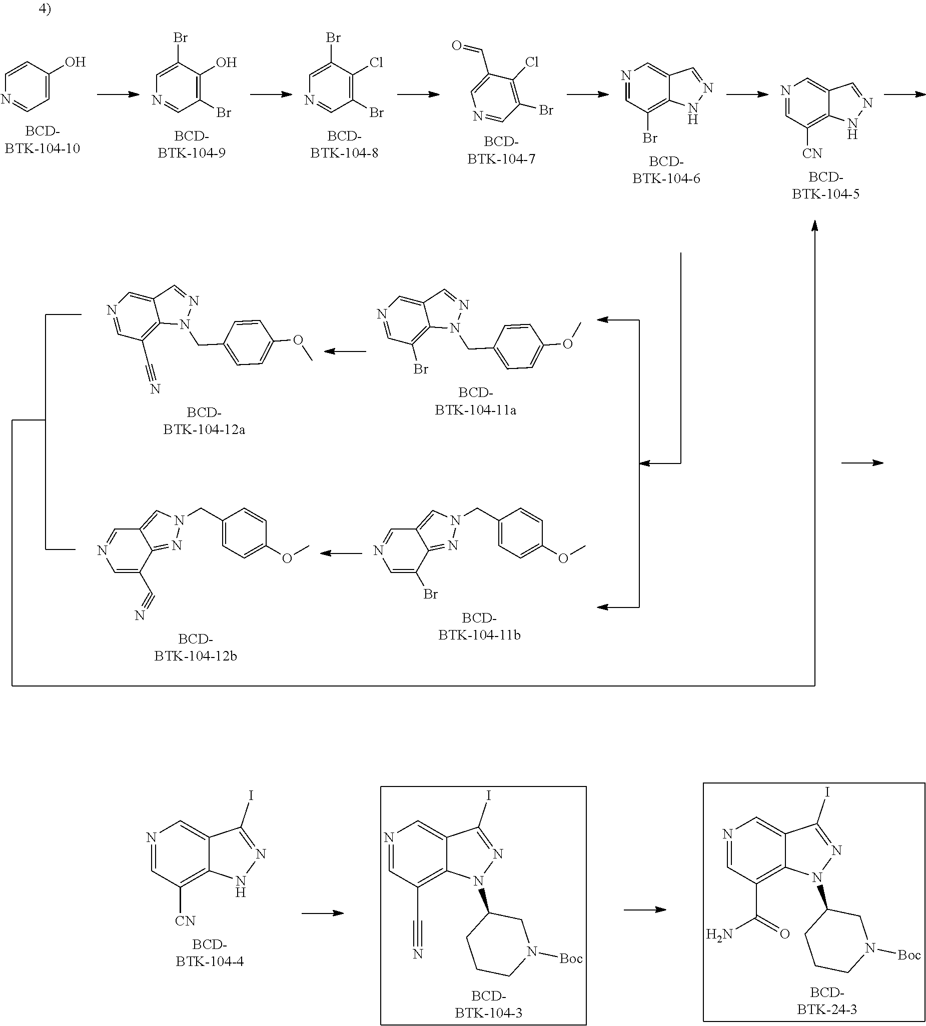

BCD-BTK-104-9.

In a round-bottom flask, equipped with a stirrer, thermometer and reflux condenser, mix under nitrogen in the specified order: 500 mL of tetrachloromethane, 23.75 g (250 mmol) of 4-hydroxypyridine, and 89 g (500 mmol) of N-bromosuccinimide. Stir at 25° C. for 30 hours. Filter the precipitate, wash with 50 mL of tetrachloromethane; stir the precipitate in a mixture of 500 mL of acetone and 150 mL of methanol for 15 minutes. Filter the suspension; stir the precipitate in a mixture of 400 mL of acetone and 400 mL of dichloromethane for 15 minutes. Filter the suspension; mix the precipitate vigorously in 400 mL of acetonitrile for 20 minutes. Filter the suspension; dry the precipitate under vacuum at 40° C. Yield: 56 g (88%).

BCD-BTK-104-8.

In a round-bottom flask, equipped with a stirrer, thermometer and reflux condenser, mix under nitrogen in the specified order: 120 mL of phosphoryl chloride and 38 g (150 mmol) of BCD-BTK-104-9. Stir the reaction mass at 70° C. for 3 hours, cool it to 40° C., and pour on ice, while stirring vigorously. Filter the precipitate, wash with water, and dry under vacuum at 40° C. Yield: 36.7 g (91%).

BCD-BTK-104-7.

In a round-bottom flask, equipped with a stirrer, thermometer and reflux condenser, mix under nitrogen in the specified order: 200 mL of dry THF and 30 g (110 mmol) of BCD-BTK-104-8. Cool the mixture in the ice bath; add, dropwise, 2M i-PrMgCl in THF (60 mL, 120 mmol). Stir the suspension at 20° C. for 1 hour, cool in the ice bath; add, dropwise, 17 mL (16 g, 220 mmol) of dimethylformamide, while stirring vigorously. Stir the reaction mass at 20° C. for 4 hour, cool in the ice bath; add 15 mL of 15% NH₄Cl and 50 mL of water, while stirring vigorously. To the reaction mass add 100 mL of ethyl acetate; separate the organic layer, extract the aqueous layer with 70 mL of ethyl acetate twice. Wash the combined organic layers with 30 mL of water twice, and then with saturated NaCl solution; dry with anhydrous sodium sulfate. Distill off the solvent; treat the residue with 30 mL of hexane, filter, and dry under vacuum at 40° C. Yield: 20 g (60%).

BCD-BTK-104-6.

In a thick walled flask with a threaded neck, mix 30 mL of DMSO, 1.7 g (0.00723 mol) of BCD-BTK-104-7, and 1.3 g (0.025 mol) of hydrazine hydrate. Screw the cap on tightly, and heat the flask to 130-140° C. for 16 hours. After that, transfer the reaction mass to a flask, and distill off the solvent using a rotary evaporator. To the residue add 100 mL of water and cool to +5° C. Filter the resulting precipitate, wash twice with chilled water, and allow to dry in air. Yield: 1.2 g (86%).

BCD-BTK-104-11a, BCD-BTK-104-11b.

Add 170 ml of DMSO, then add 2.18 g (54.5 mmol) of sodium hydride (60% suspension in paraffin oil) to a 500 ml round-bottom flask. Stir the mixture at room temperature under nitrogen for 15 min. Add successively 9.00 g (45.4 mmol) BCD-BTK-104-6 and 8.17 g (52.2 mmol) 4-methoxybenzyl chloride to the mixture. Stir the mixture at room temperature for 20 h, add 900 ml of water, extract water phase with ethyl acetate (3×400 ml), wash the combined organic layers with water (3×300 ml), dry with Na₂SO₄. Purify the resulting product (as two isomers) by chromatography. Yield: 12 g (86%) of isomer mixture.

BCD-BTK-104-12a, BCD-BTK-104-12b.

Add the mixture of 12 g, (37.7 mmol) of BCD-BTK-104-11a and BCD-BTK-104-11b, 6.60 g, (56.6 mmol) of Zn(CN)₂, 0.86 g, (0.94 mmol) of Pd₂(dba)₃, 0.69 g, (0.94 mmol) of Pd(dppf)Cl₂ and 120 ml of DMF to a 500 ml round-bottom flask. Pass intensive stream of nitrogen for 5 minutes, heat at 120° C. for 2.5 hours. Distill off 4/5 of DMF volume under reduced pressure, add 200 ml of water, 400 ml of ethyl acetate to the residue, separate the organic layer, extract the water layer with ethyl acetate (2×100 ml), wash the combined organic layers with water (2×100 ml) and the saturated NaCl solution, dry with Na₂SO₄. Purify the resulting product by chromatography, eluent dichloromethane-ethyl acetate (1:1). Yield: 9.2 g (93%) of isomer mixture.

BCD-BTK-104-5.

Add the mixture of 9.2 g (35.0 mmol) of BCD-BTK-104-12a and BCD-BTK-104-12b to a 250 ml round-bottom flask. Add 59.6 g (522 mmol) of trifluoroacetic acid and heat the intensively boiling reaction mass for 3.5 h, cool to room temperature, distill off trifluoroacetic acid under reduced pressure in a rotary film evaporator to 1/5 of the initial volume. Pour into water, bring to pH=7, extract the water layer by ethyl acetate, wash the combined organic extracts with water, dry with Na₂SO₄. Purify the resulting product by column chromatography, eluent ethyl acetate-hexane (7:3). Boil the resulting 4 g of solid mass (80%) in 50 ml of methylene chloride, cool to 0° C., filter, wash with methylene chloride (10 ml) and dry. Yield: 2.9 g (58%).

BCD-BTK-104-5.

In a round-bottom flask, equipped with a stirrer, thermometer and reflux condenser, mix under nitrogen in the specified order: 70 mL of dry DFM, 7.0 g (35.4 mmol) of BCD-BTK-104-6, 0.05 g of [1,1′-bis(diphenylphosphino)ferrocene]dichloropalladium (II), 0.7 g (10.8 mmol) of zinc dust, and 4.9 g (41 mmol) of zinc cyanide. Stir the mixture under nitrogen at 100° C. for 3 hours; allow to cool, filter through celite; wash the celite with DFM (2×20 mL), and concentrate the filtrate. Purify the resulting product by column chromatography, eluent ethyl acetate. Yield: 3.0 g (59%).

BCD-BTK-104-4.

In a round-bottom flask, equipped with a stirrer, thermometer and reflux condenser, mix under nitrogen in the specified order: 10 mL of DMF, 3 g (0.021 mol) of BCD-BTK-104-5, and 5.7 g (0.025 mol) of N-iodosuccinimide. Stir the mixture at 40° C. for 5 hours; use the TLC method to ensure the completeness of the reaction. When the reaction is complete, pour the mixture into 100 mL of water; filter the resulting precipitate, wash twice with water, and dry under vacuum at 40° C. Yield: 4.8 g (85%).

BCD-BTK-104-3.

In a round-bottom flask, equipped with a stirrer, thermometer and reflux condenser, mix under nitrogen in the specified order: 25 mL of dry THF, 4.8 g (0.018 mol) of BCD-BTK-104-4, 9.4 g (0.036 mol) of triphenylphosphine, and 7.2 g (0.036 mol) of (S)-3-hydroxy-1-(tert-butoxycarbonyl)piperidine. Stir for 15 minutes. Cool the reaction mass to 0° C.; add, dropwise, 7.3 g (0.036 mol) of diisopropyl azodicarboxylate, maintaining the temperature. After that, heat the reaction mass to 40° C., and stir for 6 hours; use the TLC method to ensure the completeness of the reaction. When the reaction is complete, distill off the solvent; purify the resulting product by column chromatography, eluent dichloromethane. Yield: 3.3 g (40%).

BCD-BTK-24-3.

In a round-bottom flask, equipped with a stirrer, thermometer and reflux condenser, mix in the specified order: 5 mL of DMSO, 0.6 g (0.00128 mol) of BCD-BTK-104-3, 0.355 g (0.00257 mol) of potassium carbonate, and 0.845 g (0.0076 mol) of 30% hydrogen peroxide in water. Stir the mixture at 30° C. for 4 hours; use the TLC method to ensure the completeness of the reaction. When the reaction is complete, add 25 mL of water to the reaction mass, and extract with 20 mL of ethyl acetate five times. Wash the organic layer with NaCl solution and water, dry with sodium sulfate, and distill off the solvent. Purify the resulting product by column chromatography, eluent dichloromethane:ethyl acetate (9:1). Yield: 0.55 g (90%).

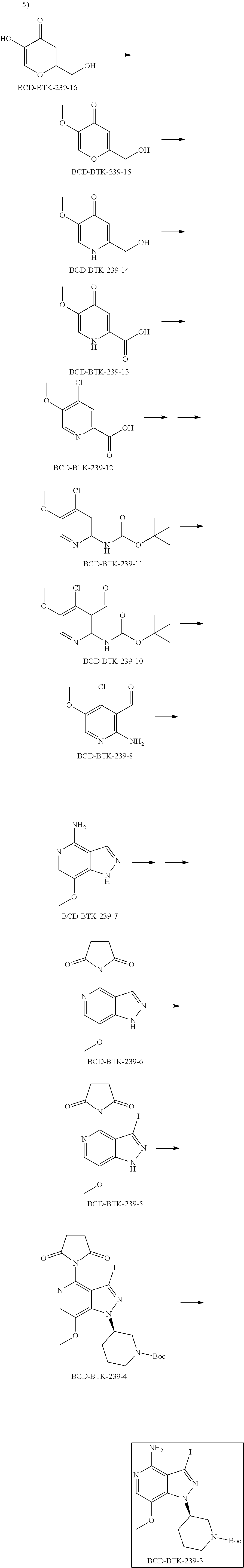

BCD-BTK-239-15.

In a round-bottom flask, mix in the specified order: solution of 2.17 g (38.3 mmol) of KOH in 22 ml of water and 5.00 g (34.8 mmol) of BCD-BTK-239-16. Stir the reaction mass at 20° C. for 20 minutes, cool in an ice bath to 5° C. and add while stirring dropwise 4.44 g (34.8 mmol) of dimethyl sulfate. Stir the reaction mass at 20° C. for 3 h, allow it to stand at 4° C. for 20 h. Filter the precipitate, wash with water, dry under vacuum at 40° C. Yield: 6.04 g (90%).

BCD-BTK-239-14.

In a round-bottom flask, mix in the specified order: 6.80 g (43.6 mmol) of BCD-BTK-239-15 and 70.0 ml (930 mmol) of aqueous ammonia. Stir the reaction mass at 90° C. for 2.5 h, cool down, distill off the solvent under reduced pressure, dissolve the residue in 100 ml of methanol, add 1 g of activated carbon, boil for 30 minutes, cool down, filter through celite and distill off the solvent to dryness. Yield: 6.50 g (96%).

BCD-BTK-239-13.

In a round-bottom flask, mix in the specified order: a mixture of 1 ml of 98% nitric acid and 5 ml of 70% nitric acid and 1 g (6.44 mmol) of BCD-BTK-239-14. Stir the reaction mass at 20° C., pour a mixture of 6 g of ice and 30 ml of water into the reaction mass. Filter the precipitate, wash with water, dry under vacuum at 40° C. Yield: 0.96 g (89%).

BCD-BTK-239-12.

In a round-bottom flask, equipped with a stirrer, thermometer and reflux condenser, mix in the specified order: 26.2 g (220 mmol) of thionyl chloride and 7.60 g (44.9 mmol) of BCD-BTK-239-13.Boil the mixture with the reflux condenser and a calcium chloride tube for 5 h. Distill the solvent off to dryness under reduced pressure, add to the residue successively 30 g of ice and 70 ml of water and bring pH to 8 with solid Na₂CO₃. Stir the reaction mass for 20 h, bring pH to 7 with 2 N hydrochloric acid, filtrate the precipitate, wash with water, dry under vacuum at 40° C. for 24 h. Yield: 4.8 g (58%).

BCD-BTK-239-11.

In a round-bottom flask, equipped with a stirrer, thermometer and reflux condenser, mix in the specified order: 130 ml of tert-butanol, 4.32 g (23.0 mmol) of BCD-BTK-239-12 and 6.42 g (23.0 mmol) of DPPA and add while stirring dropwise 2.33 g (23.0 mmol) of triethylamine. Boil the reaction mass while stirring under nitrogen for 16 h, distill off the solvent. Purify the product by column chromatography on silicagel, eluent ethyl acetate:hexane (1:9). Yield: 4.35 g (73%).

BCD-BTK-239-10.

In a round-bottom flask, equipped with a stirrer, thermometer and reflux condenser, mix in the specified order: 2.00 g (7.73 mmol) of BCD-BTK-239-11, 2.24 g (19.3 mmol) of N,N,N′,N′-tetramethylethylenediamine and 90 ml of dry THF. Cool the reaction mass to −78° C. while constant stirring under nitrogen. Add to the reaction mass with a syringe 7.72 ml (19.3 mmol) of 2.5M of a solution of n-butyllithium in hexane for 5 minutes, continue stirring at the same temperature for 1 h; add 1.68 g (23.0 mmol) of DMF to the reaction mass, continue stirring at −78° C. for another hour, then add to the reaction mass a saturated solution of NH₄Cl (20 ml), heat to 20° C. and add 100 ml of ethyl acetate, separate the organic layer, extract the water layer with ethyl acetate (2×50 ml), wash the combined organic layers with a saturated solution of NaCl, dry with Na₂SO₄. Distill off the solvent under reduced pressure, purify the product by column chromatography on silicagel, eluent ethyl acetate:hexane (2:8). Yield: 1.53 g (70%).

BCD-BTK-239-8.

In a round-bottom flask, equipped with a stirrer, thermometer and reflux condenser, mix in the specified order: 1.25 g (4.36 mmol) of BCD-BTK-239-10 and 20.0 ml (80.0 mmol) of 4N HCl solution in dioxane at 20° C. and stir for 18 h. Distill off the solvent to dryness at reduced pressure, dissolve the residue in 5 ml of water, extract the water layer with 5 ml of methyl-t-butyl ether, separate the water layer, bring pH to 8 with 3N solution of KOH, Filtrate the precipitate, wash with water, dry under vacuum at 40° C. for 24 h. Yield: 0.75 g (93%).

BCD-BTK-239-7.

Dissolve 0.70 g (3.75 mmol) of BCD-BTK-239-8 in 5 ml of DMSO, add to 0.28 g (5.63 mmol) of hydrazine hydrate. Place the reaction mass into a flask under pressure and heat at 120° C. while stirring for 15 h, distill off the solvent at reduced pressure, purify the product by column chromatography on silicagel, eluent ethyl acetate and then ethyl acetate/methanol (7:3). Yield: 0.59 g (96%).

BCD-BTK-239-6.

In a round-bottom flask, mix in the specified order: 4.4 g (44 mmol) of succinic anhydride and 3.28 g (20 mmol) of BCD-BTK-239-7. Mix the reaction mass at 160° C. for 20 min, pour into the reaction mass 6 g of ice and 30 ml of water. Filtrate the precipitate, wash with water, dry under vacuum at 40° C. Yield: 3.1 g (62%).

BCD-BTK-239-5.

In a round-bottom flask, equipped with a stirrer, thermometer and reflux condenser, mix under nitrogen in the specified order: 25 ml of DMF, 3 g (0.012 mol) of BCD-BTK-239-6 and 3.2 g (0.014 mol) of N-iodosuccinimide. Stir the mixture at 40° C. for 5 h; use the TLC method to ensure the completeness of the reaction. When the reaction is complete, pour 100 ml of water and filtrate the precipitate, wash with water (2 times) and dry at 40° C. under vacuum. Yield: 2.86 g (77%).

BCD-BTK-239-4.

In a round-bottom flask, equipped with a stirrer, thermometer and reflux condenser, mix under nitrogen in the specified order: 25 ml of dry THF, 2.86 g (0.0067 mol) of BCD-BTK-239-5, 3.93 g (0.015 mol) of triphenylphosphine and 3.0 g (0.015 mol) of (S)-3-hydroxy-1-(t-butoxycarbonyl) piperidine, and mix for 15 minutes. Then cool the reaction mass to 0° C. and add dropwise 3.0 g (0.015 mol) diisopropyl azodicarboxylate, keeping the temperature at the same level. After that, heat the reaction mass to 20° C. and mix for 6 hours; use the TLC method to ensure the completeness of the reaction. When the reaction is complete, distill off the solvent, purify the product by column chromatography, eluent: ethyl acetate-hexane (8:2). Yield: 2.08 g (56%).

BCD-BTK-239-3.

In a round-bottom flask, equipped with a stirrer, thermometer and reflux condenser, mix under nitrogen in the specified order: 25 ml of dry methanol, 2.08 g (0.0037 mol) of BCD-BTK-239-4 and 1 ml of hydrazine hydrate, stir for 6 h; use the TLC method to ensure the completeness of the reaction. When the reaction is complete, distill off the solvent at room temperature and add 20 ml of water. Filter the precipitate, wash with water and dry. Yield: 1.45 g (79%).

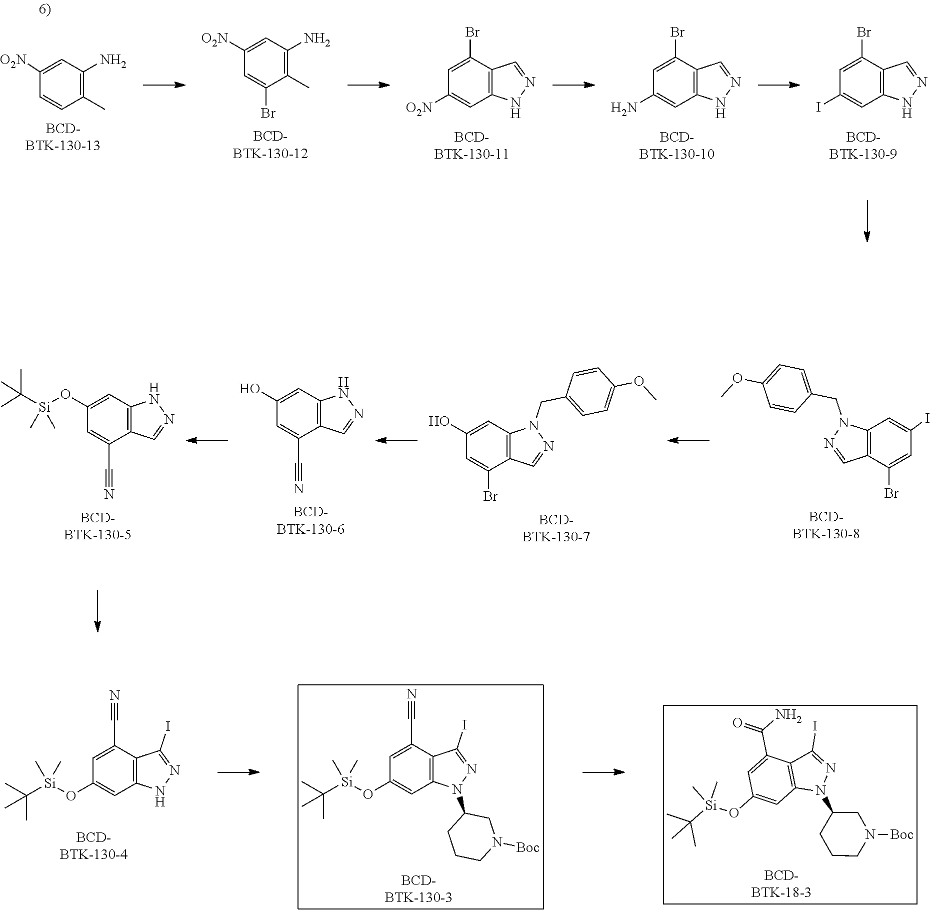

BCD-BTK-130-12.

In a round-bottom flask, equipped with a stirrer, thermometer and reflux condenser, mix under nitrogen in the specified order: 300 mL of acetic acid and 20 g (0.131 mol) of 2-amino-5-nitrotoluene. To the resulting mixture add a solution of 31 g of bromine in 20 mL of acetic acid. After that, allow the reaction mass to stand for 1 hour and pour it into 2 L of water, add 20 g of sodium hydrogen sulfite, and stir for 30 minutes. Filter the precipitate, wash with water, and re-crystallize from 1 L of ethanol. Yield: 26 g (86%).

BCD-BTK-130-11.

In a round-bottom flask, equipped with a stirrer, thermometer and reflux condenser, mix under nitrogen in the specified order: 190 mL of acetic acid and 26 g (0.113 mol) of BCD-BTK-130-12. Stir the mixture for 2 hours, cool to 15° C., and add, dropwise, a solution of 20 g (0.29 mol) of sodium nitrite in water. Stir the reaction mass for 24 hours at room temperature. Then pour it into 1 L of water; filter the precipitate, wash with 20 mL of water twice. Re-crystallize the precipitate from 200 mL of ethanol. Yield: 17.3 g (63%).

BCD-BTK-130-10.

In a round-bottom flask, equipped with a stirrer, thermometer and reflux condenser, mix under nitrogen in the specified order: 40 mL of ethanol, 2.3 g (0.04 mol) of iron powder, 4.1 g (0.076 mol) of NH₄Cl, and 1 g (0.00413 mol) of BCD-BTK-130-11. Stir the resulting mixture for 3 hours at boiling temperature; use the TLC method to ensure the completeness of the reaction. When the reaction is complete, filter the mixture through celite, concentrate the solvent and residue. Filter the precipitate, wash with 30 mL of water, and allow to dry in air. Yield: 0.8 g (91%).

BCD-BTK-130-9.

In a round-bottom flask, equipped with a stirrer, thermometer and reflux condenser, mix under nitrogen in the specified order: 20 mL of water, 5 mL of concentrated hydrochloric acid, and 3 g (0.0144 mol) of BCD-BTK-130-10. Cool the mixture to 0° C., and add, dropwise, a solution of 1.19 g (0.0173 mol) of sodium nitrite in 5 mL of water, maintaining the temperature (0° C.). Allow the reaction mass to stand for another hour, and add a solution of 11.9 g (0.072 mol) of potassium iodide in water. Allow the resulting mixture to stand at room temperature. After 3 hours, neutralize to pH 6-7 with sodium bicarbonate, and extract three times with 30 mL of ethyl acetate. Wash the organic layer with water, dry with sodium sulfate, and distill off the solvent. Purify the resulting product by column chromatography, eluent hexane:ethyl acetate (from 1:1 to 2:8). Yield: 2.76 g (60%).

BCD-BTK-130-8.

In a round-bottom flask, equipped with a stirrer, thermometer and reflux condenser, mix under nitrogen in the specified order: 60 mL of dry THF, 2.1 g (0.0065 mol) of BCD-BTK-130-9, 3.4 g (0.013 mol) of triphenylphosphine, and 1.373 g (0.00975 mol) of p-methoxybenzyl alcohol. Stir for 15 minutes. Cool the reaction mass to 0° C.; add, dropwise, 2.333 g (0.013 mol) of diethyl azodicarboxylate, maintaining the temperature. After that, allow the reaction mass to warm to room temperature, and stir for 1.5 hours; use the TLC method to ensure the completeness of the reaction. When the reaction is complete, distill off the solvent; purify the residue by column chromatography, eluent hexane:ethyl acetate (9:1). Yield: 1.72 g (60%).

BCD-BTK-130-7.

In a round-bottom flask, equipped with a stirrer, thermometer and reflux condenser, mix under nitrogen in the specified order: 120 mL of dry THF and 2.7 g (0.0061 mol) of BCD-BTK-130-8. Cool the suspension to 6° C., and add, dropwise, 1.6 mL (0.0079 mol) of 2M i-PrMgCl in THF. Stir the mixture at 5° C. After 1 hour, add 1.84 g (0.0098 mol) of triisopropyl borate, stir for 10 hours at room temperature, and cool again to 5° C. To the resulting mixture add 6 mL (0.061 mol) of 30% hydrogen peroxide, 0.244 g (0.0061 mol) of solid NaOH, and stir for another hour at room temperature. After that, add a solution of 9.6 g (0.076 mol) of sodium sulfite in 30 mL of water, stir, and extract with ethyl acetate. Wash the organic layer with water, dry with sodium sulfate, and distill off the solvent. Purify the resulting product by column chromatography, eluent hexane:ethyl acetate (2:3). Yield: 1.3 g (63%).

BCD-BTK-130-6.

In a round-bottom flask, equipped with a stirrer, thermometer and reflux condenser, mix under nitrogen in the specified order: 20 mL of dry DMF, 0.62 g (0.00186 mol) of BCD-BTK-130-7, 0.13 g (0.00112 mol) of zinc cyanide, and 0.09 g (0.000079 mol) of tetrakis(triphenylphosphine)palladium. Heat the mixture to 80° C. for 10 hours; use the TLC method to ensure the completeness of the reaction. When the reaction is complete, cool the mixture, filter through celite, and distill off the solvent. Purify the residue by column chromatography, eluent hexane:ethyl acetate (8:2). Yield: 0.46 g (90%). Dissolve the resulting product in 20 mL of trifluoroacetic acid and stir at 60° C. for 1 hour. Distill off the excess acid, neutralize the residue with a solution of sodium bicarbonate; distill off water. Purify the resulting product by column chromatography, eluent ethyl acetate. Yield: 0.23 g (56%).

BCD-BTK-130-5.

In a round-bottom flask, equipped with a stirrer, thermometer and reflux condenser, mix under nitrogen in the specified order: 10 mL of dry DMF, 1.1 g (0.0069 mol) of BCD-BTK-130-6, and 0.71 g (0.0104 mol) of imidazole. Cool the mixture to 0° C. and add 1.56 g (0.0104 mol) of TBDMSCl. Allow to stand at this temperature for 30 minutes. When the reaction is complete, pour the reaction mass into water, and extract with 20 mL of ethyl acetate three times. Wash the ethyl acetate with 30 mL of water twice, dry with sodium sulfate, and distill off the solvent. Purify the resulting product by column chromatography, eluent hexane:ethyl acetate (8:2). Yield: 1.2 g (63%).

BCD-BTK-130-4.

In a round-bottom flask, equipped with a stirrer, thermometer and reflux condenser, mix in the specified order: 3 mL of DMF, 0.3 g (0.0011 mol) of BCD-BTK-130-5, and 0.36 g (0.00165 mol) of N-iodosuccinimide. Stir the mixture at 20° C. for 18 hours; use the TLC method to ensure the completeness of the reaction. When the reaction is complete, pour the reaction mass into water, and extract with 20 mL of ethyl acetate three times. Wash the organic layer with 30 mL of water twice, dry with sodium sulfate, and distill off the solvent. Purify the resulting product by column chromatography, eluent hexane:ethyl acetate (8:2). Yield: 0.36 g (82%).

BCD-BTK-130-3.

In a round-bottom flask, equipped with a stirrer, thermometer and reflux condenser, mix under nitrogen in the specified order: 10 mL of dry THF, 0.36 g (0.0009 mol) of BCD-BTK-130-4, 0.470 g (0.0018 mol) of triphenylphosphine, and 0.37 g (0.0018 mol) of (S)-3-hydroxy-1-(tert-butoxycarbonyl)piperidine. Stir for 15 minutes. Cool the reaction mass to 0° C.; add, dropwise, 0.370 g (0.0018 mol) of diisopropyl azodicarboxylate, maintaining the temperature. After that, allow the reaction mass to warm to room temperature, and stir for 30 hours; use the TLC method to ensure the completeness of the reaction. When the reaction is complete, distill off the solvent, and treat the residue with a mixture of hexane/ethyl acetate (9:1). Filter off and discard the resulting precipitate; concentrate the mother liquor, and purify the resulting product by column chromatography, eluent dichloromethane (from 9:1 to 7:3). Yield: 0.33 g (63%).

BCD-BTK-18-3.

In a round-bottom flask, equipped with a stirrer, thermometer and reflux condenser, mix in the specified order: 5 mL of DMSO, 0.6 g (0.00128 mol) of BCD-BTK-130-3, 0.355 g (0.00257 mol) of potassium carbonate, and 0.845 g (0.0076 mol) of 30% hydrogen peroxide in water. Stir the mixture at 20° C. for 14 hours; use the TLC method to ensure the completeness of the reaction. When the reaction is complete, add 25 mL of water to the reaction mass, and extract with 20 mL of ethyl acetate five times. Wash the organic layer with NaCl solution and water, dry with sodium sulfate, and distill off the solvent. Purify the resulting product by column chromatography, eluent ethyl acetate:hexane (1:1). Yield: 0.55 g (90%).

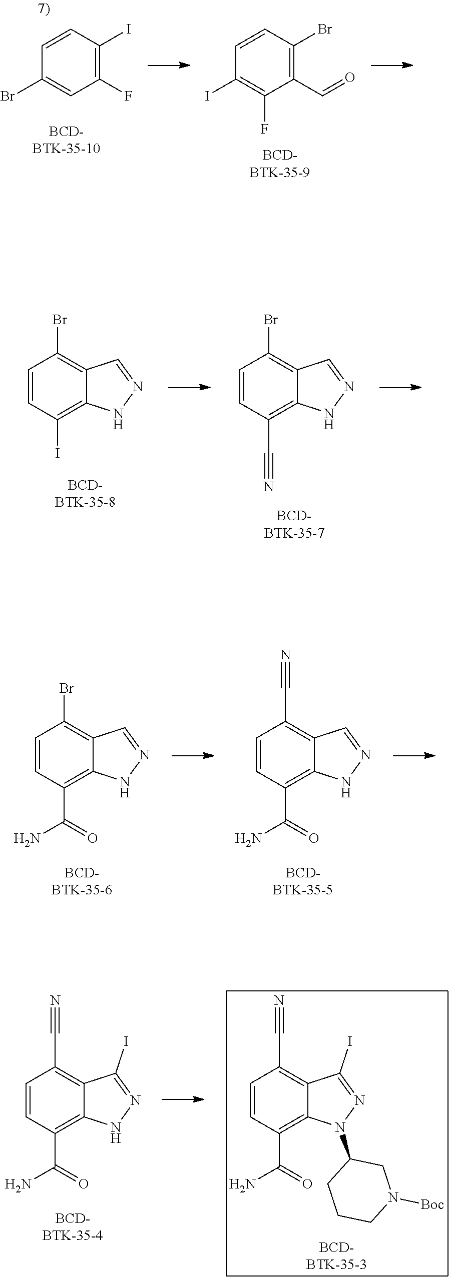

BCD-BTK-35-9.

In a round-bottom flask, equipped with a stirrer, thermometer and reflux condenser, mix under nitrogen in the specified order: 60 mL of dry THF and 3.067 g (0.0217 mol) of 2,2,6,6-tetramethylpiperidine. Cool the mixture to −78° C., add 8.69 mL (0.0217 mol) of 2.5M butyllithium in hexane, and allow to stand. After 20 minutes, add a solution of 6 g (0.01974 mol) of 3-fluoro-4-iodobromobenzene in 6 mL of THF, maintaining the temperature −78° C. Allow the mixture to stand for 1 hour and add 4.56 mL (0.059 mol) of DFM. Stop cooling and allow the mixture to warm to room temperature. Allow to stand for another hour. Add, while cooling the reaction mixture, 3 mL of methanol and 15 mL of NH₄Cl aqueous solution. Allow to stand for 30 minutes. To the reaction mass add 100 mL of water, 50 mL of dichloromethane, and transfer to a separatory funnel. Separate the organic layer; re-extract the aqueous layer twice using 25 mL of dichloromethane. Combine the organic layers, wash with water, dry with sodium sulfate, and distill off the solvent. Purify the resulting product by column chromatography, eluent hexane:dichloromethane (9:1). Yield: 3.1 g (47%).

BCD-BTK-35-8.

In a thick walled flask with a threaded neck, mix 150 mL of DMSO, 3.1 g (0.00933 mol) of BCD-BTK-35-9, and 3.5 g (0.0699 mol) of hydrazine hydrate. Heat the flask to 130-140° C. for 16 hours. After that, distill off the solvent using a rotary evaporator. To the residue add 100 mL of water and cool to 5° C. Filter the precipitate with chilled water, and allow to dry in air. Yield: 2.4 g (81%).

BCD-BTK-35-7.

In a round-bottom flask, equipped with a stirrer, thermometer and reflux condenser, mix under nitrogen in the specified order: 10 mL of dry DMF, 4.375 g (0.00654 mol) of BCD-BTK-35-8, 0.461 g (0.00393 mol) of zinc cyanide, and 0.378 g (0.00032 mol) of tetrakis(triphenylphosphine)palladium. Heat the mixture to 100° C. for 1.5 hours; use the TLC method to ensure the completeness of the reaction. When the reaction is complete, cool the mixture, filter through celite, and distill off the solvent as completely as possible. Add 20 mL of water and extract three times with 20 mL of ethyl acetate. Wash the organic layer three times with water, dry with sodium sulfate, and distill off the solvent. Purify the resulting product by column chromatography, eluent hexane:ethyl acetate (9:1). Yield: 1.35 g (92%).

BCD-BTK-35-6.

In a round-bottom flask, equipped with a stirrer, thermometer and reflux condenser, mix under nitrogen in the specified order: 10 mL of 95% sulfuric acid and 1.35 g (0.00599 mol) of BCD-BTK-35-7. Stir the resulting mixture for 2 hours at room temperature, and pour into 200 mL of iced water. Filter the resulting precipitate, wash with 20 mL of water twice, and allow to dry in air. Yield: 1.35 g (93%).

BCD-BTK-35-5.

In a round-bottom flask, equipped with a stirrer, thermometer and reflux condenser, mix under nitrogen in the specified order: 10 mL of dry DMF, 1.35 g (0.00556 mol) of BCD-BTK-35-6, 0.461 g (0.00393 mol) of zinc cyanide, and 0.643 g (0.00055 mol) of tetrakis(triphenylphosphine)palladium. Heat the reaction mixture to 110° C. for 2 hours; use the TLC method to ensure the completeness of the reaction. After that, pour the reaction mass into 200 mL of iced water, filter the resulting precipitate, wash twice with 20 mL of water, and allow to dry in air. Purify the resulting product by column chromatography, eluent ethyl acetate:methanol (9:1). Yield: 1 g (96%).

BCD-BTK-35-4.

In a round-bottom flask, equipped with a stirrer, thermometer and reflux condenser, mix under nitrogen in the specified order: 10 mL of DMF, 1 g (0.00558 mol) of BCD-BTK-35-5, and 1.5 g (0.0067 mol) of N-iodosuccinimide. Stir the mixture at 60° C. for 3 hours; use the TLC method to ensure the completeness of the reaction. When the reaction is complete, pour the mixture into 100 mL of water; filter the resulting precipitate, wash twice with 20 mL of water, and dry under vacuum at 40° C. Yield: 1.15 g (66%).

BCD-BTK-35-3.

In a round-bottom flask, equipped with a stirrer, thermometer and reflux condenser, mix under nitrogen in the specified order: 25 mL of dry THF, 1.15 g (0.00365 mol) of BCD-BTK-35-4, 1.916 g (0.0073 mol) of triphenylphosphine, and 1.468 g (0.0073 mol) of (S)-3-hydroxy-1-(tert-butoxycarbonyl)piperidine. Stir for 15 minutes. Cool the reaction mass to 0° C.; add, dropwise, 1.271 g (0.0073 mol) of diethyl azodicarboxylate, maintaining the temperature. After that, allow the reaction mass to warm to room temperature, and stir for 6 hours; use the TLC method to ensure the completeness of the reaction. When the reaction is complete, distill off the solvent; purify the resulting product by column chromatography, eluent ethyl acetate:hexane (1:1). Yield: 0.65 g (36%).

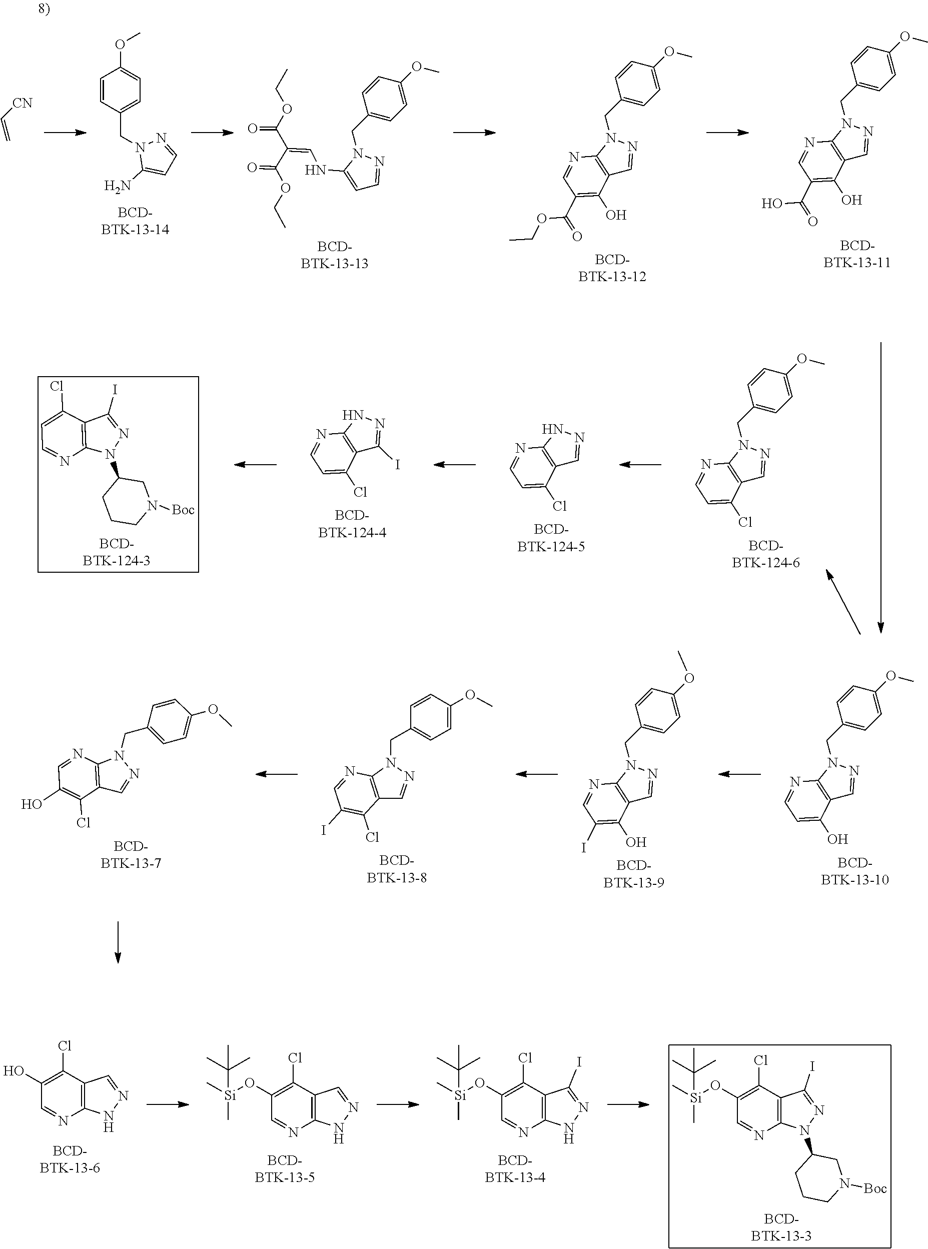

BCD-BTK-13-14.

In a round-bottom flask, equipped with a stirrer, thermometer and reflux condenser, mix under nitrogen in the specified order: 120 mL of ethanol and 23.4 g (0.436 mol) of acrylonitrile. Cool the mixture to 0° C.; add, dropwise, 21 g (0.414 mol) of hydrazine hydrate, maintaining the temperature. Stir the mixture for 24 hours at room temperature. Cool the reaction mass to 0° C., add 60 g (0.436 mol) of para-methoxybenzaldehyde, and stir at room temperature for 24 hours. After that, distill off the solvent, dissolve the residue in 130 mL of 2-propanol, add 9.2 g (0.23 mol) of NaOH, and boil for 2.5 hours. After that, concentrate the reaction mass, dissolve the residue in water, and extract with 125 mL of ethyl acetate four times. Combine the organic layers and wash with 200 mL of 2M hydrochloric acid. Separate the aqueous layer. Neutralize the aqueous layer with 2M NaOH and extract the product with 100 mL of dichloromethane four times. Wash the organic layer with water, dry with sodium sulfate, and distill off the solvent. Purify the resulting product by column chromatography, eluent hexane:ethyl acetate (1:1). Yield: 10.58 g (12.8%).

BCD-BTK-13-13.

In a round-bottom flask, equipped with a stirrer, thermometer and reflux condenser, mix under nitrogen in the specified order: 1 g (0.0048 mol) of BCD-BTK-13-14 and 1.17 g (0.0051 mol) of diethyl ethoxymethylenemalonate. Heat the mixture for 2 hours at 125-130° C. Distill off the solvent using a rotary evaporator. Take the mixture to the next step without additional purification. Yield: 1.88 g (99%).

BCD-BTK-13-12.

In a round-bottom flask, equipped with a stirrer, thermometer and reflux condenser, mix under nitrogen in the specified order: 1.88 g (0.005 mol) of raw compound BCD-BTK-13-13 obtained from the previous step and 10 mL of diphenyl ether. Stir the mixture for 3 hours at 250° C.; use the TLC method to ensure the completeness of the reaction. Allow the reaction mass to cool and add 30 mL of hexane. Filter the resulting precipitate, wash with 30 mL of hexane twice, and allow to dry in air. Yield: 1.55 g (92%).

BCD-BTK-13-11.

In a round-bottom flask, equipped with a stirrer, thermometer and reflux condenser, mix under nitrogen in the specified order: 10 mL of ethanol, 1.55 g (0.0046 mol) of BCD-BTK-13-12, and 10 mL of 10% NaOH. Stir the resulting mixture at boiling temperature; use the TLC method to ensure the completeness of the reaction. When the reaction is complete, acidify the mixture to pH 1-2 with 1M hydrochloric acid, filter the resulting precipitate. Wash the precipitate twice with 10 mL of water, allow to dry in air, and take to the next step without additional purification. Yield: 1.394 g (79%).

BCD-BTK-13-10.

In a round-bottom flask, equipped with a stirrer, thermometer and reflux condenser, mix under nitrogen in the specified order: 2 g (0.005 mol) of raw compound BCD-BTK-13-11 obtained from the previous step and 12 mL of diphenyl ether. Stir the resulting mixture for 1 hour at 120° C.; use the TLC method to ensure the completeness of the reaction. Allow the reaction mass to cool and add 30 mL of hexane. Filter the resulting precipitate, wash with 30 mL of hexane twice, and allow to dry in air. Yield: 1.45 g (84%).

BCD-BTK-13-9.

In a round-bottom flask, equipped with a stirrer, thermometer and reflux condenser, mix in the specified order: 100 mL of DMF, 10 g (0.038 mol) of BCD-BTK-13-10, and 9.6 g (0.042 mol) of N-iodosuccinimide. Stir the mixture at 80° C. for 2 hours; use the TLC method to ensure the completeness of the reaction. When the reaction is complete, pour the mixture into 100 mL of water; filter the resulting precipitate, wash twice with water, and dry under vacuum at 40° C. Yield: 10.2 g (71%).

BCD-BTK-13-8.

In a round-bottom flask, equipped with a stirrer, thermometer and reflux condenser, mix under nitrogen in the specified order: 30 mL of phosphoryl chloride and 3.67 g (0.0095 mol) of BCD-BTK-13-9. Stir the mixture at 60° C. After 1 hour, pour the mixture on ice while stirring. Filter the resulting precipitate and wash three times with 30 mL of water. Yield: 2.66 g (70%).

BCD-BTK-13-7.

In a round-bottom flask, equipped with a stirrer, thermometer and reflux condenser, mix under nitrogen in the specified order: 120 mL of dry THF and 4.778 g (0.0118 mol) of BCD-BTK-13-8. Cool the suspension to 0° C., and add, dropwise, 3.6 mL (0.0177 mol) of 2M i-PrMgCl in THF. Stir the mixture at 0° C. After 1 hour, add 5.622 g (0.02959 mol) of triisopropyl borate, stir for 16 hours at room temperature, and cool again to 0° C. To the resulting mixture add 16.97 g (0.1183 mol) of 30% hydrogen peroxide, 0.478 g (0.0118 mol) of solid NaOH, and stir for another hour at room temperature. After that, add 15.07 g (0.1183 mol) of sodium sulfite, stir, and extract three times with 100 mL of ethyl acetate. Wash combined organic layer with water, dry with sodium sulfate, and distill off the solvent using a rotary evaporator. Purify the resulting product by column chromatography, eluent hexane:ethyl acetate (from 9:1 to 8:2). Yield: 1.13 g (33%).

BCD-BTK-13-6.

In a round-bottom flask, equipped with a stirrer, thermometer and reflux condenser, mix under nitrogen in the specified order: 20 mL of trifluoroacetic acid and 2 g (0.0068 mol) of BCD-BTK-13-7. Boil the resulting mixture for 1 hour; use the TLC method to ensure the completeness of the reaction. After that, distill off most of the trifluoroacetic acid using a rotary evaporator; neutralize the residue to pH 7 with a solution of sodium bicarbonate. Concentrate the mixture to dryness. Purify the resulting product by column chromatography, eluent hexane:ethyl acetate (from 9:1 to 8:2). Yield: 0.63 g (55%).

BCD-BTK-13-5.

In a round-bottom flask, equipped with a stirrer, thermometer and reflux condenser, mix under nitrogen in the specified order: 10 mL of dry DMF, 0.6 g (0.0035 mol) of BCD-BTK-13-6, and 0.36 g (0.0053 mol) of imidazole. Cool the mixture to 0° C., add 0.8 g (0.0053 mol) of TBDMSCl, and allow to stand at this temperature for 30 minutes. When the reaction is complete, pour the reaction mass into water, and extract with 20 mL of ethyl acetate three times. Wash the combined extract with water, dry with sodium sulfate, and distill off the solvent. Take the resulting product to the next step without additional purification. Yield: 0.7 g (75%).

BCD-BTK-13-4.

In a round-bottom flask, equipped with a stirrer, thermometer and reflux condenser, mix in the specified order: 10 mL of DMF, 0.6 g (0.0021 mol) of raw compound BCD-BTK-13-5 obtained from the previous step and 0.7 g (0.0031 mol) of N-iodosuccinimide. Stir the mixture at 50° C. for 3 hours; use the TLC method to ensure the completeness of the reaction. When the reaction is complete, pour the mixture into 100 mL of water; filter the resulting precipitate, wash twice with 20 mL of water, and dry under vacuum at 40° C. Yield: 0.73 g (85%).

BCD-BTK-13-3.

In a round-bottom flask, equipped with a stirrer, thermometer and reflux condenser, mix under nitrogen in the specified order: 25 mL of dry THF, 2.71 g (0.00956 mol) of BCD-BTK-13-4, 5.064 g (0.01912 mol) of triphenylphosphine, and 3.889 g (0.01912 mol) of (S)-3-hydroxy-1-(tert-butoxycarbonyl)piperidine. Stir under nitrogen for 15 minutes. Cool the reaction mass to 0° C.; add, dropwise, 3.907 g (0.01912 mol) of diisopropyl azodicarboxylate, maintaining the temperature. After that, allow the reaction mass to warm to room temperature, and stir for 10 hours; use the TLC method to ensure the completeness of the reaction. Purify the resulting product by column chromatography, eluent hexane:ethyl acetate (from 9:1 to 7:3). Yield: 1.8 g (41%).

BCD-BTK-124-6.

In a round-bottom flask, equipped with a stirrer, thermometer and reflux condenser, mix under nitrogen in the specified order: 20 mL of phosphoryl chloride and 2.42 g (0.0095 mol) of BCD-BTK-13-10. Stir the mixture at 60° C. After 2 hours, pour the mixture on ice while stirring. Filter the resulting precipitate, wash three times with 30 mL of water, and allow to dry in air. Yield: 2.0 g (77%).

BCD-BTK-124-5.

In a round-bottom flask, equipped with a stirrer, thermometer and reflux condenser, mix under nitrogen in the specified order: 20 mL of trifluoroacetic acid and 4.923 g (0.0178 mol) of BCD-BTK-124-6. Boil the resulting mixture for 2 hours; use the TLC method to ensure the completeness of the reaction. After that, distill off most of the trifluoroacetic acid using a rotary evaporator; neutralize the residue to pH 7 with a solution of sodium bicarbonate. Extract the aqueous solution with 20 mL of ethyl acetate three times; wash the combined extract with water, dry with sodium sulfate, and distill off the solvent. Purify the resulting product by column chromatography, eluent hexane:ethyl acetate (from 9:1 to 8:2). Yield: 0.63 g (55%).

BCD-BTK-124-4.

In a round-bottom flask, equipped with a stirrer, thermometer and reflux condenser, mix under nitrogen in the specified order: 10 mL of DMF, 1 g (0.0064 mol) of BCD-BTK-30-5, and 1.9 g (0.0083 mol) of N-iodosuccinimide. Stir the mixture at 80° C. for 3 hours; use the TLC method to ensure the completeness of the reaction. When the reaction is complete, pour the mixture into 100 mL of water; filter the resulting precipitate, wash twice with 20 mL of water, and dry under vacuum at 40° C. Yield: 1.75 g (94%).

BCD-BTK-124-3.

In a round-bottom flask, equipped with a stirrer, thermometer and reflux condenser, mix under nitrogen in the specified order: 25 mL of dry THF, 2.67 g (0.00956 mol) of BCD-BTK-30-4, 5.064 g (0.01912 mol) of triphenylphosphine, and 3.889 g (0.01912 mol) of (S)-3-hydroxy-1-(tert-butoxycarbonyl)piperidine. Stir under nitrogen for 15 minutes. Cool the reaction mass to 0° C.; add, dropwise, 3.907 g (0.01912 mol) of diisopropyl azodicarboxylate, maintaining the temperature. After that, allow the reaction mass to warm to room temperature, and stir for 10 hours. When the reaction is complete, distill off the solvent; purify the residue by column chromatography, eluent hexane:ethyl acetate (from 9:1 to 7:3). Yield: 2 g (44%).

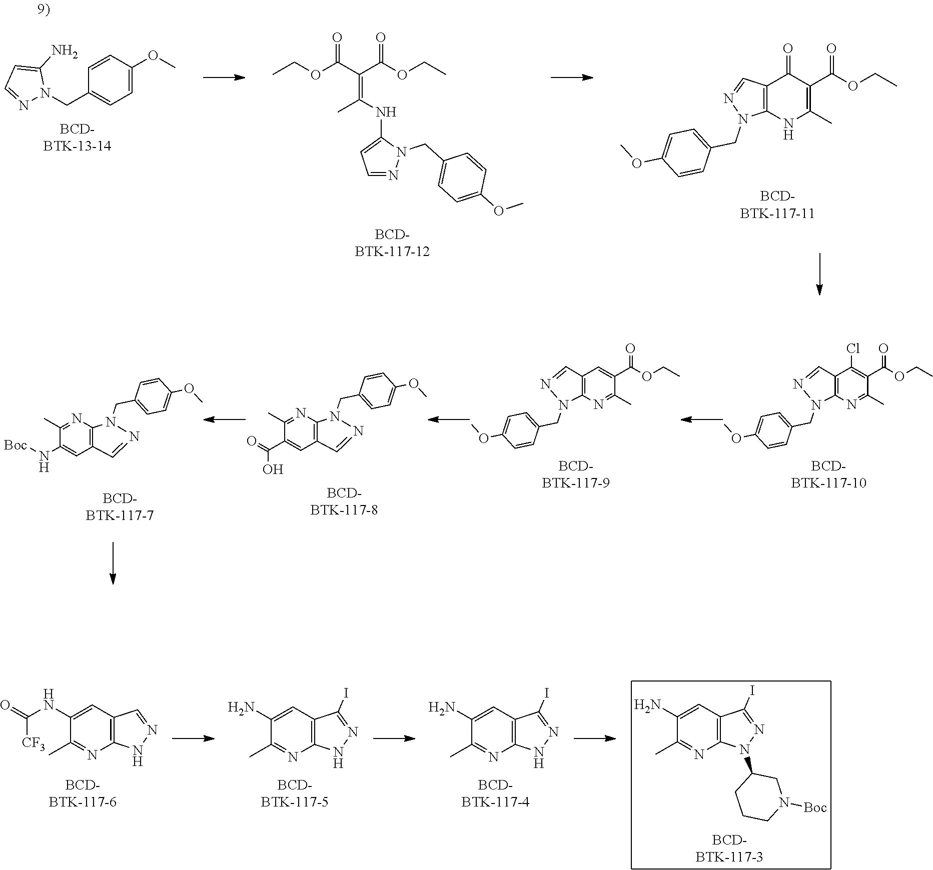

BCD-BTK-117-12.

In a round-bottom flask, equipped with a stirrer, thermometer and reflux condenser, mix under nitrogen in the specified order: 1 g (0.0048 mol) of BCD-BTK-13-14 and 1.18 g (0.0051 mol) of diethyl-2-(1-ethoxyethylidene)malonate. Heat the mixture for 2 hours at 125-130° C. Distill off the residual solvent using a rotary evaporator. Take the mixture to the next step without additional purification. Yield: 1.88 g (990).

BCD-BTK-117-11.

In a round-bottom flask, equipped with a stirrer, thermometer and reflux condenser, mix under nitrogen in the specified order: 8.25 g (0.0213 mol) of raw compound BCD-BTK-117-12 obtained from the previous step and 50 mL of diphenyl ether. Stir the mixture at 120° C. for 3 hours. After that, allow the reaction mass to cool and add 110 mL of hexane. Filter the resulting precipitate, wash with 30 mL of hexane twice, and allow to dry in air. Purify the resulting product by column chromatography, eluent hexane:ethyl acetate (from 95:5 to 7:3). Yield: 5.54 g (76%).

BCD-BTK-117-10.

In a round-bottom flask, equipped with a stirrer, thermometer and reflux condenser, mix under nitrogen in the specified order: 30 mL of phosphoryl chloride and 5.5 g (0.0095 mol) of BCD-BTK-117-11. Stir the mixture at 100° C. After 3 hours, distill off most of the phosphoryl chloride, and pour the residue on ice while stirring. Extract the resulting product with 30 mL of ethyl acetate three times. Wash the combined organic extract with water and NaCl solution, dry with sodium sulfate, and distill off the solvent using a rotary evaporator. Yield: 5.74 g (93%).

BCD-BTK-117-9.

In a stainless steel autoclave equipped with a stirrer and thermometer, place in the specified order: 100 mL of ethanol, 5.7 g (0.0159 mol) of BCD-BTK-117-10, 3.3 mL of triethylamine, and 1 g of 10% Pd/C. Close the autoclave lid, blow with nitrogen, and then introduce hydrogen at room temperature for 3 hours at 5 bar pressure. When the reaction is complete, filter the reaction mass through celite, and distill off the solvent. Yield: 4.79 g (93%).

BCD-BTK-117-8.

In a round-bottom flask, equipped with a stirrer, thermometer and reflux condenser, mix under nitrogen in the specified order: 33 mL of methanol, 3.28 g (0.0105 mol) of BCD-BTK-117-9, and 33 mL of 10% NaOH. Stir the mixture at the boiling temperature. When the reaction is complete, acidify the mixture to pH 1-2 with 2M hydrochloric acid, and filter the resulting precipitate. Wash the precipitate twice with 10 mL of water, allow to dry in air, and take to the next step without additional purification. Yield: 3.2 g (96%).

BCD-BTK-117-7.

In a round-bottom flask, equipped with a stirrer, thermometer and reflux condenser, mix under nitrogen in the specified order: 150 mL of absolute tert-butanol, 5 g (0.017 mol) of raw compound BCD-BTK-117-9 obtained from the previous step, and 2.4 mL of triethylamine (0.017 mol). After that, add 3.7 g (0.017 mol) of DPPA, and stir the mixture at the boiling temperature for 12 hours. When the reaction is complete, distill off the solvent; purify the resulting product by column chromatography, eluent hexane:ethyl acetate (from 99:1 to 7:3). Yield: 5.25 g (85%).

BCD-BTK-117-6.

In a round-bottom flask, equipped with a stirrer, thermometer and reflux condenser, mix under nitrogen in the specified order: 20 mL of trifluoroacetic acid and 3.5 g (0.0068 mol) of BCD-BTK-117-7. Allow the resulting mixture to stand at 80° C. for 2 hours. After that, distill off most of the trifluoroacetic acid using a rotary evaporator; neutralize the residue to pH 7 with 5% sodium bicarbonate. Extract the resulting solution three times with 30 mL of ethyl acetate, wash twice with 30 mL of water, and distill off the solvent. Purify the resulting product by column chromatography, eluent hexane:ethyl acetate (2:3). Yield: 0.63 g (55%).

BCD-BTK-117-5.

In a round-bottom flask, equipped with a stirrer, thermometer and reflux condenser, mix under nitrogen in the specified order: 10 mL of DMF, 2.44 g (0.01 mol) of BCD-BTK-117-6, and 2.5 g (0.011 mol) of N-iodosuccinimide. Stir the mixture at 60° C. for 5 hours. When the reaction is complete, pour the mixture into 100 mL of water; filter the resulting precipitate, wash twice with 20 mL of water, and dry under vacuum at 40° C. Yield: 3 g (82%).

BCD-BTK-117-4.

In a round-bottom flask, equipped with a stirrer, thermometer and reflux condenser, mix under nitrogen in the specified order: 40 mL of 2-propanol, 2 g (0.0054 mol) of BCD-BTK-117-5, and 0.756 g (0.0135 mol) of NaOH. Stir the mixture at the boiling temperature. When the reaction is complete, acidify the mixture to pH 7-8 with 1M hydrochloric acid. Extract the resulting solution five times with 30 mL of ethyl acetate, wash the organic extract twice with 30 mL of water, and distill off the solvent. Purify the resulting product by column chromatography, eluent hexane:ethyl acetate (1:1). Yield: 0.96 g (64%).

BCD-BTK-117-3.

In a round-bottom flask, equipped with a stirrer, thermometer and reflux condenser, mix under nitrogen in the specified order: 10 mL of dry THF, 0.4 g (0.00146 mol) of BCD-BTK-117-4, 0.78 g (0.003 mol) of triphenylphosphine, and 0.6 g (0.003 mol) of (S)-3-hydroxy-1-(tert-butoxycarbonyl)piperidine, and 0.25 mL (0.0015 mol) of diisopropylethylamine. Stir under nitrogen for 15 minutes. Cool the reaction mass to 0° C.; add, dropwise, 0.6 g (0.003 mol) of diisopropyl azodicarboxylate, maintaining the temperature. After that, stir the reaction mass at 0° C. for 4 hours. When the reaction is complete, distill off the solvent; purify the resulting product by column chromatography, eluent hexane:ethyl acetate (from 9:1 to 7:3). Yield: 0.34 g (50%).

### Example 4. Methods for Synthesis of Intermediates X1(a-h), X2(a-f), X3(a-m)

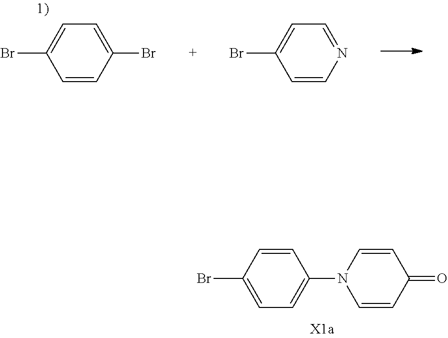 [SMILES: Brc1ccc(Br)cc1 ; Brc1ccncc1 ; O=c1ccn(-c2ccc(Br)cc2)cc1]

X1a.

In a round-bottom flask, equipped with a stirrer, thermometer and reflux condenser, mix in the specified order: 200 mL of DFM, 24 g (0.1 mol) of p-dibromobenzene, 9.675 g (0.1 mol) 4-hydroxypyridine, 66.3 g (0.2 mol) of cesium carbonate, and 1.94 g (0.01 mol) of copper(I) iodide. Stir the mixture at 120° C. under nitrogen for 6 hours; use the TLC method to ensure the completeness of the reaction. Distill off most of the solvent, add 200 mL of ethyl acetate, and filter the suspension through celite. Concentrate the filtrate. Purify the resulting product by column chromatography, eluent ethyl acetate:methanol (9:1). Yield: 11.74 g (61%). Similarly synthesize the compounds X1(b-h). Their structures are presented in the table below.

| | |
|---|---|
| 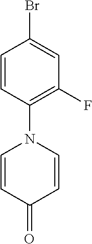 [SMILES: O=c1ccn(-c2ccc(Br)cc2F)cc1] | X1b |
| 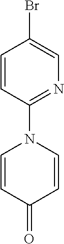 [SMILES: O=c1ccn(-c2ccc(Br)cn2)cc1] | X1c |
| 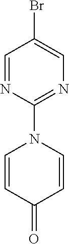 [SMILES: O=c1ccn(-c2ncc(Br)cn2)cc1] | X1d |
| 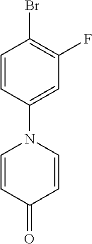 [SMILES: O=c1ccn(-c2ccc(Br)c(F)c2)cc1] | X1e |
| 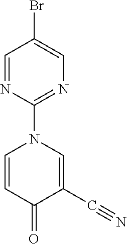 [SMILES: N#Cc1cn(-c2ncc(Br)cn2)ccc1=O] | X1f |
| 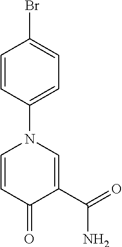 [SMILES: NC(=O)c1cn(-c2ccc(Br)cc2)ccc1=O] | X1g |
| 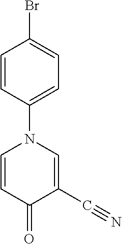 [SMILES: N#Cc1cn(-c2ccc(Br)cc2)ccc1=O] | X1h |

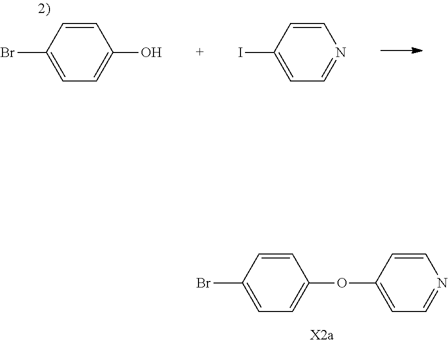 [SMILES: Oc1ccc(Br)cc1 ; Ic1ccncc1 ; Brc1ccc(Oc2ccncc2)cc1]

X2a.

In a round-bottom flask, equipped with a stirrer, thermometer and reflux condenser, mix in the specified order: 10 mL of dry DFM, 1.0 g (0.0048 mol) of 4-iodopyridine, 1.26 g (0.00725 mol) of p-bromophenol, 0.093 g (0.00048 mol) of copper(I) iodide, 3.17 g (0.00965 mol) of cesium carbonate, and 0.89 g (0.0048 mol) of 2,2,6,6-tetramethyl-3,5-heptanedione. Heat the mixture to 60° C., and allow to stand at this temperature for 8 hours. Filter the reaction mass through celite, dilute the filtrate with water, and extract the product with ethyl acetate. Wash the combined extract with 50 mL of NaCl solution and 50 mL of water, dry with sodium sulfate, and distill off. Purify the resulting product by column chromatography, eluent ethyl acetate:hexane (2:8). Yield: 0.7 g (58%).

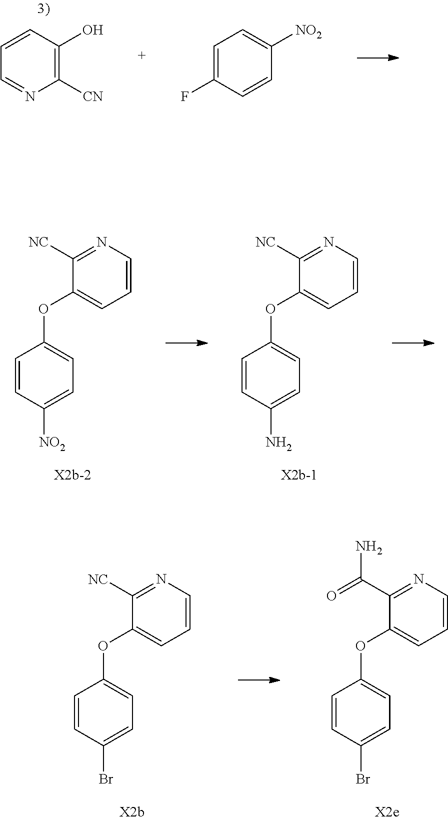 [SMILES: N#Cc1ncccc1O ; O=[N+]([O-])c1ccc(F)cc1 ; N#Cc1ncccc1Oc1ccc([N+](=O)[O-])cc1 ; N#Cc1ncccc1Oc1ccc(N)cc1 ; N#Cc1ncccc1Oc1ccc(Br)cc1 ; NC(=O)c1ncccc1Oc1ccc(Br)cc1]

X2b-2.

In a round-bottom flask, mix 1.8 g (0.015 mol) of 3-hydroxypridine-2-carbonitrile, 30 ml of DMF, 9.77 g (0.30 mol) of Cs₂CO₃ and 2.54 g (0.018 mol) of 1-fluoro-4-nitrobenzene. Heat the reaction mass to 140° C. and stir at this temperature for 4 h; use the TLC method to ensure the completeness of the reaction. Distill off the solvent under redused pressure and add 40 mL of water. Filter the precipitate, wash with 15 ml of water and 15 ml of hexane. Yield: 2.86 g (79%).

X2b-1.

In a round-bottom flask, mix 3 g (0.012 mol) of the compound 3, 45 ml of ethanol, 5 ml of water, 0.32 g (0.006 mol) of NH₄Cl, 3.35 g (0.06 mol) of Fe. Heat the reaction mass to 120° C. and stir at this temperature for 2 h; use the TLC method to ensure the completeness of the reaction. Distill off the solvent under redused pressure and add 30 mL of water. Filter the precipitate, wash with 10 ml of water. Yield: 2.21 g (87%).

X2b.

Add 7 ml of 33% solution of HBr in acetic acid to a solution of 2.7 g (0.013 mol) of 3-(4-aminophenoxy)pyridine-2-carbonitrile in 50 ml of water, cooled to 0° C. Stir the reaction mass at this temperature for 1 h. Add to the reaction mass 1 g (0.014 mol) of NaNO₂ dissolved in 10 ml of water and mix for 2 h. Add 1.86 g (0.013 mol) of preliminary cruched CuBr and a solution of 26.8 g (0.26 mol) of NaBr in 20 ml of water; stir the mixture for another hour, then heat to room temperature; use the TLC method to ensure the completeness of the reaction. Add 25 ml of saturated solution of NaHCO₃ to pH 8, extract the water layer with 20 ml of ethyl acetate thrice. Dry the combined organic layers with Na₂SO₄, distill off the solvent under reduced pressure. Purify the resulting product (3-(4-bromophenoxy)pyridine-2-carbonitrile) by column chromatography, eluent ethyl acetate:hexane (1:9). Yield: 2.65 g (74%).

X2e.

Add 2.21 g (0.016 mol) of K₂CO₃ to a solution of 1.10 g (0.004 mol) of 3-(4-bromophenoxy)pyridine-2-carbonitrile in 10 ml of DMSO, stir the reaction mass for 20 min. Add 0.27 g (0.008 mol) of H₂O₂ and stir for 2 h; use the TLC method to ensure the completeness of the reaction. Add 40 ml of water, mix the reaction mass for 20 min, filtrate the resulting precipitate, wash with 10 ml of water. Yield: 0.72 g (62%).

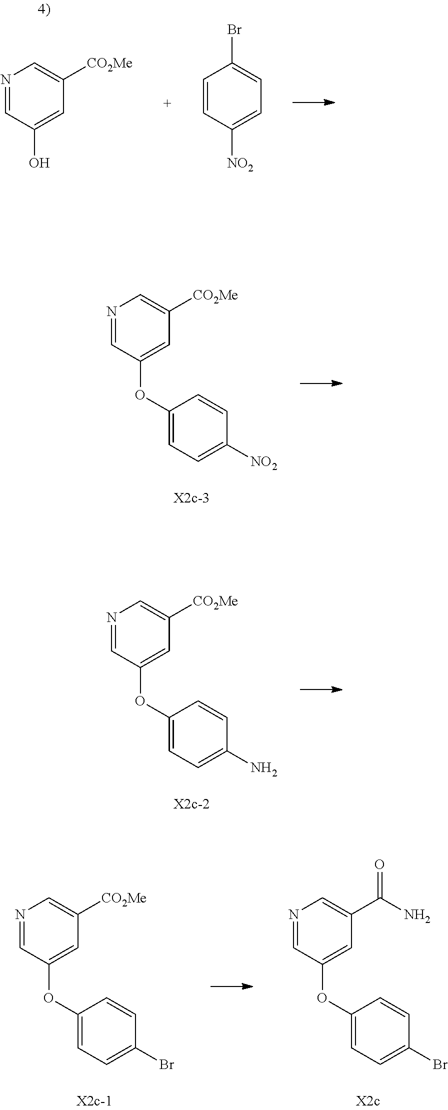 [SMILES: COC(=O)c1cncc(O)c1 ; O=[N+]([O-])c1ccc(Br)cc1 ; COC(=O)c1cncc(Oc2ccc([N+](=O)[O-])cc2)c1 ; COC(=O)c1cncc(Oc2ccc(N)cc2)c1 ; COC(=O)c1cncc(Oc2ccc(Br)cc2)c1 ; NC(=O)c1cncc(Oc2ccc(Br)cc2)c1]

X2c-3.

In a round-bottom flask, mix 4.2 g (0.02 mol) of methyl 5-hydroxypyridine-3-carboxylate, 50 ml of DMF, 13 g (0.04 mol) of Cs₂CO₃ and 4.44 g (0.022 mol) of 1-bromo-4-nitrobenzene. Heat the reaction mass to 140° C. and stir at this temperature for 4 h; use the TLC method to ensure the completeness of the reaction. Distill off the solvent under the reduced pressure, add 50 ml of water. Filter the resulting precipitate, wash with 15 ml of water and 15 ml of hexane. Yield: 4.66 g (85%).

X2c-2.

In a round-bottom flask, mix 4.66 g (0.017 mol) of the compound 3, 45 ml of ethanol, 5 ml of water, 0.45 g (0.008 mol) of NH₄Cl, 4.75 g (0.085 mol) of Fe. Heat the reaction mass to 120° C. and stir at this temperature for 2 h; use the TLC method to ensure the completeness of the reaction. Distill off the solvent under the reduced pressure, add 30 ml of water. Filter the resulting precipitate, wash with 10 ml of water. Yield: 3.07 g (74%).

X2c-1.

Add 7 ml of 33% solution of HBr in acetic acid to 3.18 g (0.013 mol) of methyl 5-(4-aminophenoxy)pyridine-3-carboxylate in 50 ml of water. Stir the reaction mass at this temperature for 1 h. Then add 1 g (0.014 mol) of NaNO₂ in 10 ml of water and stir for 2 h. Then add 1.86 g (0.013 mol) of preliminary crushed CuBr and a solution of 26.8 g (0.26 mol) of NaBr in 20 ml of water, stir the mixture for 1 h, then heat to room temperature; use the TLC method to ensure the completeness of the reaction. Add 25 ml of a saturated solution of NaHCO₃ to pH 8, extract the water layer with 20 ml of ethyl acetate thrice. Dry the combined organic layers with Na₂SO₄. Distill off the solvent under reduced pressure. Purify the resulting product by column chromatography, eluent ethyl acetate:hexane (1:9) Yield: 0.6 g (15%).

X2c.

Add 5 ml of a methanol solution of NH₃ to a methanol solution of 0.2 g (0.0006 mol) of 5-methyl-(4-bromophenoxy)pyridine-3-carboxylate). Stir the reaction mass at room temperature for 24 h; use the TLC method to ensure the completeness of the reaction. Distill off the extra solvent under reduced pressure. Yield: 0.16 g (91%).

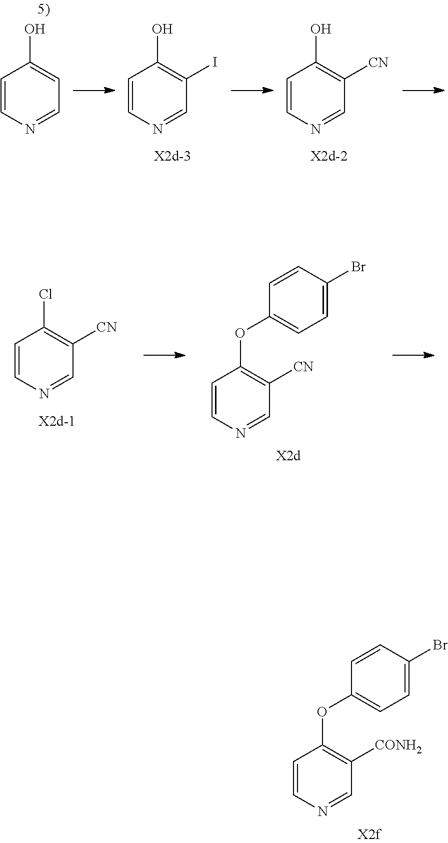 [SMILES: Oc1ccncc1 ; Oc1ccncc1I ; N#Cc1cnccc1O ; N#Cc1cnccc1Cl ; N#Cc1cnccc1Oc1ccc(Br)cc1 ; NC(=O)c1cnccc1Oc1ccc(Br)cc1]

X2d-3.

Add 10.6 g (0.10 mol) of Na₂CO₃ and 12.7 g (0.05 mol) of I₂ to a solution of 4.76 g (0.05 mol) pyridin-4-ol in 200 ml of water. Stir the reaction mass at room temperature for 12 h; use the TLC method to ensure the completeness of the reaction. Add 12 ml of HCl to pH=5, Na₂S₂O₃ till color removal. Filtrate the resulting precipitate, mix the precipitate with 200 ml of boiling ethanol and filtrate one more time. Concentrate the filtrate under reduced pressure, re-crystallize residue from methanol. Yield: 3.4 g (31%).

X2d-2.

Add 1.41 g (0.012 mol) of Zn(CN)₂ and 1.15 g (0.001 mol) of Pd(PPh₃)₄ to a solution of 2.21 g (0.01 mol) of 3-iodopyridin-4-ol in 20 ml of DMF. Heat the reaction mass to 100° C. and stir at this temperature for 2 h; use the TLC method to ensure the completeness of the reaction. Filtrate the resulting precipitate and wash it with DMF. Concentrate the filtrate under reduced pressure. Purify the resulting product by column chromatography, eluent ethyl acetate:methanol (9:1) Yield: 1.1 g (92%).

X2d-1.

Add 2.46 g (0.016 mol) of POCl₃ to 0.24 g (0.002 mol) of 4-hydroxypyridine-3-carbonitrile. Stir the reaction mass for 1 h at room temperature, heat to 70° C. and mix for 1.5 h; use the TLC method to ensure the completeness of the reaction. Pour the mixture into ice, extract with 10 ml of ethyl acetate twice. Wash the combined organic extracts with 10 ml of NaHCO₃ and 10 ml of water, dry with Na₂SO₄, distill off the solvent under reduced pressure. Yield: 0.25 g (90%). X2d. Add 0.11 g (0.00077 mol) of K₂CO₃ and 0.12 g (0.0007 mol) of 4-bromophenol to a solution of 0.1 g (0.0007 mol) of 4-chloropyridine-3-carbonitrile in 1 ml of DMF. Stir the reaction mass under reactionless gas at 70° C. for 2.5 h; use the TLC method to ensure the completeness of the reaction. Add 20 ml of water, stir the reaction mass while cooling down in an ice bath for 10 min. Filter the precipitate. Yield: 0.155 g (78%).

X2f.

Add 2.21 g (0.016 mol) K₂CO₃ to a solution of 1.10 g (0.004 mol) of 4-(4-bromophenoxy)pyridine-3-carbonitrile in 10 ml of DMSO. Add 0.27 g (0.008 mol) of H₂O₂ and stir for 2 h; use the TLC method to ensure the completeness of the reaction. Add 40 ml of water, stir the reaction mass for 20 minutes, filter the precipitate, wash with 10 ml of water. Yield: 0.96 g (82%).

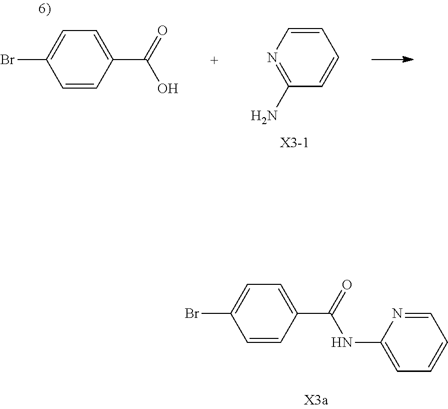 [SMILES: O=C(O)c1ccc(Br)cc1 ; Nc1ccccn1 ; O=C(Nc1ccccn1)c1ccc(Br)cc1]

X3a.

In a round-bottom flask, equipped with a stirrer, thermometer and reflux condenser, mix in the specified order: 20 mL of dichloromethane, 2.1 g (0.0106 mol) of p-bromobenzoic acid, and 1 g (0.0106 mol) of oxalyl chloride. Boil the mixture for 1 hour, distill off the solvent, and dissolve the resulting solid residue in 10 mL of dichloromethane. With constant stirring, add the resulting solution at 5° C. to a pre-prepared solution of 2-aminopyridine in 10 mL of pyridine. Stir the mixture for 1 hour, distill off the solvent, and treat the residue with water. Filter the resulting precipitate, wash with 20 mL of water, and allow to dry in air. Yield: 2.3-2.5 g (80-86%). Similarly synthesize the compounds X3(b-o) shown in the table below:

| | |
|---|---|
| 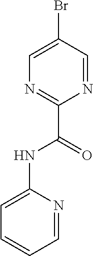 [SMILES: O=C(Nc1ccccn1)c1ncc(Br)cn1] | X3b |
| 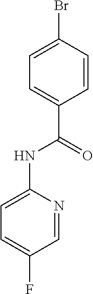 [SMILES: O=C(Nc1ccc(F)cn1)c1ccc(Br)cc1] | X3c |
| 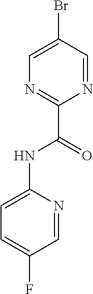 [SMILES: O=C(Nc1ccc(F)cn1)c1ncc(Br)cn1] | X3d |
| 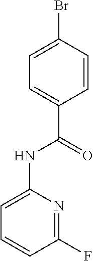 [SMILES: O=C(Nc1cccc(F)n1)c1ccc(Br)cc1] | X3e |
| 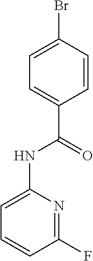 [SMILES: O=C(Nc1cccc(F)n1)c1ccc(Br)cc1] | X3f |
| 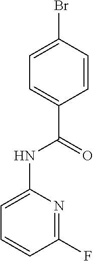 [SMILES: O=C(Nc1cccc(F)n1)c1ccc(Br)cc1] | X3g |
| 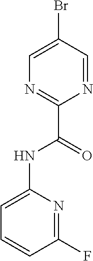 [SMILES: O=C(Nc1cccc(F)n1)c1ncc(Br)cn1] | X3h |
| 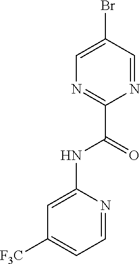 [SMILES: O=C(Nc1cc(C(F)(F)F)ccn1)c1ncc(Br)cn1] | X3i |
| 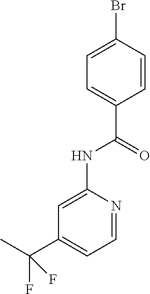 [SMILES: CC(F)(F)c1ccnc(NC(=O)c2ccc(Br)cc2)c1] | X3j |
| 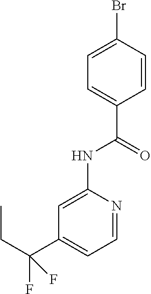 [SMILES: CCC(F)(F)c1ccnc(NC(=O)c2ccc(Br)cc2)c1] | X3k |
| 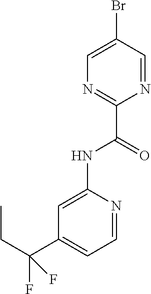 [SMILES: CCC(F)(F)c1ccnc(NC(=O)c2ncc(Br)cn2)c1] | X3l |
| 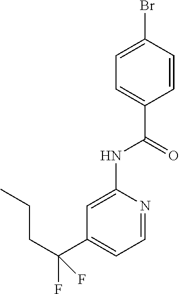 [SMILES: CCCC(F)(F)c1ccnc(NC(=O)c2ccc(Br)cc2)c1] | X3m |
| 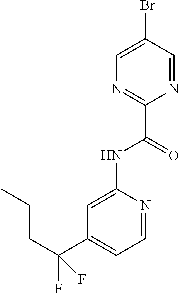 [SMILES: CCCC(F)(F)c1ccnc(NC(=O)c2ncc(Br)cn2)c1] | X3n |
| 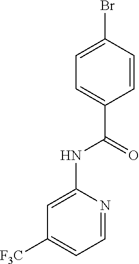 [SMILES: O=C(Nc1cc(C(F)(F)F)ccn1)c1ccc(Br)cc1] | X3o |

### Example 5

Methods for synthesis of compounds BCD-BTK-4, BCD-BTK-6, BCD-BTK-9, BCD-BTK-13, BCD-BTK-18, BCD-BTK-24, BCD-BTK-30, BCD-BTK-35, BCD-BTK-36, BCD-BTK-38, BCD-BTK-54, BCD-BTK-56, BCD-BTK-74, BCD-BTK-76, BCD-BTK-86, BCD-BTK-88, BCD-BTK-98, BCD-BTK-100, BCD-BTK-104, BCD-BTK-105, BCD-BTK-107, BCD-BTK-117, BCD-BTK-118, BCD-BTK-119, BCD-BTK-120, BCD-BTK-121, BCD-BTK-122, BCD-BTK-127, BCD-BTK-130, BCD-BTK-131, BCD-BTK-136. BCD-BTK-204, BCD-BTK-205, BCD-BTK-206, BCD-BTK-207, BCD-BTK-208.

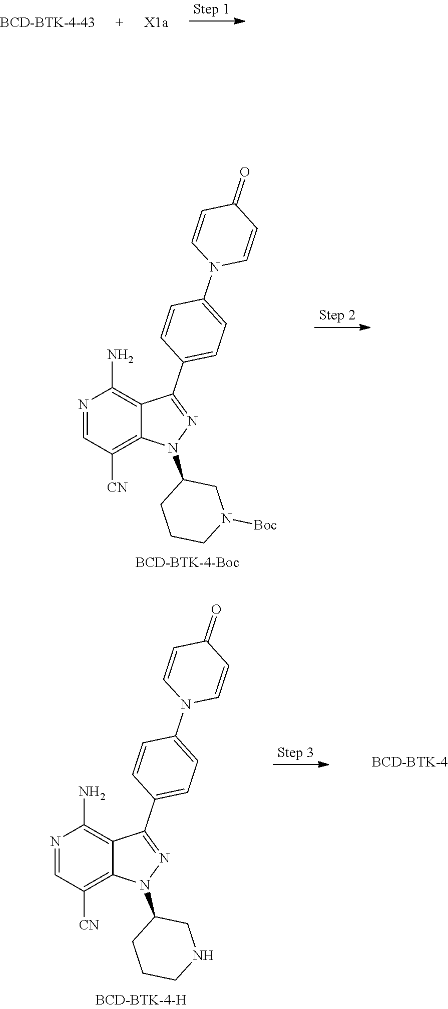 [SMILES: CC(C)(C)OC(=O)N1CCC[C@@H](n2nc(-c3ccc(-n4ccc(=O)cc4)cc3)c3c(N)ncc(C#N)c32)C1 ; N#Cc1cnc(N)c2c(-c3ccc(-n4ccc(=O)cc4)cc3)nn([C@@H]3CCCNC3)c12]

BCD-BTK-4-Boc.

In a three-neck flask, equipped with a stirrer and thermometer, mix under nitrogen in the specified order: 20 mL of 1,4-dioxane, 0.5 g (0.002 mol) of compound X1a, 0.8 g (0.003 mol) of bis(pinacolato)diboron, 0.05 g of XPhos (2-dicyclohexylphosphino-2′,4′,6′-triisopropylbiphenyl), 0.588 g (0.006 mol) of dry potassium acetate, 0.07 g of palladium(II) acetate. Pass nitrogen through the mixture while stirring. Stir the reaction mass under nitrogen at 90° C. for 3 hours. When the reaction is complete, cool the mixture to 40° C.; add a solution of 1.7 g (0.016 mol) of sodium carbonate in 10 mL of water, 0.231 g (0.0002 mol) of tetrakis(triphenylphosphine)palladium, and 0.2 g of BCD-BTK-4-3. Stir the mixture at 80° C. for 5 hours. When the reaction is complete, allow the mixture to cool, filter through celite; wash the celite with 15 mL of ethyl acetate and 10 mL of water. Concentrate the filtrate under vacuum in a rotary evaporator. To the resulting residue add 40 mL of water, and extract the product with 30 mL of ethyl acetate five times. Wash the combined organic extract with water and NaCl solution, dry with sodium sulfate, and distill off the solvent. Purify the resulting product by column chromatography, eluent ethyl acetate:methanol (from 99:1 to 9:1). 0.1 g of BCD-BTK-4-Boc is obtained (yield 50%).

BCD-BTK-4-H.

In a three-neck flask, equipped with a stirrer and thermometer, mix under nitrogen in the specified order: 10 mL of 1,4-dioxane, 0.1 g of BCD-BTK-4-Boc obtained from the previous step, and 3 mL of 4M hydrogen chloride in 1,4-dioxane. Allow the mixture to stand at room temperature. After 6 hours, distill off the solvent. 0.15 g of light yellow powder is obtained. Take it to the next step without additional purification.

BCD-BTK-4.

In a three-neck flask, equipped with a stirrer and thermometer, mix under an inert gas in the specified order: 15 mL of dry DMF, 0.15 g of BCD-BTK-4-H obtained from the previous step, and 0.3 mL of diisopropylethylamine. Cool the mixture to −30° C. and add at this temperature 0.03 g of acryloyl chloride. Allow the reaction mass to stand at room temperature. After 1 hour, concentrate the solvent under vacuum using a rotary evaporator; add 20 mL of ethyl acetate and 60 mL of water. Separate ethyl acetate from the aqueous layer, and wash the aqueous layer with ethyl acetate one more time. Wash the combined organic extract with saturated NaCl solution, dry with sodium sulfate, and distill off the solvent. Purify the resulting product by column chromatography, eluent hexane:ethyl acetate (from 3:7 to 0:100). 0.07 g of BCD-BTK-4 is obtained (yield 45%). Final purification is performed using Akta Explorer 100 with an Inertsil ODS-3 column, R—10 m, L*d—250*30 mm.

The synthesis of compounds BCD-BTK-6, BCD-BTK-9, BCD-BTK-13, BCD-BTK-18, BCD-BTK-24, BCD-BTK-30, BCD-BTK-35, BCD-BTK-36, BCD-BTK-38, BCD-BTK-54, BCD-BTK-56, BCD-BTK-74, BCD-BTK-76, BCD-BTK-86, BCD-BTK-88, BCD-BTK-98, BCD-BTK-100, BCD-BTK-104, BCD-BTK-105, BCD-BTK-107, BCD-BTK-117, BCD-BTK-118, BCD-BTK-119, BCD-BTK-120, BCD-BTK-121, BCD-BTK-122, BCD-BTK-127, BCD-BTK-130, BCD-BTK-131, BCD-BTK-136 BCD-BTK-204, BCD-BTK-205, BCD-BTK-206, BCD-BTK-207, BCD-BTK-208 is performed in the same manner from the corresponding compounds BCD-BTK-4-3, BCD-BTK-6-3, BCD-BTK-9-3, BCD-BTK-13-3, BCD-BTK-18-3, BCD-BTK-24-3, BCD-BTK-30-3, BCD-BTK-35-3, BCD-BTK-104-3, BCD-BTK-211-3, BCD-BTK-239-3, BCD-BTK-241-3 and compounds X1a, X1b, X1c, X1d, X1e, X1f, X1g, X1h.

### Example 6

Methods for synthesis of compounds BCD-BTK-123, BCD-BTK-123, BCD-BTK-124, BCD-BTK-125, BCD-BTK-129, BCD-BTK-133, BCD-BTK-134, BCD-BTK-135, BCD-BTK-137, BCD-BTK-138, BCD-BTK-139, BCD-BTK-140, BCD-BTK-202, BCD-BTK-203, BCD-BTK-211, BCD-BTK-213, BCD-BTK-216, BCD-BTK-217, BCD-BTK-218, BCD-BTK-220, BCD-BTK-222, BCD-BTK-230, BCD-BTK-232, BCD-BTK-236, BCD-BTK-239, BCD-BTK-241, BCD-BTK-246, BCD-BTK-255, BCD-BTK-259, BCD-BTK-261, BCD-BTK-263, BCD-BTK-264, BCD-BTK-265, BCD-BTK-266, BCD-BTK-270, BCD-BTK-272, BCD-BTK-274, BCD-BTK-281, BCD-BTK-282.

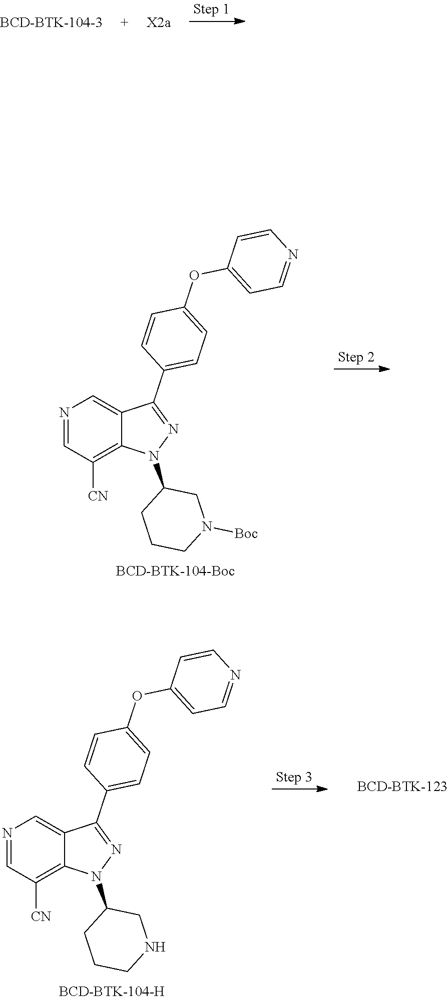 [SMILES: CC(C)(C)OC(=O)N1CCC[C@@H](n2nc(-c3ccc(Oc4ccncc4)cc3)c3cncc(C#N)c32)C1 ; N#Cc1cncc2c(-c3ccc(Oc4ccncc4)cc3)nn([C@@H]3CCCNC3)c12]

BCD-BTK-104-Boc.

In a three-neck flask, equipped with a stirrer and thermometer, mix under nitrogen in the specified order: 10 mL of 1,4-dioxane, 0.3 g (0.0012 mol) of compound X2a, 0.3 g (0.00132 mol) of bis(pinacolato)diboron, 0.02 g of XPhos, 0.23 g (0.0024 mol) of dry potassium acetate, 0.03 g of palladium(II) acetate. Stir the reaction mass under nitrogen at 90° C. for 2 hours. When the reaction is complete, cool the mixture to 20° C.; add a solution of 0.3 g (0.003 mol) of sodium carbonate in 10 mL of water, 0.03 g of tetrakis(triphenylphosphine)palladium, and 0.48 g (0.00108 mol) of BCD-BTK-104-3. Stir the mixture at 80° C. for 5 hours. When the reaction is complete, allow the mixture to cool, filter through celite; wash the celite with 10 mL of ethyl acetate and 30 mL of water. Concentrate the filtrate under vacuum in a rotary evaporator. To the resulting residue add 60 mL of water, and extract the product with 20 mL of ethyl acetate five times. Wash the combined organic extract with water and NaCl solution, dry with sodium sulfate, and distill off the solvent. Purify the resulting product by column chromatography, eluent ethyl acetate:methanol (from 99:1 to 8:2). 0.3 g of BCD-BTK-104-Boc is obtained (yield 55%).

BCD-BTK-104-H.

In a three-neck flask, equipped with a stirrer and thermometer, mix under nitrogen in the specified order: 15 mL of 1,4-dioxane, 0.3 g of BCD-BTK-104-Boc obtained from the previous step, and 6 mL of 4M hydrogen chloride in 1,4-dioxane. Allow the mixture to stand at room temperature. After 8 hours, distill off the solvent. 0.4 g of BCD-BTK-104-H is obtained. Take it to the next step without additional purification.

BCD-BTK-123.

In a three-neck flask, equipped with a stirrer and thermometer, mix under an inert gas in the specified order: 20 mL of dry dichloromethane, 0.4 g of BCD-BTK-104-H obtained from the previous step, and 0.5 mL of diisopropylethylamine. Cool the mixture to −30° C. and add at this temperature 0.09 g of acryloyl chloride. Allow the reaction mass to stand at room temperature. After 1.5 hour, concentrate the solvent under vacuum in a rotary evaporator; add 20 mL of ethyl acetate and 60 mL of water. Separate ethyl acetate from the aqueous layer, and wash the aqueous layer with ethyl acetate one more time. Wash the combined organic extract with saturated NaCl solution, dry with sodium sulfate, and distill off the solvent. Purify the resulting product by column chromatography, eluent hexane:ethyl acetate (from 3:7 to 1:9). 0.27 g of the product is obtained (yield 71%). Final purification is performed using Akta Explorer 100 with the Inertsil ODS-3 column, R—10 m, L*d—250*30 mm.

The synthesis of compounds BCD-BTK-123, BCD-BTK-124, BCD-BTK-125, BCD-BTK-129, BCD-BTK-133, BCD-BTK-134, BCD-BTK-135, BCD-BTK-137, BCD-BTK-138, BCD-BTK-139, BCD-BTK-140, BCD-BTK-202, BCD-BTK-203, BCD-BTK-211, BCD-BTK-213, BCD-BTK-216, BCD-BTK-217, BCD-BTK-218, BCD-BTK-220, BCD-BTK-222, BCD-BTK-230, BCD-BTK-232, BCD-BTK-236, BCD-BTK-239, BCD-BTK-241, BCD-BTK-246, BCD-BTK-255, BCD-BTK-259, BCD-BTK-261, BCD-BTK-263, BCD-BTK-264, BCD-BTK-265, BCD-BTK-266, BCD-BTK-270, BCD-BTK-272, BCD-BTK-274, BCD-BTK-281, BCD-BTK-282 is performed in the same manner from the corresponding compounds BCD-BTK-4-3, BCD-BTK-9-3, BCD-BTK-30-3, BCD-BTK-104-3, BCD-BTK-211-3, BCD-BTK-239-3, BCD-BTK-241-3 and compounds X2a, X2b, X2c, X2d, X2e, X2f.

### Example 7

Methods for synthesis of compounds BCD-BTK-201, BCD-BTK-210, BCD-BTK-212, BCD-BTK-214, BCD-BTK-215, BCD-BTK-219, BCD-BTK-221, BCD-BTK-223, BCD-BTK-224, BCD-BTK-225, BCD-BTK-226, BCD-BTK-227, BCD-BTK-228, BCD-BTK-229, BCD-BTK-231, BCD-BTK-233, BCD-BTK-234, BCD-BTK-235, BCD-BTK-237, BCD-BTK-238, BCD-BTK-240, BCD-BTK-242, BCD-BTK-243, BCD-BTK-244, BCD-BTK-245, BCD-BTK-247, BCD-BTK-248, BCD-BTK-249, BCD-BTK-250, BCD-BTK-251, BCD-BTK-252, BCD-BTK-253, BCD-BTK-254, BCD-BTK-258, BCD-BTK-260, BCD-BTK-262, BCD-BTK-267, BCD-BTK-268, BCD-BTK-269, BCD-BTK-271, BCD-BTK-273, BCD-BTK-275, BCD-BTK-276, BCD-BTK-277, BCD-BTK-278, BCD-BTK-279, BCD-BTK-280, BCD-BTK-283, BCD-BTK-284, BCD-BTK-285, BCD-BTK-286, BCD-BTK-287, BCD-BTK-288, BCD-BTK-289, BCD-BTK-290, BCD-BTK-291, BCD-BTK-292, BCD-BTK-293, BCD-BTK-295.

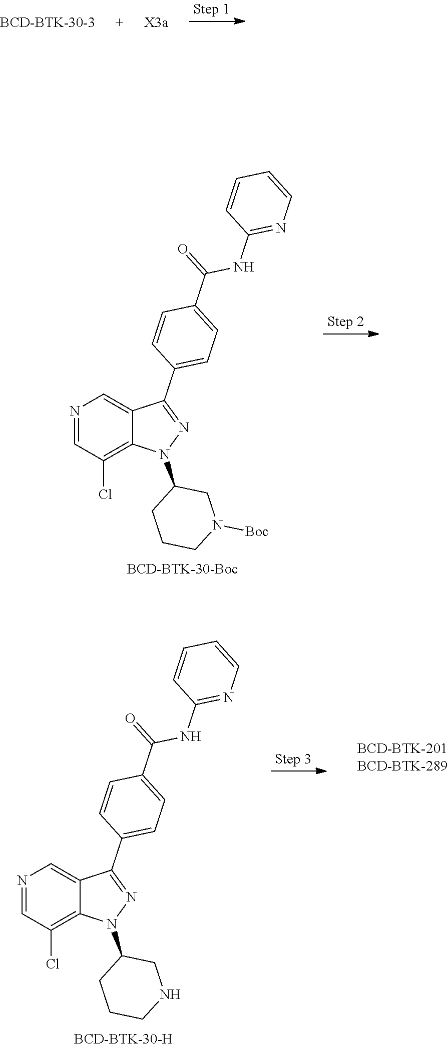 [SMILES: CC(C)(C)OC(=O)N1CCC[C@@H](n2nc(-c3ccc(C(=O)Nc4ccccn4)cc3)c3cncc(Cl)c32)C1 ; O=C(Nc1ccccn1)c1ccc(-c2nn([C@@H]3CCCNC3)c3c(Cl)cncc23)cc1]

BCD-BTK-30-Boc.

In a three-neck flask, equipped with a stirrer and thermometer, mix under nitrogen in the specified order: 10 mL of 1,4-dioxane, 0.2 g (0.0007 mol) of compound X3a, 0.2 g (0.00079 mol) of bis(pinacolato)diboron, 0.015 g of XPhos, 0.1 g (0.0008 mol) of dry potassium acetate, 0.02 g of palladium(II) acetate. Stir the reaction mass under nitrogen at 90° C. for 2 hours. When the reaction is complete, cool the mixture to 20° C.; add a solution of 0.17 g (0.00165 mol) of sodium carbonate in 10 mL of water, 0.04 g of tetrakis(triphenylphosphine)palladium, and 0.29 g (0.00063 mol) of BCD-BTK-30-3. Stir the mixture at 70° C. for 8 hours. When the reaction is complete, allow the mixture to cool, filter through celite; wash the celite with 20 mL of ethyl acetate and 50 mL of water. Concentrate the filtrate under vacuum in a rotary evaporator. To the resulting residue add 50 mL of water and extract the product with 20 mL of ethyl acetate three times. Wash the combined organic extract with water and NaCl solution, dry with sodium sulfate, and distill off the solvent. Purify the resulting product by column chromatography, eluent ethyl acetate:methanol (from 100:0 to 95:5). 0.16 g of BCD-BTK-30-Boc is obtained (yield 47%).

BCD-BTK-30-H.

In a three-neck flask, equipped with a stirrer and thermometer, mix under nitrogen in the specified order: 8 mL of 1,4-dioxane, 0.16 g of the compound obtained from the previous step, and 3 mL of 4M hydrogen chloride in 1,4-dioxane. Allow the mixture to stand at room temperature. After 4 hours, distill off the solvent. 0.23 g of BCD-BTK-30-H is obtained. Take it to the next step without additional purification.

BCD-BTK-201.

In a three-neck flask, equipped with a stirrer and thermometer, mix under an inert gas in the specified order: 20 mL of dry dichloromethane, 0.23 g of BCD-BTK-30-H obtained from the previous step, and 0.2 mL of diisopropylethylamine. Cool the mixture to −30° C. and add at this temperature 0.03 g of acryloyl chloride. Allow the reaction mass to stand at room temperature. After 30 minutes, concentrate the solvent under vacuum using a rotary evaporator; add 10 mL of ethyl acetate and 30 mL of water. Separate ethyl acetate from the aqueous layer, and extract the product from the aqueous layer with ethyl acetate one more time. Wash the combined organic extract with saturated NaCl solution, dry with sodium sulfate, and distill off the solvent. Purify the resulting product by column chromatography, eluent hexane:ethyl acetate (from 3:7 to 1:9). 0.074 g of the product is obtained (yield 34%). Final purification is performed using Akta Explorer 100 with the Inertsil ODS-3 column, R—10 μm, L*d—250*30 mm.

BCD-BTK-289.

Add 0.2 g (0.00052 mol) of HATU and 0.094 g (0.0012 mol) of diisopropylethylamine to a suspension of 0.032 g (0.00038 mol) of tetrolic acid in dry methylene chloride (20 ml). Cool the reaction mass to 0° C. and add a solution of 0.164 g (0.00038 mol) of amine BCD-BTK-30-H in dry methylene chloride if the solubility allows in such a way that the temperature of the mixture would not exceed 5° C. Afterwards, allow the reaction mass stand at room temperature for 1 h, then distill off the solvent under vacuum, add 50 ml of ethyl acetate and 50 ml of water. Separate ethyl acetate from the water layer, wash the water layer with ethyl acetate one more time and combine the ethyl acetate layers. Then wash them with 10% solution of citric acid and a solution of NaCl. Dry ethyl acetate layer with sodium sulphate and distill off the solvent. Yield 0.08 g (47%). Final purification is performed using Akta Explorer 100 with the Inertsil ODS-3 column, R—10 μm, L*d—250*30 mm.

Compounds BCD-BTK-210, BCD-BTK-212, BCD-BTK-214, BCD-BTK-215, BCD-BTK-219, BCD-BTK-221, BCD-BTK-223, BCD-BTK-224, BCD-BTK-225, BCD-BTK-226, BCD-BTK-227, BCD-BTK-228, BCD-BTK-229, BCD-BTK-231, BCD-BTK-233, BCD-BTK-234, BCD-BTK-235, BCD-BTK-237, BCD-BTK-238, BCD-BTK-240, BCD-BTK-242, BCD-BTK-243, BCD-BTK-244, BCD-BTK-245, BCD-BTK-247, BCD-BTK-248, BCD-BTK-249, BCD-BTK-250, BCD-BTK-251, BCD-BTK-252, BCD-BTK-253, BCD-BTK-254, BCD-BTK-258, BCD-BTK-260, BCD-BTK-262, BCD-BTK-267, BCD-BTK-268, BCD-BTK-269, BCD-BTK-271, BCD-BTK-273, BCD-BTK-275, BCD-BTK-276, BCD-BTK-277, BCD-BTK-278, BCD-BTK-279, BCD-BTK-280, BCD-BTK-283, BCD-BTK-284, BCD-BTK-285, BCD-BTK-286, BCD-BTK-287, BCD-BTK-288, BCD-BTK-290, BCD-BTK-291, BCD-BTK-292, BCD-BTK-293, BCD-BTK-295 are synthesized similarly from the corresponding compounds BCD-BTK-4-3, BCD-BTK-9-3, BCD-BTK-30-3, BCD-BTK-104-3, BCD-BTK-211-3, BCD-BTK-239-3, BCD-BTK-241-3 and compounds X3a, X3b, X3c, X3d, X3e, X3f, X3g, X3h, X3i, X3j, X3k, X3l, X3m, X3n, X3o.

### Example 8. Analysis of the Obtained Compounds

To confirm the purity and structure of the obtained compounds, liquid chromatography-mass spectrometry (LC/MS) and ¹H NMR were used (Table 1).

### Equipment Characteristics:

### Liquid Chromatography-Mass Spectrometry

| | Manufacturer, |
|---|---|
| Name | country |
| Agilent Triple Quad LC/MS System | Agilent, USA |
| Agilent 1200 Autosampler | |
| Agilent 1200 Column Thermostat | |
| Agilent 1200 Degasser | |
| Agilent 1200 Autosampler Thermostat | |
| Agilent 6410 QQQ MS Detector | |
| Agilent 1200 UV-detector | |
| Agilent 1200 Pump | |

### NMR Spectrometer

| | Manufacturer, | Model, main |
|---|---|---|
| Name | country | characteristics |
| NMR | Germany | AVANCE III, |
| Spectrometer | | 400 MHz |

TABLE 1
Analytical characteristics for example compounds

| | ESI-MS, | NMR ¹H |
|---|---|---|
| Compound | [M + H]⁺ | (DMSO-d₆), δ, ppm |
| BCD-BTK-4 | 466.2 | 8.33 (s, 1H), 8.07 (d, J = 7.8 Hz, 2H), 7.89-7.73 (m, |
| | | 4H), 6.82 (dd, J = 24.1, 13.9 Hz, 3H), 6.29 (d, J = 7.8 |
| | | Hz, 2H), 6.10 (dd, J = 30.5, 16.4 Hz, 1H), 5.66 (dd, |
| | | J = 49.4, 10.0 Hz, 1H), 4.91 (d, J = 33.2 Hz, 1H), 4.63 |
| | | (d, J = 11.1 Hz, 1H), 4.41-4.21 (m, 1H), 4.12-4.02 |
| | | (m, 1H), 3.73 (d, J = 11.4 Hz, 1H), 3.47 (s, 1H), 2.99 |
| | | (s, 1H), 2.35-2.08 (m, 2H), 1.96 (d, J = 31.8 Hz, |
| | | 1H), 1.57 (s, 1H) |
| BCD-BTK-6 | 484.2 | 8.04 (t, J = 18.8 Hz, 4H), 7.76 (dd, J = 26.5, 8.3 Hz, |
| | | 5H), 7.39 (s, 1H), 6.82 (dd, J = 16.5, 10.6 Hz, 1H), |
| | | 6.29 (d, J = 7.7 Hz, 2H), 6.10 (dd, J = 34.5, 19.6 Hz, |
| | | 2H), 5.64 (dd, J = 44.7, 10.1 Hz, 1H), 5.07 (s, 1H), |
| | | 4.56 (s, 1H), 4.39-4.24 (m, 1H), 4.04 (s, 1H), 3.51 |
| | | (d, J = 11.8 Hz, 1H), 2.82 (s, 1H), 2.22 (dd, J = 70.7, |
| | | 23.5 Hz, 3H), 1.93 (d, J = 13.4 Hz, 1H), 1.50 (s, 1H) |
| BCD-BTK-9 | 475.0 | 8.08 (d, J = 7.7 Hz, 2H), 7.87-7.65 (m, 5H), 6.93-6.66 |
| | | (m, 1H), 6.30 (d, J = 7.7 Hz, 2H), 6.21-5.97 |
| | | (m, 3H), 5.78-5.54 (m, 2H), 5.20 (s, 1H), 4.72 (d, |
| | | J = 10.0 Hz, 1H), 4.34 (d, J = 11.3 Hz, 1H), 4.19-3.94 |
| | | (m, 1H), 3.76-3.53 (m, 1H), 2.92 (s, J = 88.1 |
| | | Hz, 1H), 2.26 (s, 2H), 1.96 (dd, J = 22.5, 8.8 Hz, |
| | | 1H), 1.62 (s, 1H) |
| BCD-BTK-13 | 476.1 | 10.48 (s, 1H), 8.39 (s, 1H), 8.18-8.02 (m, 2H), 7.85 |
| | | (d, J = 8.1 Hz, 2H), 7.73-7.62 (m, 2H), 6.92-6.54 |
| | | (m, 1H), 6.36-6.22 (m, 2H), 6.10 (dd, J = 35.2, 16.7 |
| | | Hz, 1H), 5.64 (dd, J = 61.5, 10.5 Hz, 1H), 4.88 (s, |
| | | 1H), 4.61 (d, J = 12.5 Hz, 1H), 4.20 (s, 1H), 4.09 (d, |
| | | J = 13.7 Hz, 1H), 3.75 (t, J = 11.4 Hz, 1H), 3.12 (d, |
| | | J = 11.3 Hz, 1H), 2.40-2.14 (m, 2H), 1.99 (d, J = 13.2 |
| | | Hz, 1H), 1.65 (s, 1H) |
| BCD-BTK-18 | 484.3 | 9.60 (s, 1H), 8.25 (s, 1H), 8.12-8.05 (m, 3H), 8.02 |
| | | (d, J = 8.3 Hz, 3H), 7.80 (d, J = 39.7 Hz, 1H), 7.74-7.67 |
| | | (m, 2H), 7.39 (d, J = 2.2 Hz, 1H), 7.08 (d, J = |
| | | 2.2 Hz, 1H), 6.80 (dd, J = 16.8, 10.9 Hz, 1H), 6.33-6.25 |
| | | (m, 2H), 6.09 (dd, J = 34.7, 16.6 Hz, 1H), 5.64 |
| | | (dd, J = 56.9, 10.4 Hz, 1H), 4.81 (s, 1H), 4.65-3.99 |
| | | (m, 2H), 3.60 (t, J = 11.5 Hz, 1H), 3.25-2.83 (m, |
| | | 1H), 2.37-1.88 (m, 2H), 1.48 (s, 1H) |
| BCD-BTK-24 | 469.4 | 9.50 (s, 1H), 8.55 (s, 1H), 8.42 (s, 1H), 8.27-8.18 |
| | | (m, 2H), 8.12-8.04 (m, 2H), 7.96 (d, J = 35.8 Hz, |
| | | 1H), 7.78-7.71 (m, 2H), 6.93-6.73 (m, 1H), 6.32-6.25 |
| | | (m, 2H), 6.10 (dd, J = 32.1, 16.7 Hz, 1H), 5.64 |
| | | (dd, J = 59.2, 10.5 Hz, 1H), 4.97 (s, 1H), 4.65-3.98 |
| | | (m, 2H), 3.75-3.39 (m, 1H), 2.97 (t, J = 12.2 Hz, |
| | | 1H), 2.41-2.09 (m, 2H), 2.04-1.93 (m, 1H), 1.53 |
| | | (s, 1H) |
| BCD-BTK-30 | 460.1 | 9.40 (s, 1H), 8.50 (d, J = 2.1 Hz, 1H), 8.26-8.16 (m, |
| | | 2H), 8.13-8.04 (m, 2H), 7.74 (d, J = 8.6 Hz, 2H), |
| | | 6.95-6.71 (m, 1H), 6.35-6.25 (m, 2H), 6.12 (dd, |
| | | J = 24.6, 16.6 Hz, 1H), 5.66 (dd, J = 53.2, 10.5 Hz, |
| | | 1H), 5.28 (d, J = 22.4 Hz, 1H), 4.56 (dd, J = 123.2, |
| | | 13.0 Hz, 1H), 4.19 (dd, J = 82.3, 13.4 Hz, 1H), 3.59 |
| | | (dt, J = 130.4, 11.4 Hz, 1H), 3.04 (t, J = 12.7 Hz, |
| | | 1H), 2.44-2.26 (m, 2H), 2.01 (d, J = 13.4 Hz, 1H), |
| | | 1.66 (s, 1H) |
| BCD-BTK-35 | 493.2 | 8.52 (d, J = 9.3 Hz, 1H), 8.13 (dd, J = 6.6, 3.8 Hz, |
| | | 4H), 7.98-7.76 (m, 5H), 6.69 (ddd, J = 70.9, 16.6, |
| | | 10.5 Hz, 1H), 6.39-6.23 (m, 2H), 6.06 (dd, J = 34.2, |
| | | 16.6 Hz, 1H), 5.64 (dd, J = 32.8, 10.5 Hz, 1H), |
| | | 4.69-4.20 (m, 3H), 4.14-3.75 (m, 1H), 3.51-2.88 |
| | | (m, 1H), 2.46-2.29 (m, 1H), 2.20 (s, 1H), 1.97-1.82 |
| | | (m, 1H), 1.47 (s, 1H) |
| BCD-BTK-36 | 468.3 | 9.04 (s, 2H), 8.98-8.88 (m, 2H), 8.37 (s, 1H), 7.31 |
| | | (s, 2H), 6.82 (dd, J = 28.2, 14.3 Hz, 1H), 6.38 (d, J = |
| | | 7.9 Hz, 2H), 6.10 (dd, J = 26.5, 16.5 Hz, 1H), 5.66 |
| | | (dd, J = 46.7, 10.5 Hz, 1H), 4.94 (d, J = 35.4 Hz, |
| | | 1H), 4.49 (dd, J = 108.4, 13.1 Hz, 1H), 4.30-3.99 |
| | | (m, 1H), 3.76-3.44 (m, 1H), 3.03 (s, 1H), 2.32-2.10 |
| | | (m, 2H), 1.98 (s, 1H), 1.56 (s, 1H) |
| BCD-BTK-38 | 467.3 | 8.72 (d, J = 2.2 Hz, 1H), 8.63-8.52 (m, 2H), 8.34 (s, |
| | | 1H), 8.22 (dd, J = 8.6, 2.3 Hz, 1H), 7.98 (d, J = 8.5 |
| | | Hz, 1H), 7.16 (s, 2H), 6.95-6.76 (m, 1H), 6.41-6.29 |
| | | (m, 2H), 6.09 (td, J = 18.3, 17.6, 8.6 Hz, 1H), |
| | | 5.66 (dd, J = 48.7, 10.5 Hz, 1H), 4.92 (d, J = 34.6 |
| | | Hz, 1H), 4.48 (dd, J = 107.8, 13.0 Hz, 1H), 4.15 (dd, |
| | | J = 78.7, 13.2 Hz, 1H), 3.62 (dt, J = 100.0, 11.4 Hz, |
| | | 1H), 3.00 (d, J = 12.5 Hz, 1H), 2.28 (dq, J = 9.7, 4.8 |
| | | Hz, 2H), 1.98 (s, 1H), 1.57 (s, 1H) |
| BCD-BTK-54 | 462.2 | 9.51 (d, J = 3.2 Hz, 3H), 8.96-8.83 (m, 2H), 8.54 (s, |
| | | 1H), 7.04-6.67 (m, 1H), 6.45-6.32 (m, 2H), 6.22-6.01 |
| | | 6.01 (m, 1H), 5.66 (dd, J = 57.4, 10.5 Hz, 1H), 5.33 |
| | | (d, J = 28.0 Hz, 1H), 4.53 (dd, J = 118.6, 13.1 Hz, |
| | | 1H), 4.30-3.97 (m, 1H), 3.66 (dt, J = 128.7, 11.3 |
| | | Hz, 1H), 3.22-3.03 (m, 1H), 2.34 (d, J = 4.4 Hz, |
| | | 2H), 2.00 (s, 1H), 1.67 (s, 1H) |
| BCD-BTK-56 | 461.1 | 9.46 (s, 1H), 9.19 (d, J = 2.3 Hz, 1H), 8.72-8.47 (m, |
| | | 4H), 8.01 (d, J = 8.7 Hz, 1H), 6.83 (dt, J = 39.5, 13.5 |
| | | Hz, 1H), 6.39-6.25 (m, 2H), 6.21-6.01 (m, 1H), |
| | | 5.65 (dd, J = 55.9, 10.5 Hz, 1H), 5.31 (d, J = 26.0 |
| | | Hz, 1H), 4.54 (dd, J = 114.0, 12.9 Hz, 1H), 4.15 (d, |
| | | J = 70.2 Hz, 1H), 3.79-3.10 m, 1H), 3.10 (s, 0H), |
| | | 2.33 (s, 2H), 1.95 (d, J = 31.0 Hz, 1H), 1.65 (s, 1H) |
| BCD-BTK-74 | 477.1 | 9.05 (s, 2H), 8.93 (d, J = 7.9 Hz, 2H), 7.84 (s, 1H), |
| | | 6.94-6.69 (m, 1H), 6.44-6.30 (m, 5H), 6.23-6.02 |
| | | (m, 1H), 5.67 (dd, J = 42.5, 10.6 Hz, 1H), 5.23 (d, |
| | | J = 24.7 Hz, 1H), 4.52 (dd, J = 140.7, 13.6 Hz, 1H), |
| | | 4.18 (dd, J = 81.4, 14.7 Hz, 1H), 3.69 (t, J = 11.9 Hz, |
| | | 1H), 2.98 (s, 1H), 2.30 (d, J = 26.1 Hz, 3H), 1.97 (d, |
| | | J = 13.4 Hz, 1H), 1.61 (s, 1H) |
| BCD-BTK-76 | 476.2 | 8.74 (d, J = 2.2 Hz, 1H), 8.64-8.51 (m, 2H), 8.24 |
| | | (d, J = 8.5 Hz, 1H), 7.98 (d, J = 8.5 Hz, 1H), 7.82 (s, |
| | | 1H), 6.82 (ddd, J = 32.7, 17.1, 10.9 Hz, 1H), 6.40-6.26 |
| | | (m, 2H), 6.25-6.03 (m, 3H), 5.82-5.55 (m, 1H), |
| | | 5.22 (d, J = 22.6 Hz, 1H), 4.83-4.32 (m, 1H), |
| | | 4.31-4.01 (m, 1H), 3.67 (t, J = 11.8 Hz, 1H), 2.95 |
| | | (t, J = 12.0 Hz, 1H), 2.30 (d, J = 26.3 Hz, 2H), |
| | | 2.06-1.90 (m, 1H), 1.61 (s, 1H) |
| BCD-BTK-86 | 486.2 | 9.04 (d, J = 3.2 Hz, 2H), 8.99-8.89 (m, 2H), 8.10 |
| | | (d, J = 8.7 Hz, 2H), 7.48 (d, J = 26.5 Hz, 1H), |
| | | 6.90-6.72 (m, 3H), 6.41-6.32 (m, 2H), 6.09 (dd, J = |
| | | 29.1, 16.6 Hz, 1H), 5.65 (dd, J = 46.4, 10.6 Hz, 1H), |
| | | 5.10 (d, J = 8.0 Hz, 1H), 4.31 (s, 1H), 4.29 (s, 1H), |
| | | 3.58 (dd, J = 13.1, 9.8 Hz, 1H), 2.96-2.80 (m, 1H), |
| | | 2.38-2.04 m, 2H), 2.02-1.88 (m, 1H), 1.50 (s, 1H) |
| BCD-BTK-88 | 485.2 | 8.74 (s, 1H), 8.57 (d, J = 7.9 Hz, 2H), 8.23 (d, J = 8.5 |
| | | Hz, 1H), 8.15-7.93 (m, 3H), 7.39 (d, J = 39.9 Hz, |
| | | 1H), 6.83 (dd, J = 16.7, 10.5 Hz, 1H), 6.33 (t, J = 6.2 |
| | | Hz, 4H), 6.09 (dd, J = 30.2, 16.7 Hz, 1H), 5.64 (dd, |
| | | J = 46.0, 10.4 Hz, 1H), 5.09 (td, J = 10.4, 5.0 Hz, 1H), |
| | | 4.63-3.97 (m, 2H), 3.55 (t, J = 11.6 Hz, 1H), 2.87 |
| | | (dd, J = 24.5, 13.0 Hz, 1H), 2.29 (dd, J = 24.0, 12.3 |
| | | Hz, 1H), 2.20-2.03 (m, 1H), 1.99-1.82 (m, 1H), |
| | | 1.48 (d, J = 14.3 Hz, 1H) |
| BCD-BTK-98 | 471.2 | 9.59 (s, 1H), 9.52 (s, 2H), 8.94-8.87 (m, 2H), 8.59 |
| | | (s, 1H), 8.45 (s, 1H), 7.98 (d, J = 33.3 Hz, 1H), 6.82 |
| | | (ddd, J = 33.8, 16.5, 10.5 Hz, 1H), 6.44-6.33 (m, |
| | | 2H), 6.10 (dd, J = 31.1, 16.5 Hz, 1H), 5.64 (dd, J = |
| | | 62.6, 10.5 Hz, 1H), 5.17-4.97 (m, 1H), 4.64-4.30 |
| | | (m, 1H), 4.28-4.00 (m, 1H), 3.78-3.48 (m, 1H), |
| | | 3.03 (td, J = 10.8, 9.1, 5.9 Hz, 1H), 2.39-2.13 (m, |
| | | 2H), 1.99 (tq, J = 8.5, 4.6 Hz, 1H), 1.61-1.44 (m, 1H) |
| BCD-BTK-100 | 4701.2 | 9.56 (s, 1H), 9.21 (d, J = 2.4 Hz, 1H), 8.68 (dd, J = |
| | | 8.6, 2.4 Hz, 1H), 8.63-8.54 (m, 3H), 8.46 (s, 1H), |
| | | 8.01 (q, J = 10.8, 8.5 Hz, 2H), 6.84 (ddd, J = 37.1, |
| | | 16.6, 10.5 Hz, 1H), 6.48-6.29 (m, 2H), 6.09 (dd, J = |
| | | 31.0, 16.5 Hz, 1H), 5.64 (dd, J = 63.1, 10.5 Hz, 1H), |
| | | 4.99 (s, 1H), 4.74-3.98 (m, 2H), 3.61 (dt, J = 80.9, |
| | | 11.3 Hz, 1H), 3.03 (m, 1H), 2.42-2.14 (m, 2H), |
| | | 1.97 (dt, J = 13.5, 3.8 Hz, 1H), 1.51 (s, 1H) |
| BCD-BTK-104 | 451.2 | 9.75 (s, 1H), 9.01 (s, 1H), 8.27-8.18 (m, 2H), |
| | | 8.12-8.04 (m, 2H), 7.78-7.71 (m, 2H), 6.93-6.73 |
| | | (m, 1H), 6.35-6.32 (m, 2H), 6.15 (t, 2H), 5.64 (dd, |
| | | J = 59.2, 10.5 Hz, 1H), 5.12 (d, 1H), 4.65-3.98 (m, |
| | | 2H), 3.75-3.39 (m, 1H), 2.97 (t, J = 12.2 Hz, 1H), |
| | | 2.41-2.09 (m, 2H), 2.04-1.93 (m, 1H), 1.53 (s, 1H) |
| BCD-BTK-105 | 453.2 | 9.79 (s, 1H), 9.53 (s, 2H), 9.03 (s, 1H) 8.94-8.87 (d, |
| | | 2H), 6.88 (ddd, J = 33.8, 16.5, 10.5 Hz, 1H), 6.33-6.30 |
| | | (d, 2H), 6.12 (t, 16.5 Hz, 1H), 5.64 (dd, J = 62.6, |
| | | 10.5 Hz, 1H), 5.10 (d, 1H), 4.63-4.31 (m, 1H), |
| | | 4.27-4.01 (m, 1H), 3.78-3.48 (m, 1H), 3.03 (td, |
| | | J = 10.8, 9.1, 5.9 Hz, 1H), 2.39-2.13 (m, 2H), |
| | | 1.99 (tq, J = 8.5, 4.6 Hz, 1H), 1.61-1.44 (m, 1H) |
| BCD-BTK-107 | 452.1 | 9.77 (s, 1H), 9.22 (d, J = 2.3 Hz, 1H), 9.01 (s, 1H), |
| | | 8.69 (dd, J = 8.7, 2.4 Hz, 1H), 8.63-8.55 (m, 2H), |
| | | 8.02 (d, J = 8.6 Hz, 1H), 6.81 (dd, J = 29.3, 15.6 Hz, |
| | | 1H), 6.41-6.26 (m, 2H), 6.11 (t, J = 17.8 Hz, 1H), |
| | | 5.65 (dd, J = 60.8, 10.5 Hz, 1H), 5.10 (d, J = 26.3 |
| | | Hz, 1H), 4.43 (d, J = 13.8 Hz, 1H), 4.27-3.98 (m, |
| | | 1H), 3.92-3.60 (m, 1H), 2.35 (t, J = 4.4 Hz, 1H), |
| | | 2.05 (d, J = 14.7 Hz, 1H), 1.86-1.44 (m, 2H) |
| BCD-BTK-117 | 509.3 | 9.87 (s, 1H), 8.61 (s, 1H), 8.10 (dd, J = 21.9, 7.9 Hz, |
| | | 4H), 7.70 (d, J = 8.4 Hz, 2H), 6.95-6.67 (m, 1H), |
| | | 6.60 (dd, J = 17.0, 10.2 Hz, 1H), 6.39-6.25 (m, 3H), |
| | | 6.12 (dd, J = 30.2, 16.5 Hz, 1H), 5.82 (dd, J = 10.1, |
| | | 2.0 Hz, 1H), 5.78-5.53 (m, 1H), 4.92 (d, J = 12.8 |
| | | Hz, 1H), 4.68-4.24 (m 1H), 4.21-3.73 (m, 1H), |
| | | 3.26-3.09 (m, 1H), 2.60 (s, 3H), 2.40-2.13 (m, |
| | | 2H), 2.07-1.95 (m, 1H), 1.67 (s, 1H) |
| BCD-BTK-118 | 520.3 | 8.06 (dd, J = 11.0, 7.7 Hz, 4H), 7.70 (d, J = 8.4 Hz, |
| | | 2H), 7.64 (s, 1H), 6.97-6.61 (m, 1H), 6.32-6.25 |
| | | (m, 2H), 6.21-6.02 (m, 1H), 5.65 (dd, J = 59.6, 10.5 |
| | | Hz, 1H), 5.03 (s, 2H), 4.82 (s, 1H), 4.62-4.05 (m, |
| | | 2H), 3.76 (s, 1H), 3.16 (d, J = 11.3 Hz, 1H), 2.48 (s, |
| | | 3H), 2.36-2.25 (m, 1H), 2.16 (s, 1H), 1.96 (s, 1H) |
| BCD-BTK-119 | 455.3 | 8.06 (dd, J = 11.0, 7.7 Hz, 4H), 7.70 (d, J = 8.4 Hz, |
| | | 2H), 7.64 (s, 1H), 6.96-6.60 (m, 1H), 6.34-6.24 |
| | | (m, 2H), 6.10 (dd, J = 31.8, 16.6 Hz, 1H), 5.65 (dd, |
| | | J = 59.6, 10.5 Hz, 1H), 5.03 (s, 2H), 4.82 (s, 1H), |
| | | 4.66-4.19 (m, 1H), 4.17-3.67 (m, 1H), 3.16 (d, |
| | | J = 11.3 Hz, 1H), 2.48 (s, 3H), 2.39-2.23 (m, 1H), |
| | | 2.16 (s, 1H), 1.96 (s, 1H), 1.64 (s, 1H) |
| BCD-BTK-120 | 485.3 | 9.67 (s, 1H), 9.01 (d, J = 2.2 Hz, 1H), 8.60-8.52 (m, |
| | | 2H), 8.47 (dd, J = 8.7, 2.3 Hz, 1H), 8.28 (s, 1H), 7.97 |
| | | (d, J = 8.6 Hz, 1H), 7.83 (d, J = 36.7 Hz, 1H), 7.40 |
| | | (d, J = 2.2 Hz, 1H), 7.11 (d, J = 2.2 Hz, 1H), |
| | | 6.95-6.73 (m, 1H), 6.46-6.28 (m, 2H), 6.09 (dd, |
| | | J = 34.6, 16.6 Hz, 1H), 5.64 (dd, J = 60.0, 10.6 Hz, |
| | | 1H), 4.84 (s, 1H), 4.26 (s, 2H), 3.64 (t, J = 11.5 Hz, |
| | | 1H), 2.97 (t, J = 12.1 Hz, 1H), 2.40-2.00 (m, 2H), 1.92 |
| | | (s, 0H), 1.48 (s, 1H) |
| BCD-BTK-121 | 486.3 | 9.33 (s, 2H), 8.88 (d, J = 7.8 Hz, 2H), 8.29 (d, J = 7.4 |
| | | Hz, 1H), 7.84 (d, J = 35.2 Hz, 1H), 7.42 (s, 1H), 7.14 |
| | | (s, 1H), 6.98-6.59 (m, 1H), 6.37 (d, J = 7.7 Hz, |
| | | 2H), 6.09 (dd, J = 34.5, 16.7 Hz, 1H), 5.64 (dd, J = |
| | | 61.2, 10.5 Hz, 1H), 4.86 (s, 1H), 4.69-3.96 (m, 2H), |
| | | 3.66 (t, J = 11.5 Hz, 1H), 3.01 (d, J = 13.3 Hz, 1H), |
| | | 2.41-1.85 (m, 3H), 1.48 (s, 1H) |
| BCD-BTK-122 | 466.4 | 10.08 (s, 1H), 8.20-7.98 (m, 4H), 7.87-7.68 (m, |
| | | 3H), 7.55 (d, J = 2.1 Hz, 1H), 6.84 (dd, J = 29.3, 14.4 |
| | | Hz, 1H), 6.42-6.23 (m, 2H), 6.09 (dd, J = 30.4, 16.7 |
| | | Hz, 1H), 5.65 (dd, J = 61.2, 10.4 Hz, 1H), 5.05 (d, |
| | | J = 33.0 Hz, 1H), 4.83-4.35 (m, 1H), 4.16 (d, J = |
| | | 63.9 Hz, 1H), 3.87-3.50 (m, 1H), 3.13 (d, J = 40.9 |
| | | Hz, 1H), 2.41-1.95 (m, 3H), 1.58 (s, 1H) |
| BCD-BTK-123 | 451.3 | 9.68 (s, 1H), 8.98 (s, 1H), 8.55 (s, 2H), 8.21-8.14 |
| | | (m, 2H), 7.43-7.34 (m, 2H), 7.15-6.80 (m, 3H), |
| | | 6.11 (t, J = 16.8 Hz, 1H), 5.65 (dd, J = 52.7, 10.5 Hz, |
| | | 1H), 5.08 (s, 1H), 4.75-4.00 (m, 2H), 3.88-3.52 |
| | | (m, 1H), 3.07 (s, 1H), 2.34 (q, J = 6.1, 4.6 Hz, 2H), |
| | | 2.03 (s, 1H), 1.62 (s, 1H) |
| BCD-BTK-124 | 460.2 | 8.56 (d, J = 5.0 Hz, 1H), 8.54-8.44 (m, 2H), 7.82 |
| | | (d, J = 8.2 Hz, 2H), 7.43 (d, J = 4.9 Hz, 1H), 7.34-7.26 |
| | | (m, 2H), 7.09-6.96 (m, 2H), 6.92-6.58 (m, 1H), |
| | | 6.11 (dd, J = 33.3, 16.8 Hz, 1H), 5.64 (dd, J = 61.7, |
| | | 10.5 Hz, 1H), 4.98 (s, 1H), 4.63 (d, J = 12.4 Hz, 1H), |
| | | 4.11 (d, J = 13.7 Hz, 1H), 3.77 (d, J = 12.4 Hz, 1H), |
| | | 3.08 (d, J = 4.1 Hz, 1H), 2.43-2.29 (m, 1H), 2.24 |
| | | (s, 1H), 2.01 (d, J = 13.4 Hz, 1H), 1.67 (s, 1H) |
| BCD-BTK-125 | 460.1 | 9.39 (s, 1H), 8.61-8.39 (m, 3H), 8.17 (d, J = 8.4 Hz, |
| | | 2H), 7.59-7.28 (m, 2H), 7.18-7.04 (m, 2H), 6.85 |
| | | (ddd, J = 28.2, 16.5, 10.3 Hz, 1H), 6.11 (t, J = 18.9 |
| | | Hz, 1H), 5.66 (dd, J = 49.2, 10.5 Hz, 1H), 5.29 (s, |
| | | 1H), 4.85-4.39 (m, 1H), 4.18 (dd, J = 85.1, 13.4 Hz, |
| | | 1H), 3.70 (dd, J = 24.1, 12.6 Hz, 1H), 3.02 (t, J = |
| | | 12.3 Hz, 1H), 2.47-2.24 (m, 3H), 1.99 (d, J = 13.4 |
| | | Hz, 1H), 1.64 (s, 1H) |
| BCD-BTK-127 | 467.2 | 10.10 (s, 1H), 9.02 (d, J = 2.2 Hz, 1H), 8.56 (d, J = |
| | | 7.9 Hz, 2H), 8.49 (dd, J = 8.7, 2.3 Hz, 1H), 7.98 (d, |
| | | J = 8.7 Hz, 1H), 7.72 (d, J = 2.2 Hz, 1H), 7.57 (d, J = |
| | | 2.1 Hz, 1H), 6.83 (dd, J = 32.0, 15.5 Hz, 1H), |
| | | 6.47-6.28 (m, 2H), 6.09 (dd, J = 27.7, 16.4 Hz, 1H), |
| | | 5.64 (dd, J = 62.0, 10.4 Hz, 1H), 5.07 (d, J = 32.4 Hz, |
| | | 1H), 4.71-4.31 (m, 1H), 4.29-3.96 (m, 1H), 3.71 |
| | | (d, J = 92.2 Hz, 1H), 3.16 (d, J = 16.8 Hz, 1H), |
| | | 2.35-2.10 (m, 1H), 1.99 (m, 1H), 1.59 (s, 1H) |
| BCD-BTK-129 | 476.1 | 10.42 (s, 1H), 8.50 (d, J = 5.4 Hz, 2H), 8.38 (d, J = |
| | | 2.0 Hz, 1H), 7.78 (d, J = 8.1 Hz, 2H), 7.28 (dd, J = |
| | | 8.6, 2.1 Hz, 2H), 7.01 (d, J = 5.5 Hz, 2H), 6.92-6.60 |
| | | (m, 1H), 6.14 (d, J = 16.6 Hz, 1H), 5.65 (dd, J = |
| | | 57.3, 10.5 Hz, 1H), 4.87 (s, 1H), 4.70-4.01 (m, 2H) |
| | | 3.74 (t, J = 11.4 Hz, 0H), 3.06 (s, 0H), 2.39-2.15 |
| | | (m, 2H), 1.99 (d, J = 13.5 Hz, 1H), 1.64 (s, 1H) |
| BCD-BTK-130 | 468.2 | 10.13 (s, 1H), 9.35 (s, 2H), 9.13-8.63 (m, 2H), 7.75 |
| | | (d, J = 2.2 Hz, 1H), 7.57 (d, J = 2.2 Hz, 1H), 6.98-6.72 |
| | | (m, 1H), 6.45-6.33 (m, 2H), 6.10 (dd, J = 29.4, 16.6 Hz, |
| | | 1H), 5.56 (d, J = 10.6 Hz, 1H), 4.49 (dd, J = 100.7, |
| | | 13.2 Hz, 1H), 4.25-3.99 (m, 1H), 3.96-3.54 (m, 1H), |
| | | 3.16 (s, 1H), 2.40-2.15 (m, 2H), 2.04 (d, J = 29.0 Hz, |
| | | 1H), 1.61 (s, 1H) |
| BCD-BTK-131 | 460.1 | 8.56 (d, J = 5.0 Hz, 1H), 8.15-7.99 (m, 2H), 7.89 |
| | | (d, J = 8.1 Hz, 2H), 7.78-7.64 (m, 2H), 7.43 (d, J = |
| | | 5.0 Hz, 1H), 6.95-6.55 (m, 1H), 6.42-6.23 (m, 2H), |
| | | 6.11 (dd, J = 34.9, 16.6 Hz, 1H), 5.64 (dd, J = 63.0, |
| | | 10.5 Hz, 1H), 5.00 (s, 1H), 4.18 (m, 2H), 3.79 |
| | | (t, J = 11.5 Hz, 1H), 3.21-3.00 (m, 1H), 2.35 (dd, |
| | | J = 11.2, 3.9 Hz, 1H), 2.24 (s, 1H), 2.02 (d, J = 13.4 |
| | | Hz, 1H), 1.68 (s, 1H) |
| BCD-BTK-133 | 484.2 | 9.47 (s, 1H), 8.50 (s, 2H), 8.24 (d, J = 23.6 Hz, 1H), |
| | | 7.96 (d, J = 8.2 Hz, 2H), 7.72 (d, J = 48.2 Hz, 1H), |
| | | 7.40-7.28 (m, 3H), 7.07 (d, J = 2.1 Hz, 1H), 7.02 |
| | | (d, J = 5.3 Hz, 2H), 6.82 (dd, J = 16.7, 10.0 Hz, 1H), |
| | | 6.10 (dd, J = 32.9, 16.7 Hz, 1H), 5.64 (dd, J = 49.1, |
| | | 10.5 Hz, 1H), 5.46-4.55 (m, 2H), 4.33 (dd, J = 40.9, |
| | | 12.9 Hz, 1H), 3.83 (dt, J = 216.9, 12.6 Hz, 1H), 2.84 |
| | | (t, J = 12.2 Hz, 1H), 2.31 (d, J = 22.6 Hz, 1H), 2.19 |
| | | (s, 1H), 1.93 (d, J = 9.9 Hz, 1H), 1.51 (s, 1H) |
| BCD-BTK-134 | 466.2 | 8.52 (s, 2H), 8.30 (s, 1H), 7.71 (d, J = 8.2 Hz, 2H), |
| | | 7.47-7.28 (m, 2H), 7.12 (d, J = 5.2 Hz, 2H), 6.79 |
| | | (d, J = 26.2 Hz, 2H), 6.10 (dd, J = 29.8, 16.7 Hz, |
| | | 1H), 5.66 (dd, J = 45.8, 10.6 Hz, 1H), 4.90 (d, J = |
| | | 33.0 Hz, 2H), 4.35 (d, J = 16.0 Hz, 1H), 3.69 (d, J = |
| | | 11.9 Hz, 1H), 2.95 (s, 1H), 2.34-2.11 (m, 2H), 1.95 |
| | | (d, J = 27.1 Hz, 1H), 1.58 (s, 1H) |
| BCD-BTK-135 | 484.2 | 8.50 (s, 2H), 8.35 (s, 1H), 8.04 (s, 1H), 7.71 (d, J = |
| | | 8.2 Hz, 2H), 7.46-7.25 (m, 3H), 7.11 (d, J = 5.2 Hz, |
| | | 2H), 6.77 (d, J = 26.2 Hz, 2H), 6.11 (dd, J = 29.8, |
| | | 16.7 Hz, 1H), 5.65 (dd, J = 45.8, 10.6 Hz, 1H), 4.97 |
| | | (s, 1H), 4.34 (d, J = 16.0 Hz, 1H), 3.68 (d, J = 11.9 |
| | | Hz, 1H), 2.94 (s, 1H), 2.34-2.11 (m, 2H), 1.93 (d, |
| | | J = 27.1 Hz, 1H), 1.59 (s, 1H) |
| BCD-BTK-136 | 530.2 | 8.69 (d, J = 3.7 Hz, 1H), 8.14 (d, J = 7.3 Hz, 2H), |
| | | 7.89 (d, J = 8.1 Hz, 2H), 7.72 (d, J = 8.4 Hz, 2H), |
| | | 6.88 (dd, J = 16.5, 10.4 Hz, 1H), 6.68 (d, J = 17.2 |
| | | Hz, 1H), 6.55 (dd, J = 17.4, 10.3 Hz, 1H), 6.31 (dd, |
| | | J = 11.7, 9.0 Hz, 3H), 6.10 (dd, J = 39.7, 16.7 Hz, 1H), |
| | | 5.64 (dd, J = 68.0, 10.6 Hz, 1H), 4.99 (d, J = 16.7 |
| | | Hz, 1H), 4.46 (dd, J = 137.7, 13.0 Hz, 1H), 4.09 (d, |
| | | J = 13.4 Hz, 1H), 3.80 (s, 1H), 3.16 (d, J = 16.1 Hz, |
| | | 1H), 2.41-2.18 (m, 2H), 2.00 (d, J = 13.1 Hz, 1H), |
| | | 1.66 (s, 1H) |
| BCD-BTK-137 | 466.2 | 10.02 (s, 1H), 8.51 (d, J = 5.5 Hz, 2H), 7.97 (d, J = |
| | | 8.4 Hz, 2H), 7.69 (d, J = 2.3 Hz, 1H), 7.53 (d, J = 2.2 |
| | | Hz, 1H), 7.44-7.26 (m, 2H), 7.16-6.96 (m, 2H), |
| | | 6.98-6.69 (m, 1H), 6.10 (dd, J = 29.7, 16.6 Hz, 1H), |
| | | 5.65 (dd, J = 55.4, 10.5 Hz, 1H), 5.03 (d, J = 33.8 |
| | | Hz, 1H), 4.80-3.93 (m, 2H), 3.62 (dt, J = 99.7, 11.5 |
| | | Hz, 1H), 3.02 (t, J = 12.1 Hz, 1H), 2.41-2.16 (m, 2H), |
| | | 1.95 (d, J = 30.0 Hz, 1H), 1.59 (s, 1H) |
| BCD-BTK-138 | 493.1 | 8.53 (d, J = 13.5 Hz, 3H), 8.24-8.05 (m, 2H), 7.83 |
| | | (dd, J = 7.3, 1.1 Hz, 1H), 7.75 (s, 2H), 7.41 (d, J = |
| | | 8.0 Hz, 2H), 7.03 (d, J = 12.5 Hz, 2H), 6.70 (ddd, J = |
| | | 79.3, 16.6, 10.6 Hz, 1H), 6.06 (dd, J = 33.1, 16.6 Hz, |
| | | 1H), 5.64 (dd, J = 47.6, 10.6 Hz, 1H), 4.73-3.94 (m, |
| | | 3H), 3.80 (t, J = 11.9 Hz, 1H), 3.03 (d, J = 12.4 Hz, |
| | | 1H), 2.43 (s, 1H), 2.23 (s, 1H), 2.02-1.79 (m, 1H), |
| | | 1.52 (s, 1H) |
| BCD-BTK-139 | 475.2 | 8.54 (s, 2H), 7.78 (s, 1H), 7.72 (d, J = 8.1 Hz, 2H), |
| | | 7.41-7.29 (m, 2H), 7.10 (s, 2H), 6.80 (td, J = 16.7, |
| | | 8.3 Hz, 1H), 6.13 (d, J = 32.6 Hz, 3H), 5.67 (dd, J = |
| | | 38.0, 10.6 Hz, 1H), 4.85-3.97 (m, 2H), 2.88 (t, J = |
| | | 12.4 Hz, 1H), 2.38-2.14 (m, 3H), 1.97 (dt, J = 13.6, |
| | | 3.6 Hz, 1H), 1.61 (s, 1H) |
| BCD-BTK-140 | 469.2 | 9.46 (s, 1H), 8.47 (d, J = 50.3 Hz, 4H), 8.14 (d, J = |
| | | 8.5 Hz, 2H), 7.97 (t, J = 26.1 Hz, 1H), 7.35 (d, J = |
| | | 8.6 Hz, 2H), 7.02 (d, J = 5.1 Hz, 2H), 6.91-6.73 (m, |
| | | 1H), 6.10 (dd, J = 30.2, 16.6 Hz, 1H), 5.64 (dd, J = |
| | | 53.7, 10.0 Hz, 1H), 4.96 (s, 1H), 4.60 (d, J = 10.4 |
| | | Hz, 1H), 4.33 (s, 1H), 4.07 (d, J = 12.9 Hz, 1H), |
| | | 3.67-3.57 (m, 1H), 2.97-2.84 (m, 1H), 2.12 (tt, J = |
| | | 22.9, 16.5 Hz, 4H), 1.53 (s, 1H) |
| BCD-BTK-201 | 487.1 | 10.86 (s, 1H), 9.42 (s, 1H), 8.49 (s, 1H), 8.40 (d, J = |
| | | 3.6 Hz, 1H), 8.28-8.09 (m, 5H), 7.90-7.79 (m, |
| | | 1H), 7.18 (dd, J = 6.9, 5.1 Hz, 1H), 6.91-6.71 (m, |
| | | 1H), 6.13 (s, 1H), 5.78-5.54 (m, 1H), 5.39-5.17 |
| | | (m, 1H), 4.81-4.66 (m, 1H), 4.50-4.27 (m, 1H), |
| | | 4.16-4.02 (m, 1H), 3.81-3.67 (m, 1H), 3.48-3.37 |
| | | (m, 1H), 3.05-2.94 (m, 1H), 2.33 (s, 3H), 2.00 (s, |
| | | 1H), 1.77-1.55 (m, 1H) |
| BCD-BTK-202 | 485.2 | 9.39 (s, 1H), 9.00 (s, 1H), 8.67 (d, J = 6.0 Hz, 1H), |
| | | 8.50 (s, 1H), 8.21 (d, J = 8.6 Hz, 2H), 7.50 (d, J = 8.6 |
| | | Hz, 2H), 6.95 (d, J = 6.0 Hz, 1H), 6.85 (d, J = 11.6 |
| | | Hz, 1H), 6.22-5.99 (m, 1H), 5.66 (dd, J = 48.4, 9.9 |
| | | Hz, 1H), 5.31 (s, 1H), 4.70 (s, 1H), 4.47-4.22 (m, |
| | | 1H), 4.16-4.02 (m, 1H), 3.73 (s, 1H), 3.01 (s, 1H), |
| | | 2.32 (d, J = 4.4 Hz, 2H), 2.00 (d, J = 11.3 Hz, 1H), |
| | | 1.65 (s, 1H) |
| BCD-BTK-203 | 503.0 | 9.37 (s, 1H), 8.78 (s, 1H), 8.49 (d, J = 4.0 Hz, 2H), |
| | | 8.16 (d, J = 8.6 Hz, 2H), 7.79 (d, J = 24.6 Hz, 2H), |
| | | 7.49-7.34 (m, 2H), 6.81 (d, J = 5.8 Hz, 2H), 6.12 (s, |
| | | 1H), 5.79-5.51 (m, 1H), 5.38-5.15 (m, 1H), |
| | | 4.79-4.65 (m, 1H), 4.48-4.26 (m, 1H), 4.17-4.02 |
| | | (m, 1H), 3.80-3.66 (m, 1H), 3.08-2.89 (m, 1H), 2.32 |
| | | (d, J = 4.9 Hz, 2H), 1.99 (s, 1H), 1.65 (s, 1H) |
| BCD-BTK-204 | 478.00 | δ = 9.07 (s, 1H), 8.51 (s, 1H), 8.18-8.09 (m, 2H), |
| | | 8.00 (t, J = 8.2 Hz, 1H), 7.83 (dd, J = 11.6, 1.8 Hz, |
| | | 1H), 7.63 (d, J = 8.4 Hz, 1H), 6.94-6.71 (m, 1H), |
| | | 6.32-6.27 (m, 2H), 6.18-6.01 (m, 1H), 5.72, 5.57 |
| | | (2d, J = 10.0 Hz, 1H, rotamers), 5.32 (s, 1H), 4.70, |
| | | 4.42 (2d, J = 11.9 Hz, 1H, rotamers), 4.27, 4.06 (2d, |
| | | J = 13.2 Hz, 1H, rotamers), 3.76-3.38 (m, 1H), |
| | | 3.33-3.01 (m, 1H), 2.32 (s, 1H), 2.06-1.93 (m, 1H), |
| | | 1.63 (s, 1H) |
| BCD-BTK-205 | 478.10 | δ = 9.42 (s, 1H), 8.48 (s, 1H), 8.12-8.03 (m, 2H), |
| | | 7.85 (dd, J = 7.7, 1.5 Hz, 2H), 7.77 (t, J = 8.4 Hz, |
| | | 1H), 6.90-6.67 (m, 1H), 6.31-6.22 (m, 2H), |
| | | 6.19 -6.04 (m, 1H), 5.71, 5.58 (2d, J = 10.1 Hz, 1H, |
| | | rotamers), 5.27 (s, 1H), 4.69, 4.37 (2d, J = 12.2 Hz, |
| | | 1H, rotamers), 4.27, 4.07 (2d, J = 12.9 Hz, 1H, |
| | | rotamers), 3.82-3.40 (m, 1H), 3.33-3.06 (m, 1H), |
| | | 2.33 (s, 2H), 2.06-1.94 (m, 1H), 1.67 (s, 1H) |
| BCD-BTK-206 | — | δ = 9.56 (s, 1H), 9.52-9.49 (m, 1H), 8.98 (dd, J = |
| | | 8.3, 2.4 Hz, 1H), 8.54 (s, 1H), 6.94-6.69 (m, 1H), |
| | | 6.19-6.02 (m, 1H), 5.72, 5.57 (2d, J = 10.1 Hz, 1H, |
| | | rotamers), 5.42-5.24 (m, 1H), 4.67, 4.37 (2d, J = |
| | | 13.0 Hz, 1H, rotamers), 4.19, 4.06 (2d, J = 13.1 Hz, |
| | | 1H, rotamers), 3.87-3.46 (m, 1H), 3.33-3.10 |
| | | (m, 1H), 2.40-2.29 (m, 1H), 2.06-1.96 (m, 1H), 1.66 |
| | | (s, 1H) |
| BCD-BTK-207 | — | δ = 9.75 (d, J = 2.4 Hz, 1H), 9.57 (s, 2H), 9.51 (s, |
| | | 1H), 9.17 (d, J = 4.2 Hz, 1H), 8.98 (dd, J = 8.0, 2.5 |
| | | Hz, 1H), 8.53 (s, 1H), 7.69 (d, J = 4.1 Hz, 1H), |
| | | 6.94-6.72 (m, 1H), 6.65 (d, J = 8.0 Hz, 1H), 6.18-6.04 |
| | | (m, 1H), 5.72, 5.58 (2d, J = 10.5 Hz, 1H, rotamers), |
| | | 5.42-5.23 (m, 1H), 4.68, 4.39 (2d, J = 11.1 Hz, 1H, |
| | | rotamers), 4.22, 4.06 (2d, J = 13.0 Hz, 1H, rotamers), |
| | | 3.86-3.45 (m, 1H), 3.33-3.09 (m, 1H), 2.41-2.27 |
| | | (m, 2H), 2.00 (s, 1H), 1.65 (s, 1H) |
| BCD-BTK-208 | — | δ = 9.46 (s, 1H), 8.86 (s, 1H), 8.52 (s, 1H), 8.17-8.05 |
| | | (m, 3H), 7.90 (t, J = 8.2 Hz, 1H), 6.93-6.70 |
| | | (m, 1H), 6.50 (d, J = 7.9 Hz, 1H), 6.20-6.03 (m, |
| | | 1H), 5.72, 5.57 (2d, J = 10.1 Hz, 1H, rotamers), |
| | | 5.39-5.23 (m, 1H), 4.66, 4.38 (2d, J = 12.1 Hz, 1H), |
| | | 4.22, 4.05 (d, J = 12.7 Hz, 1H, rotamers), 3.83-3.44 |
| | | (m, 1H), 3.33-3.06 (m, 1H), 2.40-2.27 (m, 2H), |
| | | 2.04-1.94 (m, 1H), 1.65 (s, 1H) |
| BCD-BTK-210 | 555.20 | δ = 11.43 (s, 1H), 9.46 (s, 1H), 8.71 (d, J = 5.1 Hz, |
| | | 1H), 8.58 (s, 1H), 8.54 (s, 1H), 8.27-8.19 (m, 4H), |
| | | 7.57 (d, J = 5.0 Hz, 1H), 6.94-6.78 (m, 1H), |
| | | 6.18-6.05 (m, 1H), 5.72, 5.60 (2d, J = 10.1 Hz, 1H, |
| | | rotamers), 5.31 (s, 1H), 4.69, 4.43 (2d, J = 11.7 Hz, |
| | | 1H, rotamers), 4.28, 4.07 (2d, J = 10.1 Hz, 1H, |
| | | rotamers), 3.80-3.41 (m, 1H), 3.33-2.99 (m, 1H), |
| | | 2.41-2.27 (m, 2H), 1.98 (s, 1H), 1.63 (s, 1H) |
| BCD-BTK-211 | 444.10 | δ = 9.29 (d, J = 1.9 Hz, 1H),8.51 (d, J = 6.0 Hz, 2H), |
| | | 8.47 (d, J = 3.6 Hz, 1H), 8.15 (d, J = 8.3 Hz, 2H), |
| | | 7.36 (d, J = 8.7 Hz, 2H), 7.03 (dd, J = 4.8, 1.4 Hz, |
| | | 2H), 6.92-6.73 (m, 1H), 6.17-6.03 (m, 1H), 5.71, |
| | | 5.59 (2d, J = 10.4 Hz, 1H, rotamers), 4.78 (s, 1H), |
| | | 4.63, 4.40 (2d, J = 12.9 Hz, 1H, rotamers), 4.21, 4.04 |
| | | (2d, J = 12.9 Hz, 1H, rotamers), 3.75-3.46 (m, 1H), |
| | | 3.33-3.02 (m, 1H), 2.39-2.26 (m, 2H), 1.97 (s, 1H), |
| | | 1.62 (s, 1H) |
| BCD-BTK-212 | 505.10 | δ = 11.01 (s, 1H), 9.42 (s, 1H), 8.49 (s, 1H), 8.39 (d, |
| | | J = 3.0 Hz, 1H), 8.27 (dd, J = 9.2, 4.2 Hz, 1H),8.19 |
| | | (q, J = 8.5 Hz, 4H), 7.78 (td, J = 8.7, 3.1 Hz, 1H), |
| | | 6.94-6.72 (m, 1H), 6.21-6.03 (m, 1H), 5.72, 5.60 |
| | | (2d, J = 10.2 Hz, 1H, rotamers), 5.29 (s, 1H), 4.72, |
| | | 4.42 (2d, J = 11.2 Hz, 1H, rotamers), 4.32, 4.08 (d, |
| | | J = 12.2 Hz, 1H, rotamers), 3.81-3.36 (m, 1H), 3.33-2.94 |
| | | (m, 1H), 2.43-2.26 (m, 2H), 1.99 (s, 1H), 1.65 |
| | | (s, 1H) |
| BCD-BTK-213 | 476.20 | δ = 9.71 (s, 1H), 9.02 (d, J = 8.0 Hz, 2H), 8.68 (d, J = |
| | | 6.0 Hz, 1H), 8.23 (d, J = 8.6 Hz, 2H), 7.53 (d, J = 8.6 |
| | | Hz, 2H), 6.96 (d, J = 6.0 Hz, 1H), 6.94-6.78 (m, |
| | | 1H), 6.16-6.03 (m, 1H), 5.71, 5.58 (2d, J=10.2 Hz, |
| | | 1H, rotamers), 5.08 (s, 1H), 4.62, 4.46 (2d, J = 11.8 |
| | | Hz, 1H, rotamers), 4.26, 4.04 (d, J = 12.8 Hz, 1H, |
| | | rotamers), 3.84-3.58 (m, 1H), 3.33-3.01 (m, 1H), |
| | | 2.38-2.19 (m, 2H), 2.01 (s, 1H), 1.59 (s, 1H) |
| BCD-BTK-214 | 549.20 | δ = 11.22 (s, 1H), 9.37 (d, J = 2.0 Hz, 1H), 8.56 (d, |
| | | J = 5.1 Hz, 1H), 8.50 (d, J = 3.6 Hz, 1H), 8.39 (s, 1H), |
| | | 8.22 (s, 4H), 7.33 (d, J = 5.1 Hz, 1H), 6.95-6.75 (m, |
| | | 1H), 6.17-6.05 (m, 1H), 5.72, 5.59 (2d, J = 10.1 Hz, |
| | | 1H, rotamers), 4.80 (s, 1H), 4.63, 4.41 (2d, J = 10.1 |
| | | Hz, 1H, rotamers), 4.21, 4.05 (2d, J = 12.3 Hz, 1H, |
| | | rotamers), 3.79-3.39 (m, 2H), 3.33-3.06 (m, 1H), |
| | | 2.39-2.16 (m, 4H), 1.99 (s, 1H), 1.64 (s, 1H), 0.96 |
| | | (t, J = 7.4 Hz, 3H) |
| BCD-BTK-215 | 565.20 | δ = 11.22 (s, 1H), 9.46 (s, 1H), 8.58-8.52 (m, 2H), |
| | | 8.40 (s, 1H), 8.25-8.17 (m, 4H), 7.33 (dd, J = 5.1, |
| | | 1.2 Hz, 1H), 6.95-6.77 (m, 1H), 6.21-6.05 (m, |
| | | 1H), 5.72, 5.60 (2d, J = 10.4 Hz, 1H, rotamers), 5.32 |
| | | (s, 1H), 4.69, 4.44 (2d, J = 12.6 Hz, 1H, rotamers), |
| | | 4.29, 4.07 (d, J = 12.6 Hz, 1H), 3.79-3.41 (m, 1H), |
| | | 3.33-2.99 (m, 1H), 2.41-2.16 (m, 4H), 1.99 (s, |
| | | 1H), 1.63 (s, 1H), 0.96 (t, J = 7.4 Hz, 3H) |
| BCD-BTK-216 | 476.30 | δ = 9.68 (s, 1H), 9.00 (s, 1H), 8.55 (dd, J = 4.5, 1.0 |
| | | Hz, 1H), 8.18 (d, J = 8.7 Hz, 2H), 7.76 (dd, J = 8.7, |
| | | 4.5 Hz, 1H), 7.67-7.63 (m, 1H), 7.46-7.40 (m, |
| | | 2H), 6.96-6.78 (m, 1H), 6.19-6.02 (m, 1H), 5.71 |
| | | 5.58 (2d, J = 9.8, 1H, rotamers), 5.08 (s, 1H), 4.62, |
| | | 4.46 (2d, J = 11.2 Hz, 1H, rotamers), 4.26, 4.04 (d, |
| | | J = 12.7 Hz, 1H, rotamers), 3.82-3.57 (m, 1H), 3.33- |
| | | 3.02 (m, 1H), 2.38-2.16 (m, 2H), 2.01 (s, 1H), 1.58 |
| | | (s, 1H) |
| BCD-BTK-217 | 469.20 | δ = 9.32 (s, 1H), 9.02 (s, 1H), 8.68 (d, J = 6.0 Hz, |
| | | 1H), 8.49 (d, J = 3.6 Hz, 1H), 8.22 (d, J = 8.2 Hz, |
| | | 2H), 7.52 (d, J = 8.6 Hz, 2H), 6.96 (d, J = 6.0 Hz, |
| | | 1H), 6.94-6.72 (m, 1H), 6.17-6.03 (m, 1H), 5.72, |
| | | 5.59 (2d, J = 10.2 Hz, 1H, rotamers), 4.79 (s, 1H), |
| | | 4.63, 4.41 (2d, J = 11.5 Hz, 1H, rotamers), 4.21, 4.05 |
| | | (2d, J = 10.5 Hz, 1H, rotamers), 3.77-3.40 (m, 1H), |
| | | 3.35-3.09(m, 1H), 2.39-2.27 (m, 2H), 1.99 (s, |
| | | 1H), 1.63 (s, 1H), 1.24 (s, 1H) |
| BCD-BTK-218 | 485.20 | δ = 9.38 (s, 1H), 8.57-8.53 (m, 1H), 8.51 (s, 1H), |
| | | 8.15 (d, J = 8.5 Hz, 2H), 7.76 (dd, J = 8.7, 4.5 Hz, |
| | | 1H), 7.65 (d, J = 8.7 Hz, 1H), 7.42 (d, J = 8.7 Hz, |
| | | 2H), 6.95-6.75 (m, 1H), 6.17-6.04 (m, 1H), 5.71, |
| | | 5.59 (2d, J = 10.3 Hz, 1H, rotamers), 5.30 (s, 1H), |
| | | 4.68, 4.41 (2d, J = 12.2 Hz, 1H, rotamers), 4.28, 4.07 |
| | | (2d, J = 11.0 Hz, 1H, rotamers), 3.75-3.39 (m, 1H), |
| | | 3.31-2.97 (m, 1H), 2.31 (s, 2H), 1.96 (s, 1H), 1.62 |
| | | (s, 1H) |
| BCD-BTK-219 | 556.30 | δ = 11.24 (s, 1H), 9.77 (s, 1H), 9.02 (s, 1H), 8.56 (d, |
| | | J = 5.1 Hz, 1H), 8.40 (s, 1H), 8.23 (s, 4H), 7.33 (dd, |
| | | J = 5.1, 1.2 Hz, 1H), 6.95-6.79 (m, 1H), 6.16-6.04 |
| | | (m, 1H), 5.72, 5.59 (2d, J = 9.8 Hz, 1H, rotamers), |
| | | 5.09 (s, 1H), 4.62, 4.47 (2d, J = 12.3 Hz, 1H, |
| | | rotamers), 4.27, 4.04 (2d, J = 13.2 Hz, 1H, rotamers), |
| | | 3.85-3.62 (m, 1H), 3.33-3.05 (m, 1H), 2.39-2.17 |
| | | (m, 4H), 2.02 (s, 1H), 1.59 (s, 1H), 0.96 (t, J = 7.4 |
| | | Hz, 3H) |
| BCD-BTK-220 | 503.20 | δ = 9.34 (s, 1H), 8.49 (s, J = 8.2 Hz, 1H), 8.46 (dd, |
| | | J = 4.0, 1.3 Hz, 1H), 8.03 (d, J = 8.6 Hz, 2H), 7.96 (s, |
| | | 1H), 7.65-7.50 (m, 3H), 7.13 (d, J = 8.7 Hz, 2H), |
| | | 6.92-6.77 (m, 1H), 6.18-6.03 (m, 1H), 5.71, 5.59 |
| | | (2d, J = 10.5 Hz, 1H, rotamers), 5.27 (s, 1H), 4.67, |
| | | 4.41 (2d, J = 11.5 Hz, 1H, rotamers), 4.29, 4.06 (2d, |
| | | J = 12.9 Hz, 1H, rotamers), 3.74-3.40 (m, 1H), |
| | | 3.33-2.94 (m, 2H), 2.35-2.26 (m2H), 1.96 (s, 1H), |
| | | 1.61 (s, 1H) |
| BCD-BTK-221 | 546.30 | δ = 11.46 (s, 1H), 9.77 (s, 1H), 9.02 (s, 1H), 8.71 (d, |
| | | J = 5.1 Hz, 1H), 8.58 (s, 1H), 8.24 (s, 4H), 7.57 (d, |
| | | J = 4.8 Hz, 1H), 6.97-6.77 (m, 1H), 6.17-6.03 (m, |
| | | 1H), 5.72, 5.59 (2d, J = 10.0 Hz, 1H, rotamers), 5.09 |
| | | (s, 1H), 4.62, 4.47 (2d, J = 11.4 Hz, 1H, rotamers), |
| | | 4.26, 4.03 (2d, J = 12.2 Hz, 1H, rotamers), 3.87-3.59 |
| | | (m, 1H), 3.33-3.03 (m, 1H), 2.34 (s, 2H), 2.02 |
| | | (s, 1H), 1.59 (s, 1H) |
| BCD-BTK-222 | 503.20 | δ = 9.34 (s, 1H), 8.86 (s, 1H), 8.55 (d, J = 2.6 Hz, |
| | | 1H), 8.46 (s, 1H), 8.16 (s, 1H), 8.08 (d, J = 8.7 Hz, |
| | | 2H), 7.90-7.86 (m, 1H), 7.59 (s, 1H), 7.26 (d, J = |
| | | 8.7 Hz, 2H), 6.90-6.69 (m, 1H), 6.18-6.05 (m, |
| | | 1H), 5.71, 5.59 (2d, J = 9.7 Hz, 1H, rotamers), 5.27 |
| | | (s, 1H), 4.77-4.36 (m, 1H), 4.36-4.06 (m, 1H), |
| | | 3.38-3.34 (m, 1H), 3.30-2.91 (m, 1H), 2.31 (s, |
| | | 2H), 1.99 (s, 1H), 1.66 (s, 1H) |
| BCD-BTK-223 | 496.20 | δ = 11.15 (s, 1H), 9.76 (s, 1H), 9.02 (s, 1H), 8.27-8.14 |
| | | (m, 5H), 8.05 (q, J = 8.2 Hz, 1H), 6.95 (dd, J = |
| | | 7.9, 2.3 Hz, 1H), 6.92-6.78 (m, 1H), 6.17-6.04 (m, |
| | | 1H), 5.72, 5.59 (2d, J = 9.8 Hz, 1H, rotamers), 5.09 |
| | | (s, 1H), 4.63, 4.47 (2d, J = 13.3 Hz, 1H, rotamers), |
| | | 4.27, 4.03 (2d, J = 11.6 Hz, 1H, rotamers), 3.86-3.60 |
| | | (m, 1H), 3.33-3.02 (m, 1H), 2.34 (s, 1H), 2.01 |
| | | (s, 1H), 1.59 (s, 1H) |
| BCD-BTK-224 | 542.20 | δ = 11.24 (s, 1H), 9.77 (s, 1H), 9.02 (s, 1H), 8.56 (d, |
| | | J = 5.1 Hz, 1H), 8.43 (s, 1H), 8.23 (s, 4H), 7.37 (d, |
| | | J = 3.8 Hz, 1H), 6.96-6.79 (m, 1H), 6.17-6.05 (m, |
| | | 1H), 5.72, 5.59 (2d, J = 10.4 Hz, 1H, rotamers), 5.09 |
| | | (s, 1H), 4.62, 4.47 (2d, J = 11.0 Hz, 1H, rotamers), |
| | | 4.27, 4.03 (2d, J = 12.9 Hz, 1H, rotamers), 3.86-3.61 |
| | | (m, 1H), 3.39-3.04 (m, 1H), 2.39-2.23 (m, |
| | | 2H), 2.09-1.94 (m, 4H), 1.60 (s, 1H) |
| BCD-BTK-225 | 505.20 | δ = 11.13 (s, 1H), 9.47 (s, 1H), 8.55 (s, 1H), 8.25-8.14 |
| | | (m, 5H), 8.05 (q, J = 8.2 Hz, 1H), 6.94 (dd, J = |
| | | 8.0, 2.5 Hz, 1H), 6.91-6.78 (m, 1H), 6.18-6.05 (m, |
| | | 1H), 5.72, 5.60 (2d, J = 10.4 Hz, 1H, rotamers), |
| | | 5.37-5.21 (m, 1H), 4.69, 4.44 (2d, J = 13.0 Hz, 1H, |
| | | rotamers), 4.29, 4.07 (2d, J = 13.3 Hz, 1H, rotamers), |
| | | 3.71-3.40 (m, 1H), 3.39-2.94 (m, 1H), 2.41-2.27 |
| | | (m, 2H), 1.98 (s, 1H), 1.63 (s, 1H) |
| BCD-BTK-226 | 505.30 | δ = 10.87 (s, 1H), 9.43 (s, 1H), 8.49 (s, 1H), 8.32 (d, |
| | | J = 4.6 Hz, 1H), 8.20 (s, 4H), 7.84-7.76 (m, 1H), |
| | | 7.45-7.38 (m, 2H), 6.91-6.73 (m, 1H), 6.20-6.05 |
| | | (m, 1H), 5.72, 5.60 (2d, J = 9.8 Hz, 1H, rotamers), |
| | | 5.30 (s, 1H), 4.73, 4.42 (2d, J = 11.8 Hz, 1H, |
| | | rotamers), 4.34, 4.09 (2d, J = 12.9 Hz, 1H), 3.81-3.34 |
| | | (m, 1H), 3.33-2.95 (m, 1H), 2.33 (s, 2H), 2.00 |
| | | (s, 1H), 1.66 (s, 1H) |
| BCD-BTK-227 | 496.30 | δ = 11.00 (s, 1H), 9.72 (s, 1H), 8.95 (s, 1H), 8.36 (d, |
| | | J = 3.1 Hz, 1H), 8.29 (dd, J = 9.2, 4.2 Hz, 1H), |
| | | 8.24-8.17 (m, 4H), 7.75 (td, J = 8.6, 3.1 Hz, 1H), |
| | | 6.92-6.73 (m, 1H), 6.20-6.02 (m, 1H), 5.71, 5.58 (2d, |
| | | J = 10.1 Hz, 1H, rotamers), 5.18-4.97 (m, 1H), 4.67, |
| | | 4.45 (2d, J = 11.7 Hz, 1H, rotamers), 4.32, 4.07 (2d, |
| | | J = 11.9 Hz, 1H, rotamers), 3.89-3.56 (m, 1H), |
| | | 3.33-2.98 (m, 1H), 2.40-2.23 (m, 2H), 2.04 (s, 1H), |
| | | 1.65 (s, 1H) |
| BCD-BTK-228 | 478.30 | δ = 11.06 (s, 1H), 9.77 (s, 1H), 9.02 (s, 1H), 8.43 (d, |
| | | J = 3.7 Hz, 1H), 8.27-8.17 (m, 4H), 7.95-7.89 (m, |
| | | 1H), 7.26-7.20 (m, 1H), 6.98-6.77 (m, 1H), 6.16-6.04 |
| | | (m, 1H), 5.72, 5.59 (2d, J = 9.7 Hz, 1H, rotamers), |
| | | 5.09 (s, 1H), 4.62, 4.48 (2d, J = 13.3 Hz, 1H, |
| | | rotamers), 4.27 (m, 1H), 3.80-3.62 (m, 1H), |
| | | 3.46-3.01 (m, 1H), 2.40-2.21 (m, 2H), 2.02 (s, |
| | | 1H), 1.59 (s, 1H) |
| BCD-BTK-229 | 489.20 | δ = 10.55 (s, 1H), 9.67 (s, 2H), 9.57 (s, 1H), 8.58 (s, |
| | | 1H), 8.43 (d, J = 4.0 Hz, 2H), 8.30 (d, J = 8.2 Hz, |
| | | 1H), 7.98-7.88 (m, 1H), 7.24 (dd, J = 6.9, 5.2 Hz, |
| | | 2H), 6.94-6.78 (m, 2H), 6.21-6.02 (m, 1H), 5.72, |
| | | 5.60 (2d, J = 10.5 Hz, 1H, rotamers), 5.42-5.24 (m, |
| | | 1H), 4.66, 4.42 (2d, J = 10.8 Hz, 1H, rotamers), 4.22, |
| | | 4.05 (2d, J = 11.7 Hz, 1H, rotamers), 3.88-3.49 (m, |
| | | 1H), 3.33-3.07 (m, 1H), 2.43-2.28 (m, 2H), 1.99 |
| | | (s, 1H), 1.64 (s, 1H) |
| BCD-BTK-230 | 444.20 | δ = 9.29 (d, J = 1.9 Hz, 1H), 8.56-8.53 (m, 1H), |
| | | 8.48 (d, J = 3.6 Hz, 1H), 8.16 (d, J = 8.2 Hz, 2H), |
| | | 7.76 (dd, J = 8.7, 4.5 Hz, 1H), 7.68-7.62 (m, 1H), |
| | | 7.42 (d, J = 8.7 Hz, 1H), 6.95-6.71 (m, 1H), 6.18-6.03 |
| | | (m, 1H), 5.71, 5.58 (2d, J = 10.1 Hz, 1H, rotamers), |
| | | 4.78 (s, 1H), 4.63, 4.40 (2d, J = 11.6 Hz, 1H, |
| | | rotamers), 4.21, 4.05 (2d, J = 13.6 Hz, 1H, |
| | | rotamers), 3.78-3.33 (m, 1H), 3.29-3.01 (m, 1H), |
| | | 2.39-2.28 (m, 2H), 1.99 (s, 1H), 1.61 (s, 1H). |
| BCD-BTK-231 | 580.20 | δ = 11.20 (s, 1H), 8.56 (d, J = 5.1 Hz, 1H), 8.39 (s, |
| | | 1H), 8.22 (d, J = 8.3 Hz, 2H), 7.90 (s, 1H), 7.79 (d, |
| | | J = 8.0 Hz, 2H), 7.33 (d, J = 5.1 Hz, 1H), 6.94-6.76 |
| | | (m, 1H), 6.65-6.46 (m, 2H), 6.18-6.03 (m, 1H), |
| | | 5.72, 5.62 (2d, J = 10.3 Hz, 1H, rotamers), 5.20 (s, |
| | | 1H), 4.68, 4.38 (2d, J = 11.9 Hz, 1H, rotamers), 4.31, |
| | | 4.06 (2d, J = 12.8 Hz, 1H, rotamers), 3.72-3.28 (m, |
| | | 1H), 3.28-2.85 (m, 1H), 2.37-2.16 (m, 4H), 1.93 |
| | | (s, 1H), 1.59 (s, 1H), 0.96 (t, J = 7.4 Hz, 3H) |
| BCD-BTK-232 | 500.10 | δ = 8.92 (s, 1H), 8.65 (d, J = 6.0 Hz, 1H), 7.80-7.71 |
| | | (m, 3H), 7.50-7.39 (m, 2H), 7.09 (d, J = 6.0 Hz, |
| | | 1H), 6.90-6.66 (m, 1H), 6.21-6.05 (m, 1H), 6.01 |
| | | (s, 2H), 5.70, 5.61 (2d, J = 10.6 Hz, 1H, rotamers), |
| | | 5.19 (s, 1H), 4.81-4.35 (m, 1H), 4.37-4.05 (m, |
| | | 1H), 3.69-3.10 (m, 1H), 2.94-2.78 (m, 1H), 2.41-2.21 |
| | | (m, 2H), 2.05-1.92 (m, 1H), 1.62 (m, 1H) |
| BCD-BTK-233 | 520.20 | δ = 10.90 (s, 1H), 8.35 (dt, J = 4.7, 1.3 Hz, 1H), |
| | | 8.23-8.17 (m, 2H), 7.93-7.76 (m, 4H), 7.45 (ddd, J = |
| | | 8.3, 4.7, 3.8 Hz, 1H), 6.94-6.75 (m, 1H), 6.46 (s, |
| | | 2H), 6.19-6.04 (m, 1H), 5.72, 5.62 (2d, J = 10.6 Hz, |
| | | 1H, rotamers), 5.22 (s, 1H), 4.69, 4.38 (2d, J = 12.9 |
| | | Hz, 1H, rotamers), 4.31, 4.06 (2d, J = 13.3 Hz, 1H, |
| | | rotamers), 3.73-3.29 (m, 1H), 3.28-2.87 (m, 1H), |
| | | 2.31-2.21 (m, 2H), 1.94 (s, 1H), 1.60 (s, 1H) |
| BCD-BTK-234 | 493.20 | δ = 10.79 (s, 1H), 8.38 (d, J = 3.9 Hz, 2H), 8.32-8.17 |
| | | (m, 4H), 7.87-7.78 (m, 2H), 7.74 (d, J = 8.2 Hz, |
| | | 2H), 7.21-7.11 (m, 1H), 6.79 (s, 3H), 6.11 (m, |
| | | 1H), 5.65 (m, 1H), 4.91 (m, 1H), 4.73-4.33 (m, |
| | | 1H), 4.35-4.02 (m, 1H), 3.59 (m, 1H), 3.33-2.94 |
| | | (m, 1H), 2.28 (s, 2H), 1.99 (s, 1H), 1.62 (s, 1H) |
| BCD-BTK-235 | 496.20 | δ = 10.95 (s, 1H), 9.77 (s, 1H), 9.02 (s, 1H), 8.35 (d, |
| | | J = 4.7 Hz, 1H), 8.23 (q, J = 8.5 Hz, 4H), 7.92-7.78 |
| | | (m, 1H), 7.45 (dt, J = 8.3, 4.1 Hz, 1H), 6.98-6.72 (m, |
| | | 1H), 6.16-6.03 (m, 1H), 5.72, 5.59 (2d, J = 10.1 Hz, |
| | | 1H, rotamers), 5.10 (s, 1H), 4.63, 4.48 (2d, J = 12.2 |
| | | 1H, rotamers), 4.28, 4.03 (2d, J = 11.7 Hz, 1H, |
| | | rotamers), 3.85-3.59 (m, 1H), 3.16-3.02 (m, 1H), |
| | | 2.43-2.19 (m, 2H), 2.01 (s, 1H), 1.59 (s, 1H) |
| BCD-BTK-236 | 500.20 | δ = 8.53 (dd, J = 4.3, 1.5 Hz, 1H), 7.81 (s, 1H), |
| | | 7.78-7.69 (m, 4H), 7.44-7.39 (m, 2H), 6.92-6.75 (m, |
| | | 1H), 6.24-6.03 (m, 3H), 5.71, 5.61 (2d, J = 10.2 Hz, |
| | | 1H, rotamers), 5.18 (s, 1H), 4.71-4.34 (m, 1H), |
| | | 4.34-4.04 (m, 1H), 3.69-3.25 (m, 1H), 3.24-2.86 (m, |
| | | 1H), 2.24 (s, 2H), 1.94 (m, 1H), 1.58 (s, 1H) |
| BCD-BTK-237 | 520.10 | δ = 11.02 (s, 1H), 8.42 (d, J = 3.1 Hz, 1H), 8.26 (dd, |
| | | J = 9.2, 4.2 Hz, 1H), 8.20 (d, J = 8.3 Hz, 2H), |
| | | 7.86-7.75 (m, 4H), 6.92-6.76 (m, 1H), 6.19-5.98 (m, |
| | | 3H), 5.71, 5.62 (2d, J = 10.4 Hz, 1H, rotamers), 5.19 |
| | | (s, 1H), 4.69, 4.37 (2d, J = 12.7 Hz, 1H, rotamers), |
| | | 4.31, 4.07 (2d, J = 11.9 Hz, 1H, rotamers), 3.70-3.26 |
| | | (m, 1H), 3.26-2.86 (m, 1H), 2.25 (s, 2H), 1.94 (s, |
| | | 1H), 1.58 (s, 1H) |
| BCD-BTK-238 | 502.10 | δ = 10.90 (s, 1H), 8.44-8.39 (m, 1H), 8.22 (dd, J = |
| | | 8.4, 2.0 Hz, 3H), 7.92-7.84 (m, 2H), 7.78 (d, J = 8.1 |
| | | Hz, 2H), 7.23-7.17 (m, 1H), 6.92-6.76 (m, 1H), |
| | | 6.62-6.44 (m, 2H), 6.20-6.04 (m, 1H), 5.71, 5.62 |
| | | (2d, J = 10.2 Hz, 1H, rotamers), 5.21 (s, 1H), 4.69, |
| | | 4.38 (2d, J = 12.4 Hz, 1H, rotamers), 4.31, 4.06 (2d, |
| | | J = 13.0 Hz, 1H, rotamers), 3.71-3.29 (m, 1H), |
| | | 3.27-2.88 (m, 1H), 2.36-2.20 (m, 2H), 1.93 (s, 1H), |
| | | 1.59 (s, 1H) |
| BCD-BTK-239 | 471.20 | δ = 8.56-8.50 (m, 2H), 7.73 (d, J = 8.3 Hz, 2H), 7.50 |
| | | (s, 1H), 7.40-7.33 (m, 2H), 7.14-7.01 (m, 4H), |
| | | 6.92-6.70 (m, 1H), 6.14, 6.10 (2d, J = 7.3 Hz, 1H, |
| | | rotamers), 5.72, 5.65 (2d, J = 10.8 Hz, 1H, rotamers), |
| | | 5.02 (s, 1H), 4.64, 4.33 (2d, J = 13.5 Hz, 1H, |
| | | rotamers), 4.28, 4.05 (2d, J = 12.2 Hz, 1H, rotamers), |
| | | 3.62-3.28 (m, 1H), 3.28-2.84 (m, 1H), 2.37-2.16 |
| | | (m, 2H), 1.94 (s, 1H), 1.57 (s, 1H) |
| BCD-BTK-240 | 498.20 | δ = 10.87 (s, 1H), 8.41 (dd, J = 5.2, 1.8 Hz, 1H), |
| | | 8.26-8.15 (m, 3H), 7.89-7.84 (m, 1H), 7.79 (d, J = 8.0 |
| | | Hz, 2H), 7.51 (s, 1H), 7.21-7.17 (m, 1H), 6.93-6.73 |
| | | (m, 1H), 6.18-6.06 (m, 1H), 5.71, 5.65 (2d, J = 10.6 |
| | | Hz, 1H, rotamers), 5.52 (s, 2H), 4.97 (s, 1H), 4.66, |
| | | 4.34 (2d, J = 12.5 Hz, 1H, rotamers), 4.31, 4.06 (2d, |
| | | J = 13.7 Hz, 1H, rotamers), 3.91 (s, 3H), 3.58 -3.31 |
| | | (m, 1H), 3.25-2.81 (m, 1H), 2.39-2.17 (m, 2H), |
| | | 1.97-1.88 (m, 1H), 1.56 (s, 1H) |
| BCD-BTK-241 | 441.20 | δ = 8.54-8.47 (m, 2H), 7.81-7.72 (m, 3H), 7.37-7.30 |
| | | (m, 2H), 7.09-7.04 (m, 2H), 6.97 (s, 1H), 6.93-6.68 |
| | | (m, 1H), 6.15, 6.06 (2d, J = 16.4 Hz, 1H, rotamers), |
| | | 5.88 (s, 2H), 5.72, 5.59 (2d, J = 10.3 Hz, 1H, |
| | | rotamers), 4.73-4.49 (m, 2H), 4.34-4.03 (m, 2H), |
| | | 3.70-3.32 (m, 1H), 3.23-2.91 (m, 1H), 2.16 (s, 2H), |
| | | 1.99-1.85 (m, 1H), 1.62 (s, 1H) |
| BCD-BTK-242 | 468.20 | δ = 10.86 (s, 1H), 8.43-8.40 (m, 1H), 8.24-8.18 (m, |
| | | 3H), 7.89-7.84 (m, 1H), 7.83-7.77 (m, 3H), 7.21-7.16 |
| | | (m, 1H), 7.03-6.97 (m, 1H), 6.93-6.70 (m, 1H), |
| | | 6.20-6.03 (m, 1H), 5.91 (s, 2H), 5.73, 5.60 (2d, |
| | | J = 10.2 Hz, 1H, rotamers), 4.75-4.51 (m, 2H), |
| | | 4.34-4.05 (m, 2H), 3.73-3.32 (m, 1H), 3.26-2.87 (m, |
| | | 1H), 2.27-2.09 (m, 2H), 1.91 (s, 1H), 1.63 (s, 1H) |
| BCD-BTK-243 | 471.20 | δ = 10.90 (s, 1H), 9.38 (s, 1H), 8.60-8.35 (m, 2H), |
| | | 8.31-8.15 (m, 5H), 7.87 (t, J = 7.9 Hz, 1H), 7.24-7.15 |
| | | (m, 1H), 6.94-6.74 (m, 1H), 6.17-6.05 (m, 1H), |
| | | 5.72, 5.59 (2d, J = 10.5 Hz, 1H, rotamers), 4.79 |
| | | (s, 1H), 4.64, 4.42 (2d, J = 12.6 Hz, 1H, rotamers), |
| | | 4.22, 4.04 (2d, J = 13.3 Hz, 1H, rotamers), 3.82-3.37 |
| | | (m, 1H), 3.33-3.03 (m, 1H), 2.42-2.26 (m, 2H), |
| | | 1.99 (s, 1H), 1.63 (s, 1H) |
| BCD-BTK-244 | 489.20 | δ = 11.05 (s, 1H), 9.36 (d, J = 2.2 Hz, 1H), 8.49 (d, |
| | | J = 3.6 Hz, 1H), 8.43 (d, J = 3.1 Hz, 1H), 8.26 (dd, |
| | | J = 9.2, 4.2 Hz, 1H), 8.20 (s, 4H), 7.82 (td, J = 8.7, 3.1 |
| | | Hz, 1H), 6.94-6.74 (m, 1H), 6.17-6.00 (m, 1H), 5.72, |
| | | 5.59 (2d, J = 10.4 Hz, 1H, rotamers), 4.80 (s, 1H), |
| | | 4.64, 4.22 (2d, J = 12.7 Hz, 1H, rotamers), 4.42, 4.04 |
| | | (2d, J = 13.7 Hz, 1H, rotamers), 3.80-3.37 (m, 1H), |
| | | 3.33-3.05 (m, 1H), 2.43-2.27 (m, 2H), 1.99 (s, 1H), |
| | | 1.63 (s, 1H) |
| BCD-BTK-245 | 539.10 | δ = 11.42 (s, 1H), 9.37 (d, J = 2.2 Hz, 1H), 8.70 (d, |
| | | J = 5.2 Hz, 1H), 8.58 (s, 1H), 8.50 (d, J = 3.6 Hz, 1H), |
| | | 8.23 (s, 4H), 7.56 (dd, J = 5.1, 1.6 Hz, 1H), 6.92-6.75 |
| | | (m, 1H), 6.16-6.05 (m, 1H), 5.72, 5.59 (2d, J = 10.3 |
| | | Hz, 1H, rotamers), 4.80 (s, 1H), 4.63, 4.21 (2d, J = |
| | | 12.6 Hz, 1H, rotamers), 4.42, 4.04 (2d, J = 13.6 Hz, |
| | | 1H, rotamers), 3.81-3.38 (m, 1H), 3.33-3.02 (m, |
| | | 1H), 2.41-2.27 (m, 2H), 1.99 (s, 1H), 1.64 (s, 1H) |
| BCD-BTK-246 | 487.20 | δ = 9.30 (d, J = 2.3 Hz, 1H), 8.77 (s, 1H), 8.50 (d, J = |
| | | 5.7 Hz, 1H), 8.47 (d, J = 3.6 Hz, 1H), 8.17 (d, J = 8.5 |
| | | Hz, 2H), 7.86 (s, 1H), 7.77 (s, 1H), 7.44-7.35 (m, |
| | | 2H), 6.93-6.73 (m, 2H), 6.17-6.02 (m, 1H), 5.71, |
| | | 5.59 (2d, J = 10.4 Hz, 1H rotamers), 4.77 (s, 1H), |
| | | 4.63, 4.22 (d, J = 12.7 Hz, 1H, rotamers), 4.40, 4.05 |
| | | (2d, J = 13.6 Hz, 1H, rotamers), 3.76-3.35 (m, 1H), |
| | | 3.14-3.03 (m, 1H), 2.41-2.24 (m, 2H), 1.98 (s, 1H), |
| | | 1.63 (s, 1H) |
| BCD-BTK-247 | 563.20 | δ = 11.20 (s, 1H), 9.37 (d, J = 2.2 Hz, 1H), 8.55 (d, |
| | | J = 5.1 Hz, 1H), 8.50 (d, J = 3.6 Hz, 1H), 8.39 (s, 1H), |
| | | 8.22 (s, 4H), 7.32 (dd, J = 5.2, 1.5 Hz, 1H), 6.93-6.75 |
| | | (m, 1H), 6.19-6.02 (m, 1H), 5.72, 5.59 (2d, J = 10.5 |
| | | Hz, 1H, rotamers), 4.80 (s, 1H), 4.63, 4.22 (2d, J = |
| | | 12.6 Hz, 1H, rotamers), 4.42, 4.04 (2d, J = 13.6 Hz, |
| | | 1H, rotamers), 3.81-3.37 (m, 1H), 3.33-3.05 (m, |
| | | 1H), 2.41-2.29 (m, 2H), 2.29-2.13 (m, 2H), 1.99 (s, |
| | | 1H), 1.63 (s, 1H), 1.47-1.34 (m, 2H), 0.94 (t, J = 7.4 |
| | | Hz, 3H) |
| BCD-BTK-248 | 489.20 | δ = 11.11 (s, 1H), 9.37 (d, J = 2.2 Hz, 1H), 8.50 (d, |
| | | J = 3.6 Hz, 1H), 8.20 (s, 4H), 8.17 (dd, J = 8.1, 2.4 Hz, |
| | | 1H), 8.05 (q, J = 8.2 Hz, 1H), 6.94 (dd, J = 8.0, 2.5 |
| | | Hz, 1H), 6.94-6.74 (m, 1H), 6.19-6.02 (m, 1H), |
| | | 5.72, 5.59 (2d, J = 10.5 Hz, 1H, rotamers), 4.79 (s, |
| | | 1H), 4.64, 4.22 (2d, J = 12.6 Hz, 1H, rotamers), 4.42, |
| | | 4.04 (d, J = 13.6 Hz, 1H, rotamers), 3.80-3.37 (m, |
| | | 1H), 3.33-3.00 (m, 1H), 2.44-2.26 (m, 2H), 1.99 |
| | | (s, 1H), 1.63 (s, 1H) |
| BCD-BTK-249 | 489.20 | δ = 10.91 (s, 1H), 9.38 (d, J = 2.2 Hz, 1H), 8.50 (d, |
| | | J = 3.6 Hz, 1H), 8.35 (dt, J = 4.7, 1.3 Hz, 1H), |
| | | 8.27-8.18 (m, 4H), 7.88-7.82 (m,1H), 7.47-7.41 (m, |
| | | 1H), 6.93-6.74 (m, 1H), 6.17-6.05 (m, 1H), 5.72, |
| | | 5.59 (2d, J = 10.4 Hz, 1H, rotamers), 4.80 (s, 1H), |
| | | 4.64, 4.23 (2d, J = 12.7 Hz, 1H, rotamers), 4.42, 4.05 |
| | | (2d, J = 13.6 Hz, 1H, rotamers), 3.79-3.36 (m, 1H), |
| | | 3.32-3.04 (m, 1H), 2.42-2.27 (m, 2H), 1.99 (s, 1H), |
| | | 1.64 (s, 1H) |
| BCD-BTK-250 | 520.20 | δ = 11.39 (s, 1H), 8.70 (d, J = 5.1 Hz, 1H), 8.58 (s, |
| | | 1H), 8.24-8.20 (m, 2H), 7.82 (s, 1H), 7.79 (d, J = 8.1 |
| | | Hz, 2H), 7.56 (dd, J = 5.2, 1.5 Hz, 1H), 6.92-6.76 |
| | | (m, 1H), 6.17-6.01 (m, 3H), 5.71, 5.62 (2d, J = 10.4 |
| | | Hz, 1H, rotamers), 5.20 (s, 1H), 4.68, 4.31 (2d, J = |
| | | 12.5 Hz, 1H, rotamers), 4.37, 4.07 (2d, J = 13.5 Hz, |
| | | 1H, rotamers), 3.70-3.33 (m, 1H), 3.27-2.86 (m, |
| | | 1H), 2.35-2.21 (m, 2H), 1.98-1.87 (m, 1H), 1.59 |
| | | (s, 1H) |
| BCD-BTK-251 | 570.20 | δ = 11.09 (s, 1H), 8.22-8.15 (m, 3H), 8.05 (q, J = 8.2 |
| | | Hz, 1H), 7.82 (s, 1H), 7.77 (d, J = 8.0 Hz, 2H), 6.94 |
| | | (dd, J = 8.0, 2.5 Hz, 1H), 6.92-6.76 (m, 1H), |
| | | 6.18-5.99 (m, 3H), 5.71, 5.62 (d, J = 10.5 Hz, 1H, |
| | | rotamers), 5.20 (s, 1H), 4.69, 4.31 (2d, J = 12.6 Hz, |
| | | 1H, rotamers), 4.37, 4.06 (2d, J = 13.6 Hz, 1H, |
| | | rotamers), 3.68-3.33 (m, 1H), 3.27-2.83 (m, 1H), |
| | | 2.35-2.20 (m, 2H), 1.92 (s, 1H), 1.59 (s, 1H) |
| BCD-BTK-252 | 579.20 | δ = 11.20 (s, 1H), 9.46 (s, 1H), 8.58-8.50 (m, 2H), |
| | | 8.39 (s, 1H), 8.25-8.17 (m, 4H), 7.32 (d, J = 5.1 Hz, |
| | | 1H), 6.94-6.75 (m, 1H), 6.20-6.03 (m, 1H), 5.72, |
| | | 5.60 (2d, J = 10.4 Hz, 1H, rotamers), 5.29 (s, 1H), |
| | | 4.69, 4.29 (2d, J = 12.2 Hz, 1H, rotamers), 4.43, 4.06 |
| | | (2d, J = 13.4 Hz, 1H), 3.81-3.41 (m, 1H), 3.30-2.98 |
| | | (m, 1H), 2.40-2.12 (m, 6H), 1.98 (s, 1H), 1.64 (s, |
| | | 1H), 1.48-1.33 (m, 2H), 0.94 (t, J = 7.4 Hz, 3H) |
| BCD-BTK-253 | 507.10 | δ = 10.69 (s, 1H), 9.67 (s, 2H), 9.57 (s, 1H), 8.59 (s, |
| | | 1H), 8.45 (d, J = 2.8 Hz, 1H), 8.34 (dd, J = 8.8, 3.7 |
| | | Hz, 1H), 7.90 (td, J = 8.7, 3.0 Hz, 1H), 6.96-6.71 (m, |
| | | 1H), 6.19-5.99 (m, 1H), 5.72, 5.59 (2d, J = 10.4 Hz, |
| | | 1H, rotamers), 5.37 (s, 1H), 4.66, 4.19 (2d, J = 11.8 |
| | | 1H, rotamers), 4.42, 4.04 (2d, J = 14.2 Hz, 1H, |
| | | rotamers), 3.86-3.47 (m, 1H), 3.33-3.04 (m, 1H), |
| | | 2.40-2.28 (m, 2H), 1.99 (s, 1H), 1.65 (s, 1H) |
| BCD-BTK-254 | 594.10 | δ = 11.17 (s, 1H), 8.55 (d, J = 5.1 Hz, 1H), 8.39 (s, |
| | | 1H), 8.21 (d, J = 8.4 Hz, 2H), 7.84-7.75 (m, 3H), |
| | | 7.32 (dd, J = 5.1, 1.4 Hz, 1H), 6.93-6.74 (m, 1H), |
| | | 6.19-5.98 (m, 3H), 5.71, 5.62 (2d, J = 10.3 Hz, 1H, |
| | | rotamers), 5.19 (s, 1H), 4.68, 4.31 (2d, J = 12.2 Hz, |
| | | 1H, rotamers), 4.37, 4.07 (2d, J = 13.3 Hz, 1H, |
| | | rotamers), 3.68-3.25 (m, 1H), 3.26-2.87 (m, 1H), |
| | | 2.34-2.14 (m, 4H), 1.92 (d, J = 7.4 Hz, 1H), 1.58 (s, |
| | | 1H), 1.46-1.35 (m, 2H), 0.93 (t, J = 7.4 Hz, 3H) |
| BCD-BTK-255 | 487.20 | δ = 9.24 (d, J = 2.2 Hz, 1H), 8.48-8.43 (m, 2H), 8.04 |
| | | (d, J = 8.6 Hz, 2H), 7.94 (s, 1H), 7.64-7.54 (m, 2H), |
| | | 7.52 (s, 1H), 7.16-7.10 (m, 2H), 6.92-6.72 (m, 1H), |
| | | 6.17-6.03 (m, 1H), 5.71, 5.58 (2d, J = 10.4 Hz, 1H, |
| | | rotamers), 4.74 (s, 1H), 4.62, 4.22 (2d, J = 12.5 Hz, |
| | | 1H, rotamers), 4.39, 4.04 (d, J = 13.6 Hz, 1H, |
| | | rotamers), 3.76-3.32 (m, 1H), 3.29-2.98 (m, 1H), |
| | | 2.39-2.24 (m, 2H), 1.97 (s, 1H), 1.61 (s, 1H) |
| BCD-BTK-258 | 511.10 | δ = 10.89 (s, 1H), 8.38-8.32 (m, 2H), 8.22-8.18 (m, |
| | | 2H), 7.88-7.82 (m, 1H), 7.79 (2d, J = 8.1 Hz, 2H), |
| | | 7.47-7.42 (m, 1H), 7.29-6.62 (m, 2H), 6.19-6.00 |
| | | (m, 1H), 5.72, 5.61 (2d, J = 10.5 Hz, 1H, rotamers), |
| | | 5.02-4.82 (m, 1H), 4.62, 4.29 (2d, J = 12.7 Hz, 1H, |
| | | rotamers), 4.39, 4.05 (2d, J = 13.4 Hz, 1H, rotamers), |
| | | 3.78-3.43 (m, 1H), 3.33-2.85 (m, 1H), 2.31-2.10 |
| | | (m, 2H), 1.96 (s, 1H), 1.55 (s, 1H) |
| BCD-BTK-259 | 472.20 | ¹H NMR (400 MHz, DMSO, mixture of amide |
| | | rotamers 60/40): δ = 9.39, 9.35 (2s, 1H, rotamers), |
| | | 8.53 (s, 1H), 8.52 (s, 1H), 8.48 (s, 1H), 8.17-8.11 (m, |
| | | 2H), 7.37 (dd, J = 8.7, 1.8 Hz, 2H), 7.08-7.03 (m, |
| | | 2H), 4.97-4.88 (m, 1H), 4.85-4.50 (m, 2H), |
| | | 3.68-3.39 (m, 2H), 2.12-1.88 (m, 3H), 1.87-1.69 (m, |
| | | 1H), 1.99, 1.32 (2s, 3H, rotamers) |
| BCD-BTK-260 | 487.20 | ¹H NMR (400 MHz, DMSO, mixture of amide |
| | | rotamers 60/40): δ = 10.92 (s, 1H), 9.42, 9.37 |
| | | (2s, 1H), 8.50, 8.49 (2s, 1H), 8.43-8.40 (m, 1H), |
| | | 8.25-8.19 (m, 3H), 8.16 (d, J = 8.5 Hz, 2H), |
| | | 7.90-7.84 (m, 1H), 7.22-7.16 (m, 1H), 6.60-6.48, |
| | | 5.91-5.82 (2m, 1H), 6.03-5.96, 5.63-5.55 (2m, 1H), |
| | | 5.03-4.94 (m, 1H) 5.63-5.55, 5.03-4.94 (2m, 1H), |
| | | 4.87-4.79 (m, 1H), 4.74-4.65 (m, 1H), 3.64-3.48 |
| | | (m, 2H), 2.11-1.77 (m, 4H) |
| BCD-BTK-261 | 458.20 | δ = 9.38 (s, 1H), 8.54-8.49 (m, 3H), 8.19-8.10 (m, |
| | | 2H), 7.37 (d, J = 8.7 Hz, 2H), 7.07-7.01 (m, 2H), |
| | | 5.06-4.97 (m, 2H), 4.32-4.19 (m, 1H), 4.14-4.06 |
| | | (m, 1H), 4.05-3.96 (m, 1H), 3.89-3.81 (m, 1H), |
| | | 3.31-3.20 (m, 1H), 1.97 (s, 3H) |
| BCD-BTK-262 | 485.20 | δ = 10.91 (s, 1H), 9.46 (d, J = 5.2 Hz, 1H), 8.54 (d, |
| | | J = 2.6 Hz, 1H), 8.43-8.39 (m, 1H), 8.24-8.15 (m, |
| | | 5H), 7.90-7.83 (m, 1H), 7.22-7.16 (m, 1H), |
| | | 6.09-6.04 (m, 1H), 4.27-4.14 (m, 1H), 4.02-3.83 |
| | | (m, 1H), 3.73-3.53 (m, 1H), 2.61-2.53 (m, 2H), |
| | | 2.05, 1.98 (2s, 3H, rotamers) |
| BCD-BTK-263 | 460.10 | ¹H NMR (400 MHz, DMSO, mixture of amide |
| | | rotamers 60/40): δ = 9.35, 9.30 (2s, 1H, rotamers), |
| | | 8.57-8.49 (m, 2H), 8.47 (d, J = 5.1 Hz, 1H), |
| | | 8.15-8.09 (m, 2H), 7.40-7.32 (m, 2H), 7.09-7.01 |
| | | (m, 2H), 6.60-6.49, 5.92-5.82 (2m, 1H, rotamers), |
| | | 6.05-5.95, 5.05-5.01 (2m, 1H, rotamers), 5.66-5.57 |
| | | (m, 1H), 5.00-4.91, 4.82-4.75 (2m, 2H, rotamers), |
| | | 4.73-4.61 (m, 1H), 3.63-3.47 (m, 2H), 2.09-1.75 |
| | | (m, 4H) |
| BCD-BTK-264 | 446.20 | δ = 9.38 (s, 1H), 8.54-8.49 (m, 3H), 8.18-8.11 (m, |
| | | 2H), 7.40-7.33 (m, 2H), 7.07-7.02 (m, 2H), |
| | | 6.39-6.25 (m, 1H), 6.13-6.04 (m, 1H), 5.64 (dd, J = |
| | | 10.3, 2.3 Hz, 1H), 5.03 (d, J = 7.3 Hz, 2H), 4.35 |
| | | (t, J = 8.61 Hz, 1H), 4.22-4.14 (m, 1H), 4.08-3.99 |
| | | (m, 1H), 3.90 (dd, J = 10.2, 5.6 Hz, 1H), 3.35-3.21 |
| | | (m, 1H) |
| BCD-BTK-265 | 458.20 | δ = 9.39 (d, J = 5.2 Hz, 1H), 8.55-8.48 (m, 3H), |
| | | 8.17-8.08 (m, 2H), 7.43-7.30 (m, 2H), 7.07-7.00 |
| | | (m, 2H), 6.10-5.97 (m, 1H), 4.27-4.11 (m, 1H), |
| | | 4.03-3.77 (m, 2H), 3.75-3.51 (m, 1H), 2.63-2.51 |
| | | (m, 2H), 2.03, 1.97 (2s, 3H, rotamers) |
| BCD-BTK-266 | 446.20 | δ = 9.39 (d, J = 2.6 Hz, 1H), 8.57-8.46 (m, 3H), |
| | | 8.18-8.04 (m, 2H), 7.39-7.30 (m, 2H), 7.08-6.97 (m, |
| | | 2H), 6.75-6.50 (m, 1H), 6.22-5.95 (m, 2H), |
| | | 5.75-5.58 (m, 1H), 4.22-4.10 (m, 1H), 4.05-3.56 |
| | | (m, 3H), 2.71-2.51 (m, 2H) |
| BCD-BTK-267 | 485.30 | δ = 10.92 (s, 1H), 9.45 (s, 1H), 8.54 (s, 1H), |
| | | 8.45-8.37 (m, 1H), 8.26-8.13 (m, 5H), 7.91-7.81 |
| | | (m, 1H), 7.23 -7.15 (m, 1H), 5.05 (d, J = 7.2 Hz, 2H), |
| | | 4.28-4.19 (m, 1H), 4.11 (dd, J = 9.2, 5.4 Hz, 1H), |
| | | 4.05-3.98 (m, 1H), 3.87 (dd, J = 10.2, 5.5 Hz, 1H), |
| | | 3.39-3.23 (m, 1H), 1.98 (s, 3H) |
| BCD-BTK-268 | 570.30 | δ = 11.22 (s, 1H), 9.76 (s, 1H), 9.02 (s, 1H), 8.55 (d, |
| | | J = 5.1 Hz, 1H), 8.39 (d, J = 1.4 Hz, 1H), 8.23 (s, 4H), |
| | | 7.33 (dd, J = 5.2, 1.6 Hz, 1H), 6.96-6.76 (m, 1H), |
| | | 6.18-6.02 (m, 1H), 5.72, 5.59 (2d, J = 10.5 Hz, 1H, |
| | | rotamers), 5.09 (s, 1H), 4.62, 4.27 (2d, J = 12.9 Hz, |
| | | 1H, rotamers), 4.47, 4.03 (2d, J = 13.4 Hz, 1H, |
| | | rotamers), 3.87-3.61 (m, 1H), 3.47-3.01 (m, 1H), |
| | | 2.40-2.31 (m, 2H), 2.30-2.13 (m, 2H), 2.01 (s, 1H), |
| | | 1.60 (s, 1H), 1.49-1.34 (m, 2H), 0.94 (t, J = 7.4 Hz, |
| | | 3H) |
| BCD-BTK-269 | 499.20 | ¹H NMR (400 MHz, DMSO, mixture of amide |
| | | rotamers 60/40): δ = 10.91 (s, 1H), 9.46, 9.42 |
| | | (2s, 1H, rotamers), 8.54, 8.51 (2s, 1H, rotamers), 8.42 |
| | | (dd, J = 4.9, 1.8 Hz, 1H), 8.25-8.11 (m, 5H), 7.91-7.82 |
| | | (m, 1H), 7.19 (dd, J = 7.3, 4.9 Hz, 1H), 5.01-4.89 (m, |
| | | 1H), 4.89-4.73 (m, 1H), 4.70-4.50 (m, 1H), 3.70-3.40 |
| | | (m, 2H), 2.16-1.68 (m, 4H), 2.00, 1.31 (2s, 3H, |
| | | rotamers) |
| BCD-BTK-270 | 458.20 | ¹H NMR (400 MHz, DMSO, mixture of amide |
| | | rotamers 50/50): δ = 9.41, 9.39 (2s, 1H, rotamers), |
| | | 8.61-8.55 (m, 2H), 8.53 (d, J = 2.8 Hz, 1H), 8.18-8.12 |
| | | (m, 2H), 7.44-7.38 (m, 2H), 7.19-7.11 (m, 2H), |
| | | 6.11-6.00 (m, 1H), 4.26-4.11 (m, 1H), 4.02-3.81 |
| | | (m, 2H), 3.74-3.52 (m, 1H), 2.65-2.50 (m, 2H), |
| | | 2.03, 1.97 (2s, 3H, rotamers) |
| BCD-BTK-271 | 485.20 | ¹H NMR (400 MHz, DMSO, mixture of amide |
| | | rotamers 50/50): δ = 10.91, (s, 1H), 9.47, 9.45 |
| | | (2s, 1H, rotamers), 8.55, 8.54 (2s, 1H, rotamers), |
| | | 8.43-8.40 (m, 1H), 8.25-8.14 (m, 5H), 7.90-7.84 |
| | | (m, 1H), 7.24-7.16 (m, 1H), 6.15-6.01 (m, 1H), |
| | | 4.29-4.11 (m, 1H), 4.07-3.80 (m, 2H), 3.75-3.51 |
| | | (m, 1H), 2.63-2.53 (m, 2H), 2.05, 1.98 (2s, 3H, |
| | | rotamers) |
| BCD-BTK-272 | 446.20 | δ = 9.39, 9.38 (2s, 1H, rotamers), 8.56-8.49 (m, 3H), |
| | | 8.15-8.10 (m, 2H), 7.41-7.26 (m, 2H), 7.10-7.01 |
| | | (m, 2H), 6.75-6.45 (m, 1H), 6.23-6.11 (m, 1H), |
| | | 6.12-5.97 (m, 1H), 5.75-5.59 (m, 1H), 4.19-4.13 |
| | | (m, 1H), 4.05-3.82 (m, 2H), 3.79-3.59 (m, 1H), |
| | | 2.65-2.56 (m, 2H) |
| BCD-BTK-273 | 473.20 | δ = 10.91 (s, 1H), 9.46 (d, J = 2.3 Hz, 1H), 8.55 (s, |
| | | 1H), 8.43-8.39 (m, 1H), 8.26-8.11 (m, 5H), |
| | | 7.91-7.82 (m, 1H), 7.23-7.16 (m, 1H), 6.75-6.57 |
| | | (m, 1H), 6.23-6.14 (m, 1H), 6.13-5.99 (m, 1H), |
| | | 5.75-5.63 (m, 1H), 4.26-4.12 (m, 1H), 4.08-3.82 |
| | | (m, 2H), 3.82-3.59 (m, 1H), 2.71-2.52 (m, 2H) |
| BCD-BTK-274 | 487.20 | δ = 9.28 (d, J = 2.2 Hz, 1H), 8.88 (d, J = 1.7 Hz, |
| | | 1H), 8.62 (d, J = 2.7 Hz, 1H), 8.46 (d, J = 3.7 Hz, |
| | | 1H), 8.21 (s, 1H), 8.12 (d, J = 8.4 Hz, 2H), 7.88 |
| | | (dd, J = 2.8, 1.8 Hz, 1H), 7.68 (s, 1H), 7.32-7.24 |
| | | (m, 2H), 6.95-6.73 (m, 1H), 6.18-6.02 (m, 1H), |
| | | 5.71, 5.59 (2d, J = 10.4 Hz, 1H, rotamers), 4.76 |
| | | (s, 1H), 4.63, 4.22 (2d, J = 12.8 Hz, 1H, rotamers), |
| | | 4.40, 4.04 (2d, J = 13.8 Hz, 1H, rotamers), 3.77-3.35 |
| | | (m, 1H), 3.27-2.99 (m, 1H), 2.41-2.17 (m, 2H), |
| | | 1.97 (s, 1H), 1.62 (s, 1H) |
| BCD-BTK-275 | 473.20 | δ = 10.91 (s, 1H), 9.46 (d, J = 2.4 Hz, 1H), 8.55 (s, |
| | | 1H), 8.41 (dd, J = 4.9, 1.8 Hz, 1H), 8.24-8.12 (m, |
| | | 5H), 7.91-7.83 (m, 1H), 7.23-7.15 (m, 1H), |
| | | 6.74-6.55 (m, 1H), 6.22-6.14 (m, 1H), 6.13-5.99 |
| | | (m, 1H), 5.77-5.63 (m, 1H), 4.29-4.11 (m, 1H), |
| | | 4.07-3.83 (m, 2H), 3.81-3.60 (m, 1H), 2.69-2.53 |
| | | (m, 2H) |
| BCD-BTK-276 | 473.20 | δ = 10.92 (s, 1H), 9.45 (s, 1H), 8.54 (s, 1H), |
| | | 8.45-8.39 (m, 1H), 8.25-8.16 (m, 5H), 7.92-7.81 |
| | | (m, 1H), 7.24-7.14 (m, 1H), 6.39-6.24 (m, 1H), |
| | | 6.13-6.04 (m, 1H), 5.68-5.62 (m, 1H), 5.05 (d, |
| | | J = 7.3 Hz, 2H), 4.39-4.31 (m, 1H), 4.23-4.16 |
| | | (m, 1H), 4.10-4.00 (m, 1H), 3.97-3.83 (m, 1H), |
| | | 3.38-3.25 (m, 1H) |
| BCD-BTK-277 | 507.10 | δ = 10.70 (s, 1H), 9.67 (s, 2H), 9.57 (s, 1H), 8.59 (s, |
| | | 1H), 8.20 (dd, J = 7.9, 2.2 Hz, 1H), 8.12 (q, J = 8.1 |
| | | 1H), 7.01 (dd, J = 7.9, 2.2 Hz, 1H), 6.95-6.75 |
| | | (m, 1H), 6.19-6.03 (m, 1H), 5.72, 5.59 (2d, J = 10.4 |
| | | Hz, 1H, rotamers), 5.42-5.24 (m, 1H), 4.66, 4.20 |
| | | (2d, J = 12.6 Hz, 1H,rotamers), 4.41, 4.04 (2d, |
| | | J = 13.8 Hz, 1H, rotamers), 3.89-3.48 (m, 1H), |
| | | 3.33-3.09 (m, 1H), 2.41-2.29 (m, 2H), 1.99 |
| | | (s, 1H), 1.65 (s, 1H) |
| BCD-BTK-278 | 513.30 | ¹H NMR (400 MHz, DMSO, mixture of amide |
| | | rotamers 50/50): δ = 10.93, 10.92 (2s, 1H, rotamers), |
| | | 9.48, 9.45 (2s, 1H, rotamers), 8.56, 8.53 (2s, 1H, |
| | | rotamers), 8.44-8.37 (m, 1H), 8.29-8.16 (m, 5H), |
| | | 7.90-7.83 (m, 1H), 7.26-7.12 (m, 1H), 4.83-4.66 |
| | | (m, 2H), 4.15-3.90 (m, 2H), 3.25-3.10 (m, 1H), |
| | | 2.95-2.73 (m, 1H), 2.26-2.03 (m, 1H), 2.00, 1.54 |
| | | (2s, 3H, rotamers), 1.87-1.62 (m, 2H), 1.52-1.26 |
| | | (m, 2H) |
| BCD-BTK-279 | 501.20 | δ = 10.95 (s, 1H), 9.46 (s, 1H), 8.54 (s, 1H), |
| | | 8.44-8.39 (m, 1H), 8.25-8.16 (m, 5H), 7.92-7.84 (m, |
| | | 1H), 7.23-7.17 (m, 1H), 6.82-6.65 (m, 1H), 6.05, |
| | | 6.00 (2d, J = 2.5 Hz, 1H, rotamers), 5.69-5.54 (m, |
| | | 1H), 4.86-4.63 (m, 2H), 4.26-3.85 (m, 2H), |
| | | 3.24-3.04 (m, 1H), 3.02-2.71 (m, 1H), 2.25-2.06 (m, |
| | | 1H), 1.81-1.60 (m, 2H), 1.50-1.27 (m, 2H) |
| BCD-BTK-280 | 471.10 | δ = 10.94 (s, 1H), 9.46 (s, 1H), 8.54 (s, 1H), |
| | | 8.46-8.37 (m, 1H), 8.27-8.17 (m, 5H), 7.96-7.79 (m, |
| | | 1H), 7.27-7.13 (m, 1H), 6.27 (p, J = 6.4 Hz, 1H), |
| | | 4.74 (d, J = 6.6 Hz, 2H), 4.52 (d, J = 6.5 Hz, 2H), |
| | | 2.03 (s, 3H) |
| BCD-BTK-281 | 444.0 | δ = 9.39 (s, 1H), 8.54-8.50 (m, 3H), 8.22-8.17 (m, |
| | | 2H), 7.42-7.35 (m, 2H), 7.08-7.01 (m, 2H), |
| | | 6.32-6.18 (m, 1H), 4.79-4.66 (m, 2H), 4.56-4.43 |
| | | (m, 2H), 2.02 (s, 3H) |
| BCD-BTK-282 | 432.20 | δ = 9.39 (s, 1H), 8.55-8.48 (m, 3H), 8.23-8.16 (m, |
| | | 2H), 7.42-7.35 (m, 2H), 7.08-7.01 (m, 2H), |
| | | 6.46-6.35 (m, 1H), 6.31-6.22 (m, 1H), 6.21-6.12 (m, |
| | | 1H), 5.77-5.68 (m, 1H), 4.88-4.76 (m, 2H), |
| | | 4.58-4.50 (m, 2H) |
| BCD-BTK-283 | 513.20 | ¹H NMR (400 MHz, DMSO, mixture of amide |
| | | rotamers 50/50): δ = 10.93, 10.92 (2s, 1H, rotamers), |
| | | 9.48, 9.45 (2s, 1H, rotamers), 8.56, 8.53 (2s, 1H, |
| | | rotamers), 8.43-8.39 (m, 1H), 8.25-8.17 (m, 5H), |
| | | 7.90-7.84 (m, 1H), 7.21-7.17 (m, 1H), 4.78-4.66 |
| | | (m, 2H), 4.13-3.92 (m, 2H), 3.25-3.10 (m, 1H), |
| | | 2.90-2.75 (m, 1H), 2.23-2.06 (m, 1H), 2.00, 1.54 |
| | | (2s, 3H, rotamers), 1.86-1.65 (m, 2H), 1.52-1.27 |
| | | (m, 2H) |
| BCD-BTK-284 | 487.20 | δ = 10.92 (s, 1H), 9.45 (s, 1H), 8.53 (s, 1H), 8.42 (dd, |
| | | J = 4.9, 1.9 Hz, 1H), 8.29-8.12 (m, 5H), 7.93-7.81 |
| | | (m, 1H), 7.19 (dd, J = 7.3, 4.9 Hz, 1H), 6.63-6.45 |
| | | (m, 1H), 6.19-6.06 (m, 1H), 5.70-5.61 (m, 1H), |
| | | 4.90-4.79 (m, 2H), 3.78-3.66 (m, 1H), 3.62-3.43 |
| | | (m, 2H), 3.41-3.34 (m, 1H), 3.30-3.20 (m, 1H), |
| | | 3.05-2.80 (m, 1H), 2.09-1.93 (m, 1H), 1.91-1.72 |
| | | (m, 1H) |
| BCD-BTK-285 | 487.20 | δ = 10.92 (s, 1H), 9.45 (s, 1H), 8.54 (s, 1H), |
| | | 8.45-8.39 (m, 1H), 8.27-8.13 (m, 5H), 7.92-7.82 (m, |
| | | 1H), 7.25-7.11 (m, 1H), 6.62-6.45 (m, 1H), 6.19-6.06 |
| | | (m, 1H), 5.69-5.59 (m, 1H), 4.93-4.79 (m, 2H), |
| | | 3.78-3.66 (m, 1H), 3.63-3.43 (m, 2H), 3.43-3.32 |
| | | (m, 1H), 3.28-3.25 (m, 1H), 3.03-2.78 (m, 1H), |
| | | 2.14-1.70 (m, 2H) |
| BCD-BTK-286 | 499.20 | ¹H NMR (400 MHz, DMSO, mixture of amide |
| | | rotamers 50/50): δ = 10.93, 10.92 (2s, 1H, rotamers), |
| | | 8.55, 8.54 (2s, 1H, rotamers), 8.42, 8.41 (2s, 1H, |
| | | rotamers), 8.26-8.13 (m, 5H), 7.91-7.83 (m, 1H), |
| | | 7.19 (dd, J = 7.3, 4.8 Hz, 1H), 4.92-4.75 (m, 2H), |
| | | 3.80-3.63 (m, 1H), 3.59-3.41 (m, 2H), 3.31-3.17 |
| | | (m, 1H), 2.99-2.82 (m, 1H), 2.03-1.92 (m, 1H), |
| | | 2.00, 1.94 (2s, 3H, rotamers), 1.88-1.75 (m, 1H) |
| BCD-BTK-287 | 499.30 | ¹H NMR (400 MHz, DMSO, mixture of amide |
| | | rotamers 50/50): δ = 10.92 (2s, 1H, rotamers), 9.46 |
| | | 9.45 (2s, 1H, rotamers), 8.54, 8.53 (2s, 1H, rotamers), |
| | | 8.42 (dd, J = 5.1, 1.8 Hz, 1H), 8.27-8.15 (m, 5H), |
| | | 7.93-7.82 (m, 1H), 7.23-7.14 (m, 1H), 4.94-4.75 |
| | | (m, 2H), 3.85-3.64 (m, 1H), 3.60-3.41 (m, 2H), |
| | | 3.43-3.33 (m, 1H), 3.27-3.17 (m, 1H), 2.96-2.85 |
| | | (m, 1H), 2.05-1.96 (m, 1H), 2.00, 1.94 (s, 3H, |
| | | rotamers), 1.89-1.76 (m, 1H) |
| BCD-BTK-288 | 507.20 | δ = 11.01 (s, 1H), 9.64 (s, 2H), 9.56 (s, 1H), 8.59 (s, |
| | | 1H), 8.34 (s, 1H), 7.93-7.82 (m, 1H), 7.51-7.41 (m, |
| | | 1H), 6.97-6.71 (m, 1H), 6.18-6.02 (m, 1H), 5.78-5.54 |
| | | 5.54 (m, 1H), 5.42-5.26 (m, 1H), 4.66, 4.19 (2s, 1H, |
| | | rotamers) 4.41, 4.04 (2s, 1H, rotamers), 3.88-3.44 |
| | | (m, 1H), 3.33-3.09 (m, 1H), 2.34 (s, 2H), 1.99 (s, |
| | | 1H), 1.65 (s, 1H) |
| BCD-BTK-289 | 499.20 | ¹H NMR (400 MHz, DMSO, mixture of amide |
| | | rotamers 50/50): δ = 10.93 (s, 1H), 9.47, 9.45 (2 s, |
| | | 1H, rotamers), 8.56, 8.53 (2 s, 1H, rotamers), 8.44-8.39 |
| | | (m, 1H), 8.25-8.14 (m, 5H), 7.91-7.84 (m, 1H), |
| | | 7.24-7.16 (m, 1H), 5.48-5.32, 5.30-5.20 (2 m, 1H, |
| | | rotamers), 4.60-4.43 (m, 1H), 4.26-3.97 (m, 1H), |
| | | 3.95-3.84, 3.54-3.47 (2 m, 1H, rotamers), 3.33-3.15 |
| | | (m, 1H), 2.47-2.25 (m, 1H), 2.11-1.98 (m, 1H), |
| | | 2.07, 1.79 (2 s, 3H, rotamers), 1.72-1.55 (m, 1H) |
| BCD-BTK-290 | 501.20 | δ = 10.92 (s, 1H), 9.46 (s, 1H), 8.53 (s, 1H), 8.43-8.40 |
| | | (m, 1H), 8.25-8.16 (m, 5H), 7.90-7.84 (m, 1H), |
| | | 7.21-7.17 (m, 1H), 6.82-6.66 (m, 1H), 6.05, |
| | | 6.00 (2 d, J = 2.5 Hz, 1H, rotamers), 5.66-5.54 (m, |
| | | 1H), 4.82-4.62 (m, 2H), 4.25-3.87 (m, 2H), 3.24-2.72 |
| | | (m, 2H), 2.27-2.07 (m, 1H), 1.77-1.64 (m, 2H), |
| | | 1.38 (s, 2H) |
| BCD-BTK-291 | 557.20 | δ = 10.96 (s, 1H), 9.69 (s, 2H), 9.58 (s, 1H), 8.73 (d, |
| | | J = 5.1 Hz, 1H), 8.59 (d, J = 2.1 Hz, 2H), 7.63 (dd, |
| | | J = 5.2, 1.6 Hz, 1H), 6.94-6.76 (m, 1H), 6.25-6.02 |
| | | (m, 1H), 5.72, 5.59 (2d, J = 10.5 Hz, 1H, rotamers), |
| | | 5.45-5.20 (m, 1H), 4.66, 4.21 (2d, J = 12.7 Hz, 1H, |
| | | rotamers), 4.41, 4.04 (2d, J = 13.8 Hz, 1H, rotamers), |
| | | 3.91-3.49 (m, 1H), 3.33-3.09 (m, 1H), 2.42-2.28 |
| | | (m, 2H), 2.00 (s, 1H), 1.65 (s, 1H) |
| BCD-BTK-292 | 581.30 | δ = 10.77 (s, 1H), 9.68 (s, 2H), 9.58 (s, 1H), 8.59-8.54 |
| | | (m, 2H), 8.43 (s, 1H), 7.39 (dd, J = 5.2, 1.5 Hz, |
| | | 1H), 7.02-6.63 (m, 1H), 6.22-5.99 (m, 1H), 5.72, |
| | | 5.59 (2 d, J = 10.4 Hz, 1H, rotamers), 5.43-5.22 (m, |
| | | 1H), 4.66, 4.21 (2 d, J = 12.6 Hz, 1H, rotamers), 4.42, |
| | | 4.04 (2 d, J = 13.7 Hz, 1H, rotamers), 3.91-3.48 (m, |
| | | 1H), 3.33-3.03 (m, 1H), 2.44-2.30 (m, 2H), 2.29-2.14 |
| | | (m, 2H), 1.99 (s, 1H), 1.65 (s, 1H), 1.49-1.34 |
| | | (m, 2H), 0.94 (t, J = 7.4 Hz, 3H) |
| BCD-BTK-293 | 567.30 | δ = 10.78 (s, 1H), 9.69 (s, 2H), 9.58 (s, 1H), 8.61-8.55 |
| | | (m, 2H), 8.43 (s, 1H), 7.39 (dd, J = 5.2, 1.6 Hz, |
| | | 1H), 6.97-6.70 (m, 1H), 6.23-6.01 (m, 1H), 5.72, |
| | | 5.59 (2 d, J = 10.4 Hz, 1H, rotamers), 5.45-5.25 (m, |
| | | 1H), 4.66, 4.21 (2 d, J = 12.6 Hz, 1H, rotamers), 4.42, |
| | | 4.04 (2 d, J = 13.9 Hz, 1H, rotamers), 3.89-3.47 (m, |
| | | 1H), 3.33-3.08 (m, 1H), 2.39-2.19 (m, 4H), 1.99 (s, |
| | | 1H), 1.65 (s, 1H), 0.97 (t, J = 7.5 Hz, 3H) |
| BCD-BTK-295 | 514.30 | ¹H NMR (400 MHz, DMSO, mixture of amide |
| | | rotamers 70/80): δ = 10.88 (s, 1H), 8.45-8.37 (m, |
| | | 1H), 8.27-8.16 (m, 3H), 7.93-7.84 (m, 1H), 7.84, |
| | | 7.82 (2 s, 1H, rotamers), 7.78 (dd, J = 8.4, 1.7 Hz, 2H), |
| | | 7.23-7.16 (m, 1H), 6.04, 6.05 (2 d, 2H, rotamers), |
| | | 5.29, 5.15 (2tt, J = 9.0, 4.4 Hz, 1H, rotamers), 4.54, |
| | | 4.45 (2dd, J = 13.0, 4.1 Hz, 1H, rotamers), 4.21, 4.03 |
| | | (2 d, J = 13.0 Hz, 1H, rotamers), 3.78, 3.35 (2dd, J = |
| | | 12.7, 10.1 Hz, 1H), 3.33-3.06 (m, 1H), 2.41- 2.19 |
| | | (m, 2H), 2.06, 1.84 (2 s, 3H, rotamers), 2.04-1.94 (m, |
| | | 1H), 1.70-1.46 (m, 1H) |

### Example 9. Metabolic Stability Assay

Assessment of the metabolic stability of candidate compounds allows to evaluate the resistance of the compounds to the action of biotransformation enzymes. To assess the metabolic stability of the drug candidates, we used pooled human liver S9 fraction.

1) Metabolic Stability in Human Liver S9 Fractions

The rate of enzymatic decomposition of a compound was detected by incubating the reaction mixture in a dry block heater at 37° C.; the reaction mixture contained 0.5 mg/mL of pooled human liver S9 fraction (XenoTech, USA, cat#H0610), 10 mM of a drug candidate, 2 mM β-nicotinamide adenine dinucleotide (Carbosynth, UK, cat#NN10871) and 4 mM of magnesium chloride in 0.1M sodium phosphate buffer pH=7.4. The reaction was terminated by adding 100 μL of acetonitrile for each 100 L of the reaction mixture. After the reaction was terminated, the samples were centrifuged for 10 min at 10000 rpm. The supernatant was chromatographed using Agilent 1200 chromatograph (Agilent, USA). We used gradient elution (1 mL/min flow rate). We plotted a calibration curve of the logarithm of the peak area vs. time. The gradient of the line corresponded to the elimination rate constant k. Based on the constant, determined using the curve, we calculated the drug's half-life (T_{1/2}) and metabolism rate (CLᵢₙₜ).

$$Elimination rate constant ( k ) = ( - gradient )$$

$$Half life ( {t}_{1 / 2} ) ( min ) = \frac{0.693}{k}$$

$$V ( µL \text{/} mg ) = \frac{volume of incubation ( µL )}{protein in the incubation ( mg )}$$

$$Intrinsic Clearance ( {CL}_{int} ) ( µL \text{/} min \text{/} mg protein ) = \frac{V \times 0.693}{{t}_{1 / 2}}$$

The results characterized S9 stability of the drug candidates. The compounds demonstrate sufficient S9 stability. The results are provided in Table 2 and Table 3.

2) Metabolic Stability in Human Liver Microsomes

The rate of enzymatic decomposition of a compound was detected by incubating the reaction mixture in a dry block heater at 37° C.; the reaction mixture contained 0.5 mg/mL of pooled human liver microsomes (XenoTech, USA, cat# H6010), 10 mM of a drug candidate, 2 mM β-nicotinamide adenine dinucleotide (Carbosynth, UK, cat#NN10871) and 4 mM of magnesium chloride in 0.1M sodium phosphate buffer pH=7.4. The reaction was terminated by adding 100 μL of acetonitrile for each 100 μL of the reaction mixture. After the reaction was terminated, the samples were centrifuged for 10 min at 10000 rpm. The supernatant was chromatographed using Agilent 1200 chromatograph (Agilent, USA). We used gradient elution (1 mL/min flow rate). We plotted a calibration curve of the logarithm of the peak area vs. time. The gradient of the line corresponded to the elimination rate constant k. Based on the constant, determined using the curve, we calculated the drug's half-life (T_{1/2}) and metabolism rate (CLᵢₙₜ).

$$Elimination rate constant ( k ) = ( - gradient )$$

$$Half life ( {t}_{1 / 2} ) ( min ) = \frac{0.693}{k}$$

$$V ( µL \text{/} mg ) = \frac{volume of incubation ( µL )}{protein in the incubation ( mg )}$$

$$Intrinsic Clearance ( {CL}_{int} ) ( µL \text{/} min \text{/} mg protein ) = \frac{V \times 0.693}{{t}_{1 / 2}}$$

The results characterized microsomal stability of the drug candidates. The compounds demonstrate sufficient microsomal stability and their rate of enzymatic decomposition Clᵢₙₜ is less than 47 μL/min/mg. The results are provided in Table 2 and Table 4.

TABLE 2
Results of metabolic stability.

| | Stability | |
|---|---|---|
| Candidate No. | S9, Clᵢₙₜ μL/min/mg | Microsomes, Clᵢₙₜ μL/min/mg |
| Ibrutinib | 15.8 | 111.8 |
| BCD-BTK-123 | 6.64 | 51.0 |
| BCD-BTκ-125 | 3.12 | 22.8 |
| BCD-BTK-134 | 7.56 | 32.2 |
| BCD-BTK-139 | 11.04 | 31.6 |

TABLE 3
Results of S9 metabolic stability.

| Candidate No. | Clᵢₙₜ μL/min/mg |
|---|---|
| BCD-BTK-13 | 3.32 |
| BCD-BTK-30 | 3.48 |
| BCD-BTK-56 | 1.7 |
| BCD-BTK-74 | 2.6 |
| BCD-BTK-76 | 1.48 |
| BCD-BTK-104 | 1.92 |
| BCD-BTK-117 | 1.4 |
| BCD-BTK-118 | 3.32 |
| BCD-BTK-204 | 1.98 |
| BCD-BTK-201 | 5.44 |
| BCD-BTK-205 | 1.76 |
| BCD-BTK-202 | 6.8 |
| BCD-BTK-203 | 2.96 |
| BCD-BTK-206 | 1.64 |
| BCD-BTK-207 | 1.16 |
| BCD-BTK-208 | 3.16 |
| BCD-BTK-215 | 9.3 |
| BCD-BTK-218 | 7.6 |
| BCD-BTK-220 | 4.8 |
| BCD-BTK-228 | 7.9 |
| BCD-BTK-229 | 5.4 |
| BCD-BTκ-124 | 4.0 |
| BCD-BTκ-128 | 6.68 |
| BCD-BTκ-129 | 1.44 |
| BCD-BTκ-131 | 1.16 |
| BCD-BTK-132 | 13.0 |
| BCD-BTK-137 | 3.32 |
| BCD-BTK-140 | 2.28 |
| BCD-BTK-119 | 0.92 |
| BCD-BTK-230 | 10.5 |
| BCD-BTK-235 | 8.1 |
| BCD-BTk-232 | 14 |
| BCD-BTK-233 | 3.1 |
| BCD-BTK-236 | 12.4 |
| BCD-BTK-238 | 13 |
| BCD-BTK-240 | 4.8 |
| BCD-BTK-242 | 14.1 |
| BCD-BTK-243 | 12 |
| BCD-BTK-244 | 9.6 |
| BCD-BTK-258 | 11.2 |
| BCD-BTK-269 | 10 |
| BCD-BTk-292 | 1.9 |

TABLE 4
Results of microsome metabolic stability.

| Candidate No. | Clᵢₙₜ μL/min/mg |
|---|---|
| BCD-BTK-204 | 30.4 |
| BCD-BTK-205 | 31.1 |
| BCD-BTK-206 | 24.1 |
| BCD-BTK-207 | 33.1 |
| BCD-BTK-210 | 3.0 |
| BCD-BTK-212 | 11.2 |
| BCD-BTK-215 | 24.2 |
| BCD-BTK-223 | 26 |
| BCD-BTK-227 | 18.4 |
| BCD-BTK-228 | 10 |
| BCD-BTK-229 | 3.5 |
| BCD-BTK-233 | 36 |
| BCD-BTK-239 | 27.3 |
| BCD-BTK-240 | 24.5 |
| BCD-BTK-242 | 12.4 |
| BCD-BTK-243 | 21.4 |
| BCD-BTK-244 | 39.2 |
| BCD-BTK-245 | 32.8 |
| BCD-BTK -241 | 27.4 |
| BCD-BTK -247 | 42.2 |
| BCD-BTK -252 | 11.2 |
| BCD-BTK-258 | 39 |
| BCD-BTK-261 | 41 |
| BCD-BTK-262 | 16.8 |
| BCD-BTK-264 | 18.2 |
| BCD-BTK-265 | 37.6 |
| BCD-BTK-266 | 37.6 |
| BCD-BTK-268 | 7.4 |
| BCD-BTK-269 | 41.8 |
| BCD-BTK-272 | 28.6 |
| BCD-BTK-273 | 16 |
| BCD-BTK -292 | 12.2 |

### Example 10. Stability in Human Blood Plasma

Assessment of the stability of candidate compounds in human blood plasma allows to evaluate the resistance of the compounds to the action of blood plasma enzymes, for example, esterases.

To assess the blood plasma stability of the drug candidates, we used pooled human blood plasma taken from ten healthy donors. The initial solution (10 Mm in DMSO) was diluted with pooled blood plasma to 10 μM (test solution). The test solution had been incubated for 4 hours in a dry block heater at 37° C. We determined peak areas of the compounds corresponding to the start of the test (before incubating) and the end of the test (after incubating in a dry block heater at 37° C.) by HPLC using Agilent1200 chromatograph (Agilent, USA) with preliminary protein precipitation with acetonitrile. We used gradient elution (1 mL/min flow rate). We determined amount of substance in the sample in % after incubation.

We evaluated the stability of the compounds. Compounds described herein have the stability level in plasma more than 80%. Enzymes of blood plasma potentially will not reduce the concentration of compounds in the bloodstream and thus will not influence the effectiveness in vivo. The results are presented in table 5.

TABLE 5
Results of stability in human blood plasma.

| Candidate No. | Amount of substance in the sample, % |
|---|---|
| BCD-BTK-204 | 91.9 |
| BCD-BTK-205 | 88.6 |
| BCD-BTK-203 | 84.7 |
| BCD-BTK-206 | 87.6 |
| BCD-BTK-207 | 82.6 |
| BCD-BTK-208 | 91.4 |
| BCD-BTK-211 | 94.8 |
| BCD-BTK-212 | 90.8 |
| BCD-BTK-216 | 80.4 |
| BCD-BTK-217 | 90.8 |
| BCD-BTK-218 | 92.0 |
| BCD-BTK-220 | 90.2 |
| BCD-BTK-221 | 80.5 |
| BCD-BTK-222 | 100.0 |
| BCD-BTK-226 | 84.5 |
| BCD-BTK-228 | 85.2 |
| BCD-BTK-229 | 83.5 |
| BCD-BTK-230 | 93.3 |
| BCD-BTK-231 | 93.1 |
| BCD-BTK-234 | 83.5 |
| BCD-BTk-232 | 88.6 |
| BCD-BTK-236 | 96.8 |
| BCD-BTK-239 | 80.3 |
| BCD-BTK-240 | 100.0 |
| BCD-BTK-242 | 100.0 |
| BCD-BTK -237 | 95.5 |
| BCD-BTK -241 | 80.6 |
| BCD-BTK -247 | 87.3 |
| BCD-BTK -250 | 84.5 |
| BCD-BTK -251 | 89.2 |
| BCD-BTK -252 | 85.2 |
| BCD-BTK -254 | 93.0 |
| BCD-BTK-259 | 90.7 |
| BCD-BTK-260 | 83.2 |
| BCD-BTK-261 | 100.0 |
| BCD-BTK-263 | 86.7 |
| BCD-BTK-265 | 81.7 |
| BCD-BTK-266 | 87.2 |
| BCD-BTK-268 | 91.0 |
| BCD-BTK-269 | 95.4 |
| BCD-BTK-267 | 100.0 |
| BCD-BTK-274 | 91.3 |
| BCD-BTK-278 | 96.2 |
| BCD-BTK-283 | 97.4 |
| BCD-BTK-289 | 100.0 |
| BCD-BTK-290 | 100.0 |
| BCD-BTK -292 | 93.3 |
| BCD-BTK -293 | 85.7 |
| BCD-BTK -295 | 100.0 |
| BCD-BTK -125 | 97.8 |
| BCD-BTK -134 | 89.7 |
| BCD-BTK -139 | 87.1 |

### Example 11. Chemical Stability

Assessment of chemical stability of the compounds allows to assess their stability in gastric fluid.

A concentrate of SGF without enzymes, pH=1.4 (Sigma Ireland, cat#01651) was used as artificial gastric fluid. The initial candidate solution (10 mM in DMSO) was diluted with the working solution of SGF to the concentration of 10 μm (test solution). The test solution was incubated in a dry block heater at 37° C. We determined peak areas of the compounds corresponding to the start of the test (before incubating) and the end of the test (after incubating in a dry block heater at 37° C.) by HPLC using Agilent1200 chromatograph (Agilent, USA). We used gradient elution (1 mL/min flow rate). We determined amount of substance in the sample in % after incubation.

We evaluated the stability of the compounds. Compounds described herein are chemically stable in acidic environment of artificial gastric fluid. The results are presented in table 6.

TABLE 6
The results of chemical stability.

| Candidate No. | Amount of substance in the sample in % |
|---|---|
| BCD-BTK-9 | 100.0 |
| BCD-BTK-13 | 91.0 |
| BCD-BTK-30 | 97.7 |
| BCD-BTK-56 | 99.0 |
| BCD-BTK-74 | 98.7 |
| BCD-BTK-76 | 99.7 |
| BCD-BTK-104 | 99.4 |
| BCD-BTK-117 | 99.7 |
| BCD-BTK-118 | 95.4 |
| BCD-BTK-119 | 100.0 |
| BCD-BTK -204 | 100 |
| BCD-BTK -201 | 100 |
| BCD-BTK -205 | 100 |
| BCD-BTK -202 | 95.5 |
| BCD-BTK -203 | 100 |
| BCD-BTK -206 | 100 |
| BCD-BTK -207 | 100 |
| BCD-BTK -208 | 100 |
| BCD-BTK -210 | 96.1 |
| BCD-BTK -211 | 100 |
| BCD-BTK -212 | 100 |
| BCD-BTK -213 | 97.4 |
| BCD-BTK -214 | 100 |
| BCD-BTK -215 | 100 |
| BCD-BTK -216 | 96.3 |
| BCD-BTK -217 | 95.3 |
| BCD-BTK -218 | 96 |
| BCD-BTK-219 | 96.7 |
| BCD-BTK-220 | 100 |
| BCD-BTK-221 | 91.3 |
| BCD-BTK-222 | 100 |
| BCD-BTK-223 | 96.7 |
| BCD-BTK-224 | 100 |
| BCD-BTK-225 | 100 |
| BCD-BTK-226 | 100 |
| BCD-BTK-227 | 100 |
| BCD-BTK-228 | 99.9 |
| BCD-BTK-229 | 99.8 |
| BCD-BTK-230 | 99.9 |
| BCD-BTK-231 | 100 |
| BCD-BTK-234 | 100 |
| BCD-BTK-235 | 100 |
| BCD-BTk-232 | 95.9 |
| BCD-BTK-233 | 100 |
| BCD-BTK-236 | 100 |
| BCD-BTK-238 | 100 |
| BCD-BTK-239 | 100 |
| BCD-BTK-240 | 100 |
| BCD-BTK-242 | 100 |
| BCD-BTK-243 | 99.6 |
| BCD-BTK-244 | 100 |
| BCD-BTK-245 | 100 |
| BCD-BTK-246 | 100 |
| BCD-BTK -237 | 100 |
| BCD-BTK -241 | 100 |
| BCD-BTK -247 | 100 |
| BCD-BTK-122 | 97.5 |
| BCD-BTκ-124 | 95.8 |
| BCD-BTκ-128 | 100.0 |
| BCD-BTκ-129 | 97.8 |
| BCD-BTκ-131 | 97.7 |
| BCD-BTK-132 | 97.6 |
| BCD-BTκ-133 | 100.0 |
| BCD-BTK-136 | 89.9 |
| BCD-BTK-137 | 94.3 |
| BCD-BTK-140 | 99.4 |
| BCD-BTK - 248 | 100 |
| BCD-BTK - 249 | 100 |
| BCD-BTK - 250 | 100 |
| BCD-BTK - 251 | 100 |
| BCD-BTK - 252 | 94.4 |
| BCD-BTK - 254 | 100 |
| BCD-BTK - 255 | 100 |
| BCD-BTK-258 | 100 |
| BCD-BTK-259 | 100 |
| BCD-BTK-260 | 100 |
| BCD-BTK-261 | 100 |
| BCD-BTK-262 | 100 |
| BCD-BTK-263 | 100 |
| BCD-BTK-264 | 97.8 |
| BCD-BTK-265 | 94.8 |
| BCD-BTK-266 | 100 |
| BCD-BTK-268 | 99.3 |
| BCD-BTK-269 | 100 |
| BCD-BTK-270 | 99.1 |
| BCD-BTK-271 | 100 |
| BCD-BTK-267 | 100 |
| BCD-BTK-272 | 100 |
| BCD-BTK-273 | 100 |
| BCD-BTK-274 | 100 |
| BCD-BTK-275 | 100 |
| BCD-BTK-276 | 100 |
| BCD-BTK-277 | 99.8 |
| BCD-BTK-278 | 100 |
| BCD-BTK-279 | 100 |
| BCD-BTK-283 | 100 |
| BCD-BTK-284 | 100 |
| BCD-BTK-285 | 100 |
| BCD-BTK-289 | 100 |
| BCD-BTK-290 | 100 |
| BCD-BTK - 281 | 100 |
| BCD-BTK - 282 | 100 |
| BCD-BTK - 286 | 100 |
| BCD-BTK - 287 | 100 |
| BCD-BTK - 292 | 100 |
| BCD-BTK - 293 | 98.4 |
| BCD-BTK - 294 | 100 |
| BCD-BTK - 295 | 89.5 |
| BCD-BTK - 123 | 97 |
| BCD-BTK - 125 | 100 |
| BCD-BTK - 134 | 100 |
| BCD-BTK - 139 | 100 |

### Example 12. Permeability Through the Monolayer of Caco-2 Cells

Assessment of permeability through the monolayer of Caco-2 cells allows to evaluate the ability of the candidate compounds to penetrate through biological membranes by active and passive transport.

Caco-2, the cells of the intestinal epithelium, had been cultured in transwell plate inserts with the filters (with pores of 0.4 μm, BD Falcon with High Density, #353495) for 21 days, and then the integrity of the monolayer were estimated with Lucifer Yellow dye (Sigma-Aldrich, USA) by standard protocol. When setting the A→B transfer, solutions of test substances were added in a buffer with pH 6.5 (Hanks solution, 10 mM HEPES, 15 mM glucose solution) with the concentration of 10 μM into the upper chamber; the lower chamber was filled with a buffer with pH 7.4 (Hanks solution, 10 mM HEPES, 15 mM glucose solution, 1% BSA). When setting B→A transfer, the upper chamber was filled with the buffer with pH 6.5, and solutions of the test substances were added in the buffer with pH 7.4 at the concentration of 10 μM in the lower chamber. Propranolol was used as a control substance (as it has high permeability).

After incubating for 2 h at 37° C. under 5% CO₂, the amounts of test compounds were determined in the upper and lower chambers by HPLC using Agilent1200 chromatograph (Agilent, USA) with preliminary protein precipitation with acetonitrile. We used gradient elution (1 mL/min flow rate). We determined the areas of peaks corresponding to the compounds. On the basis of peak areas in the calibration standards we determined the concentration of compound in the initial solution and in the samples from the wells of the upper and lower chambers.

Pₐₚₚ, permeability through the cell layer, was calculated using the following formula:

Pₐₚₚ=(C₍ₜ₎*V)/(C₍₀₎*t*Area), where

Pₐₚₚ is the effective constant of permeability, m/s
V is the volume of solution (0.8 ml in A→B test, 0.2 ml in B→A test), ml
Area is the surface area of the membrane (0.33 cm²), cm²
t is the time of incubation (7200 sec), sec
C₍₀₎ is the concentration of the initial solution, μM
C₍ₜ₎ is the concentration of the solution after 2 hours (the concentration in the sample from the well of the lower chamber in A→B test; the concentration in the sample from the well of the top chamber in in B→A test), μM

The efflux coefficient shows the ability of cells to eliminate the substance from the bloodstream. The value was calculated with the following formula:

efflux=P_{app B-A}/P_{app A-B}, where

P_{app A-B} is the value of the permeability in the direct test (A→B);
P_{app B-A} is the value of the permeability in the backward test in (B→A).

The compounds described herein show a high rate of the direct transport A→B Papp>5*10\hat{ }(−6)cm/s, (“the lumen of the intestine”—“bloodstream”), while the efflux coefficient does not exceed 2, which indicates that efflux (driven by such transporters as Pgp, BCRP) does not impose any restrictions on bioavailability of the compounds. The results are presented in table 7.

TABLE 7
Results of the assessment of permeability
through the monolayer of Caco-2 cells

| | Caco-2, Papp, 10\hat{ }(−6)cm/s | | |
|---|---|---|---|
| Candidate No. | A → B | B → A | efflux |
| BCD-BTK-123 | 14.8 | 7.4 | 0.5 |
| BCD-BTK-125 | 21.0 | 13.2 | 0.6 |
| BCD-BTK-134 | 25.8 | 12 | 0.4 |
| BCD-BTK-139 | 52.2 | 5.7 | 0.1 |
| BCD-BTK-202 | 20.3 | 1.91 | 0.09 |
| BCD-BTK-203 | 9.31 | 8.4 | 0.9 |
| BCD-BTK-210 | 11.88 | 0.21 | 0.02 |
| BCD-BTK-211 | 19.21 | 3.55 | 0.18 |
| BCD-BTK-213 | 8.39 | 5.17 | 0.62 |
| BCD-BTK-214 | 17.81 | 0.87 | 0.05 |
| BCD-BTK-216 | 29.64 | 8.72 | 0.29 |
| BCD-BTK-217 | 37.26 | 18.94 | 0.51 |
| BCD-BTK-218 | 20.2 | 17.38 | 0.86 |
| BCD-BTK-222 | 5.06 | 6.97 | 1.38 |
| BCD-BTK-223 | 5.9 | 0.3 | 0.05 |
| BCD-BTK-226 | 12.1 | 5.5 | 0.45 |
| BCD-BTK-228 | 8.0 | 1.4 | 0.17 |
| BCD-BTK-229 | 10.7 | 12.2 | 1.14 |
| BCD-BTK-230 | 26.5 | 2.68 | 0.1 |
| BCD-BTK-231 | 9.8 | 1.84 | 0.19 |
| BCD-BTK-234 | 8.2 | 9.2 | 1.1 |
| BCD-BTk-232 | 6.27 | 3.58 | 0.57 |
| BCD-BTK-236 | 6.51 | 2.87 | 0.44 |
| BCD-BTK-238 | 6.75 | 4.78 | 0.71 |
| BCD-BTK-240 | 18.2 | 11.1 | 0.61 |
| BCD-BTk-237 | 6.9 | 3.5 | 0.5 |
| BCD-BTk-251 | 8.3 | 1.5 | 0.2 |
| BCD-BTK-261 | 8.3 | 0.95 | 0.11 |
| BCD-BTK-266 | 9.5 | 2.2 | 0.23 |
| BCD-BTK-272 | 6.3 | 1.1 | 0.18 |
| BCD-BTK-292 | 11.6 | 0.07 | 0.006 |
| BCD-BTK-293 | 6.17 | 0.37 | 0.06 |

### Example 13. In Vitro Inhibition of Kinase Activity

To assess Btk kinase activity described herein, SignalChem kinase system was used. Btk kinase activity was determined in the reaction between recombinant Btk kinase enzyme (SignalChem #B10-10H) and Poly (4:1 Glu, Tyr) peptide substrate in the presence of the inhibitor.

The measurements were carried out in a 25 μL reaction volume using a 96-well plate (Corning, #3642). The kinase enzyme and inhibitor were pre-incubated for 10 minutes in the reaction buffer containing 25 mM of MOPS (pH 7.2), 12.5 mM of 0-glycerophosphate, 27 mM of MgCl₂, 2 mM of MnCl₂, 5 mM of EGTA, 2 mM of EDTA, 0.3 mM of DTT, and 1.2 mg/mL of bovine serum albumin. Staurosporine (Abcam Biochemicals, ab146588) was used as a reference inhibitor and 0.1% DMSO in the reaction buffer—as a negative control. The solution of 0.5 mg/mL peptide substrate and 50 &M ATP in the same buffer were added; the solution was incubated for 180 minutes at 37° C. To detect the amount of ATP taken up during the kinase reaction, the equivalent amount of ADP (from the ADP Glo Detection Kit (Promega, #V9101)) was used according to the protocol. The reaction mixture was equilibrated to room temperature. 25 L of ADP-Glo Reagent were added into each well; the plate was incubated for 40 minutes. 50 μL of Kinase Detection Reagent were added; the plate was incubated for 30 minutes. The luminescence was measured with multimode plate reader (Tecan Infinite M200Pro, Switzerland). IC₅₀ values were calculated using Magellan 7.2 software (Tecan, Switzerland) approximating experimental points by four-parameter logistic model with the optimization by Levenberg-Marquardt. The results are presented in the tables 8, 9 and 11.

For active compounds selected by screening using the target enzyme BTK, the values of IC₅₀ were determined on a kinase panel: EGFR (SignalChem, #E10-11G), ITK (SignalChem, #I13-10G) and TEC (SignalChem, #T03-10G). The results are presented in the table 9.

TABLE 8
Results of in vitro tests of inhibition of BTK kinase activity

| Candidate No. | IC₅₀ kinase BTK, nM |
|---|---|
| BCD-BTK-135 | 6.28 |
| BCD-BTK-128 | 4.94 |
| BCD-BTK-201 | 19.06 |
| BCD-BTK-202 | 74.56 |
| BCD-BTK-203 | 1103.50 |
| BCD-BTK-214 | 34.34 |
| BCD-BTK-215 | 35.78 |
| BCD-BTK-218 | 55.46 |
| BCD-BTK-220 | 53.55 |
| BCD-BTK-228 | 38.91 |
| BCD-BTK-229 | 43.95 |
| BCD-BTK-230 | 10.51 |
| BCD-BTK-234 | 1.17 |
| BCD-BTK-235 | 13.89 |
| BCD-BTK-233 | 11.47 |
| BCD-BTK-236 | 17.38 |
| BCD-BTK-238 | 18.89 |
| BCD-BTK-239 | 2.38 |
| BCD-BTK-240 | 2.25 |
| BCD-BTK-242 | 1.77 |
| BCD-BTK-243 | 29.31 |
| BCD-BTK-244 | 55.74 |
| BCD-BTK-237 | 12.65 |
| BCD-BTK-241 | 10.01 |
| BCD-BTK-247 | 14.70 |
| BCD-BTK-250 | 15.26 |
| BCD-BTK-251 | 2.71 |
| BCD-BTK-252 | 14.55 |
| BCD-BTK-254 | 11.24 |
| BCD-BTK-258 | 1.17 |
| BCD-BTK-260 | 17.65 |
| BCD-BTK-266 | 13.15 |
| BCD-BTK-272 | 54.75 |
| BCD-BTK-273 | 26.08 |
| BCD-BTK-290 | 19.60 |
| BCD-BTK-292 | 7.34 |
| BCD-BTK-295 | 65.21 |
| — | — |

TABLE 9
Results of in vitro tests on the kinase panel

| | IC₅₀ kinase | IC₅₀ kinase | IC₅₀ kinase | IC₅₀ kinase |
|---|---|---|---|---|
| Candidate No. | BTK, nM | EGFR, nM | ITK, nM | TEC, nM |
| BCD-BTκ-125 | 108.74 | >1000 | >1000 | — |
| BCD-BTK-134 | 1.16 | 127.1 | 300 | — |
| BCD-BTK-139 | 0.85 | 48.83 | 200 | — |
| BCD-BTK-201 | 19.06 | 172.41 | >300 | 55.22 |
| BCD-BTK-202 | 74.56 | >500 | >500 | >500 |
| BCD-BTK-220 | 53.55 | 820.3 | >300 | >100 |
| BCD-BTK-230 | 10.51 | >500 | >1000 | >200 |
| BCD-BTK-234 | 1.17 | 1.8 | 25.44 | 14.82 |
| BCD-BTK-239 | 2.38 | 10.48 | 24.95 | 6 |
| BCD-BTK-240 | 2.25 | 2.41 | 7.78 | 3.81 |
| BCD-BTK-241 | 10.01 | 25.23 | — | — |
| BCD-BTK-242 | 1.77 | 1.32 | 11.03 | 6.61 |
| BCD-BTK-251 | 2.71 | 8.83 | 10.1 | 7.21 |
| BCD-BTK-252 | 14.55 | >500 | >1000 | >100 |
| BCD-BTK-258 | 1.17 | 18.6 | 33.32 | 2.92 |
| BCD-BTK-292 | 7.34 | >500 | 63.45 | 77.85 |

These compounds are effective inhibitors of the kinase activity of Btk. Several compounds described herein show a high selectivity to the kinases EGFR, ITK, and TEC similar in structure.

### Example 14. Antiproliferative Activity Against BTK-Sensitive Cell Lines in Vitro

Antiproliferative activity of BTK inhibitors was measured in cell-based bioassay on B-cells cultures: Mino (mantle cell lymphoma, ATCC® CRL-3000™), Z-138 (mantle cell lymphoma, ATCC® CRL-3001™) and DOHH2 (follicular lymphoma, Creative Bioarray CSC-C0219) using cell viability reagent Alamar Blue (Invitrogen, #DAL 1100). Cells were cultured in 10% FB S-supplemented (HyClone, #SH3008803/Gibco, #16140-071) RPMI-1640 (PanEco, #S330p) for at least 1 passage after thawing, washed with PBS and passaged in 96-well culture plates (Corning, #3599) with growth medium with 10% FBS (HyClone, #SH3008803/Gibco, #16140-071) and antibiotic (50 μg/ml of gentamicin (Biolot, #1.3.16))≈3*10⁴ cells in 50 μl of medium per well.

The compounds were dissolved in DMSO and diluted with the assay medium to final concentrations ranging from 0 to 100 rpm. 150 μl of each diluted compound were then added to each well (final concentration of DMSO was less than 1%) and incubated at 37° C. in an incubator under 5% of CO2 for 72 h. After incubation, 20 μl of Alamar Blue reagent (Invitrogen, #DAL1100) were added to each well. The plates were shaked on an orbital shaker (Biosan, Latvia) and then incubated for 14-16 hours at 37° C. in the incubator.

The number of living cells were estimated, measuring the fluorescence signal at the excitation wavelength (REx) of 540 nm and the emission wavelength (λEm) of 590 nm on a microplate reader (Tecan Infinite M200Pro, Switzerland).

For each compound, IC₅₀ was calculated using Magellan 7.2 software (Tecan, Switzerland) approximating experimental points by four-parameter logistic model with the optimization by Levenberg-Marquardt. The results are presented in the tables 10 and 11.

The CC₅₀ values were determined in the test for General cytotoxicity on HepG2 cells (hepatocellular carcinoma, ATCC® HB-8065™). 2*10⁴ cells (in 50 μl) per well were seeded in 96-well plates (Corning, #3599) in DMEM medium (PanEco, #S420p), after 24 h of incubation 150 μl of candidate compounds were added to each well in the range of final concentrations from 200 μM to 4 μM and the plate was incubated in a total volume of 200 μl for 72 hours. Viability of the cells was assessed using Alamar Blue dye (Invitrogen, #DAL 1100). CC₅₀ was determined similarly (table 10).

The relationship between toxic (CC₅₀) and a therapeutic (IC₅₀) effects of the dose is the therapeutic index, which can be expressed as the ratio between CC₅₀ (HepG2) (general cytotoxicity of the candidate) and IC₅₀ (Mino) (antiproliferative activity on the target cells):

$$Terapeutic index = \frac{{CC}_{50} ( HepG 2 )}{{IC}_{50} ( Mino )}$$

TABLE 10
The results of the assessment of the specific activity of the
compounds in the cell-based antiproliferative test using the cell
line panel (Mino. Z-138. DOHH2) and the results of the assessment
of the general toxicity using HepG2 cell line are presented as
average values of activity obtained in several tests.

| | | | | | CC50 |
|---|---|---|---|---|---|
| | IC50 | IC50 | IC50 | CC50 | (HepG2)/ |
| | Mino, | DOHH2, | Z-138, | HepG2, | IC50 |
| Candidate No. | mkM | mkM | mkM | mkM | (Mino) |
| BCD-BTK-9 | 23.53 | — | 44.05 | >100 | >4.25 |
| BCD-BTK-13 | 37.09 | — | >50 | >100 | >2.70 |
| BCD-BTK-30 | 15.62 | — | 22.7 | 61.49 | 3.94 |
| BCD-BTK-56 | 32.51 | — | 30.51 | >50 | >1.54 |
| BCD-BTK-74 | 21.9 | — | 17.62 | 100 | 4.57 |
| BCD-BTK-76 | 15.96 | — | 19.63 | >100 | >6.27 |
| BCD-BTK-104 | 20.16 | — | 32.62 | >100 | >4.96 |
| BCD-BTK-117 | 13.29 | — | 23.37 | >50 | >3.76 |
| BCD-BTK-118 | 13.71 | — | 7.84 | 66.151 | 4.83 |
| BCD-BTK-119 | 12.26 | — | 25.48 | >100 | >8.16 |
| BCD-BTK-122 | 35.57 | — | 36.45 | >100 | >2.81 |
| BCD-BTκ-124 | 5.22 | — | 17.23 | 36.67 | 7.02 |
| BCD-BTκ-128 | 19.1 | — | 28.82 | >100 | >5.24 |
| BCD-BTκ-129 | 29.23 | — | 27.73 | 63.83 | 2.18 |
| BCD-BTκ-131 | 5.42 | — | 13.01 | 28.6 | 5.28 |
| BCD-BTK-132 | 18.65 | — | >100 | 57.65 | 3.09 |
| BCD-BTκ-133 | 19.75 | — | 38.25 | 99.67 | 5.05 |
| BCD-BTK-136 | 12.54 | — | >50 | >100 | >7.97 |
| BCD-BTK-137 | 11.55 | — | 25.6 | 65.87 | 5.70 |
| BCD-BTK-140 | 16.85 | — | >50 | >100 | >5.93 |
| BCD-BTK-201 | 11.90 | 7.97 | 6.54 | >50 | >4.20 |
| BCD-BTK-202 | 7.79 | 8.87 | 12.64 | 31.99 | 4.10 |
| BCD-BTK-203 | 13.61 | 13.97 | 16.01 | 61.88 | 4.55 |
| BCD-BTK-204 | 21.04 | 23.50 | 20.94 | 62.04 | 2.95 |
| BCD-BTK-205 | 16.58 | 20.18 | 24.89 | 57.16 | 3.45 |
| BCD-BTK-208 | 21.50 | — | 32.22 | 111.97 | 5.21 |
| BCD-BTK-211 | 5.67 | 23.01 | 17.28 | 45.23 | 7.97 |
| BCD-BTK-212 | 3.50 | — | 14.81 | 28.05 | 8.01 |
| BCD-BTK-213 | 3.19 | 19.72 | 9.70 | >100 | >31.3 |
| BCD-BTK-214 | 1.28 | >100 | 89.37 | >100 | >77.94 |
| BCD-BTK-215 | 2.93 | >100 | 8.00 | >100 | >34.11 |
| BCD-BTK-216 | 2.14 | 9.52 | 12.12 | 40.15 | 18.77 |
| BCD-BTK-217 | 17.37 | 16.06 | 13.87 | 40.15 | 2.31 |
| BCD-BTK-218 | 1.10 | 3.55 | 8.58 | 26.21 | 23.92 |
| BCD-BTK-219 | 10.05 | — | ~50 | >100 | >9.95 |
| BCD-BTK-220 | 10.51 | 9.46 | 14.18 | >50 | >9.51 |
| BCD-BTK-221 | 10.46 | 3.05 | 4.98 | 5.32 | 0.51 |
| BCD-BTK-222 | 19.35 | 26.30 | 26.29 | 50.25 | 2.60 |
| BCD-BTK-224 | 11.03 | | >50 | 73.14 | 6.63 |
| BCD-BTK-226 | 5.97 | 8.05 | 7.24 | 19.04 | 3.19 |
| BCD-BTK-229 | 36.34 | 41.23 | 77.11 | >100 | >2.75 |
| BCD-BTK-230 | 9.81 | 14.93 | 14.41 | 57.60 | 5.87 |
| BCD-BTK-231 | 2.26 | — | 3.39 | 9.08 | 4.01 |
| BCD-BTK-234 | 9.60 | 8.87 | 16.84 | 36.56 | 3.81 |
| BCD-BTK-235 | 18.64 | >100 | >100 | >100 | >5.37 |
| BCD-BTK-232 | 15.26 | — | 10.75 | 31.07 | 2.04 |
| BCD-BTK-233 | 5.44 | 1.11 | 4.11 | 18.44 | 3.39 |
| BCD-BTK-236 | 8.93 | 3.79 | 11.10 | 27.07 | 3.03 |
| BCD-BTK-238 | 10.58 | 4.05 | 9.44 | 24.80 | 2.34 |
| BCD-BTK-239 | 14.21 | 8.18 | 15.97 | 60.22 | 4.24 |
| BCD-BTK-240 | 14.07 | 2.00 | 10.49 | >100 | >7.11 |
| BCD-BTK-242 | 17.33 | 8.38 | 16.49 | 52.53 | 3.03 |
| BCD-BTK-246 | 32.38 | — | 23.74 | >100 | >3.09 |
| BCD-BTK-237 | 6.25 | 3.25 | 6.99 | 28.35 | 4.54 |
| BCD-BTK-241 | 21.32 | 11.53 | 15.49 | 45.36 | 2.13 |
| BCD-BTK-247 | 5.81 | >100 | >50 | >100 | >17.21 |
| BCD-BTK-249 | 28.83 | — | >50 | ≈100 | >3.47 |
| BCD-BTK-250 | 2.38 | — | 3.26 | 12.21 | 5.13 |
| BCD-BTK-251 | 3.65 | 3.38 | 4.46 | >50 | >13.70 |
| BCD-BTK-252 | 4.46 | 19.60 | 14.89 | >100 | >22.43 |
| BCD-BTK-254 | 2.95 | — | 6.65 | 12.34 | 4.18 |
| BCD-BTK-258 | 17.31 | 15.09 | 35.80 | >50 | >2.89 |
| BCD-BTK-259 | 16.34 | 16.96 | 27.47 | 47.61 | 2.91 |
| BCD-BTK-260 | 49.87 | >100 | >100 | >100 | >2.00 |
| BCD-BTK-261 | 4.27 | — | 5.01 | 8.33 | 1.95 |
| BCD-BTK-263 | 20.64 | — | 35.24 | >50 | >2.42 |
| BCD-BTK-264 | 4.92 | — | 14.32 | 10.47 | 2.13 |
| BCD-BTK-265 | 7.91 | — | 16.57 | 16.54 | 2.09 |
| BCD-BTK-266 | 3.61 | 4.99 | 12.64 | 12.01 | 3.33 |
| BCD-BTK-268 | 10.32 | 26.63 | 78.29 | >100 | >9.69 |
| BCD-BTK-270 | 6.79 | — | 14.32 | 23.42 | 3.45 |
| BCD-BTK-272 | 6.83 | 5.23 | 14.93 | 17.55 | 2.57 |
| BCD-BTK-274 | 40.10 | - | >100 | 38.01 | 0.95 |
| BCD-BTK-277 | 17.95 | 20.58 | 30.05 | 41.87 | 2.33 |
| BCD-BTK-284 | 27.88 | — | 114.98 | >100 | >3.59 |
| BCD-BTK-285 | 10.09 | — | >34.6 | >100 | >9.91 |
| BCD-BTK-289 | 10.18 | — | 8.29 | 18.83 | 1.85 |
| BCD-BTK-281 | 5.63 | — | 4.67 | 15.82 | 2.81 |
| BCD-BTK-282 | 4.02 | — | 4.30 | 9.45 | 2.35 |
| BCD-BTK-292 | 3.35 | 20.66 | >50 | >100 | >29.81 |
| BCD-BTK-293 | 4.30 | >100 | >50 | >100 | >23.23 |
| BCD-BTK-295 | 2.00 | 1.94 | 1.19 | 32.53 | 16.23 |

TABLE 11
Results of the inhibition of BTK kinase
activity in vitro and cell tests results

| | | | | | CC₅₀ |
|---|---|---|---|---|---|
| | IC₅₀ | IC₅₀ | IC₅₀ | CC₅₀ | (HepG2)/ |
| | kinase, | Mino, | Z-138, | HepG2, | IC₅₀ |
| Candidate | nM | mkM | mkM | mkM | (Mino) |
| Ibrutinib | 1.73 | 9.18 | 15.124 | 54.89 | 5.98 |
| BCD-BTK-125 | 108.74 | 7.297 | 14.62 | 27.13 | 4.18 |
| BCD-BTK-134 | 1.16 | 11.586 | 21.18 | 63.99 | 5.80 |
| BCD-BTK-139 | 0.85 | 10.22 | 21.13 | 39.65 | 3.88 |
| BCD-BTK-123 | 1512.1 | 13.22 | 28.03 | 50.35 | 3.81 |

1. A compound of Formula I: 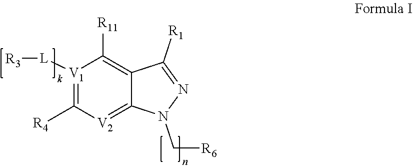
or pharmaceutically acceptable salt, solvate or stereoisomer thereof,
wherein:
V₁ is C or N,
V₂ is C(R₂) or N,
whereby if V₁ is C then V₂ is N,
if V₁ is C then V₂ is C(R₂), or
if V₁ is N then V₂ is C(R₂);
each n, k is independently 0, 1;
each R₂, R₁₁ is independently H, D, Hal, CN, NR′R″, C(O)NR′R″, C₁-C₆ alkoxy;
R₃ is H, D, hydroxy, C(O)C₁-C₆ alkyl, C(O)C₂-C₆ alkenyl, C(O)C₂-C₆ alkynyl, C₁-C₆ alkyl;
R₄ is H, Hal, CN, CONR′R″, hydroxy, C₁-C₆ alkyl, C₁-C₆ alkoxy;
L is CH₂, NH, O or chemical bond;
R₁ is selected from the group of the fragments, consisting of: 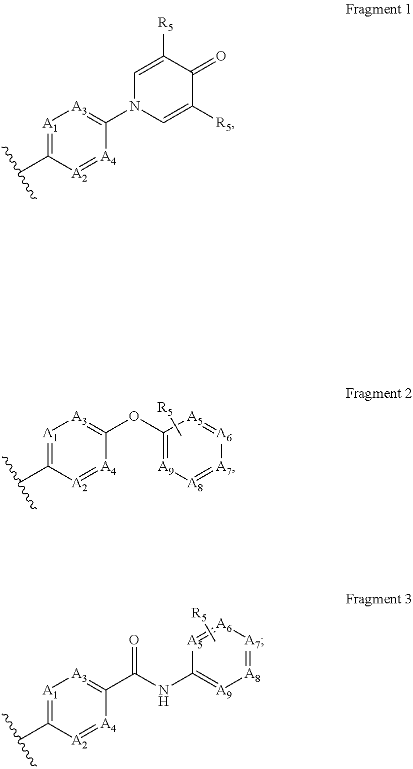
each A₁, A₂, A₃, A₄ is independently CH, N, CHal;
each A₅, A₆, A₇, A₈, A₉ is independently C, CH or N;
R₅ is H, CN, Hal, CONR′R″, C₁-C₆ alkyl, non-substituted or substituted by one or more halogens;
each R′ and R″ is independently selected from the group, comprising H, C₁-C₆ alkyl, C₁-C₆ cycloalkyl, aryl;
R₆ is selected from the group: 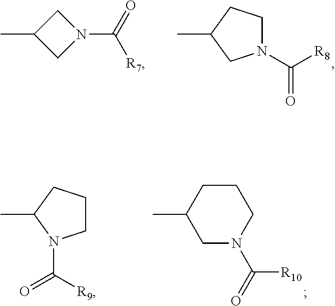
each R₇, R₈, R₉, R₁₀ is independently vinyl, methylacetylenyl;
Hal is Cl, Br, I, F.

2. The compound according to claim 1, wherein R₁ is selected from the group including: 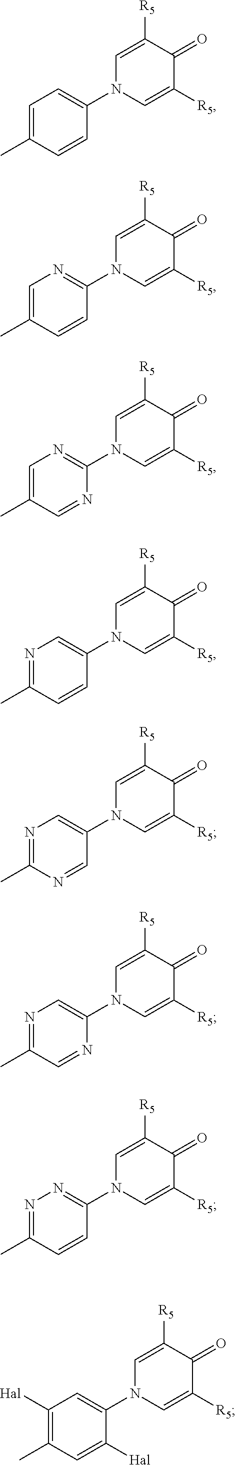 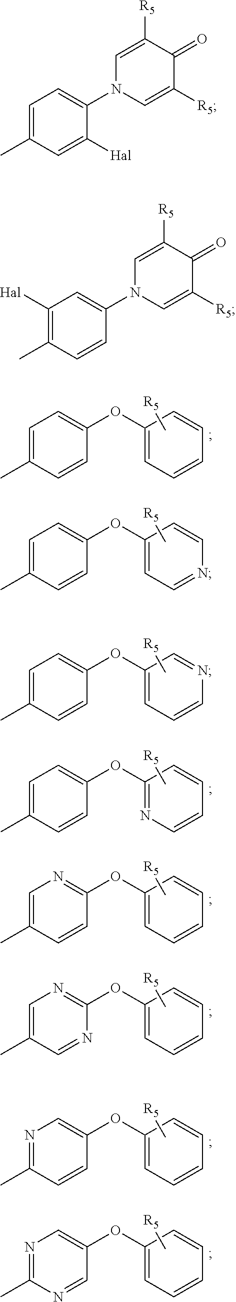 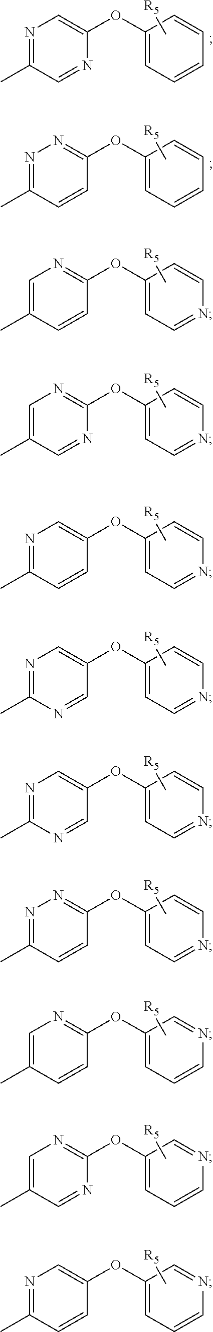 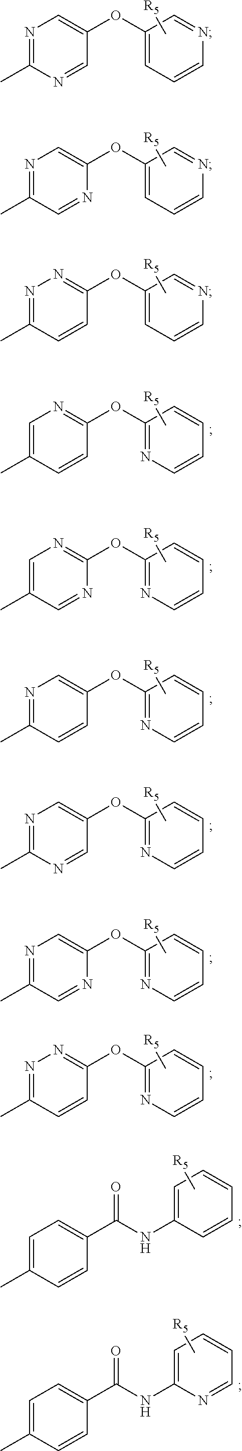 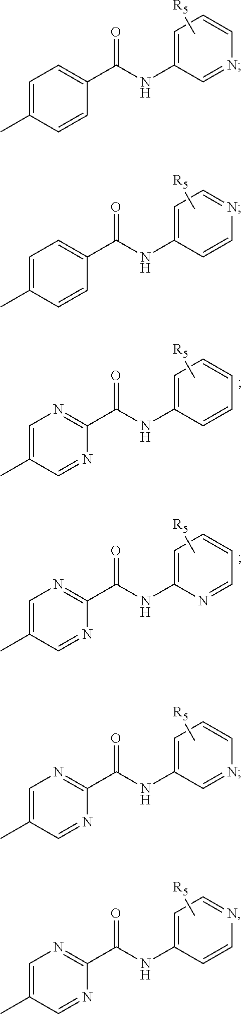
wherein R₅, Hal have the above meanings.

3. The compound according to claims 1-2, selected from the group including:
(R)-1-(1-acryloylpiperidin-3-yl)-4-amino-3-(4-(4-oxopyridin-1 (4H)-yl)phenyl)-1H-pyrazolo[4,3-c]pyridin-7-carbonitrile (BCD-BTK-4)
(R)-1-(1-acryloylpiperidin-3-yl)-4-amino-3-(4-(4-oxopyridin-1 (4H)-yl)phenyl)-1H-pyrazolo[4,3-c]pyridin-7-carboxamide (BCD-BTK-6)
(R)-1-(4-(1-(1-acryloylpiperidin-3-yl)-4-amino-7-chloro-1H-pyrazolo[4,3-c]pyridin-3-yl)phenyl)pyridin-4(1H)-one (BCD-BTK-9)
(R)-1-(4-(1-(1-acryloylpiperidin-3-yl)-4-chloro-5-hydroxy-1H-pyrazolo[3,4-b]pyridin-3-yl)phenyl)pyridin-4(1H)-one (BCD-BTK-13)
(R)-1-(1-acryloylpiperidin-3-yl)-5-hydroxy-3-(4-(4-oxopyridin-1(4H)-yl)phenyl)-1H-indazol-7-carboxamide (BCD-BTK-18)
(R)-1-(1-acryloylpiperidin-3-yl)-3-(4-(4-oxopyridin-1 (4H)-yl)phenyl)-1H-pyrazolo[4,3-c]pyridin-7-carboxamide (BCD-BTK-24)
(R)-1-(4-(1-(1-acryloylpiperidin-3-yl)-7-chloro-1H-pyrazolo[4,3-c]pyridin-3-yl) phenyl)pyridin-4(1H)-one (BCD-BTK-30)
(R)-1-(1-acryloylpiperidin-3-yl)-4-cyano-3-(4-(4-oxopyridin-1(4H)-yl)phenyl)-1H-indazol-7-carboxamide (BCD-BTK-35)
(R)-1-(1-acryloylpiperidin-3-yl)-4-amino-3-(2-(4-oxopyridin-1(4H)-yl)pyrimidin-5-yl)-1H-pyrazolo[4,3-c]pyridin-7-carbonitrile (BCD-BTK-36)
(R)-1-(1-acryloylpiperidin-3-yl)-4-amino-3-(4-oxo-4H-[1,2′-bipyridin]-5′-yl)-1H-pyrazolo[4,3-c]pyridin-7-carbonitrile (BCD-BTK-38)
(R)-1-(5-(1-(1-acryloylpiperidin-3-yl)-7-chloro-1H-pyrazolo[4,3-c]pyridin-3-yl) pyrimidin-2-yl)pyridin-4(1H)-one (BCD-BTK-54)
(R)-5′-(1-(1-acryloylpiperidin-3-yl)-7-chloro-1H-pyrazolo[4,3-c]pyridin-3-yl)-4H-[1,2′-bipyridin]-4-one (BCD-BTK-56)
(R)-1-(5-(1-(1-acryloylpiperidin-3-yl)-4-amino-7-chloro-1H-pyrazolo[4,3-c]pyridin-3-yl)pyrimidin-2-yl)pyridin-4(1H)-one (BCD-BTK-74)
(R)-5′-(1-(1-acryloylpiperidin-3-yl)-4-amino-7-chloro-1H-pyrazolo[4,3-c]pyridin-3-yl)-4H-[1,2′-bipyridin]-4-one (BCD-BTK-76)
(R)-1-(1-acryloylpiperidin-3-yl)-4-amino-3-(2-(4-oxopyridin-1(4H)-yl)pyrimidin-5-yl)-1H-pyrazolo[4,3-c]pyridine-7-carboxamide (BCD-BTK-86)
(R)-1-(1-acryloylpiperidin-3-yl)-4-amino-3-(4-oxo-4H-[1,2′-bipyridin]-5′-yl)-1H-pyrazolo[4,3-c]pyridin-7-carboxamide (BCD-BTK-88)
(R)-1-(1-acryloylpiperidin-3-yl)-3-(2-(4-oxopyridin-1(4H)-yl)pyrimidin-5-yl)-1H-pyrazolo[4,3-c]pyridin-7-carboxamide (BCD-BTK-98)
(R)-1-(1-acryloylpiperidin-3-yl)-3-(4oxo-4H[1,2′-bipyridin]-5′-yl)-1H-pyrazolo[4,3-c]pyridin-7-carboxamide (BCD-BTK-100)
(R)-1-(1-acryloylpiperidin-3-yl)-3-(4-(pyridin-4-yloxy)phenyl)-1H-pyrazolo[4,3-c]pyridin-7-carbonitrile (BCD-BTK-104)
(R)-1-(1-acryloylpiperidin-3-yl)-3-(2-(4-oxopyridin-1 (4H)-yl)pyrimidin-5-yl)-1H-pyrazolo[4,3-c]pyridin-7-carbonitrile (BCD-BTK-105)
(R)-1-(1-acryloylpiperidin-3-yl)-3-(4-oxo-4H[1,2′-bipyridin]-5′-yl)-1H-pyrazolo[4,3-c]pyridin-7-carbonitrile (BCD-BTK-107)
(R)—N-(1-(1-acryloylpiperidin-3-yl)-6-methyl-3-(4-(4-oxopyridin-1(4H)-yl) phenyl)-1H-pyrazolo[3,4-b]pyridin5-yl) acrylamide (BCD-BTK-117)
(R)-1-(1-acryloylpiperidin-3-yl)-7-cyano-3-(4-(4-oxopyridin-1(4H)-yl)phenyl)-1H-indazol-5-yl acrylate (BCD-BTK-118)
(R)-1-(4-(1-(1-acryloylpiperidin-3-yl)-5-amino-6-methyl-1H-pyrazolo[3,4-b]pyridin-3-yl)phenyl)pyridin-4(1H)-one (BCD-BTK-119)
(R)-1-(1-acryloylpiperidin-3-yl)-5-hydroxy-3-(4-oxo-4H-[1,2′-bipyridin]-5′-yl)-1H-indazol-7-carboxamide (BCD-BTK-120)
(R)-1-(1-acryloylpiperidin-3-yl)-5-hydroxy-3-(2-(4-oxopyridin-1(4H)-yl) pyrimidin-5-yl)-1H-indazol-7-carboxamide (BCD-BTK-121)
(R)-1-(1-acryloylpiperidin-3-yl)-5-hydroxy-3-(4-(4-oxopyridin-1(4H)-yl)phenyl)-1H-indazol-7-carbonitrile (BCD-BTK-122)
(R)-1-(1-acryloylpiperidin-3-yl)-3-(4-(pyridin-4-yloxy)phenyl)-1H-pyrazolo[4,3-c]pyridin-7-carbonitrile (BCD-BTK-123)
(R)-1-(1-acryloylpiperidin-3-yl)-4-chloro-3-(4-(pyridin-4-yloxy)phenyl)-1H-pyrazolo[3,4-b]pyridine (BCD-BTK-124)
(R)-1-(1-acryloylpiperidin-3-yl)-7-chloro-3-(4-(pyridin-4-yloxy)phenyl)-1H-pyrazolo[4,3-c]pyridine (BCD-BTK-125)
(R)-1-(1-acryloylpiperidin-3-yl)-5-hydroxy-3-(6-(4-oxopyridin-1(4H)-yl)pyridin-3-yl)-1H-indazol-7-carbonitrile (BCD-BTK-127)
(R)-1-(1-acryloylpiperidin-3-yl)-5-hydroxy-4-chloro-3-(4-(pyridin-4-yloxy) phenyl)-1H-pyrazolo[3,4-b]pyridine (BCD-BTK-129)
(R)-1-(1-acryloylpiperidin-3-yl)-5-hydroxy-3-(2-(4-oxopyridin-1(4H)-yl) pyrimidin-5-yl)-1H-indazol-7-carbonitrile (BCD-BTK-130)
(R)-1-(4-(1-(1-acryloylpiperidin-3-yl)-4-chloro-1H-pyrazolo[3,4-b]pyridin-3-yl) phenyl)pyridin-4(1H)-one (BCD-BTK-131)
(R)-1-(1-acryloylpiperidin-3-yl)-5-hydroxy-3-(4-pyridin-4-yloxy)phenyl)-1H-indazol-7-carboxamide (BCD-BTK-133)
(R)-1-(1-acryloylpiperidin-3-yl)-4-amino-3-(4-(pyridin-4-yloxy)phenyl)-1H-pyrazolo[4,3-c]pyridin-7-carbonitrile (BCD-BTK-134)
(R)-1-(1-acryloylpiperidin-3-yl)-4-amino-3-(4-(pyridin-4-yloxy)phenyl)-1H-pyrazolo[4,3-c]pyridin-7-carboxamide (BCD-BTK-135)
(R)-1-(1-acryloylpiperidin-3-yl)-4-chloro-3-(4-(4-oxopyridin-1 (4H)phenyl)-1H-pyrazolo[3,4-b]pyridin-5-yl acrylate (BCD-BTK-136)
(R)-1-(1-acryloylpiperidin-3-yl)-5-hydroxy-3-(4-pyridin-4-yloxy)phenyl)-1H-indazol-7-carbonitrile (BCD-BTK-137)
(R)-1-(1-acryloylpiperidin-3-yl)-4-cyano-3-(4-(pyridin-4-yloxy)phenyl)-1H-indazol-7-carboxamide (BCD-BTK-138)
(R)-1-(1-acryloylpiperidin-3-yl)-7-chloro-3-(4-(pyridin-4-yloxy)phenyl)-1H-pyrazolo[4,3-c]pyridin-4-amine (BCD-BTK-139)
(R)-1-(1-acryloylpiperidin-3-yl)-3-(4-(pyridin-4-yloxy)phenyl)-1H-pyrazolo[4,3-c]pyridin-7-carboxamide (BCD-BTK-140)
(R)-4-(1-(1-(1-acryloylpiperidin-3-yl)-7-chloro-1H-pyrazolo[4,3-c]pyridin-3-yl)-N-(pyridin-2-yl)benzamide (BCD-BTK-201)
(R)-4-(4-(1-(1-acryloylpiperidin-3-yl)-7-chloro-1H-pyrazolo[4,3-c]pyridin-3-yl) phenoxy)nicotinonitrile (BCD-BTK-202)
(R)-4-(4-(1-(1-acryloylpiperidin-3-yl)-7-chloro-1H-pyrazolo[4,3-c]pyridin-3-yl) phenoxy)nicotinamide (BCD-BTK-203)
(R)-1-(4-(1-(1-acryloylpiperidin-3-yl)-7-chloro-1H-pyrazolo[4,3-c]pyridin-3-yl)-3-fluorophenyl)pyridin-4(1H)-one (BCD-BTK-204)
(R)-1-(4-(1-(1-acryloylpiperidin-3-yl)-7-chloro-1H-pyrazolo[4,3-c]pyridin-3-yl)-2-fluorophenyl)pyridin-4(1H)-one (BCD-BTK-205)
(R)-1-(5-(1-(1-acryloylpiperidin-3-yl)-7-chloro-1H-pyrazolo[4,3-c]pyridin-3-yl) pyrimidin-2-yl)-4-oxo-1,4-dihydropyridine-3-carbonitrile (BCD-BTK-206)
(R)-1-(5-(1-(1-acryloylpiperidin-3-yl)-7-chloro-1H-pyrazolo[4,3-c]pyridin-3-yl) pyrimidin-2-yl)-4-oxo-1,4-dihydropyridine-3-carboxamide (BCD-BTK-207)
(R)-1-(4-(1-(1-acryloylpiperidin-3-yl)-7-chloro-1H-pyrazolo[4,3-c]pyridin-3-yl) phenyl)-4-oxo-1,4-dihydropyridine-3-carbonitrile (BCD-BTK-208)
(R)-4-(1-(1-acryloylpiperidin-3-yl)-7-chloro-1H-pyrazolo[4,3-c]pyridin-3-yl)-N-(4-(trifluoromethyl)pyridin-2-yl)benzamide (BCD-BTK-210)
(R)-1-(3-(7-fluoro-3-(4-(pyridin-4-yloxy)phenyl)-1H-pyrazolo[4,3-c]pyridin-1-yl) piperidin-1-yl)prop-2-en-1-one (BCD-BTK-211)
(R)-4-(1-(1-acryloylpiperidin-3-yl)-7-chloro-1H-pyrazolo[4,3-c]pyridin-3-yl)-N-(5-fluoropyridin-2-yl)benzamide (BCD-BTK-212)
(R)-1-(1-acryloylpiperidin-3-yl)-3-(4-(3-cyanopyridin-4-yloxy)phenyl)-1H-pyrazolo[4,3-c]pyridine-7-carbonitrile (BCD-BTK-213)
(R)-4-(1-(1-acryloylpiperidin-3-yl)-7-fluoro-1H-pyrazolo[4,3-c]pyridin-3-yl)-N-(4-(1,1-difluoropropyl)pyridin-2-yl)benzamide (BCD-BTK-214)
(R)-4-(1-(1-acryloylpiperidin-3-yl)-7-chloro-1H-pyrazolo[4,3-c]pyridin-3-yl)-N-(4-(1,1-difluoropropyl)pyridin-2-yl)benzamide (BCD-BTK-215)
(R)-1-(1-acryloylpiperidin-3-yl)-3-(4-(2-cyanopyridin-3-yloxy)phenyl)-1H-pyrazolo[4,3-c]pyridine-7-carbonitrile (BCD-BTK-216)
(R)-4-(4-(1-(1-acryloylpiperidin-3-yl)-7-fluoro-1H-pyrazolo[4,3-c]pyridin-3-yl) phenoxy)nicotinonitrile (BCD-BTK-217)
(R)-3-(4-(1-(1-acryloylpiperidin-3-yl)-7-chloro-1H-pyrazolo[4,3-c]pyridin-3-yl) phenoxy)picolinonitrile (BCD-BTK-218)
(R)-4-(1-(1-acryloylpiperidin-3-yl)-7-cyano-1H-pyrazolo[4,3-c]pyridin-3-yl)-N-(4-(1,1-difluoropropyl)pyridin-2-yl)benzamide (BCD-BTK-219) 3-(4-(7-chloro-1-[(3R)-1-(prop-2-enoyl)piperidin-3-yl]-1H-pyrazolo[4,3-c]pyridin-3-yl)phenoxy)pyridine-2-carboxamide (BCD-BTK-220)
(R)-4-(1-(1-acryloylpiperidin-3-yl)-7-cyano-1H-pyrazolo[4,3-c]pyridin-3-yl)-N-(4-(trifluoromethyl)pyridin-2-yl)benzamide (BCD-BTK-221)
5-(4-(7-chloro-1-[(3R)-1-(prop-2-enoyl)piperidin-3-yl]-1H-pyrazolo[4,3-c]pyridin-3-yl)phenoxy)pyridine-3-carboxamide (BCD-BTK-222)
4-(7-cyano-1-[(3R)-1-(prop-2-enoyl)piperidin-3-yl]-1H-pyrazolo[4,3-c]pyridin-3-yl)-N-(6-fluoropyridin-2-yl)benzamide (BCD-BTK-223)
4-(7-cyano-1-[(3R)-1-(prop-2-enoyl)piperidin-3-yl]-1H-pyrazolo[4,3-c]pyridin-3-yl)-N-[4-(1,1-difluoroethyl)pyridin-2-yl]benzamide (BCD-BTK-224)
(R)-4-(1-(1-acryloylpiperidin-3-yl)-7-chloro-1H-pyrazolo[4,3-c]pyridin-3-yl)-N-(6-fluoropyridin-2-yl)benzamide (BCD-BTK-225)
(R)-4-(1-(1-acryloylpiperidin-3-yl)-7-chloro-1H-pyrazolo[4,3-c]pyridin-3-yl)-N-(3-fluoropyridin-2-yl)benzamide (BCD-BTK-226)
(R)-4-(1-(1-acryloylpiperidin-3-yl)-7-cyano-1H-pyrazolo[4,3-c]pyridin-3-yl)-N-(5-fluoropyridin-2-yl)benzamide (BCD-BTK-227)
(R)-4-(1-(1-acryloylpiperidin-3-yl)-7-cyano-1H-pyrazolo[4,3-c]pyridin-3-yl)-N-(pyridin-2-yl)benzamide (BCD-BTK-228)
(R)-5-(1-(1-acryloylpiperidin-3-yl)-7-chloro-1H-pyrazolo[4,3-c]pyridin-3-yl)-N-(pyridin-2-yl)pyrimidine-2-carboxamide (BCD-BTK-229)
(R)-3-(4-(1-(1-acryloylpiperidin-3-yl)-7-fluoro-1H-pyrazolo[4,3-c]pyridin-3-yl) phenoxy)picolinonitrile (BCD-BTK-230)
(R)-4-(1-(1-acryloylpiperidin-3-yl)-4-amino-7-chloro-1H-pyrazolo[4,3-c]pyridin-3-yl)-N-(4-(1,1-difluoropropyl)pyridin-2-yl)benzamide (BCD-BTK-231)
(R)-4-(4-(1-(1-acryloylpiperidin-3-yl)-4-amino-7-chloro-1H-pyrazolo[4,3-c]pyridin-3-yl)phenoxy)nicotinonitrile (BCD-BTK-232)
(R)-4-(1-(1-acryloylpiperidin-3-yl)-4-amino-7-chloro-1H-pyrazolo[4,3-c]pyridin-3-yl)-N-(3-fluoropyridin-2-yl)benzamide (BCD-BTK-233)
(R)-4-(1-(1-acryloylpiperidin-3-yl)-4-amino-7-cyano-1H-pyrazolo[4,3-c]pyridin-3-yl)-N-(pyridine-2-yl)benzamide (BCD-BTK-234) 4-(7-cyano-1-[(3R)-1-(prop-2-enoyl)piperidin-3-yl]-1H-pyrazolo[4,3-c]pyridin-3-yl)-N-(3-fluoropyridin-2-yl)benzamide (BCD-BTK-235)
3-(4-(4-amino-7-chloro-1-[(3R)-1-(prop-2-enoyl)piperidin-3-yl]-1H-pyrazolo[4,3-c]pyridin-3-yl)phenoxy)pyridine-2-carbonitrile (BCD-BTK-236)
4-(4-amino-7-chloro-1-[(3R)-1-(prop-2-enoyl)piperidin-3-yl]-1H-pyrazolo[4,3-c]pyridin-3-yl)-N-(5-fluoropyridin-2-yl)benzamide (BCD-BTK-237)
4-(4-amino-7-chloro-1-[(3R)-1-(prop-2-enoyl)piperidin-3-yl]-1H-pyrazolo[4,3-c]pyridin-3-yl)-N-(pyridin-2-yl)benzamide (BCD-BTK-238)
(R)-1-(1-acryloylpiperidin-3-yl)-4-amine-7-methoxy-3-(4-(pyridin-4-yloxy) phenyl)-1H-pyrazolo[4,3-c]pyridine (BCD-BTK-239)
(R)-4-(1-(1-acryloylpiperidin-3-yl)-4-amino-7-methoxy-1H-pyrazolo[4,3-c]pyridin-3-yl)-N-(pyridin-2-yl)benzamide (BCD-BTK-240)
(R)-1-(1-acryloylpiperidin-3-yl)-4-amine-3-(4-(pyridin-4-yloxy)phenyl)-1H-pyrazolo[4,3-c]pyridine (BCD-BTK-241)
(R)-4-(1-(1-acryloylpiperidin-3-yl)-4-amino-1H-pyrazolo[4,3-c]pyridin-3-yl)-N-(pyridin-2-yl)benzamide (BCD-BTK-242)
(R)-4-(1-(1-acryloylpiperidin-3-yl)-7-fluoro-1H-pyrazolo[4,3-c]pyridin-3-yl)-N-(pyridin-2-yl)benzamide (BCD-BTK-243)
(R)-4-(1-(1-acryloylpiperidin-3-yl)-7-fluoro-1H-pyrazolo[4,3-c]pyridin-3-yl)-N-(5-fluoropyridin-2-yl)benzamide (BCD-BTK-244)
(R)-4-(1-(1-acryloylpiperidin-3-yl)-7-fluoro-1H-pyrazolo[4,3-c]pyridin-3-yl)-N-(4-(trifluoromethyl)pyridin-2-yl)benzamide (BCD-BTK-245)
(R)-4-(4-(1-(1-acryloylpiperidin-3-yl)-7-fluoro-1H-pyrazolo[4,3-c]pyridin-3-yl) phenoxy)nicotinamide (BCD-BTK-246)
(R)-4-(1-(1-acryloylpiperidin-3-yl)-7-fluoro-1H-pyrazolo[4,3-c]pyridin-3-yl)-N-(4-(1,1-difluorobutyl)pyridin-2-yl)benzamide (BCD-BTK-247)
(R)-4-(1-(1-acryloylpiperidin-3-yl)-7-fluoro-1H-pyrazolo[4,3-c]pyridin-3-yl)-N-(6-fluoropyridin-2-yl)benzamide (BCD-BTK-248)
(R)-4-(1-(1-acryloylpiperidin-3-yl)-7-fluoro-1H-pyrazolo[4,3-c]pyridin-3-yl)-N-(3-fluoropyridin-2-yl)benzamide (BCD-BTK-249)
(R)-4-(1-(1-acryloylpiperidin-3-yl)-4-amino-7-chloro-H-pyrazolo[4,3-c]pyridin-3-yl)-N-(6-fluoropyridin-2-yl)benzamide (BCD-BTK-250)
(R)-4-(1-(1-acryloylpiperidin-3-yl)-4-amino-7-chloro-H-pyrazolo[4,3-c]pyridin-3-yl)-N-(4-(trifluoromethyl)pyridin-2-yl)benzamide (BCD-BTK-251)
(R)-4-(1-(1-acryloylpiperidin-3-yl)-7-chloro-1H-pyrazolo[4,3-c]pyridin-3-yl)-N-(4-(1,1-difluorobutyl)pyridin-2-yl)benzamide (BCD-BTK-252)
(R)-5-(1-(1-acryloylpiperidin-3-yl)-7-chloro-1H-pyrazolo[4,3-c]pyridin-3-yl)-N-(5-fluoropyridin-2-yl)pyrimidine-2-carboxamide (BCD-BTK-253)
(R)-4-(1-(1-acryloylpiperidin-3-yl)-4-amino-7-chloro-1H-pyrazolo[4,3-c]pyridin-3-yl)-N-(4-(1,1-difluorobutyl)pyridin-2-yl)benzamide (BCD-BTK-254)
(R)-3-(4-(1-(1-acryloylpiperidin-3-yl)-7-fluoro-1H-pyrazolo[4,3-c]pyridin-3-yl) phenoxy)picolinamide (BCD-BTK-255)
4-(4-amino-7-cyano-1-[(3R)-1-(prop-2-enoyl)piperidin-3-yl]-1H-pyrazolo[4,3-c]pyridin-3-yl)-N-(3-fluoropyridin-2-yl)benzamide (BCD-BTK-258)
(S)-1-(2-((7-chloro-3-(4-(pyridin-4-yloxy)phenyl)-1H-pyrazolo[4,3-c]pyridin-1-yl) methyl)pyrrolidin-1-yl)but-2-yn-1-one (BCD-BTK-259)
(S)-4-(1-((1-acryloylpyrrolidin-2-yl)methyl)-7-chloro-1H-pyrazolo[4,3-c]pyridin-3-yl)-N-phenylbenzamide (BCD-BTK-260)
1-(3-((7-chloro-3-(4-(pyridin-4-yloxy)phenyl)-1H-pyrazolo[4,3-c]pyridin-1-yl) methyl)azetidin-1-yl)but-2-yn-1-one (BCD-BTK-261)
(R)-4-(1-(1-(but-2-ynoyl)pyrrolidin-3-yl)-7-chloro-1H-pyrazolo[4,3-c]pyridin-3-yl)-N-(pyridin-2-yl)benzamide (BCD-BTK-262)
(S)-1-(2-((7-chloro-3-(4-(pyridin-4-yloxy)phenyl)-1H-pyrazolo[4,3-c]pyridin-1-yl) methyl)pyrrolidin-1-yl)prop-2-en-1-one (BCD-BTK-263)
1-(3-((7-chloro-3-(4-(pyridin-4-yloxy)phenyl)-1H-pyrazolo[4,3-c]pyridin-1-yl) methyl)azetidin-1-yl)prop-2-en-1-one (BCD-BTK-264)
(R)-1-(3-(7-chloro-3-(4-(pyridin-4-yloxy)phenyl)-1H-pyrazolo[4,3-c]pyridin-1-yl) pyrrolidin-1-yl)but-2-yn-1-one (BCD-BTK-265)
(S)-1-(3-(7-chloro-3-(4-(pyridin-4-yloxy)phenyl)-1H-pyrazolo[4,3-c]pyridin-1-yl) pyrrolidin-1-yl)prop-2-en-1-one (BCD-BTK-266)
4-(1-((1-(but-2-ynoyl)azetidin-3-yl)methyl)-7-chloro-1H-pyrazolo[4,3-c]pyridin-3-yl)-N-(pyridin-2-yl)benzamide (BCD-BTK-267)
(R)-4-(1-(1-acryloylpiperidin-3-yl)-7-cyano-1H-pyrazolo[4,3-c]pyridin-3-yl)-N-(4-(1,1-difluorobutyl)pyridin-2-yl)benzamide (BCD-BTK-268)
(R)-4-(1-((1-(but-2-ynoyl)pyrrolidin-2-yl)methyl)-7-chloro-1H-pyrazolo[4,3-c]pyridin-3-yl)-N-(pyridin-2-yl)benzamide (BCD-BTK-269)
(S)-1-(3-(7-chloro-3-(4-(pyridin-4-yloxy)phenyl)-1H-pyrazolo[4,3-c]pyridin-1-yl) pyrrolidin-1-yl)but-2-yn-1-one (BCD-BTK-270)
(S)-4-(1-(1-(but-2-ynoyl)pyrrolidin-3-yl)-7-chloro-1H-pyrazolo[4,3-c]pyridin-3-yl)-N-(pyridin-2-yl)benzamide (BCD-BTK-271)
(R)-1-(3-(7-chloro-3-(4-(pyridin-4-yloxy)phenyl)-1H-pyrazolo[4,3-c]pyridin-1-yl) pyrrolidin-1-yl)prop-2-en-1-one (BCD-BTK-272)
(S)-4-(1-(1-acryloylpyrrolidin-3-yl)-7-chloro-1H-pyrazolo[4,3-c]pyridin-3-yl)-N-(pyridin-2-yl)benzamide (BCD-BTK-273)
(R)-5-(4-(1-(1-acryloylpiperidin-3-yl)-7-fluoro-1H-pyrazolo[4,3-c]pyridin-3-yl)phenoxy)nicotinamide (BCD-BTK-274)
(R)-4-(1-(1-acryloylpyrrolidin-3-yl)-7-chloro-1H-pyrazolo[4,3-c]pyridin-3-yl)-N-(pyridin-2-yl)benzamide (BCD-BTK-275)
4-(7-chloro-1-([1-(prop-2-enoyl)azetidin-3-yl]methyl)-1H-pyrazolo[4,3-c]pyridin-3-yl)-N-(pyridin-2-yl)benzamide (BCD-BTK-276)
(R)-5-(1-(1-acryloylpiperidin-3-yl)-7-chloro-1H-pyrazolo[4,3-c]pyridin-3-yl)-N-(6-fluoropyridin-2-yl)pyrimidine-2-carboxamide (BCD-BTK-277)
(R)-4-(1-((1-(but-2-ynoyl)piperidin-3-yl)methyl)-7-chloro-1H-pyrazolo[4,3-c]pyridin-3-yl)-N-(pyridin-2-yl)benzamide (BCD-BTK-278)
(R)-4-(1-((1-acryloylpiperidin-3-yl)methyl)-7-chloro-1H-pyrazolo[4,3-c]pyridin-3-yl)-N-(pyridin-2-yl)benzamide (BCD-BTK-279)
4-(1-(1-(but-2-ynoyl)azetidin-3-yl)-7-chloro-1H-pyrazolo[4,3-c]pyridin-3-yl)-N-(pyridin-2-yl)benzamide (BCD-BTK-280)
1-(3-(7-chloro-3-(4-(pyridin-4-yloxy)phenyl)-1H-pyrazolo[4,3-c]pyridin-1l-yl) azetidin-1-yl)but-2-yn-1-one (BCD-BTK-281)
1-(3-(7-chloro-3-(4-(pyridin-4-yloxy)phenyl)-1H-pyrazolo[4,3-c]pyridin-1-yl) azetidin-1-yl)prop-2-en-1-one (BCD-BTK-282)
(S)-4-(1-((1-(but-2-ynoyl)piperidin-3-yl)methyl)-7-chloro-1H-pyrazolo[4,3-c]pyridin-3-yl)-N-(pyridin-2-yl)benzamide (BCD-BTK-283)
(R)-4-(1-((1-acryloylpyrrolidin-3-yl)methyl)-7-chloro-1H-pyrazolo[4,3-c]pyridin-3-yl)-N-(pyridin-2-yl)benzamide (BCD-BTK-284)
(S)-4-(1-((1-acryloylpyrrolidin-3-yl)methyl)-7-chloro-1H-pyrazolo[4,3-c]pyridin-3-yl)-N-(pyridin-2-yl)benzamide (BCD-BTK-285)
(R)-4-(1-((1-(but-2-ynoyl)pyrrolidin-3-yl)methyl)-7-chloro-1H-pyrazolo[4,3-c]pyridin-3-yl)-N-(pyridin-2-yl)benzamide (BCD-BTK-286)
(S)-4-(1-((1-(but-2-ynoyl)pyrrolidin-3-yl)methyl)-7-chloro-1H-pyrazolo[4,3-c]pyridin-3-yl)-N-(pyridin-2-yl)benzamide (BCD-BTK-287)
(R)-5-(1-(1-acryloylpiperidin-3-yl)-7-chloro-1H-pyrazolo[4,3-c]pyridin-3-yl)-N-(3-fluoropyridin-2-yl)pyrimidine-2-carboxamide (BCD-BTK-288)
(R)-4-(1-(1-(but-2-ynoyl)piperidin-3-yl)-7-chloro-H-pyrazolo[4,3-c]pyridin-3-yl)-N-(pyridin-2-yl)benzamide (BCD-BTK-289)
(S)-4-(1-((1-acryloylpiperidin-3-yl)methyl)-7-chloro-H-pyrazolo[4,3-c]pyridin-3-yl)-N-(pyridin-2-yl)benzamide (BCD-BTK-290)
(R)-5-(1-(1-acryloylpiperidin-3-yl)-7-chloro-1H-pyrazolo[4,3-c]pyridin-3-yl)-N-(4-(trifluoromethyl)pyridin-2-yl)pyrimidine-2-carboxamide (BCD-BTK-291)
(R)-5-(1-(1-acryloylpiperidin-3-yl)-7-chloro-1H-pyrazolo[4,3-c]pyridin-3-yl)-N-(4-(1,1-difluorobutyl)pyridin-2-yl)pyrimidine-2-carboxamide (BCD-BTK-292)
(R)-5-(1-(1-acryloylpiperidin-3-yl)-7-chloro-1H-pyrazolo[4,3-c]pyridin-3-yl)-N-(4-(1,1-difluoropropyl)pyridin-2-yl)pyrimidine-2-carboxamide (BCD-BTK-293)
(R)-4-(4-amino-1-(1-(but-2-ynoyl)piperidin-3-yl)-7-chloro-1H-pyrazolo[4,3-c]pyridin-3-yl)-N-(pyridin-2-yl)benzamide (BCD-BTK-295)

4. A method for inhibiting biological activity of Bruton's tyrosine kinase (Btk) in a subject, comprising contacting the tyrosine kinase with the compound according to any claims 1 to 3.

5. A pharmaceutical composition for the prevention or treatment of a disease or disorder mediated by Bruton's tyrosine kinase (Btk), comprising a therapeutically effective amount of the compound according to any claims 1 to 3, or pharmaceutically acceptable salt thereof, and one or more pharmaceutically acceptable excipients.

6. The pharmaceutical composition according to claim 5, wherein the disease or disorder mediated by Bruton's tyrosine kinase (Btk) is tumors of blood and lymphatic system, immune disorders, cancer, autoimmune and inflammatory diseases, or allergic disorders.

7. The pharmaceutical composition according to claim 6, wherein tumors of blood and lymphatic system are selected from the group comprising chronic lymphocytic leukemia, mantle cell lymphoma, follicular lymphoma, diffuse large B-cell lymphoma, Waldenstrom macroglobulinemia, B-cell prolymphocytic leukemia, central nervous system lymphoma, multiple myeloma; immune disorders are selected from the group comprising graft-versus-host disease, chronic graft-versus-host disease; cancer is pancreatic cancer; autoimmune diseases are selected from the group comprising rheumatoid arthritis, systemic lupus erythematosus; inflammatory disease is asthma; allergic disorder is atopic dermatitis.

8. A method for treating a disease or disorder mediated by Bruton's tyrosine kinase (Btk) comprising administering a therapeutically effective amount of the compound according to any claims 1 to 3, or pharmaceutically acceptable salt thereof, or the pharmaceutical composition according to claim 5 in a subject in need thereof.

9. The method for treating according to claim 8, wherein the disease or disorder mediated by Bruton's tyrosine kinase (Btk) is tumors of blood and lymphatic system, immune disorders, cancer, autoimmune and inflammatory diseases, or allergic disorders.

10. The method for treating according to claim 9, wherein tumors of blood and lymphatic system are selected from the group comprising chronic lymphocytic leukemia, mantle cell lymphoma, follicular lymphoma, diffuse large B-cell lymphoma, Waldenstrom macroglobulinemia, B-cell prolymphocytic leukemia, central nervous system lymphoma, multiple myeloma; immune disorders are selected from the group comprising graft-versus-host disease, chronic graft-versus-host disease; cancer is pancreatic cancer; autoimmune diseases are selected from the group comprising rheumatoid arthritis, systemic lupus erythematosus; inflammatory disease is asthma; allergic disorder is atopic dermatitis.

11. Use of the compound according to any claims 1 to 3, or pharmaceutically acceptable salt thereof, or a pharmaceutical composition according to claim 5 for the prevention or treatment of a disease or disorder mediated by Bruton's tyrosine kinase (Btk) in a subject in need thereof.

12. The use according to claim 11, wherein the disease or disorder mediated by Bruton's tyrosine kinase (Btk) is tumors of blood and lymphatic system, immune disorders, cancer, autoimmune and inflammatory diseases, or allergic disorders.

13. The use according to claim 12, wherein tumors of blood and lymphatic system are selected from the group comprising chronic lymphocytic leukemia, mantle cell lymphoma, follicular lymphoma, diffuse large B-cell lymphoma, Waldenstrom macroglobulinemia, B-cell prolymphocytic leukemia, central nervous system lymphoma, multiple myeloma; immune disorders are selected from the group comprising graft-versus-host disease, chronic graft-versus-host disease; cancer is pancreatic cancer; autoimmune diseases are selected from the group comprising rheumatoid arthritis, systemic lupus erythematosus; inflammatory disease is asthma; allergic disorder is atopic dermatitis.
